SEQUENCE LISTING (115 sequences)

SEQ ID NO: 1 - protein, 244 aa, Homo sapiens
  DOMAIN (1)...(17): signal sequence
  DOMAIN (18)...(41): non-homologous domain
  DOMAIN (42)...(107): collagen domain
  DOMAIN (108)...(244): globular domain
MLLLGAVLLLLALPGHDQETTTQGPGVLLPLPKGACTGWMAGIPGHPGHNGAPGRDGRDGTPGEKGEKGDPGLIGPKGDIGETGVPGAEGPRGFPGIQGRKGEPGEGAYVYRSAFSVGLETYVTIPNMPIRFTKIFYNQQNHYDGSTGKFHCNIPGLYYFAYHITVYMKDVKVSLFKKDKAMLFTYDQYQENNVDQASGSVLLHLEVGDQVWLQVYGEGERNGLYADNDNDSTFTGFLLYHDTN

SEQ ID NO: 2 - protein, 226 aa, Homo sapiens
  apM1(19-244)
ETTTQGPGVLLPLPKGACTGWMAGIPGHPGHNGAPGRDGRDGTPGEKGEKGDPGLIGPKGDIGETGVPGAEGPRGFPGIQGRKGEPGEGAYVYRSAFSVGLETYVTIPNMPIRFTKIFYNQQNHYDGSTGKFHCNIPGLYYFAYHITVYMKDVKVSLFKKDKAMLFTYDQYQENNVDQASGSVLLHLEVGDQVWLQVYGEGERNGLYADNDNDSTFTGFLLYHDTN

SEQ ID NO: 3 - protein, 203 aa, Homo sapiens
  apM1(42-244)
GIPGHPGHNGAPGRDGRDGTPGEKGEKGDPGLIGPKGDIGETGVPGAEGPRGFPGIQGRKGEPGEGAYVYRSAFSVGLETYVTIPNMPIRFTKIFYNQQNHYDGSTGKFHCNIPGLYYFAYHITVYMKDVKVSLFKKDKAMLFTYDQYQENNVDQASGSVLLHLEVGDQVWLQVYGEGERNGLYADNDNDSTFTGFLLYHDTN

SEQ ID NO: 4 - protein, 157 aa, Homo sapiens
  apM1(88-244)
AEGPRGFPGIQGRKGEPGEGAYVYRSAFSVGLETYVTIPNMPIRFTKIFYNQQNHYDGSTGKFHCNIPGLYYFAYHITVYMKDVKVSLFKKDKAMLFTYDQYQENNVDQASGSVLLHLEVGDQVWLQVYGEGERNGLYADNDNDSTFTGFLLYHDTN

SEQ ID NO: 5 - protein, 144 aa, Homo sapiens
  apM1(101-244)
KGEPGEGAYVYRSAFSVGLETYVTIPNMPIRFTKIFYNQQNHYDGSTGKFHCNIPGLYYFAYHITVYMKDVKVSLFKKDKAMLFTYDQYQENNVDQASGSVLLHLEVGDQVWLQVYGEGERNGLYADNDNDSTFTGFLLYHDTN

SEQ ID NO: 6 - protein, 137 aa, Homo sapiens
  apM1(108-244)
AYVYRSAFSVGLETYVTIPNMPIRFTKIFYNQQNHYDGSTGKFHCNIPGLYYFAYHITVYMKDVKVSLFKKDKAMLFTYDQYQENNVDQASGSVLLHLEVGDQVWLQVYGEGERNGLYADNDNDSTFTGFLLYHDTN

SEQ ID NO: 7 - protein, 130 aa, Homo sapiens
  apM1(115-244)
FSVGLETYVTIPNMPIRFTKIFYNQQNHYDGSTGKFHCNIPGLYYFAYHITVYMKDVKVSLFKKDKAMLFTYDQYQENNVDQASGSVLLHLEVGDQVWLQVYGEGERNGLYADNDNDSTFTGFLLYHDTN

SEQ ID NO: 8 - protein, 113 aa, Homo sapiens
  apM1(132-144)
FTKIFYNQQNHYDGSTGKFHCNIPGLYYFAYHITVYMKDVKVSLFKKDKAMLFTYDQYQENNVDQASGSVLLHLEVGDQVWLQVYGEGERNGLYADNDNDSTFTGFLLYHDTN

SEQ ID NO: 9 - DNA, 735 bp, Homo sapiens
  apM1(1-244) coding sequence
atgctgttgctgggagctgttctactgctattagctctgcccggtcatgaccaggaaaccacgactcaagggcccggagtcctgcttcccctgcccaagggggcctgcacaggttggatggcgggcatcccagggcatccgggccataatggggccccaggccgtgatggcagagatggcacccctggtgagaagggtgagaaaggagatccaggtcttattggtcctaagggagacatcggtgaaaccggagtacccggggctgaaggtccccgaggctttccgggaatccaaggcaggaaaggagaacctggagaaggtgcctatgtataccgctcagcattcagtgtgggattggagacttacgttactatccccaacatgcccattcgctttaccaagatcttctacaatcagcaaaaccactatgatggctccactggtaaattccactgcaacattcctgggctgtactactttgcctaccacatcacagtctatatgaaggatgtgaaggtcagcctcttcaagaaggacaaggctatgctcttcacctatgatcagtaccaggaaaataatgtggaccaggcctccggctctgtgctcctgcatctggaggtgggcgaccaagtctggctccaggtgtatggggaaggagagcgtaatggactctatgctgataatgacaatgactccaccttcacaggctttcttctctaccatgacaccaactga

SEQ ID NO: 10 - protein, 163 aa, Homo sapiens
  apM1(82-244)
ETGVPGAEGPRGFPGIQGRKGEPGEGAYVYRSAFSVGLETYVTIPNMPIRFTKIFYNQQNHYDGSTGKFHCNIPGLYYFAYHITVYMKDVKVSLFKKDKAMLFTYDQYQENNVDQASGSVLLHLEVGDQVWLQVYGEGERNGLYADNDNDSTFTGFLLYHDTN

SEQ ID NO: 11 - protein, 145 aa, Homo sapiens
  apM1(100-244)
RKGEPGEGAYVYRSAFSVGLETYVTIPNMPIRFTKIFYNQQNHYDGSTGKFHCNIPGLYYFAYHITVYMKDVKVSLFKKDKAMLFTYDQYQENNVDQASGSVLLHLEVGDQVWLQVYGEGERNGLYADNDNDSTFTGFLLYHDTN

SEQ ID NO: 12 - protein, 193 aa, Homo sapiens
  apM1(52-244)
APGRDGRDGTPGEKGEKGDPGLIGPKGDIGETGVPGAEGPRGFPGIQGRKGEPGEGAYVYRSAFSVGLETYVTIPNMPIRFTKIFYNQQNHYDGSTGKFHCNIPGLYYFAYHITVYMKDVKVSLFKKDKAMLFTYDQYQENNVDQASGSVLLHLEVGDQVWLQVYGEGERNGLYADNDNDSTFTGFLLYHDTN

SEQ ID NO: 13 - protein, 187 aa, Homo sapiens
  apM1(58-244)
RDGTPGEKGEKGDPGLIGPKGDIGETGVPGAEGPRGFPGIQGRKGEPGEGAYVYRSAFSVGLETYVTIPNMPIRFTKIFYNQQNHYDGSTGKFHCNIPGLYYFAYHITVYMKDVKVSLFKKDKAMLFTYDQYQENNVDQASGSVLLHLEVGDQVWLQVYGEGERNGLYADNDNDSTFTGFLLYHDTN

SEQ ID NO: 14 - DNA, 582 bp, Homo sapiens
  apM1(52-244) coding sequence
gccccaggccgtgatggcagagatggcacccctggtgagaagggtgagaaaggagatccaggtcttattggtcctaagggagacatcggtgaaaccggagtacccggggctgaaggtccccgaggctttccgggaatccaaggcaggaaaggagaacctggagaaggtgcctatgtataccgctcagcattcagtgtgggattggagacttacgttactatccccaacatgcccattcgctttaccaagatcttctacaatcagcaaaaccactatgatggctccactggtaaattccactgcaacattcctgggctgtactactttgcctaccacatcacagtctatatgaaggatgtgaaggtcagcctcttcaagaaggacaaggctatgctcttcacctatgatcagtaccaggaaaataatgtggaccaggcctccggctctgtgctcctgcatctggaggtgggcgaccaagtctggctccaggtgtatggggaaggagagcgtaatggactctatgctgataatgacaatgactccaccttcacaggctttcttctctaccatgacaccaactga

SEQ ID NO: 15 - DNA, 564 bp, Homo sapiens
  apM1(58-244) coding sequence
agagatggcacccctggtgagaagggtgagaaaggagatccaggtcttattggtcctaagggagacatcggtgaaaccggagtacccggggctgaaggtccccgaggctttccgggaatccaaggcaggaaaggagaacctggagaaggtgcctatgtataccgctcagcattcagtgtgggattggagacttacgttactatccccaacatgcccattcgctttaccaagatcttctacaatcagcaaaaccactatgatggctccactggtaaattccactgcaacattcctgggctgtactactttgcctaccacatcacagtctatatgaaggatgtgaaggtcagcctcttcaagaaggacaaggctatgctcttcacctatgatcagtaccaggaaaataatgtggaccaggcctccggctctgtgctcctgcatctggaggtgggcgaccaagtctggctccaggtgtatggggaaggagagcgtaatggactctatgctgataatgacaatgactccaccttcacaggctttcttctctaccatgacaccaactga

SEQ ID NO: 16 - DNA, 492 bp, Homo sapiens
  apM1(82-244) coding sequence
gaaaccggagtacccggggctgaaggtccccgaggctttccgggaatccaaggcaggaaaggagaacctggagaaggtgcctatgtataccgctcagcattcagtgtgggattggagacttacgttactatccccaacatgcccattcgctttaccaagatcttctacaatcagcaaaaccactatgatggctccactggtaaattccactgcaacattcctgggctgtactactttgcctaccacatcacagtctatatgaaggatgtgaaggtcagcctcttcaagaaggacaaggctatgctcttcacctatgatcagtaccaggaaaataatgtggaccaggcctccggctctgtgctcctgcatctggaggtgggcgaccaagtctggctccaggtgtatggggaaggagagcgtaatggactctatgctgataatgacaatgactccaccttcacaggctttcttctctaccatgacaccaactga

SEQ ID NO: 17 - protein, 163 aa, Artificial Sequence
  Synthetic construct T121C-apM1(82-244)
ETGVPGAEGPRGFPGIQGRKGEPGEGAYVYRSAFSVGLECYVTIPNMPIRFTKIFYNQQNHYDGSTGKFHCNIPGLYYFAYHITVYMKDVKVSLFKKDKAMLFTYDQYQENNVDQASGSVLLHLEVGDQVWLQVYGEGERNGLYADNDNDSTFTGFLLYHDTN

SEQ ID NO: 18 - protein, 163 aa, Artificial Sequence
  Synthetic construct S146C-apM1(82-244)
ETGVPGAEGPRGFPGIQGRKGEPGEGAYVYRSAFSVGLETYVTIPNMPIRFTKIFYNQQNHYDGCTGKFHCNIPGLYYFAYHITVYMKDVKVSLFKKDKAMLFTYDQYQENNVDQASGSVLLHLEVGDQVWLQVYGEGERNGLYADNDNDSTFTGFLLYHDTN

SEQ ID NO: 19 - protein, 163 aa, Artificial Sequence
  Synthetic construct T243C-apM1(82-244)
ETGVPGAEGPRGFPGIQGRKGEPGEGAYVYRSAFSVGLETYVTIPNMPIRFTKIFYNQQNHYDGSTGKFHCNIPGLYYFAYHITVYMKDVKVSLFKKDKAMLFTYDQYQENNVDQASGSVLLHLEVGDQVWLQVYGEGERNGLYADNDNDSTFTGFLLYHDCN

SEQ ID NO: 20 - protein, 203 aa, Artificial Sequence
  Synthetic construct T121C-apM1(42-244)
GIPGHPGHNGAPGRDGRDGTPGEKGEKGDPGLIGPKGDIGETGVPGAEGPRGFPGIQGRKGEPGEGAYVYRSAFSVGLECYVTIPNMPIRFTKIFYNQQNHYDGSTGKFHCNIPGLYYFAYHITVYMKDVKVSLFKKDKAMLFTYDQYQENNVDQASGSVLLHLEVGDQVWLQVYGEGERNGLYADNDNDSTFTGFLLYHDTN

SEQ ID NO: 21 - protein, 203 aa, Artificial Sequence
  Synthetic construct S146C-apM1(42-244)
GIPGHPGHNGAPGRDGRDGTPGEKGEKGDPGLIGPKGDIGETGVPGAEGPRGFPGIQGRKGEPGEGAYVYRSAFSVGLETYVTIPNMPIRFTKIFYNQQNHYDGCTGKFHCNIPGLYYFAYHITVYMKDVKVSLFKKDKAMLFTYDQYQENNVDQASGSVLLHLEVGDQVWLQVYGEGERNGLYADNDNDSTFTGFLLYHDTN

SEQ ID NO: 22 - protein, 203 aa, Artificial Sequence
  Synthetic construct T243C-apM1(42-244)
GIPGHPGHNGAPGRDGRDGTPGEKGEKGDPGLIGPKGDIGETGVPGAEGPRGFPGIQGRKGEPGEGAYVYRSAFSVGLETYVTIPNMPIRFTKIFYNQQNHYDGSTGKFHCNIPGLYYFAYHITVYMKDVKVSLFKKDKAMLFTYDQYQENNVDQASGSVLLHLEVGDQVWLQVYGEGERNGLYADNDNDSTFTGFLLYHDCN

SEQ ID NO: 23 - protein, 146 aa, Artificial Sequence
  Synthetic construct T121C-apM1(101-244)
KGEPGEGAYVYRSAFSVGLECYVTIPNMPIRFTKIFYNQQNHYDGSTGKFHCNIPGLYYFAYHITVYMKDVKVSLFKKDKAMLFTYDQYQENNVASPQASGSVLLHLEVGDQVWLQVYGEGERNGLYADNDNDSTFTGFLLYHDTN

SEQ ID NO: 24 - protein, 146 aa, Artificial Sequence
  Synthetic construct S146C-apM1(101-244)
KGEPGEGAYVYRSAFSVGLETYVTIPNMPIRFTKIFYNQQNHYDGCTGKFHCNIPGLYYFAYHITVYMKDVKVSLFKKDKAMLFTYDQYQENNVASPQASGSVLLHLEVGDQVWLQVYGEGERNGLYADNDNDSTFTGFLLYHDTN

SEQ ID NO: 25 - protein, 144 aa, Artificial Sequence
  Synthetic construct T243C-apM1(101-244)
KGEPGEGAYVYRSAFSVGLETYVTIPNMPIRFTKIFYNQQNHYDGSTGKFHCNIPGLYYFAYHITVYMKDVKVSLFKKDKAMLFTYDQYQENNVDQASGSVLLHLEVGDQVWLQVYGEGERNGLYADNDNDSTFTGFLLYHDCN

SEQ ID NO: 26 - protein, 145 aa, Artificial Sequence
  Synthetic construct T121C-apM1(100-244)
RKGEPGEGAYVYRSAFSVGLECYVTIPNMPIRFTKIFYNQQNHYDGSTGKFHCNIPGLYYFAYHITVYMKDVKVSLFKKDKAMLFTYDQYQENNVDQASGSVLLHLEVGDQVWLQVYGEGERNGLYADNDNDSTFTGFLLYHDTN

SEQ ID NO: 27 - protein, 145 aa, Artificial Sequence
  Synthetic construct S146C-apM1(100-244)
RKGEPGEGAYVYRSAFSVGLETYVTIPNMPIRFTKIFYNQQNHYDGCTGKFHCNIPGLYYFAYHITVYMKDVKVSLFKKDKAMLFTYDQYQENNVDQASGSVLLHLEVGDQVWLQVYGEGERNGLYADNDNDSTFTGFLLYHDTN

SEQ ID NO: 28 - protein, 145 aa, Artificial Sequence
  Synthetic construct T243C-apM1(100-244)
RKGEPGEGAYVYRSAFSVGLETYVTIPNMPIRFTKIFYNQQNHYDGSTGKFHCNIPGLYYFAYHITVYMKDVKVSLFKKDKAMLFTYDQYQENNVDQASGSVLLHLEVGDQVWLQVYGEGERNGLYADNDNDSTFTGFLLYHDCN

SEQ ID NO: 29 - protein, 193 aa, Artificial Sequence
  Synthetic construct T121C-apM1(52-244)
APGRDGRDGTPGEKGEKGDPGLIGPKGDIGETGVPGAEGPRGFPGIQGRKGEPGEGAYVYRSAFSVGLECYVTIPNMPIRFTKIFYNQQNHYDGSTGKFHCNIPGLYYFAYHITVYMKDVKVSLFKKDKAMLFTYDQYQENNVDQASGSVLLHLEVGDQVWLQVYGEGERNGLYADNDNDSTFTGFLLYHDTN

SEQ ID NO: 30 - protein, 193 aa, Artificial Sequence
  Synthetic construct  S146C-apM1(52-244)
APGRDGRDGTPGEKGEKGDPGLIGPKGDIGETGVPGAEGPRGFPGIQGRKGEPGEGAYVYRSAFSVGLETYVTIPNMPIRFTKIFYNQQNHYDGCTGKFHCNIPGLYYFAYHITVYMKDVKVSLFKKDKAMLFTYDQYQENNVDQASGSVLLHLEVGDQVWLQVYGEGERNGLYADNDNDSTFTGFLLYHDTN

SEQ ID NO: 31 - protein, 193 aa, Artificial Sequence
  Synthetic construct T243C-apM1(52-244)
APGRDGRDGTPGEKGEKGDPGLIGPKGDIGETGVPGAEGPRGFPGIQGRKGEPGEGAYVYRSAFSVGLETYVTIPNMPIRFTKIFYNQQNHYDGSTGKFHCNIPGLYYFAYHITVYMKDVKVSLFKKDKAMLFTYDQYQENNVDQASGSVLLHLEVGDQVWLQVYGEGERNGLYADNDNDSTFTGFLLYHDCN

SEQ ID NO: 32 - protein, 187 aa, Artificial Sequence
  Synthetic construct T121C-apM1(58-244)
RDGTPGEKGEKGDPGLIGPKGDIGETGVPGAEGPRGFPGIQGRKGEPGEGAYVYRSAFSVGLECYVTIPNMPIRFTKIFYNQQNHYDGSTGKFHCNIPGLYYFAYHITVYMKDVKVSLFKKDKAMLFTYDQYQENNVDQASGSVLLHLEVGDQVWLQVYGEGERNGLYADNDNDSTFTGFLLYHDTN

SEQ ID NO: 33 - protein, 187 aa, Artificial Sequence
  Synthetic construct S146C-apM1(58-244)
RDGTPGEKGEKGDPGLIGPKGDIGETGVPGAEGPRGFPGIQGRKGEPGEGAYVYRSAFSVGLETYVTIPNMPIRFTKIFYNQQNHYDGCTGKFHCNIPGLYYFAYHITVYMKDVKVSLFKKDKAMLFTYDQYQENNVDQASGSVLLHLEVGDQVWLQVYGEGERNGLYADNDNDSTFTGFLLYHDTN

SEQ ID NO: 34 - protein, 187 aa, Artificial Sequence
  Synthetic construct T243C-apM1(58-244)
RDGTPGEKGEKGDPGLIGPKGDIGETGVPGAEGPRGFPGIQGRKGEPGEGAYVYRSAFSVGLETYVTIPNMPIRFTKIFYNQQNHYDGSTGKFHCNIPGLYYFAYHITVYMKDVKVSLFKKDKAMLFTYDQYQENNVDQASGSVLLHLEVGDQVWLQVYGEGERNGLYADNDNDSTFTGFLLYHDCN

SEQ ID NO: 35 - protein, 163 aa, Artificial Sequence
  Synthetic construct N127C-apM1(82-244)
ETGVPGAEGPRGFPGIQGRKGEPGEGAYVYRSAFSVGLETYVTIPCMPIRFTKIFYNQQNHYDGSTGKFHCNIPGLYYFAYHITVYMKDVKVSLFKKDKAMLFTYDQYQENNVDQASGSVLLHLEVGDQVWLQVYGEGERNGLYADNDNDSTFTGFLLYHDTN

SEQ ID NO: 36 - protein, 163 aa, Artificial Sequence
  Synthetic construct  N141C-apM1(82-244)
ETGVPGAEGPRGFPGIQGRKGEPGEGAYVYRSAFSVGLETYVTIPNMPIRFTKIFYNQQCHYDGSTGKFHCNIPGLYYFAYHITVYMKDVKVSLFKKDKAMLFTYDQYQENNVDQASGSVLLHLEVGDQVWLQVYGEGERNGLYADNDNDSTFTGFLLYHDTN

SEQ ID NO: 37 - protein, 163 aa, Artificial Sequence
  Synthetic construct N228C-apM1(82-244)
ETGVPGAEGPRGFPGIQGRKGEPGEGAYVYRSAFSVGLETYVTIPNMPIRFTKIFYNQQNHYDGSTGKFHCNIPGLYYFAYHITVYMKDVKVSLFKKDKAMLFTYDQYQENNVDQASGSVLLHLEVGDQVWLQVYGEGERNGLYADCDNDSTFTGFLLYHDTN

SEQ ID NO: 38 - protein, 203 aa, Artificial Sequence
  Synthetic construct N127C-apM1(42-244)
GIPGHPGHNGAPGRDGRDGTPGEKGEKGDPGLIGPKGDIGETGVPGAEGPRGFPGIQGRKGEPGEGAYVYRSAFSVGLETYVTIPCMPIRFTKIFYNQQNHYDGSTGKFHCNIPGLYYFAYHITVYMKDVKVSLFKKDKAMLFTYDQYQENNVDQASGSVLLHLEVGDQVWLQVYGEGERNGLYADNDNDSTFTGFLLYHDTN

SEQ ID NO: 39 - protein, 203 aa, Artificial Sequence
  Synthetic construct N141C-apM1(42-244)
GIPGHPGHNGAPGRDGRDGTPGEKGEKGDPGLIGPKGDIGETGVPGAEGPRGFPGIQGRKGEPGEGAYVYRSAFSVGLETYVTIPNMPIRFTKIFYNQQCHYDGSTGKFHCNIPGLYYFAYHITVYMKDVKVSLFKKDKAMLFTYDQYQENNVDQASGSVLLHLEVGDQVWLQVYGEGERNGLYADNDNDSTFTGFLLYHDTN

SEQ ID NO: 40 - protein, 203 aa, Artificial Sequence
  Synthetic construct N228C-apM1(42-244)
GIPGHPGHNGAPGRDGRDGTPGEKGEKGDPGLIGPKGDIGETGVPGAEGPRGFPGIQGRKGEPGEGAYVYRSAFSVGLETYVTIPNMPIRFTKIFYNQQNHYDGSTGKFHCNIPGLYYFAYHITVYMKDVKVSLFKKDKAMLFTYDQYQENNVDQASGSVLLHLEVGDQVWLQVYGEGERNGLYADCDNDSTFTGFLLYHDTN

SEQ ID NO: 41 - protein, 144 aa, Artificial Sequence
  Synthetic construct N127C-apM1(101-244)
KGEPGEGAYVYRSAFSVGLETYVTIPCMPIRFTKIFYNQQNHYDGSTGKFHCNIPGLYYFAYHITVYMKDVKVSLFKKDKAMLFTYDQYQENNVDQASGSVLLHLEVGDQVWLQVYGEGERNGLYADNDNDSTFTGFLLYHDTN

SEQ ID NO: 42 - protein, 144 aa, Artificial Sequence
  Synthetic construct N141C-apM1(101-244)
KGEPGEGAYVYRSAFSVGLETYVTIPNMPIRFTKIFYNQQCHYDGSTGKFHCNIPGLYYFAYHITVYMKDVKVSLFKKDKAMLFTYDQYQENNVDQASGSVLLHLEVGDQVWLQVYGEGERNGLYADNDNDSTFTGFLLYHDTN

SEQ ID NO: 43 - protein, 144 aa, Artificial Sequence
  Synthetic construct N228C-apM1(101-244)
KGEPGEGAYVYRSAFSVGLETYVTIPNMPIRFTKIFYNQQNHYDGSTGKFHCNIPGLYYFAYHITVYMKDVKVSLFKKDKAMLFTYDQYQENNVDQASGSVLLHLEVGDQVWLQVYGEGERNGLYADCDNDSTFTGFLLYHDTN

SEQ ID NO: 44 - protein, 145 aa, Artificial Sequence
  Synthetic construct N127C-apM1(100-244)
RKGEPGEGAYVYRSAFSVGLETYVTIPCMPIRFTKIFYNQQNHYDGSTGKFHCNIPGLYYFAYHITVYMKDVKVSLFKKDKAMLFTYDQYQENNVDQASGSVLLHLEVGDQVWLQVYGEGERNGLYADNDNDSTFTGFLLYHDTN

SEQ ID NO: 45 - protein, 145 aa, Artificial Sequence
  Synthetic construct N141C-apM1(100-244)
RKGEPGEGAYVYRSAFSVGLETYVTIPNMPIRFTKIFYNQQCHYDGSTGKFHCNIPGLYYFAYHITVYMKDVKVSLFKKDKAMLFTYDQYQENNVDQASGSVLLHLEVGDQVWLQVYGEGERNGLYADNDNDSTFTGFLLYHDTN

SEQ ID NO: 46 - protein, 145 aa, Artificial Sequence
  Synthetic construct N228C-apM1(100-244)
RKGEPGEGAYVYRSAFSVGLETYVTIPNMPIRFTKIFYNQQNHYDGSTGKFHCNIPGLYYFAYHITVYMKDVKVSLFKKDKAMLFTYDQYQENNVDQASGSVLLHLEVGDQVWLQVYGEGERNGLYADCDNDSTFTGFLLYHDTN

SEQ ID NO: 47 - protein, 193 aa, Artificial Sequence
  Synthetic construct N127C-apM1(52-244)
APGRDGRDGTPGEKGEKGDPGLIGPKGDIGETGVPGAEGPRGFPGIQGRKGEPGEGAYVYRSAFSVGLETYVTIPCMPIRFTKIFYNQQNHYDGSTGKFHCNIPGLYYFAYHITVYMKDVKVSLFKKDKAMLFTYDQYQENNVDQASGSVLLHLEVGDQVWLQVYGEGERNGLYADNDNDSTFTGFLLYHDTN

SEQ ID NO: 48 - protein, 193 aa, Artificial Sequence
  Synthetic construct N141C-apM1(52-244)
APGRDGRDGTPGEKGEKGDPGLIGPKGDIGETGVPGAEGPRGFPGIQGRKGEPGEGAYVYRSAFSVGLETYVTIPNMPIRFTKIFYNQQCHYDGSTGKFHCNIPGLYYFAYHITVYMKDVKVSLFKKDKAMLFTYDQYQENNVDQASGSVLLHLEVGDQVWLQVYGEGERNGLYADNDNDSTFTGFLLYHDTN

SEQ ID NO: 49 - protein, 193 aa, Artificial Sequence
  Synthetic construct N228C-apM1(52-244)
APGRDGRDGTPGEKGEKGDPGLIGPKGDIGETGVPGAEGPRGFPGIQGRKGEPGEGAYVYRSAFSVGLETYVTIPNMPIRFTKIFYNQQNHYDGSTGKFHCNIPGLYYFAYHITVYMKDVKVSLFKKDKAMLFTYDQYQENNVDQASGSVLLHLEVGDQVWLQVYGEGERNGLYADCDNDSTFTGFLLYHDTN

SEQ ID NO: 50 - protein, 187 aa, Artificial Sequence
  Synthetic construct N127C-apM1(58-244)
RDGTPGEKGEKGDPGLIGPKGDIGETGVPGAEGPRGFPGIQGRKGEPGEGAYVYRSAFSVGLETYVTIPCMPIRFTKIFYNQQNHYDGSTGKFHCNIPGLYYFAYHITVYMKDVKVSLFKKDKAMLFTYDQYQENNVDQASGSVLLHLEVGDQVWLQVYGEGERNGLYADNDNDSTFTGFLLYHDTN

SEQ ID NO: 51 - protein, 187 aa, Artificial Sequence
  Synthetic construct N141C-apM1(58-244)
RDGTPGEKGEKGDPGLIGPKGDIGETGVPGAEGPRGFPGIQGRKGEPGEGAYVYRSAFSVGLETYVTIPNMPIRFTKIFYNQQCHYDGSTGKFHCNIPGLYYFAYHITVYMKDVKVSLFKKDKAMLFTYDQYQENNVDQASGSVLLHLEVGDQVWLQVYGEGERNGLYADNDNDSTFTGFLLYHDTN

SEQ ID NO: 52 - protein, 187 aa, Artificial Sequence
  Synthetic construct N228C-apM1(58-244)
RDGTPGEKGEKGDPGLIGPKGDIGETGVPGAEGPRGFPGIQGRKGEPGEGAYVYRSAFSVGLETYVTIPNMPIRFTKIFYNQQNHYDGSTGKFHCNIPGLYYFAYHITVYMKDVKVSLFKKDKAMLFTYDQYQENNVDQASGSVLLHLEVGDQVWLQVYGEGERNGLYADCDNDSTFTGFLLYHDTN

SEQ ID NO: 53 - protein, 163 aa, Artificial Sequence
  Synthetic construct Y111N-apM1(82-244)
ETGVPGAEGPRGFPGIQGRKGEPGEGAYVNRSAFSVGLETYVTIPNMPIRFTKIFYNQQNHYDGSTGKFHCNIPGLYYFAYHITVYMKDVKVSLFKKDKAMLFTYDQYQENNVDQASGSVLLHLEVGDQVWLQVYGEGERNGLYADNDNDSTFTGFLLYHDTN

SEQ ID NO: 54 - protein, 163 aa, Artificial Sequence
  Synthetic construct Y122N-apM1(82-244)
ETGVPGAEGPRGFPGIQGRKGEPGEGAYVYRSAFSVGLETNVTIPNMPIRFTKIFYNQQNHYDGSTGKFHCNIPGLYYFAYHITVYMKDVKVSLFKKDKAMLFTYDQYQENNVDQASGSVLLHLEVGDQVWLQVYGEGERNGLYADNDNDSTFTGFLLYHDTN

SEQ ID NO: 55 - protein, 163 aa, Artificial Sequence
  Synthetic construct R131N-apM1(82-244)
ETGVPGAEGPRGFPGIQGRKGEPGEGAYVYRSAFSVGLETYVTIPNMPINFTKIFYNQQNHYDGSTGKFHCNIPGLYYFAYHITVYMKDVKVSLFKKDKAMLFTYDQYQENNVDQASGSVLLHLEVGDQVWLQVYGEGERNGLYADNDNDSTFTGFLLYHDTN

SEQ ID NO: 56 - protein, 163 aa, Artificial Sequence
  Synthetic construct D144N+S146T-apM1(82-244)
ETGVPGAEGPRGFPGIQGRKGEPGEGAYVYRSAFSVGLETYVTIPNMPIRFTKIFYNQQNHYNGTTGKFHCNIPGLYYFAYHITVYMKDVKVSLFKKDKAMLFTYDQYQENNVDQASGSVLLHLEVGDQVWLQVYGEGERNGLYADNDNDSTFTGFLLYHDTN

SEQ ID NO: 57 - protein, 163 aa, Artificial Sequence
  Synthetic construct H151N+N153T-apM1(82-244)
ETGVPGAEGPRGFPGIQGRKGEPGEGAYVYRSAFSVGLETYVTIPNMPIRFTKIFYNQQNHYDGSTGKFNCTIPGLYYFAYHITVYMKDVKVSLFKKDKAMLFTYDQYQENNVDQASGSVLLHLEVGDQVWLQVYGEGERNGLYADNDNDSTFTGFLLYHDTN

SEQ ID NO: 58 - protein, 163 aa, Artificial Sequence
  Synthetic construct K178N+K180T-apM1(82-244)
ETGVPGAEGPRGFPGIQGRKGEPGEGAYVYRSAFSVGLETYVTIPNMPIRFTKIFYNQQNHYDGSTGKFHCNIPGLYYFAYHITVYMKDVKVSLFKNDTAMLFTYDQYQENNVDQASGSVLLHLEVGDQVWLQVYGEGERNGLYADNDNDSTFTGFLLYHDTN

SEQ ID NO: 59 - protein, 163 aa, Artificial Sequence
  Synthetic construct P129T-apM1(82-244)
ETGVPGAEGPRGFPGIQGRKGEPGEGAYVYRSAFSVGLETYVTIPNMTIRFTKIFYNQQNHYDGSTGKFHCNIPGLYYFAYHITVYMKDVKVSLFKKDKAMLFTYDQYQENNVDQASGSVLLHLEVGDQVWLQVYGEGERNGLYADNDNDSTFTGFLLYHDTN

SEQ ID NO: 60 - protein, 163 aa, Artificial Sequence
  Synthetic construct G145N-apM1(82-244)
ETGVPGAEGPRGFPGIQGRKGEPGEGAYVYRSAFSVGLETYVTIPNMPIRFTKIFYNQQNHYNGSTGKFHCNIPGLYYFAYHITVYMKDVKVSLFKKDKAMLFTYDQYQENNVDQASGSVLLHLEVGDQVWLQVYGEGERNGLYADNDNDSTFTGFLLYHDTN

SEQ ID NO: 61 - protein, 163 aa, Artificial Sequence
  Synthetic construct P155T-apM1(82-244)
ETGVPGAEGPRGFPGIQGRKGEPGEGAYVYRSAFSVGLETYVTIPNMPIRFTKIFYNQQNHYDGSTGKFHCNITGLYYFAYHITVYMKDVKVSLFKKDKAMLFTYDQYQENNVDQASGSVLLHLEVGDQVWLQVYGEGERNGLYADNDNDSTFTGFLLYHDTN

SEQ ID NO: 62 - DNA, 537 bp, Artificial Sequence
  Synthetic construct encoding T121C-apM1(82-244)
atgctgttgctgggagctgttctactgctattagctctgcccggtcatgacggagtacccggggctgaaggtccccgaggctttccgggaatccaaggcaggaaaggagaacctggagaaggtgcctatgtataccgctcagcattcagtgtgggattggagtgctacgttactatccccaacatgcccattcgctttaccaagatcttctacaatcagcaaaaccactatgatggctccactggtaaattccactgcaacattcctgggctgtactactttgcctaccacatcacagtctatatgaaggatgtgaaggtcagcctcttcaagaaggacaaggctatgctcttcacctatgatcagtaccaggaaaataatgtggaccaggcctccggctctgtgctcctgcatctggaggtgggcgaccaagtctggctccaggtgtatggggaaggagagcgtaatggactctatgctgataatgacaatgactccaccttcacaggctttcttctctaccatgacaccaactga

SEQ ID NO: 63 - DNA, 537 bp, Artificial Sequence
  Synthetic construct encoding S146C-apM1(82-244)
atgctgttgctgggagctgttctactgctattagctctgcccggtcatgacggagtacccggggctgaaggtccccgaggctttccgggaatccaaggcaggaaaggagaacctggagaaggtgcctatgtataccgctcagcattcagtgtgggattggagacttacgttactatccccaacatgcccattcgctttaccaagatcttctacaatcagcaaaaccactatgatggctgcactggtaaattccactgcaacattcctgggctgtactactttgcctaccacatcacagtctatatgaaggatgtgaaggtcagcctcttcaagaaggacaaggctatgctcttcacctatgatcagtaccaggaaaataatgtggaccaggcctccggctctgtgctcctgcatctggaggtgggcgaccaagtctggctccaggtgtatggggaaggagagcgtaatggactctatgctgataatgacaatgactccaccttcacaggctttcttctctaccatgacaccaactga

SEQ ID NO: 64 - DNA, 537 bp, Artificial Sequence
  Synthetic construct encoding T243C-apM1(82-244)
atgctgttgctgggagctgttctactgctattagctctgcccggtcatgacggagtacccggggctgaaggtccccgaggctttccgggaatccaaggcaggaaaggagaacctggagaaggtgcctatgtataccgctcagcattcagtgtgggattggagacttacgttactatccccaacatgcccattcgctttaccaagatcttctacaatcagcaaaaccactatgatggctccactggtaaattccactgcaacattcctgggctgtactactttgcctaccacatcacagtctatatgaaggatgtgaaggtcagcctcttcaagaaggacaaggctatgctcttcacctatgatcagtaccaggaaaataatgtggaccaggcctccggctctgtgctcctgcatctggaggtgggcgaccaagtctggctccaggtgtatggggaaggagagcgtaatggactctatgctgataatgacaatgactccaccttcacaggctttcttctctaccatgactgcaactga

SEQ ID NO: 65 - DNA, 537 bp, Artificial Sequence
  Synthetic construct encoding Y111N-apM1(82-244)
atgctgttgctgggagctgttctactgctattagctctgcccggtcatgacggagtacccggggctgaaggtccccgaggctttccgggaatccaaggcaggaaaggagaacctggagaaggtgcctatgtaaaccgctcagcattcagtgtgggattggagacttacgttactatccccaacatgcccattcgctttaccaagatcttctacaatcagcaaaaccactatgatggctccactggtaaattccactgcaacattcctgggctgtactactttgcctaccacatcacagtctatatgaaggatgtgaaggtcagcctcttcaagaaggacaaggctatgctcttcacctatgatcagtaccaggaaaataatgtggaccaggcctccggctctgtgctcctgcatctggaggtgggcgaccaagtctggctccaggtgtatggggaaggagagcgtaatggactctatgctgataatgacaatgactccaccttcacaggctttcttctctaccatgacaccaactga

SEQ ID NO: 66 - DNA, 537 bp, Artificial Sequence
  Synthetic construct encoding Y122N-apM1(82-244)
atgctgttgctgggagctgttctactgctattagctctgcccggtcatgacggagtacccggggctgaaggtccccgaggctttccgggaatccaaggcaggaaaggagaacctggagaaggtgcctatgtataccgctcagcattcagtgtgggattggagactaacgttactatccccaacatgcccattcgctttaccaagatcttctacaatcagcaaaaccactatgatggctccactggtaaattccactgcaacattcctgggctgtactactttgcctaccacatcacagtctatatgaaggatgtgaaggtcagcctcttcaagaaggacaaggctatgctcttcacctatgatcagtaccaggaaaataatgtggaccaggcctccggctctgtgctcctgcatctggaggtgggcgaccaagtctggctccaggtgtatggggaaggagagcgtaatggactctatgctgataatgacaatgactccaccttcacaggctttcttctctaccatgacaccaactga

SEQ ID NO: 67 - DNA, 537 bp, Artificial Sequence
  Synthetic construct encoding S146T-apM1(82-244)
atgctgttgctgggagctgttctactgctattagctctgcccggtcatgacggagtacccggggctgaaggtccccgaggctttccgggaatccaaggcaggaaaggagaacctggagaaggtgcctatgtataccgctcagcattcagtgtgggattggagacttacgttactatccccaacatgcccattcgctttaccaagatcttctacaatcagcaaaaccactataacggcactactggtaaattccactgcaacattcctgggctgtactactttgcctaccacatcacagtctatatgaaggatgtgaaggtcagcctcttcaagaaggacaaggctatgctcttcacctatgatcagtaccaggaaaataatgtggaccaggcctccggctctgtgctcctgcatctggaggtgggcgaccaagtctggctccaggtgtatggggaaggagagcgtaatggactctatgctgataatgacaatgactccaccttcacaggctttcttctctaccatgacaccaactga

SEQ ID NO: 68 - DNA, 537 bp, Artificial Sequence
  Synthetic construct encoding R131N-apM1(82-244)
atgctgttgctgggagctgttctactgctattagctctgcccggtcatgacggagtacccggggctgaaggtccccgaggctttccgggaatccaaggcaggaaaggagaacctggagaaggtgcctatgtataccgctcagcattcagtgtgggattggagacttacgttactatccccaacatgcccattaactttaccaagatcttctacaatcagcaaaaccactatgatggctccactggtaaattccactgcaacattcctgggctgtactactttgcctaccacatcacagtctatatgaaggatgtgaaggtcagcctcttcaagaaggacaaggctatgctcttcacctatgatcagtaccaggaaaataatgtggaccaggcctccggctctgtgctcctgcatctggaggtgggcgaccaagtctggctccaggtgtatggggaaggagagcgtaatggactctatgctgataatgacaatgactccaccttcacaggctttcttctctaccatgacaccaactga

SEQ ID NO: 69 - DNA, 537 bp, Artificial Sequence
  Synthetic construct encoding N127C-apM1(82-244)
atgctgttgctgggagctgttctactgctattagctctgcccggtcatgacggagtacccggggctgaaggtccccgaggctttccgggaatccaaggcaggaaaggagaacctggagaaggtgcctatgtataccgctcagcattcagtgtgggattggagacttacgttactatcccctgcatgcccattcgctttaccaagatcttctacaatcagcaaaaccactatgatggctccactggtaaattccactgcaacattcctgggctgtactactttgcctaccacatcacagtctatatgaaggatgtgaaggtcagcctcttcaagaaggacaaggctatgctcttcacctatgatcagtaccaggaaaataatgtggaccaggcctccggctctgtgctcctgcatctggaggtgggcgaccaagtctggctccaggtgtatggggaaggagagcgtaatggactctatgctgataatgacaatgactccaccttcacaggctttcttctctaccatgacaccaactga

SEQ ID NO: 70 - DNA, 537 bp, Artificial Sequence
  Synthetic construct encoding N141C-apM1(82-244)
atgctgttgctgggagctgttctactgctattagctctgcccggtcatgacggagtacccggggctgaaggtccccgaggctttccgggaatccaaggcaggaaaggagaacctggagaaggtgcctatgtataccgctcagcattcagtgtgggattggagacttacgttactatccccaacatgcccattcgctttaccaagatcttctacaatcagcaatgccactatgatggctccactggtaaattccactgcaacattcctgggctgtactactttgcctaccacatcacagtctatatgaaggatgtgaaggtcagcctcttcaagaaggacaaggctatgctcttcacctatgatcagtaccaggaaaataatgtggaccaggcctccggctctgtgctcctgcatctggaggtgggcgaccaagtctggctccaggtgtatggggaaggagagcgtaatggactctatgctgataatgacaatgactccaccttcacaggctttcttctctaccatgacaccaactga

SEQ ID NO: 71 - DNA, 537 bp, Artificial Sequence
  Synthetic construct encoding N228C-apM1(82-244)
atgctgttgctgggagctgttctactgctattagctctgcccggtcatgacggagtacccggggctgaaggtccccgaggctttccgggaatccaaggcaggaaaggagaacctggagaaggtgcctatgtataccgctcagcattcagtgtgggattggagacttacgttactatccccaacatgcccattcgctttaccaagatcttctacaatcagcaaaaccactatgatggctccactggtaaattccactgcaacattcctgggctgtactactttgcctaccacatcacagtctatatgaaggatgtgaaggtcagcctcttcaagaaggacaaggctatgctcttcacctatgatcagtaccaggaaaataatgtggaccaggcctccggctctgtgctcctgcatctggaggtgggcgaccaagtctggctccaggtgtatggggaaggagagcgtaatggactctatgctgattgcgacaatgactccaccttcacaggctttcttctctaccatgacaccaactga

SEQ ID NO: 72 - protein, 6 aa, Artificial Sequence
  Peptide tag
HHHHHH

SEQ ID NO: 73 - protein, 8 aa, Artificial Sequence
  Peptide tag
MKHHHHHH

SEQ ID NO: 74 - protein, 10 aa, Artificial Sequence
  Peptide tag
MKHHAHHQHH

SEQ ID NO: 75 - protein, 14 aa, Artificial Sequence
  Peptide tag
MKHQHQHQHQHQHQ

SEQ ID NO: 76 - protein, 10 aa, Artificial Sequence
  Peptide tag
EQKLISEEDL

SEQ ID NO: 77 - protein, 8 aa, Artificial Sequence
  Peptide tag
DYKDDDDK

SEQ ID NO: 78 - protein, 9 aa, Artificial Sequence
  Peptide tag
YPYDVPDYA

SEQ ID NO: 79 - protein, 18 aa, Artificial Sequence
  Peptide tag
MLLLGAVLLLLALPGHDG

SEQ ID NO: 80 - protein, 18 aa, Artificial Sequence
  Peptide tag
MLLLQALLFLLILPSHDG

SEQ ID NO: 81 - protein, 18 aa, Artificial Sequence
  Peptide tag
MLLLGAVLLLLALPGHDG

SEQ ID NO: 82 - DNA, 87 bp, Artificial Sequence
  Synthetic oligonucleotide
cgcggatccaccatgctgttgctgggagctgttctactgctattagctctgcccggtcatgacggcaggaaaggagaacctggagaa

SEQ ID NO: 83 - DNA, 35 bp, Artificial Sequence
  Synthetic oligonucleotide
atatatcccaagctttcagttggtgtcatggtaga

SEQ ID NO: 84 - protein, 15 aa, Artificial Sequence
  Synthetic peptide
EDDVTTTEELAPALV

SEQ ID NO: 85 - protein, 14 aa, Artificial Sequence
  Synthetic peptide
FTYDQYQEKNVDQA

SEQ ID NO: 86 - protein, 21 aa, Artificial Sequence
  Synthetic peptide
CYADNDNDSTFTGFLLYHDTN

SEQ ID NO: 87 - DNA, 87 bp, Artificial Sequence
  Synthetic oligonucleotide
cgcggatccaccatgctgttgctgggagctgttctactgctattagctctgcccggtcatgacggtgaaaccggagtacccggggct

SEQ ID NO: 88 - DNA, 87 bp, Artificial Sequence
  Synthetic oligonucleotide
cgcggatccaccatgctgttgctgggagctgttctactgctattagctctgcccggtcatgacggcagagatggcacccctggtgag

SEQ ID NO: 89 - DNA, 87 bp, Artificial Sequence
  Synthetic oligonucleotide
cgcggatccaccatgctgttgctgggagctgttctactgctattagctctgcccggtcatgacggggccccaggccgtgatggcaga

SEQ ID NO: 90 - DNA, 84 bp, Artificial Sequence
  Synthetic oligonucleotide
cgcggatccaccatgctgttgctgggagctgttctactgctattagctctgcccggtcatgacggcaaaggagaacctggagaa

SEQ ID NO: 91 - DNA, 30 bp, Artificial Sequence
  Synthetic oligonucleotide
gatagtaacgtagcactccaatcccacact

SEQ ID NO: 92 - DNA, 30 bp, Artificial Sequence
  Synthetic oligonucleotide
agtgtgggattggagtgctacgttactatc

SEQ ID NO: 93 - DNA, 36 bp, Artificial Sequence
  Synthetic oligonucleotide
atatatcccaagctttcagttggtgtcatggtagag

SEQ ID NO: 94 - DNA, 30 bp, Artificial Sequence
  Synthetic oligonucleotide
gtggaatttaccagtgcagccatcatagtg

SEQ ID NO: 95 - DNA, 30 bp, Artificial Sequence
  Synthetic oligonucleotide
cactatgatggctgcactggtaaattccac

SEQ ID NO: 96 - DNA, 35 bp, Artificial Sequence
  Synthetic oligonucleotide
atatatcccaagctttcagttgcagtcatggtaga

SEQ ID NO: 97 - DNA, 30 bp, Artificial Sequence
  Synthetic oligonucleotide
gcgaatgggcatgcaggggatagtaacgta

SEQ ID NO: 98 - DNA, 30 bp, Artificial Sequence
  Synthetic oligonucleotide
tacgttactatcccctgcatgcccattcgc

SEQ ID NO: 99 - DNA, 30 bp, Artificial Sequence
  Synthetic oligonucleotide
gccatcatagtggcattgctgattgtagaa

SEQ ID NO: 100 - DNA, 30 bp, Artificial Sequence
  Synthetic oligonucleotide
ttctacaatcagcaatgccactatgatggc

SEQ ID NO: 101 - DNA, 81 bp, Artificial Sequence
  Synthetic oligonucleotide
atatatcccaagctttcagttggtgtcatggtagagaagaaagcctgtgaaggtggagtcattgtcgcaatcagcatagag

SEQ ID NO: 102 - DNA, 30 bp, Artificial Sequence
  Synthetic oligonucleotide
gaatgctgagcggtttacataggcaccttc

SEQ ID NO: 103 - DNA, 30 bp, Artificial Sequence
  Synthetic oligonucleotide
gaaggtgcctatgtaaaccgctcagcattc

SEQ ID NO: 104 - DNA, 30 bp, Artificial Sequence
  Synthetic oligonucleotide
gttggggatagtaacgttagtctccaatcc

SEQ ID NO: 105 - DNA, 30 bp, Artificial Sequence
  Synthetic oligonucleotide
ggattggagactaacgttactatccccaac

SEQ ID NO: 106 - DNA, 30 bp, Artificial Sequence
  Synthetic oligonucleotide
gaatttaccagtagtgccgttatagtggtt

SEQ ID NO: 107 - DNA, 30 bp, Artificial Sequence
  Synthetic oligonucleotide
aaccactataacggcactactggtaaattc

SEQ ID NO: 108 - DNA, 30 bp, Artificial Sequence
  Synthetic oligonucleotide
gaagatcttggtaaagttaatgggcatgtt

SEQ ID NO: 109 - DNA, 30 bp, Artificial Sequence
  Synthetic oligonucleotide
aacatgcccattaactttaccaagatcttc

SEQ ID NO: 110 - protein, 135 aa, Mus musculus
MYRSAFSVGLETRVTVPNVPIRFTKIFYNQQNHYDGSTGKFYCNIPGLYYFSYHITVYMKDVKVSLFKKDKAVLFTYDQYQEKNVDQASGSVLLHLEVGDQVWLQVYGDGDHNGLYADNVNDSTFTGFLLYHDTN

SEQ ID NO: 111 - protein, 135 aa, Mus musculus
  Unsure 83, 108, 109, 110, 111, 112, 113, 114, 115, 135: Xaa = amino acid not resolved in crystal structure
MYRSAFSVGLETRVTVPNVPIRFTKIFYNQQNHYDGSTGKFYCNIPGLYYFSYHITVYMKDVKVSLFKKDKAVLFTYDQYQEXNVDQASGSVLLHLEVGDQVWLQVYXXXXXXXXYADNVNDSTFTGFLLYHDTX

SEQ ID NO: 112 - protein, 135 aa, Mus musculus
  Unsure 11, 12, 13, 14, 15, 16, 72, 84, 108, 109, 110, 111, 112,113, 114, 115, 116, 117, 118, 119, 120, 121, 134, 135: Xaa = amino acid not resolved in crystal structure
MYRSAFSVGLXXXXXXPNVPIRFTKIFYNQQNHYDGSTGKFYCNIPGLYYFSYHITVYMKDVKVSLFKKDKXVLFTYDQYQEKXVDQASGSVLLHLEVGDQVWLQVYXXXXXXXXXXXXXXDSTFTGFLLYHDXX

SEQ ID NO: 113 - protein, 135 aa, Mus musculus
  Unsure 16, 17, 18, 58, 59, 60, 77, 78, 79, 80, 81, 82, 83, 84,85, 86, 106, 107, 108, 109, 110, 111, 112, 113, 114, 115, 116,117, 118, 119, 120, 134, 135: Xaa = amino acid not resolved in crystal structure
MYRSAFSVGLETRVTXXXVPIRFTKIFYNQQNHYDGSTGKFYCNIPGLYYFSYHITVXXXDVKVSLFKKDKAVLFTXXXXXXXXXXQASGSVLLHLEVGDQVWLQXXXXXXXXXXXXXXXNDSTFTGFLLYHDXX

SEQ ID NO: 114 - protein, 132 aa, Homo sapiens [sequence omitted]

SEQ ID NO: 115 - protein, 29 aa, Artificial Sequence
  Synthetic peptide
PPGPPGPPGPPGPPGPPGPPGPPGPPGPP

(19) United States
(12) Patent Application Publication
(10) Pub. No.: US 2003/0176328 A1
(43) Pub. Date: Sep. 18, 2003

(54) Adiponectin fragments and conjugates

(75) Inventors: Poul Baad Rasmussen, Soeborg (DK); Kim Vilbour Andersen, Broenshoej (DK); Anders Hjelholt Pedersen, Lyngby (DK); Hans Thalsgaard Schambye, Holte (DK); Torben Halkier, Solroed Strand (DK); Are Bogsnes, Nivaa (DK)

Correspondence Address: MAXYGEN, INC., INTELLECTUAL PROPERTY DEPARTMENT, 515 GALVESTON DRIVE, RED WOOD CITY, CA, 94063, US

(73) Assignee: Maxygen ApS

(21) Appl. No.: 10/325,717

(22) Filed: Dec. 20, 2002

Related U.S. Application Data
(60) Provisional application No. 60/412,169, filed on Sep. 20, 2002.
(60) Provisional application No. 60/394,117, filed on Jul. 3, 2002.
(60) Provisional application No. 60/375,492, filed on Apr. 25, 2002.
(60) Provisional application No. 60/343,482, filed on Dec. 21, 2001.

(51) Int. Cl. A61K 38/17; C07K 14/47; C07H 21/04

(52) U.S. Cl. 514/8; 530/350; 530/395; 514/12; 536/23.5

(57) ABSTRACT

The invention relates to a conjugate comprising an adiponectin polypeptide, and a first non-polypeptide moiety covalently attached to the adiponectin polypeptide, wherein the adiponectin polypeptide comprises an amino acid residue having an attachment group for said first non-polypeptide moiety, wherein said amino acid residue has been introduced in a position that in the parent adiponectin is occupied by a surface exposed amino acid residue.

### CROSS-REFERENCE TO RELATED APPLICATIONS

This application claims priority from and benefit of U.S. Application Serial No. 60/412,169 filed Sep. 20, 2002; U.S. S No. 60/394,117 filed Jul. 3, 2002; U.S. S No. 60/375,492 filed Apr. 25, 2002; and U.S. S No. 60/343,482 filed Dec. 21, 2001, the disclosure of each of which is incorporated herein in its entirety for all purposes.

### FIELD OF THE INVENTION

The present invention relates to a novel conjugate comprising an adiponectin polypeptide, to a novel adiponectin polypeptide fragment, to a method of preparing such fragments or conjugates, to a nucleotide sequence encoding the adiponectin polypeptide fragment or part of the conjugate, to an expression vector comprising the nucleotide sequence, to a host cell comprising the nucleotide sequence, to a pharmaceutical composition comprising the conjugate, to a pharmaceutical composition comprising the fragment, to use of the conjugate for the manufacture of a medicament for treatment of type 1 diabetes; impaired glucose tolerance; type 2 diabetes; syndrome X; obesity; cardiovascular disease, such as atherosclerosis; septic shock; or dyslipidemia; or for lowering body weight without reducing food intake, and to a method of treating a mammal with type 1 diabetes; impaired glucose tolerance; type 2 diabetes; syndrome X; obesity; dyslipidemia; cardiovascular disease, such as atherosclerosis; or for lowering body weight of a mammal without reducing food intake; rheumatoid arthritis; Crohn's disease; systemic lupus erythematosus; Sjögren's disease; cachexia; septic shock; myasthenia gravis; post-traumatic brain damage; myocardial infarction; post-surgical brain-damage; and other destructive processes related to stress or activation of the inflammatory system.

### BACKGROUND OF THE INVENTION

Adiponectin (30 kDa) is a secreted protein expressed exclusively in differentiated adipocytes. Primary sequence analysis reveals four main domains: a cleaved amino-terminal signal sequence, a region without homology to known proteins, a collagen-like region, and a globular segment at the carboxy terminus. The globular domain forms homotrimers, and additional interactions between adiponectin collagenous segments cause the protein to form higher order structures. Adiponectin was cloned in 1995/96 and is also known as AdipoQ and Acrp30, and its human homologue has been designated independently as apM1 and GBP28.

Acrp30 protein shares sequence homology with a family of proteins showing a modular design containing a characteristic C-terminal complement factor Clq-like globular domain. In addition to Clq, members of this family include the human type VIII and X collagens, precerebellin, and the hibernation-regulated proteins hib 20, 25, and 27. Other than Clq, little is known regarding the function of the C-terminal globular regions of these proteins. In active and hibernating animals members of the hib family are differentially expressed in liver, suggesting a role in energy storage or mobilization. A similar function has been suggested for Acrp30 because the three-dimensional structure of its C-terminal globular domain is strikingly similar to that of tumor necrosis factor-α (TNFα), even though there is no homology at the primary sequence level. Among its various biological effects TNFα (TNF-alpha) regulates several aspects of energy homeostasis.

A variety of factors has been shown to modulate the activity of components of the insulin signalling pathway, suggesting potential roles in the aetiology of insulin resistance and type 2 diabetes. TNF-alpha, for example, has been shown to inhibit the tyrosine kinase activity of the insulin receptor in adipocytes, reducing the phosphorylation and activation of IRS-1 and so inhibiting the insulin signalling pathway. Given that obesity is associated with over-expression of TNF-alpha this suggests that TNF-alpha impairment of IRS-mediated insulin signalling may be responsible, at least in part, for obesity-associated insulin resistance. Furthermore, insulin receptors and IRS-1 are present in pancreatic beta cells, and TNF-alpha and other cytokines have been shown to alter insulin secretion. Thus, impairment of insulin signalling by TNF-alpha and/or other pro-inflammatory cytokines may be important pathogenic mechanism linking obesity and type 2 diabetes.

T. Yokota et al (Blood, 2000; 96, 1723-1732) showed that human full-length adiponectin (produced in E.coli) specifically inhibits LPS-induced TNF-alpha production in human macrophages, indicating that adiponectin also may have anti-inflammatory activity.

PPARgamma agonists can suppress the activation of macrophages and so reduce the production of cytokines by these cells. For example, they have been shown to suppress the LPS-induced TNF-alpha synthesis by human peripheral mononuclear cells (C. Jiang et al, Nature, 1998; 391, 82-86).

### BRIEF DISCLOSURE OF THE INVENTION

In the literature, both full-length adiponectin, (that is human adiponectin produced from E. Coli, and mouse adiponectin produced from E. Coli and mammalian cells), and globular fragments of adiponectin, (that is mouse adiponectin ACRP30 produced from E. Coli and mammalian cells), have been reported.

Common for the reported types of globular domains is that they are without a larger part of the collagenous domain that includes one to four lysines, thus, the known globular fragments of adiponectin do not include one or more lysines to be hydroxylated and glycosylated. Moreover, these globular fragments have been shown to be potent in muscle tissue, but they are not able to show any effect on insulin-reduced glucose output in hepatocytes. Furthermore, no reports of globular fragments of adiponectin in normalizing blood glucose levels have been made.

The reported full-length adiponectins are not as potent as globular fragments of adiponectin in muscle tissue, moreover, full-length adiponectin produced in E. Coli did not show any effect on insulin-reduced glucose output in hepatocytes. Full-length mouse adiponectin produced in mammalian cells showed effect on insulin-reduced glucose output in hepatocytes. Furthermore, full-length mouse adiponectin produced in mammalian cells have been able to reduce blood glucose in a mouse model (ob/ob) to near normal levels, when given in high dose.

Our medium sized fragments of adiponectin having a collagen domain (e.g. apM1(82′-244)) produced in mammalian cells comprises at least one lysine which is hydroxylated and glycosylated, moreover, they have been shown to transiently normalize blood glucose level in a db/db mouse model in a relatively low dose.

Without being bound by theory we believe that our medium sized fragments of adiponectin are more potent in the treatment of impaired glucose tolerance, and type 2 diabetes than the reported globular forms due to the hydroxy-glycosylation of one or more lysines in the collagenous domain.

Since our fragments transiently normalize blood glucose level in a db/db mouse model, this indicates that the adiponectin polypeptide fragment should be administered several times a day or more conveniently should be conjugated to, for instance, a polymer, such as a PEG, or a sugar moiety, to thereby increase the half-life, and reduce the frequency in administration. Another approach to deal with the transient normalization of blood glucose level would be to administer the adiponectin polypeptide fragment by gene therapy.

Accordingly, in one aspect the invention concerns an adiponectin polypeptide fragment, such as any one of SEQ ID NO:3, 4, 5, 10, 11, 12, or 13, as well as analogues thereof, which fragment comprises a globular domain, and a collagen domain, wherein at least one lysine in the collagen domain is hydroxylated and glycosylated.

Furthermore, we have analysed the structure of adiponectin, and located the amino acids which are surface exposed, and as such potential sites for introducing a non-polypeptide moiety.

Thus, in a further aspect the invention concerns a conjugate comprising an adiponectin polypeptide, and a first non-polypeptide moiety covalently attached to the adiponectin polypeptide, wherein the adiponectin polypeptide comprises an amino acid residue having an attachment group for said first non-polypeptide moiety, wherein said amino acid residue is a surface exposed amino acid residue.

Since not all the surface exposed amino acids are desired for attaching a non-polypeptide moiety, it is a further aspect of the invention to introduce suitable amino acids having an attachment group for the non-polypeptide moiety into the position of a surface exposed amino acid.

Thus, in a further aspect the invention concerns a conjugate comprising an adiponectin polypeptide, and a first non-polypeptide moiety covalently attached to the adiponectin polypeptide, wherein the adiponectin polypeptide comprises an amino acid residue having an attachment group for said first non-polypeptide moiety, wherein said amino acid residue has been introduced in a position that in the parent adiponectin is occupied by a surface exposed amino acid residue.

Furthermore, the wild type adiponectin has two conserved cysteine residues of which Cys 152 relative to SEQ ID NO:1, is non-surface exposed according to our analysis, and as such not an obvious choice when looking for a suitable attachment site for a non-polypeptide moiety.

Thus, in a further aspect the invention concerns a conjugate comprising an adiponectin polypeptide, and a first non-polypeptide moiety covalently attached to the adiponectin polypeptide, wherein the adiponectin polypeptide comprises an amino acid residue having an attachment group for said first non-polypeptide moiety, wherein said amino acid residue is a cysteine residue.

Furthermore, the N-terminal of the adiponectin may also be suitable for conjugation to a non-polypeptide, provided that activity is not lost.

Thus, in a further aspect the invention concerns a conjugate comprising an adiponectin polypeptide, and a first non-polypeptide moiety covalently attached to the adiponectin polypeptide, wherein the adiponectin polypeptide comprises an amino acid residue having an attachment group for said first non-polypeptide moiety, wherein the amino acid residue is the N-terminal amino acid residue.

Moreover, we have discovered that calcium ions are crucial for the adiponectin polypeptide to form stable trimers and that removal of such calcium ions leads to destabilization of the trimer structure.

Thus, in a further aspect the invention relates to an isolated complex comprising a) an adiponectin polypeptide or a conjugate comprising an adiponectin polypeptide, and a first non-polypeptide moiety covalently attached to the adiponectin polypeptide, and b) calcium ions.

Furthermore, we have discovered that the introduction of a polymer, such as a PEG, leads to trimers having one, two, or three polymers attached to the adiponectin polypeptide. Such trimers are further stabilized with calcium ions.

Thus, in a further aspect the invention relates to an isolated complex comprising

a) a conjugate comprising an adiponectin polypeptide trimer wherein the adiponectin polypeptide trimer contains three adiponectin polypeptide monomers, and one first polymer covalently attached to any one of the three monomers of the adiponectin polypeptide trimer in such a way that the resulting trimer only contains one polymer, and

b) calcium ions.

In a further aspect the invention concerns a nucleotide sequence encoding the adiponectin polypeptide part of the conjugate of the invention.

In a further aspect the invention concerns an expression vector comprising a nucleotide sequence of the invention.

In a further aspect the invention concerns a host cell comprising the nucleotide sequence of the invention.

In a further aspect the invention concerns a pharmaceutical composition comprising the conjugate of the invention and a pharmaceutically acceptable diluent, carrier or adjuvant.

In a further aspect the invention concerns a pharmaceutical composition comprising the adiponectin polypeptide fragment of the invention and a pharmaceutically acceptable diluent, carrier or adjuvant.

In a further aspect the invention concerns use of a conjugate of the invention for the manufacture of a medicament for treatment of type 1 diabetes; impaired glucose tolerance (herein after referred to as IGT); type 2 diabetes; syndrome X; obesity; cardiovascular disease, such as atherosclerosis; dyslipidernia; or for lowering body weight without reducing food intake; rheumatoid arthritis; Crohn's disease; systemic lupus erythematosus; Sjögren's disease; cachexia; septic shock; myasthenia gravis; post-traumatic brain damage; myocardial infarction; post-surgical brain-damage; and other destructive processes related to stress or activation of the inflammatory system; in particular IGT, type 2 diabetes, syndrome X, dyslipidemia, septic shock, or cardiovascular disease, such as atherosclerosis.

In a further aspect the invention concerns use of a conjugate or an adiponectin polypeptide fragment of the invention for preparing a medicament for treatment of a disease, disorder, or condition caused by expression or release of TNF-alpha in a human cell, wherein said medicament inhibits expression or release of TNF-alpha.

In a further aspect the invention concerns use of an adiponectin polypeptide fragment of the invention for the manufacture of a medicament for treatment of type 1 diabetes; impaired glucose tolerance (herein after referred to as IGT); type 2 diabetes; syndrome X; obesity; cardiovascular disease, such as atherosclerosis; dyslipidemia; or for lowering body weight without reducing food intake; rheumatoid arthritis; Crohn's disease; systemic lupus erythematosus; Sjögren's disease; cachexia; septic shock; myasthenia gravis; post-traumatic brain damage; myocardial infarction; post-surgical brain-damage; and other destructive processes related to stress or activation of the inflammatory system; in particular IGT, type 2 diabetes, syndrome X, dyslipidemia, septic shock, or cardiovascular disease, such as atherosclerosis.

In a further aspect the invention concerns a method of treating a mammal with type 1 diabetes; impaired glucose tolerance (herein after referred to as IGT); type 2 diabetes; syndrome X; obesity; cardiovascular disease, such as atherosclerosis; dyslipidemia; or for lowering body weight without reducing food intake; rheumatoid arthritis; Crohn's disease; systemic lupus erythematosus; Sjögren's disease; cachexia; septic shock; myasthenia gravis; post-traumatic brain damage; myocardial infarction; post-surgical brain-damage; and other destructive processes related to stress or activation of the inflammatory system; in particular IGT, type 2 diabetes, syndrome X, dyslipidemia, septic shock, or cardiovascular disease, such as atherosclerosis, which method comprises administering an effective amount of a conjugate or an adiponectin polypeptide fragment of the invention.

In a further aspect the present invention relates to a method of preparing an adiponectin polypeptide, comprising

a) preparing a nucleotide sequence encoding: a signal peptide and the adiponectin polypeptide, wherein the last three C-terminal amino acids of the signal peptide are HDG,

b) inserting the nucleotide sequence into a vector,

c) transfecting the vector into a mammalian cell,

d) expressing and optionally secreting the adiponectin polypeptide, and

e) optionally obtaining the adiponectin polypeptide.

Sequences of adiponectin polypeptides, fragments, and analogs, are provided in the Sequence Listing.

### BRIEF DESCRIPTION OF THE DRAWINGS

FIG. 1 is a Western blot probed with an anti-Acrp3O antibody which shows expression of apM1(100-244) and apM1(82-244) according to Examples 1 and 2, respectively.

FIG. 2 shows the effect of increasing amounts of adiponectin(82-244) on LPS-induced TNF-alpha production, as described in Example 23.

FIG. 3 is a Tris-Glycine native gel which shows the effect of lowered pH in the absence or presence of Ca2+ ions on the stability of the apM1(82-244) trimer complex as described in Example 24.

FIG. 4 is a Tris-Glycine native gel which shows a purified preparation of apM1(82-244) and the effect of CaCl₂, MgCl₂ and ZnCl₂ in re-stabilizing the adiponectin trimer as described in Example 25.

FIG. 5 shows the effect of apM1(82-244) on blood glucose levels in db/db mice, as described in Example 26.

### DETAILED DISCLOSURE OF THE INVENTION

In the present application a number of references are referred to. They are all intended to be incorporated herein by reference.

In the context of the present application and invention the following definitions apply:

The term “a” or “an”, eg as used in “a non-polypeptide”, “an amino acid residue”, “a substitution”, or “an attachment group”, is intended to indicate one or more, or at least one, eg a non-polypeptide means one or more non-polypeptides. “a” or “an” may be used interchangeably with “one or more” or “at least one” throughout the description.

The term “conjugate” (or interchangeably “conjugated polypeptide”) is intended to indicate a heterogeneous (in the sense of composite or chimeric) molecule formed by the covalent attachment of one or more polypeptide(s) to one or more non-polypeptide moieties. The term “covalent attachment” means that the polypeptide and the non-polypeptide moiety are either directly covalently joined to one another, or else are indirectly covalently joined to one another through an intervening moiety or moieties, such as a bridge, spacer, or linkage moiety or moieties using an attachment group present in the polypeptide. Preferably, the conjugate is soluble at relevant concentrations and conditions, i.e. soluble in physiological fluids such as blood. Examples of conjugated polypeptides of the invention include glycosylated polypeptides, PEGylated polypeptides, glycosylated and PEGylated polypeptides, as well as glycosylated polypeptides having a PEG attached to the sugar moiety. The term “non-conjugated polypeptide” may be used about the polypeptide part of the conjugate.

The term “non-polypeptide moiety” is intended to indicate a molecule that is capable of conjugating to an attachment group of the polypeptide of the invention. Preferred examples of such molecule include polymer molecules, sugar moieties, lipophilic compounds, or organic derivatizing agents. When used in the context of a conjugate of the invention it will be understood that the non-polypeptide moiety is linked to the polypeptide part of the conjugate through an attachment group of the polypeptide.

The term “polymer molecule” is defined as a molecule formed by covalent linkage of two or more monomers, wherein none of the monomers is an amino acid residue. The term “polymer” may be used interchangeably with the term “polymer molecule”. The term is intended to cover carbohydrate molecules attached by in vitro glycosylation, i.e. a synthetic glycosylation performed in vitro normally involving covalently linking a carbohydrate molecule to an attachment group of the polypeptide, optionally using a cross-linking agent. Carbohydrate molecules attached by in vivo glycosylation, such as N— or O-glycosylation (as further described below) are referred to herein as “a sugar moiety”. Except where the number of non-polypeptide moieties, such as polymer molecule(s) or sugar moieties in the conjugate is expressly indicated every reference to “a non-polypeptide moiety” contained in a conjugate or otherwise used in the present invention shall be a reference to one or more non-polypeptide moieties, such as polymer molecule(s) or sugar moieties, in the conjugate.

The term “mono-pegylated” is intended to mean that the adiponectin polypeptide has only one polymer comprising a polyethylene glycol (PEG) covalently attached to it. Mono-pegylation means that the conjugate may be homogenous, eg. mono-pegylation of the N-terminal, or it may be heterogenous, eg. mono-pegylation of one lysine residue in each adiponectin molecule, for instance, some of the adiponectin molecules may be pegylated in position K134, and some of the adiponectin molecules may be pegylated in position K149 relative to SEQ ID NO:1 (these examples are merely illustrative and are not intended to limit the invention in any way).

The term “isolated” is intended to mean that the material be removed from its original environment (e.g., the natural environment if it is naturally occurring). For example, a naturally-occurring polynucleotide or polypeptide present in a living animal is not isolated, but the same polynucleotide or DNA or polypeptide, separated from some or all of the coexisting materials in the natural system, is isolated. Such polynucleotide could be part of a vector and/or such polynucleotide or polypeptide could be part of a composition, and still be isolated in that the vector or composition is not part of its natural environment.

The term “attachment group” is intended to indicate an amino acid residue group of the polypeptide capable of coupling to the relevant non-polypeptide moiety. For instance, for polymer, in particular polyethylene glycol (PEG), conjugation a frequently used attachment group is the E-amino group of lysine or the N-terminal amino group. Other polymer attachment groups include a free carboxylic acid group (e.g. that of the C-terminal amino acid residue or of an aspartic acid or glutamic acid residue), suitably activated carbonyl groups, oxidized carbohydrate moieties and mercapto groups (eg. the sulfhydryl group of cysteine).

For in vivo N-glycosylation, the term “attachment group” is used in an unconventional way to indicate the amino acid residues constituting an N-glycosylation site (with the sequence N-X′-S/T/C-X″, wherein X′ is any amino acid residue except proline, X″ is any amino acid residue that may or may not be identical to X′ and preferably is different from proline, N is asparagine and S/T/C is either serine, threonine or cysteine, preferably serine or threonine, and most preferably threonine). Although the asparagine residue of the N-glycosylation site is the one to which the sugar moiety is attached during glycosylation, such attachment cannot be achieved unless the other amino acid residues of the N-glycosylation site is present. Accordingly, when the non-polypeptide moiety is an N-linked sugar moiety, the term “amino acid residue having an attachment group for the first non-polypeptide moiety” as used in connection with alterations of the amino acid sequence of the parent polypeptide is to be understood as amino acid residues constituting an N-glycosylation site is/are to be altered in such a manner that a functional N-glycosylation site is introduced into the amino acid sequence. For an “O-glycosylation site” the attachment group is the OH-group of a serine or threonine residue, and in that respect the non-polypeptide moiety is an O-linked sugar moiety.

In the present application, amino acid names and atom names (e.g. CA, CB, CD, CG, SG, NZ, N, O, C, etc) are used as defined by the Protein DataBank (PDB) (www.pdb.org) which are based on the IUPAC nomenclature (IUPAC Nomenclature and Symbolism for Amino Acids and Peptides (residue names, atom names e.t.c.), Eur. J. Biochem., 138, 9-37 (1984) together with their corrections in Eur. J. Biochem., 152, 1 (1985). CA is sometimes referred to as Cα, CB as Cβ. The term “amino acid residue” is intended to indicate an amino acid residue contained in the group consisting of alanine (Ala or A), cysteine (Cys or C), aspartic acid (Asp or D), glutamic acid (Glu or E), phenylalanine (Phe or F), glycine (Gly or G), histidine (His or H), isoleucine (Ile or I), lysine (Lys or K), leucine (Leu or L), methionine (Met or M), asparagine (Asn or N), proline (Pro or P), glutamine (Gin or Q), arginine (Arg or R), serine (Ser or S), threonine (Thr or T), valine (Val or V), tryptophan (Trp or W), and tyrosine (Tyr or Y) residues. The terminology used for identifying amino acid positions/substitutions is illustrated as follows: C152 (indicates position #152 occupied by a cysteine residue in the amino acid sequence e.g. shown in SEQ ID NO:1). C152S indicates that the cysteine residue of position 152 has been replaced with a serine. The numbering of amino acid residues made herein is made relative to the amino acid sequence shown in SEQ ID NO:1. Multiple substitutions are indicated with a “+”, e.g. F115N+V 117T/S means an amino acid sequence which comprises a substitution of the phenylalanine residue in position 115 with an asparagine and a substitution of the valine residue in position 117 with a threonine or serine residue, preferably a threonine residue. T/S as used about a given substitution herein means either a T or a S residue, preferably a T residue. As explained above the nomenclature X151 Y is intended to mean that amino acid X in position 151 relative to human adiponectin has been substituted with amino acid Y, such as H151N.

The term “nucleotide sequence” is intended to indicate a consecutive stretch of two or more nucleotide molecules. The nucleotide sequence may be of genomic, cDNA, RNA, semisynthetic, synthetic origin, or any combinations thereof.

The term “polymerase chain reaction” or “PCR” generally refers to a method for amplification of a desired nucleotide sequence in vitro, as described, for example, in U.S. Pat. No. 4,683,195. In general, the PCR method involves repeated cycles of primer extension synthesis, using oligonucleotide primers capable of hybridising preferentially to a template nucleic acid.

“Cell”, “host cell”, “cell line” and “cell culture” are used interchangeably herein and all such terms should be understood to include progeny resulting from growth or culturing of a cell. “Transformation” and “transfection” are used interchangeably to refer to the process of introducing DNA into a cell.

“Operably linked” refers to the covalent joining of two or more nucleotide sequences, by means of enzymatic ligation or otherwise, in a configuration relative to one another such that the normal function of the sequences can be performed. For example, the nucleotide sequence encoding a presequence or secretory leader is operably linked to a nucleotide sequence for a polypeptide if it is expressed as a preprotein that participates in the secretion of the polypeptide: a promoter or enhancer is operably linked to a coding sequence if it affects the transcription of the sequence; a ribosome binding site is operably linked to a coding sequence if it is positioned so as to facilitate translation. Generally, “operably linked” means that the nucleotide sequences being linked are contiguous and, in the case of a secretory leader, contiguous and in reading phase. Linking is accomplished by ligation at convenient restriction sites. If such sites do not exist, then synthetic oligonucleotide adaptors or linkers are used, in conjunction with standard recombinant DNA methods.

The term “introduce” is primarily intended to mean substitution of an existing amino acid residue, but may also mean insertion of an additional amino acid residue. The term “remove” is primarily intended to mean substitution of the amino acid residue to be removed by another amino acid residue, but may also mean deletion (without substitution) of the amino acid residue to be removed.

The term “immunogenicity” as used in connection with a given substance is intended to indicate the ability of the substance to induce a response from the immune system. The immune response may be a cell or antibody mediated response (see, e.g., Roitt: Essential Immunology (8^{th} Edition, Blackwell) for further definition of immunogenicity). Immunogenicity may be determined by use of any suitable method known in the art, e.g. in vivo or in vitro. The term “reduced immunogenicity” is intended to indicate that the conjugate or polypeptide of the present invention gives rise to a measurably lower immune response than a reference molecule, such as wildtype human adiponectin (apMl), or a variant of wild-type human adiponectin, as determined under comparable conditions. Normally, reduced antibody reactivity is an indication of reduced immunogenicity.

The term “functional in vivo half-life” is used in its normal meaning, i.e. the time at which 50% of a given functionality of the conjugate is retained (such as the time at which 50% of the biological activity of the conjugate is still present in the body/target organ, or the time at which the activity of the conjugate is 50% of the initial value). As an alternative to determining functional in vivo half-life, “serum half-life” may be determined, i.e. the time in which 50% of the conjugate molecules circulate in the plasma or bloodstream prior to being cleared. Determination of serum half-life is often more simple than determining functional in vivo half-life and the magnitude of serum half-life is usually a good indication of the magnitude of functional in vivo half-life. Alternative terms to serum half-life include “plasma half-life”, “circulating half-life”, “serum clearance”, “plasma clearance” and “clearance half-life”. The functionality to be retained is normally selected from antiviral, antiproliferative, immunomodulatory or receptor binding activity. Functional in vivo half-life and serum half-life may be determined by any suitable method known in the art.

The conjugate is normally cleared by the action of one or more of the reticuloendothelial systems (RES), kidney, spleen or liver, or by specific or unspecific proteolysis. Clearance taking place by the kidneys may also be referred to as “renal clearance” and is e.g. accomplished by glomerular filtration, tubular excretion or tubular elimination. Normally, clearance depends on physical characteristics of the conjugate, including molecular weight, size (diameter) (relative to the cut-off for glomerular filtration), charge, symmetry, shape/rigidity, attached carbohydrate chains, and the presence of cellular receptors for the protein. A molecular weight of about 67 kDa is considered to be an important cut-off-value for renal clearance.

Reduced renal clearance may be established by any suitable assay, e.g. an established in vivo assay. Typically, the renal clearance is determined by administering a labelled (e.g. radiolabelled or fluorescence labelled) polypeptide conjugate to a patient and measuring the label activity in urine collected from the patient. Reduced renal clearance is determined relative to the corresponding non-conjugated polypeptide or the non-conjugated corresponding wild-type polypeptide under comparable conditions.

The term “increased” as used about the functional in vivo half-life or serum half-life is used to indicate that the relevant half-life of the conjugate is statistically significantly increased relative to that of a reference molecule, such as an non-conjugated wildtype human adiponectin or an non-conjugated variant human adiponectin as determined under comparable conditions.

The term “reduced immunogenicity and/or increased functional in vivo half-life and/or increased serum half-life” is to be understood as covering any one, two or all of these properties. Preferably, a conjugate of the invention has at least two or these properties, i.e. reduced immunogenicity and increased functional in vivo half-life, reduced immunogenicity and increased serum half-life or increased functional in vivo half-life and increased serum half-life. Most preferably, the conjugate of the invention has all properties.

The conjugates of the invention are useful as inter alia (ia) insulin sensitizers based on their ability to exhibit activity in the Test Assay (described in the experimental section) by stimulating the insulin-dependent reduction in glucose output in primary hepatocytes.

The term “one difference” or “differs from” as used in connection with specific mutations is intended to allow for additional differences being present apart from the specified amino acid difference. For instance, in addition to the removal and/or introduction of amino acid residues comprising an attachment group for the non-polypeptide moiety the adiponectin polypeptide may comprise other substitutions that are not related to introduction and/or removal of such amino acid residues. The terms “mutation” and “substitution” are used interchangeably herein.

The term “adiponectin polypeptide” is intended to indicate that the polypeptide has a sequence selected from any one of SEQ ID NO:1-8, 10-12, or 13, as well as homologues, analogues, and fragments thereof. Typically, the adiponectin polypeptide is selected from any one of SEQ ID NO:1-8, 10-12, or 13, as well as sequences that differs from any one of the specified sequences, in one or more substitution(s), preferably from one to eight, eg one to six. For convenience, the single strands of the cDNA encoding apM1(52-244), apM1(58-244), and apM1(82-244), are shown in SEQ ID NO:14-16, respectively. The term “homologue” is intended to indicate that a polypeptide has at least 50% identity, such as at least 60%, 70%, 80%, 90%, or 95% identity, with any one of SEQ ID NO:1-8, 10-12, or 13. The term “fragment” or “adiponectin polypeptide fragment” is intended to indicate any one of SEQ ID NO:s 2-8, 10-12, or 13, as well as homologues, analogues, and truncated versions thereof. Such truncation may take place at the N- or C-terminal end in accordance with known procedures, eg SEQ ID NO:5 may be C-terminally truncated by cleaving off two amino acids, thereby producing a sequence having amino acid 101 to 242 of human wild type adiponectin (human adiponectin (101-242), or apM1(101-242)). Another couple of examples of the nomenclature that has been used throughout this specification is apM1(82-244) which means the sequence of human wild type adiponectin from amino acid 82 to 244; apM1(52-244) which means the sequence of human wild type adiponectin from amino acid 52 to 244; and apM1(58-244) which means the sequence of human wild type adiponectin from amino acid 58 to 244. A further nomenclature that has been used throughout this specification is, for instance, T121C-apM1(82-244) which means the sequence of human wild type adiponectin from amino acid 82 to 244, wherein Thr in position 121 has been substituted with Cys.

Typically, the term fragment means that any one of the SEQ ID NO:2-8, 10-12, or 13, is truncated N-terminally with 1, 2, 3, 4, 5, or 6 amino acid residues, or truncated C-terminally with 1, 2, 3, 4, 5, or 6 amino acid residues. In a non-limiting example, the fragment is truncated N-terminally with 6 amino acid residues, and optionally truncated C-terminally with 2 amino acid residues.

The percent identity as stated above can be determined conventionally using known computer programs. Typically, we are using the CLUSTALW program. (Thompson et al., 1994, CLUSTAL W: improving the sensitivity of progressive multiple sequence alignment through sequence weighting, position-specific gap penalties and weight matrix choice, Nucleic Acids Research, 22:46734680).

Typically the adiponectin polypeptide exhibits activity in the Test Assay (described in the experimental section): Determination of adiponectin's effect on glucose uptake in C2C12 cells.

The adiponectin polypeptide also exhibits activity in the Test Assay (described in the experimental section) by inhibiting LPS-induced TNF-alpha production in monocytic cell line.

The adiponectin polypeptide also exhibits activity in the Test Assay (described in the experimental section) by enhancing the insulin mediated suppression of glucose out-put in primary hepatocytes.

The adiponectin polypeptide also exhibits activity in db/db mice (described in the experimental section) by lowering and normalizing blood glucose level.

Human wildtype adiponectin (or interchangeably “human adiponectin”) (SEQ ID NO:1) consists of 244 amino acid residues, that is, a signal sequence from amino acid 1-17, a non-homologous domain from amino acid 18-41, a collagen domain from amino acid 42-107, and a globular domain from amino acid 108-244. The single strand of the cDNA encoding human adiponectin is shown in SEQ ID NO:9.

The term “globular domain” is intended to indicate the sequence of human adiponectin (108-244) (shown in SEQ ID NO:6) and analogues thereof. Fragments are also intended to be comprised, that is both C-terminally truncated as well as N-terminally truncated. The globular domain of human adiponectin (apM1) is known to form trimers.

The term “trimer” as used in connection with an adiponectin polypeptide trimer means that three molecules of an adiponectin polypeptide monomer forms a trimer.

The term “homotrimer” means that the trimer consists of three identical monomers.

The term “heterotrimer” means that the trimer consists of different monomers, such as, two of the monomers may be the same and the third may be different, or all three monomers may be different. The difference being that one or two monomer(s) has/have an amino acid sequence that differs from that of the other monomer(s).

The term “collagen domain” is intended to indicate the sequence of human adiponectin (42-107) (as indicated in SEQ ID NO:1) and analogues thereof. Fragments are also intended to be comprised, that is both C-terminally truncated as well as N-terminally truncated. A collagen domain is well known to have repeating sequences of Gly-X-Y, wherein X and Y are the same or different and selected from the amino acids (one letter code): A, R, N, D, C, Q, E, G, H, I, L, K, M, F, P, S, T, W, Y, and V. An example of the collagen domain is the amino acids from Gly99 to Gly107 (G99-G107). Another example of the collagen domain is the amino acids from Glu82 to Gly107 (E82-G107) The term “non-homologuous domain” is intended to indicate the sequence of human adiponectin (1841) (as indicated in SEQ ID NO:1) and analogues thereof. Fragments are also intended to be comprised, that is both C-terminally truncated as well as N-terminally truncated.

The term “signal peptide” is intended to indicate the sequence of human adiponectin (1-17) (as indicated in SEQ ID NO:1) and analogues thereof. Fragments are also intended to be comprised, that is both C-terminally truncated as well as N-terminally truncated. An example of the signal sequence is the amino acids from Met1 to Asp17 (M1-D17).

The term “parent adiponectin” (or interchangeably “parent adiponectin polypeptide”) is intended to indicate the starting molecule to be improved in accordance with the present invention. While the parent adiponectin may be of any origin, such as vertebrate or mammalian origin (e.g. any of the origins defined in WO 01/51645), or fragments thereof, the parent adiponectin is typically wild-type human adiponectin with SEQ ID NO:1, or any of the fragments of SEQ ID NO:s 2-8, 10-12, or 13, or an analogue thereof.

An “analogue” is a polypeptide, which differs in one or more amino acid residues from a parent polypeptide, normally in 1, 2, 3, 4, 5, 6, 7, 8, 9, 10, 11, 12, 13, 14, or 15 amino acid residues.

The term “functional site” as used about a polypeptide or conjugate of the invention is intended to indicate one or more amino acid residues which is/are essential for or otherwise involved in the function or performance of adiponectin, and thus “located at” the functional site.

Characterisation of the apM1(82-244) prepared in example 2 revealed that apM1(82-244) produced in CHO cells is partially hydroxylated on the Pro-residues (P95 and P104) and partially hydroxylated and subsequently glycosylated on the Lys 101-residue in the collagen-like part of the molecule (hereinafter also referred to as glyco-hydroxy-Lys). Thus, eucaryotic cells, typically, mammalian cells expressing, for instance, adiponectin polypeptide of SEQ ID NO:3, 10, 12, or 13, produces sequences with four, one, four, and four glyco-hydroxy-Lys residues, respectively. Whenever, one, two, three or four Lys are present in the collagenous domain, and the adiponectin polypeptide is produced in mammalian cells, it comprises such post-translational modifications. Moreover, if a more optimized hydroxylation of the Pro-residues is desired, then Vitamine C should be present during expression of the polypeptide. Typically, the adiponectin polypeptide is selected from any one of SEQ ID NO:2, 3, 4, 5, 10, 11, 12, or 13, as well as sequences that differs from any one of the specified sequences, in one or more substitution(s), preferably from one to eight (in the situation with SEQ ID NO:5, the hydroxylated and glycosylated N-terminal lysine, may be prepared by constructing a longer fragment, such as apM1(82-244), and subsequently cutting with a suitable enzyme, such as trypsin). If the adiponectin polypeptide differs in one or more substitutions it means that one or more amino acid residues are introduced or removed, or some may be introduced and some may be removed.

Adiponectin Polypeptide Fragment(s) of the Invention

In a first aspect the invention concerns an adiponectin polypeptide fragment comprising any one of SEQ ID NO:2, 3, 4, 5, 10, 11, 12, or 13, as well as homologues, analogues, and fragments thereof. Typically, the adiponectin polypeptide fragment is selected from any one of SEQ ID NO:2, 3, 4, 5, 10, 11, 12, or 13, as well as sequences that differs from any one of the specified sequences, in one or more substitution(s), preferably from one to eleven, such as from one to eight.

The one or more substitution(s) (as explained above) in addition to the removal and/or introduction of amino acid residues comprising an attachment group for the non-polypeptide moiety may also comprise other substitutions that are not related to introduction and/or removal of such amino acid residues. However, the adiponectin polypeptide fragment as well as sequences that differs in one or more substitution(s), should have biological activity, such activity could be tested in a relevant animal model, such as mouse models of insulin resistance and diabetes, such as the db/db mouse described in: A. E. Halseth et al, Biochemical and Biophysical Research Communications 294 (2002) 798-805) mice; or the ob/ob mouse described in: X. M. Song et al, Diabetologia 45 (2002) 56-65; or rat models such as zucker rats, or could be tested in a relevant in vitro assay, such as any one of the Test Assays A, B, or C described in the experimental section.

In a further embodiment the adiponectin polypeptide fragment is selected from any one of SEQ ID NO: 3, 10, 12, or 13, as well as sequences that differs from any one of the specified sequences in one or more substitution(s), preferably from one to eleven, such as in one to eight substitutions, eg. 1-6 substitutions.

In a further embodiment the adiponectin polypeptide fragment is selected from any one of SEQ ID NO:2, 3, 4, 5, 10, 11, 12, or 13, preferably 3, 10, 12, or 13. A typical adiponectin polypeptide fragment is SEQ ID NO:10. Another typical adiponectin polypeptide fragment is SEQ ID NO:12. A further typical adiponectin polypeptide fragment is SEQ ID NO:13.

In a alternative embodiment the adiponectin polypeptide fragment is selected from sequences that differs from any one of the SEQ ID NO:2, 3, 4, 5, 10, 11, 12, or 13, preferably 3, 10, 12, or 13, in one or more substitutions, preferably from one to eleven, such as in one to eight substitutions, eg. 1-6 substitutions.

In a further alternative embodiment the adiponectin polypeptide fragment is selected from sequences that differs from the SEQ ID NO:3 in one or more substitutions, preferably from one to eleven, such as in one to eight substitutions, eg. 1-6 substitutions.

In a further alternative embodiment the adiponectin polypeptide fragment is selected from sequences that differs from the SEQ ID NO:10 in one or more substitutions, preferably from one to eleven, such as in one to eight substitutions, eg. 1-6 substitutions.

In a further alternative embodiment the adiponectin polypeptide fragment is selected from sequences that differs from the SEQ ID NO:12 in one or more substitutions, preferably from one to eleven, such as in one to eight substitutions, eg. 1-6 substitutions.

In a further alternative embodiment the adiponectin polypeptide fragment is selected from sequences that differs from the SEQ ID NO:13 in one or more substitutions, preferably from one to eleven, such as in one to eight substitutions, eg. 1-6 substitutions.

Typically, the adiponectin polypeptide fragment is produced in a mammalian cell, eg a CHO, BHK, HEK293 cell or an SF9 cell. The lysines in the collagenous domain are hydroxylated and glycosylated, when produced in a eucaryotic cell, such as a mammalian cell.

In a further embodiment the adiponectin polypeptide fragment comprises one to four lysine residues selected from any one of the positions K65, K68, K77, or K101. In a further embodiment the adiponectin polypeptide fragment comprises at least one lysine residue selected from any one of the positions K65, K68, K77, or K101. Preferably, the lysine residues are hydroxylated and glycosylated. In a further embodiment the adiponectin polypeptide fragment comprises one lysine residue selected from any one of the positions K65, K68, K77, or K101, preferably K101, and preferably the position is hydroxylated and glycosylated, such as glyco-hydroxy-K101. In a further embodiment the adiponectin polypeptide fragment comprises two lysine residues selected from any one of the positions K65, K68, K77, or K 101, preferably K77 and K 101, and preferably both of the positions are hydroxylated and glycosylated, such as glyco-hydroxy-K77 and glyco-hydroxy-K101. In a further embodiment the adiponectin polypeptide fragment comprises three lysine residues selected from any one of the positions K65, K68, K77, or K101, preferably K68, K77 and K101, and preferably all three of the positions are hydroxylated and glycosylated, such as glyco-hydroxy-K68, glyco-hydroxy-K77 and glyco-hydroxy-K101. In a further embodiment the adiponectin polypeptide fragment comprises four lysine residues selected from positions K65, K68, K77, and K101, and preferably all four of the positions are hydroxylated and glycosylated. The N-terminal amino acid of the collagen domain is typically not a lysine, eg. K65, K68, or K77, since such a lysine will not be hydroxylated and glycosylated upon expression in a eucaryotic cell, such as a mammalian cell. However, as explained above if the desired adiponectin polypeptide fragment has the N-terminal amino acid, K101, then the lysine may be hydroxylated and glycosylated upon expression of a longer fragment in a eucaryotic cell, and subsequently cutting with a suitable enzyme, such as a trypsin.

When the adiponectin polypeptide fragment comprises a collagen domain, such as any one of SEQ ID NO:3, 4, 5, 10, 11, 12, or 13, such collagen domain comprises lysines, which when produced in a eucaryotic cell are hydroxylated and glycosylated. If the adiponectin polypeptide fragment only has 7 amino acids, or less, of the collagen domain, such as apM1(101-244) shown in SEQ ID NO:5, then the lysine will not be hydroxylated and glycosylated. However, the apM1(101-244) could be constructed so as to have a glyco-hydroxy-K101 residue, since production of, eg. apM1(82-244) in a CHO cell, and subsequently cutting with an enzyme (that cuts between arginine and lysine), such as trypsin, between position R100 and K101 would create the apM1(101-244) having the position K101 hydroxylated and glycosylated.

Thus, in a certain aspect the invention concerns an adiponectin polypeptide fragment comprising a globular domain and a collagen domain,

wherein the globular domain comprises an amino acid sequence as indicated in SEQ ID NO:1 from position A108 to N244 as well as sequences that differs from the amino acid sequence in one or more substitution(s), and

wherein the collagen domain comprises from 7 amino acids corresponding to position K101 as indicated in SEQ ID NO:1 to 66 amino acids corresponding to position G42 as indicated in SEQ ID NO:1, and wherein the collagen domain comprises a lysine which is hydroxylated and glycosylated.

The above adiponectin polypeptide fragment comprising a globular domain and a collagen domain wherein the collagen domain comprises a lysine which is hydroxylated and glycosylated is particularly preferred over adiponectin polypeptide fragments which do not have a collagen domain or which do not comprise a lysine which is hydroxylated and glycosylated. The presence of a lysine which is hydroxylated and glycosylated improves the overall performance of the molecule as a therapeutic agent useful for treating eg. impaired glucose tolerance, type 2 diabetes, syndrome X, obesity, a cardiovascular disease, such as atherosclerosis, or dyslipidemia. Moreover, we have discovered that if the adiponectin polypeptide fragment is to be expressed in acceptable yields in a eucaryotic cell, such as a mammalian cell, then the collagen domain should not comprise more than 56 amino acids, preferably not more than 50 amino acids. However, it was possible to increase the expression, with the aid of an expression enhancer, such as UCOE, when the collagen domain comprises more than 50 amino acids. Typically, so-called UCOE's may be obtained from Cobra Therapeutics Limited, or may be prepared, for instance, as described in WO 00/05393.

Thus, the above adiponectin polypeptide fragment comprising a globular domain and a collagen domain is expressed in high yields from a eucaryotic, such as a mammalian expression system so as to be reproducible in large scale culturing.

Accordingly, a preferred aspect of the invention concerns an adiponectin polypeptide fragment comprising a globular domain and a collagen domain,

wherein the globular domain comprises an amino acid sequence from position A108 to N244 as indicated in SEQ ID NO:1 as well as sequences that differs from the amino acid sequence in one or more substitution(s), and

wherein the collagen domain comprises from 7 amino acids corresponding to position K101 as indicated in SEQ ID NO:1 to 56 amino acids corresponding to position A52 as indicated in SEQ ID NO:1, and wherein the collagen domain comprises a lysine which is hydroxylated and glycosylated.

Typically, the globular domain should not contain too many amino acid changes as this may reduce the biological activity or lead to increased immunogenicity.

Accordingly, in a further embodiment of the adiponectin polypeptide fragment comprising a globular domain and a collagen domain, the globular domain comprises an amino acid sequence from position 108 to 244 as indicated in SEQ ID NO:1 as well as sequences that differs from the amino acid sequence in up to eleven substitution(s).

In a further embodiment the globular domain comprises an amino acid sequence from position A108 to N244 as indicated in SEQ ID NO:1.

In the situation were it is desired to introduce glycosylation site(s), or remove/introduce amino acid(s) in the globular domain, the globular domain differs from the amino acid sequence from position A108 to N244 as indicated in SEQ ID NO:1 in one or more substitution(s). Typically, the globular domain differs from the amino acid sequence from position A108 to N244 as indicated in SEQ ID NO:1 in one to eleven (11) substitution(s), such as 1-10, 1-9,1-8, 1-7,1-6, 1-5, 14, 1-3,1-2, 1, 2, 3, 4, 5, 6, 7, 8, 9, 10 or 11 substitution(s).

Furthermore, the adiponectin polypeptide fragment comprises any one of the above embodiments of the globular domain together with a collagen domain that comprises from 7 amino acids corresponding to position K101 as indicated in SEQ ID NO:1 to 66 amino acids corresponding to position G42 as indicated in SEQ ID NO:1, and wherein the collagen domain comprises a lysine which is hydroxylated and glycosylated. In a further embodiment the collagen domain comprises from 7 amino acids corresponding to position K101 as indicated in SEQ ID NO:1 to 56 amino acids corresponding to position A52 as indicated in SEQ ID NO:1. Typically, the collagen domain comprises from 7 amino acids corresponding to position K101 as indicated in SEQ ID NO:1 to 50 amino acids corresponding to position R58 as indicated in SEQ ID NO:1, such as from 8 amino acids corresponding to position R100 as indicated in SEQ ID NO:1 to 50 amino acids corresponding to position R58 as indicated in SEQ ID NO:1, from 8 amino acids corresponding to position R100 as indicated in SEQ ID NO:1 to 47 amino acids corresponding to position T61 as indicated in SEQ ID NO:1, from 8 amino acids corresponding to position R100 as indicated in SEQ ID NO:1 to 44 amino acids corresponding to position E64 as indicated in SEQ ID NO:1, from 8 amino acids corresponding to position R100 as indicated in SEQ ID NO:1 to 41 amino acids corresponding to position E67 as indicated in SEQ ID NO:1, from 8 amino acids corresponding to position R100 as indicated in SEQ ID NO:1 to 38 amino acids corresponding to position D70 as indicated in SEQ ID NO:1, from 8 amino acids corresponding to position R100 as indicated in SEQ ID NO:1 to 35 amino acids corresponding to position L73 as indicated in SEQ ID NO:1, from 8 amino acids corresponding to position R100 as indicated in SEQ ID NO:1 to 32 amino acids corresponding to position P76 as indicated in SEQ ID NO:1, from 8 amino acids corresponding to position R100 as indicated in SEQ ID NO:1 to 29 amino acids corresponding to position D79 as indicated in SEQ ID NO:1, from 8 amino acids corresponding to position R100 as indicated in SEQ ID NO:1 to 26 amino acids corresponding to position E82 as indicated in SEQ ID NO:1, from 8 amino acids corresponding to position R100 as indicated in SEQ ID NO:1 to 23 amino acids corresponding to position V85 as indicated in SEQ ID NO:1, from 8 amino acids corresponding to position R100 as indicated in SEQ ID NO:1 to 20 amino acids corresponding to position A88 as indicated in SEQ ID NO:1, from 8 amino acids corresponding to position R100 as indicated in SEQ ID NO:1 to 17 amino acids corresponding to position P91 as indicated in SEQ ID NO:1, from 8 amino acids corresponding to position R100 as indicated in SEQ ID NO:1 to 14 amino acids corresponding to position F94 as indicated in SEQ ID NO:1, from 8 amino acids corresponding to position R100 as indicated in SEQ ID NO:1 to 11 amino acids corresponding to position 197 as indicated in SEQ ID NO:1, from 11 amino acids corresponding to position 197 as indicated in SEQ ID NO:1 to 50 amino acids corresponding to position R58 as indicated in SEQ ID NO:1, from 14 amino acids corresponding to position F94 as indicated in SEQ ID NO:1 to 47 amino acids corresponding to position T61 as indicated in SEQ ID NO:1, from 17 amino acids corresponding to position P91 as indicated in SEQ ID NO:1 to 44 amino acids corresponding to position E64 as indicated in SEQ ID NO:1, from 20 amino acids corresponding to position A88 as indicated in SEQ ID NO:1 to 41 amino acids corresponding to position E67 as indicated in SEQ ID NO:1, from 23 amino acids corresponding to position V85 as indicated in SEQ ID NO:1 to 38 amino acids corresponding to position D70 as indicated in SEQ ID NO:1, from 26 amino acids corresponding to position E82 as indicated in SEQ ID NO:1 to 35 amino acids corresponding to position L73 as indicated in SEQ ID NO:1, including the collagen domain comprising 7 amino acids corresponding to position K101 as indicated in SEQ ID NO:1, 8 amino acids corresponding to position R100 as indicated in SEQ ID NO:1, 11 amino acids corresponding to position 197 as indicated in SEQ ID NO:1, 14 amino acids corresponding to position F94 as indicated in SEQ ID NO:1, 17 amino acids corresponding to position P91 as indicated in SEQ ID NO:1, 20 amino acids corresponding to position A88 as indicated in SEQ ID NO:1, 23 amino acids corresponding to position V85 as indicated in SEQ ID NO:1, 26 amino acids corresponding to position E82 as indicated in SEQ ID NO:1, 29 amino acids corresponding to position D79 as indicated in SEQ ID NO:1, 32 amino acids corresponding to position P76 as indicated in SEQ ID NO:1, 35 amino acids corresponding to position L73 as indicated in SEQ ID NO:1, 38 amino acids corresponding to position D70 as indicated in SEQ ID NO:1, 41 amino acids corresponding to position E67 as indicated in SEQ ID NO:1, 44 amino acids corresponding to position E64 as indicated in SEQ ID NO:1; 47 amino acids corresponding to position T61 as indicated in SEQ ID NO:1, 50 amino acids corresponding to position R58 as indicated in SEQ ID NO:1, 56 amino acids corresponding to position A52 as indicated in SEQ ID NO:1, 66 amino acids corresponding to position G42 as indicated in SEQ ID NO:1. Any one of the above collagen domains comprises a lysine which is hydroxylated and glycosylated. Typically, as is the case with the collagen domain of human adiponectin, the lysine to be hydroxylated and glycosylated should be N-terminally adjacent to a glycine, cf. also The Journal of Biological Chemistry, “Conformational Requirement for Lysine Hydroxylation in Collagen”, Vol. 266, No. 34, Issue of December 5, pp. 22960-22967, 1991.

Depending on the length of the collagen domain it may comprise one to four lysine(s), such as 1, 2, 3, or 4.

Typically, the collagen domain of the adiponectin polypeptide fragment comprises one to four lysine residues selected from any one of the positions K65, K68, K77, or K101 as indicated in SEQ ID NO:1. As mentioned above (in connection with adiponectin polypeptide having SEQ ID NO:5, having an N-terminal lysine at K101) a lysine in the collagen domain, which is the N-terminal residue, will not be hydroxylated and glycosylated upon expression of such adiponectin polypeptide fragment in a eucaryotic cell. If for instance an adiponectin polypeptide fragment having four lysine residues in the positions K65, K68, K77, and K101, wherein K65 (as indicated in SEQ ID NO:1) is the N-terminal amino acid, is desired, then expression of such fragment will lead to a fragment having a collagen domain wherein the three positions K68, K77, and K101, are hydroxylated and glycosylated, and wherein K65 is not. Another example is an adiponectin polypeptide fragment having three lysine residues in the positions K68, K77, and K101, wherein K68 (as indicated in SEQ ID NO:1) is the N-terminal amino acid, then expression of such fragment will lead to a fragment having a collagen domain wherein the two positions K77, and K101, are hydroxylated and glycosylated, and wherein K68 is not. However, if the desired adiponectin polypeptide fragment has the N-terminal amino acid, K68, then the lysine may be hydroxylated and glycosylated upon expression of a longer fragment in a eucaryotic cell, and subsequently cutting with a suitable protease, that specifically cleave proteins following a glutamic acid residue, such as the protease purified from Staphylococcus aureus V8, which is comercially available. Another example is an adiponectin polypeptide fragment having two lysine residues in the positions K77, and K101, wherein K77 (as indicated in SEQ ID NO:1) is the N-terminal amino acid, then expression of such fragment will lead to a fragment having a collagen domain wherein the position K101, is hydroxylated and glycosylated, and wherein K77 is not. However, if the desired adiponectin polypeptide fragment has the N-terminal amino acid, K77, then the lysine may be hydroxylated and glycosylated upon expression of a longer fragment in a eucaryotic cell, and subsequently cutting with a suitable Prolyl endoprotease, (in somecases also called prolyl oligopeptidases, which are widely present in microorganisms, plants and animals) which act as a post-proline cleaving enzyme, such as the enzyme from the microorganism Flavobacterium meningosepticum (which is commercially available). In the situation wherein the N-terminal amino acid is not a lysine, then an adiponectin polypeptide fragment comprising 1, 2, 3, or 4 lysine(s) will contain 1, 2, 3, or 4 lysine residues that are hydroxylated and glycosylated, respectively, upon expression in a eucaryotic cell. Thus, in a particular embodiment the adiponectin polypeptide fragment comprises one lysine residue which is hydroxylated and glycosylated, such as the position K101 as indicated in SEQ ID NO:1. In another particular embodiment the adiponectin polypeptide fragment comprises two lysine residues which are hydroxylated and glycosylated, such as the positions K77, and K101 as indicated in SEQ ID NO:1. In a further particular embodiment the adiponectin polypeptide fragment comprises three lysine residues which are hydroxylated and glycosylated, such as the positions K68, K77, and K101 as indicated in SEQ ID NO:1. In a further particular embodiment the adiponectin polypeptide fragment comprises four lysine residues which are hydroxylated and glycosylated, such as the positions K65, K68, K77, and K101 as indicated in SEQ ID NO:1.

Any one of the above adiponectin polypeptide fragment(s) of the invention may be prepared according to methods known in the art. Such method include recombinant DNA techniques, preferably the methods mentioned in the section “Methods of preparing an adiponectin polypeptide for use in the invention” are used, and a particular suitable method of preparation, is the method of preparing an adiponectin polypeptide (including a fragment thereof), comprising

a) preparing a nucleotide sequence encoding: a signal peptide and the adiponectin polypeptide, wherein the last three C-terminal amino acids of the signal peptide are HDG,

b) inserting the nucleotide sequence into a vector,

c) transfecting the vector into a mammalian cell,

d) expressing and optionally secreting the adiponectin polypeptide, and

e) obtaining the adiponectin polypeptide.

Any one of the above adiponectin polypeptide fragment(s) comprising any one of SEQ ID NO:3, 4, 5, 10, 11, 12, or 13, as well as homologues, analogues, and fragments thereof, including any one of the specified embodiments may be tested for biological activity in a suitable animal model or in vitro assay as mentioned above. Thus, in one embodiment the adiponectin polypeptide fragment normalises blood glucose concentration in a db/db mouse. In another embodiment the adiponectin polypeptide fragment enhances glucose uptake in muscle cells. A suitable in vitro assay for testing glucose uptake is Test Assay A. In a further embodiment the adiponectin polypeptide fragment inhibit LPS-induced TNF-alpha production in a monocytic cell line or in a macrophage. A suitable in vitro assay for testing inhibition of LPS-induced TNF-alpha production is Test Assay B. In a further embodiment the adiponectin polypeptide fragment enhances the insulin mediated suppression of glucose out-put in primary hepatocytes. A suitable in vitro assay for testing reduced glucose production is Test Assay C. Adiponectin polypeptide fragments which enhance glucose uptake in muscle cells and inhibit LPS-induced TNF-alpha production in a monocytic cell line or in a macrophage are preferred. Other preferred adiponectin polypeptide fragments are those which enhance glucose uptake in muscle cells and reduce glucose production in primary hepatocytes. It should be clear that in all the test models/assays the adiponectin polypeptide is tested and compared to a control group which did not receive the adiponectin polypeptide.

First Group of Conjugate(s) of the Invention

As stated above, in a further aspect the invention relates to a conjugate comprising an adiponectin polypeptide, and a first non-polypeptide moiety covalently attached to the adiponectin polypeptide, wherein the adiponectin polypeptide comprises an amino acid residue having an attachment group for said first non-polypeptide moiety, wherein said amino acid residue is a surface exposed amino acid residue.

In a second aspect the invention relates to a conjugate consisting essentially of an adiponectin polypeptide, and a first non-polypeptide moiety covalently attached to the adiponectin polypeptide, wherein the adiponectin polypeptide comprises an amino acid residue having an attachment group for said first non-polypeptide moiety, wherein said amino acid residue is a surface exposed amino acid residue.

In a further aspect the invention relates to a conjugate comprising an adiponectin polypeptide selected from SEQ ID NO:5 or 6, and one first non-polypeptide moiety covalently attached to the adiponectin polypeptide, wherein the adiponectin polypeptide comprises an amino acid residue having an attachment group for said first non-polypeptide moiety, wherein said amino acid residue is a surface exposed amino acid residue.

The amino acid residue having the attachment group for the first non-polypeptide moiety is located at the surface of the adiponectin polypeptide, and typically has more than 25% of its side chain exposed to the solvent, such as more than 50% of its side chain exposed to the solvent. We believe that such positions in the globular domain may be identified on the basis of an analysis of the 3D structure of the crystal structure of the globular domain of mouse ACRP30, cf Brief Communication, “The crystal structure of a complement-1q family protein suggets an evolutionary link to tumor necrosis factor”, Shapiro et al, pp 335-338. Typically, in the globular and collagen domains all lysine residues are surface exposed. The surface exposed amino acid residues have been identified as outlined in the experimental section herein.

By using a surface exposed amino acid residue which is already present in the wildtype molecule having an attachment group for a non-polypeptide moiety it will not be necessary to make mutations, however, this does not exclude that mutations can be made, provided that the conjugate maintain biological activity, and thereby its usefulness for treating eg. impaired glucose tolerance, type 2 diabetes, syndrome X, obesity, a cardiovascular disease, such as atherosclerosis, or dyslipidemia, such activity could be tested in a relevant animal model, such as mouse models of insulin resistance and diabetes, such as db/db or ob/ob mice, or rat models such as zucker rats, or could be tested in a relevant in vitro assay, such as any one of the Test Assays A, B, or C described in the experimental section.

In one embodiment the surface exposed amino acid residue is an amino acid residue having at least 25%, such as at least 50% of its side chain exposed to the surface. In a particular embodiment the s surface exposed amino acid residue is an amino acid residue having 100% of its side chain exposed to the surface.

In a further embodiment the surface exposed amino acid residue is selected from A108, Y109, V110, Y111, R112, L119, E120, T121, Y122, V123, T124, 1125, P126, N127, M128, 1130, R131, T133, K134, 1135, F136, Y137, N138, Q139, Q140, N141, H142, D144, G145, S146, T147, K149, H151, N153, 1154, P155, Y159, A161, H163, 1164, T165, Y167, M168, K169, D170, V171, K172, F176, K177, K178, D179, K180, A181, M182, F184, T185, Y186, D187, Q188, Y189, Q190, E191, N192, N193, V194, D195, Q196, S198, G199, S200, H204, E206, V207, G208, D209, Q210, W212, Q214, V215, Y216, G217, E218, G219, E220, R221, N222, G223, L224, Y225, A226, D227, N228, D229, N230, D231, T233, F234, F237, L238, L239, Y240, H241, D242, T243, or N244 of human adiponectin. Each of these positions is considered an embodiment and may be made the subject of a claim, moreover, any one of these positions may be combined with any one of the embodiments hereinafter.

In a further embodiment the surface exposed amino acid residue is selected from A108, Y109, V110, Y111, R112, E120, T121, Y122, V123, T124, 1125, P126, N127, M128, R131, T133, K134, 1135, Q139, N141, D144, G145, S146, T147, K149, H151, N153, P155, Y167, M168, K169, D170, K178, D179, K180, A181, F184, Y186, D187, Q188, Y189, Q190, E191, N192, N193, V194, D195, H204, E206, V207, G208, Q210, V215, Y216, G217, E218, G219, E220, R221, N222, G223, L224, Y225, A226, D227, N228, D229, N230, H241, D242, T243, or N244 of human adiponectin.

In a further embodiment the surface exposed amino acid residue is selected from A108, Y109, V110, Y111, E120, T121, Y122, V123, T124, 1125, P126, N127, M128, R131, Q139, N141, D144, G145, S146, N153, Y167, M168, K169, K178, D179, K180, A181, Y186, D187, Q188, Y189, Q190, E191, N192, N193, V194, D195, E206, V207, G208, V215, Y216, G217, E218, G219, E220, R221, N222, G223, L224, Y225, A226, D227, N228, D229, N230, H241, T243, or N244 of human adiponectin.

In a further embodiment the surface exposed amino acid residue is selected from A108, Y109, E120, T121, Y122, V123, T124, 1125, P126, N127, Y167, M168, K169, A181, Y186, D187, Q188, Y189, Q190, E191, N192, N193, V194, D195, V215, Y216, G217, E218, G219, E220, R221, N222, G223, L224, Y225, A226, D227, N228, D229, N230, T243, or N244 of human adiponectin.

The identification of surface exposed amino acids in the globular domain of human adiponectin has made it possible to select the desired target for attaching a non-polypeptide moiety. Such a non-polypeptide moiety is typically selected from a polymer molecule, a lipophilic compound, or an organic derivatizing agent. Suitable methods for attaching a non-polypeptide moiety to any one of the surface exposed amino acids in the globular domain of human adiponectin are well known to the skilled person. The preferred methods of attaching a non-polypeptide moiety selected from a polymer molecule, a lipophilic compound, or an organic derivatizing agent are described in more detail in the section “Methods of preparing a conjugate of the invention” hereinafter.

The adiponectin polypeptide should have a globular domain, such as indicated in the sequence of human adiponectin (108-244) (shown in SEQ ID NO:6). The adiponectin polypeptide part of the conjugate comprises the globular domain having the amino acid sequence shown in SEQ ID NO:6 as well as analogues thereof, including fragments. As mentioned also analogues are comprised, in particular analogues that differs in 1, 2, 3, 4, 5, 6, 7, 8, 9, 10, 11, 12, 13, 14, or 15 amino acid residues relative to the amino acid sequence shown in SEQ ID NO:6.

Thus, in a further embodiment the adiponectin polypeptide part of the conjugate comprises a globular domain, preferably a collagen and a globular domain. In a still further embodiment the adiponectin polypeptide comprises the amino acid sequence of SEQ ID NO:10. In a further embodiment the adiponectin polypeptide comprises the amino acid sequence of SEQ ID NO:1. In a further embodiment the adiponectin polypeptide comprises the amino acid sequence of SEQ ID NO:12. In a further embodiment the adiponectin polypeptide comprises the amino acid sequence of SEQ ID NO:13. In a further embodiment the adiponectin polypeptide comprises the amino acid sequence of SEQ ID NO:6. In a further embodiment the adiponectin polypeptide comprises the amino acid sequence of SEQ ID NO:5. In a further embodiment the adiponectin polypeptide comprises the amino acid sequence of SEQ ID NO:4. In a further embodiment the adiponectin polypeptide comprises the amino acid sequence of SEQ ID NO:3. In a further embodiment the adiponectin polypeptide comprises the amino acid sequence of SEQ ID NO:2. In a further embodiment the adiponectin polypeptide is consisting essentially of a globular domain. In a further embodiment the adiponectin polypeptide is consisting essentially of a collagen and a globular domain. In a further embodiment the adiponectin polypeptide is consisting essentially of the amino acid sequence of SEQ ID NO:10. In a further embodiment the adiponectin polypeptide is consisting essentially of the amino acid sequence of SEQ ID NO:1. In a further embodiment the adiponectin polypeptide is consisting essentially of the amino acid sequence of SEQ ID NO:12. In a further embodiment the adiponectin polypeptide is consisting essentially of the amino acid sequence of SEQ ID NO:13. In a further embodiment the adiponectin polypeptide is consisting essentially of the amino acid sequence of SEQ ID NO:6. In a further embodiment the adiponectin polypeptide is consisting essentially of the amino acid sequence of SEQ ID NO:5. In a further embodiment the adiponectin polypeptide is consisting essentially of the amino acid sequence of SEQ ID NO:4. In a further embodiment the adiponectin polypeptide is consisting essentially of the amino acid sequence of SEQ ID NO:3. In a further embodiment the adiponectin polypeptide is consisting essentially of the amino acid sequence of SEQ ID NO:2.

Typically, the adiponectin polypeptide is selected from any one of SEQ ID NO:2, 3, 4, 5, 10, 11, 12, or 13, as well as sequences that differs from any one of the specified sequences, in one or more substitution(s), preferably from one to eleven, more preferably from one to eight. In one embodiment the adiponectin polypeptide is selected from any one of SEQ ID NO:3, 10, 12, or 13, as well as sequences that differs from any one of the specified sequences in one to eleven substitutions. In another embodiment the adiponectin polypeptide is selected from any one of SEQ ID NO: 3, 10, 12, or 13, as well as sequences that differs from any one of the specified sequences in one to eight substitutions, such as 1-6 substitutions.

In a particular embodiment the adiponectin polypeptide is selected from any one of SEQ ID NO:2, 3, 4, 5, 10, 11, 12, or 13, as well as sequences that differs from any one of the specified sequences, in one or more substitutions, and comprises one to four lysine residues selected from any one of the positions K65, K68, K77, or K101. In a further embodiment the adiponectin polypeptide is selected from any one of SEQ ID NO:2, 3, 4, 5, 10, 11, 12, or 13, preferably 3, 10, 12, or 13. In a alternative embodiment the adiponectin polypeptide is selected from sequences that differs from any one of the SEQ ID NO:2, 3, 4, 5, 10, 11, 12, or 13, preferably 3, 10, 12, or 13, in one or more substitutions, preferably from one to eleven, more preferably from one to eight, such as 1-6. In a further embodiment the adiponectin polypeptide comprises at least one lysine residue selected from any one of the positions K65, K68, K77, or K101. As mentioned above when produced in a eucaryotic cell, such as a mammalian cell, lysine residues in the collagen domain are hydroxylated and glycosylated. Typically, the lysine residues are hydroxylated and glycosylated. In a further embodiment the adiponectin polypeptide comprises one lysine residue selected from any one of the positions K65, K68, K77, or K101, preferably K101, and preferably the position is hydroxylated and glycosylated, such as glyco-hydroxy-K101. In a further embodiment the adiponectin polypeptide comprises two lysine residues selected from any one of the positions K65, K68, K77, or K101, preferably K77 and K101, and preferably both of the positions are hydroxylated and glycosylated, such as glyco-hydroxy-K77 and glyco-hydroxy-K101. In a further embodiment the adiponectin polypeptide comprises three lysine residues selected from any one of the positions K65, K68, K77, or K101, preferably K68, K77 and K101, and preferably all three of the positions are hydroxylated and glycosylated, such as glyco-hydroxy-K68, glyco-hydroxy-K77 and glyco-hydroxy-K101. In a further embodiment the adiponectin polypeptide comprises four lysine residues selected from positions K65, K68, K77, and K101, and preferably all four of the positions are hydroxylated and glycosylated.

In a still further embodiment the adiponectin polypeptide is selected from any one of the adiponectin polypeptide fragments described in the above section “Adiponectin polypeptide fragment(s) of the invention”. Each of the described adiponectin polypeptide fragments is considered an embodiment suitable as the adiponectin polypeptide part of the conjugate.

Accordingly, one example of a preferred aspect of the conjugate is a conjugate comprising

an adiponectin polypeptide fragment comprising a globular domain and a collagen domain, wherein the globular domain comprises an amino acid sequence from position A 108 to N244 as indicated in SEQ ID NO:1 as well as sequences that differs from the amino acid sequence in one or more substitution(s), and wherein the collagen domain comprises from 7 amino acids corresponding to position K101 as indicated in SEQ ID NO:1 to 56 amino acids corresponding to position A52 as indicated in SEQ ID NO:1, and wherein the collagen domain comprises a lysine which is hydroxylated and glycosylated, and

a first non-polypeptide moiety covalently attached to the adiponectin polypeptide fragment,

wherein the adiponectin polypeptide fragment comprises an amino acid residue having an attachment group for said first non-polypeptide moiety, wherein said amino acid residue is a surface exposed amino acid residue. As mentioned above the surface exposed amino acid residue is selected from any one of E82, T83, G84, V85, P86, A88, E89, P91, R92, F94, P95, 197, Q98, R100, K101, E103, P104, G105, E106, G107, A108, Y109, V110, Y111, R112, E120, T121, Y122, V123, T124, I125, P126, N127, M128, R131, T133, K134, I135, Q139, N141, D144, G145, S146, T147, K149, H151, N153, P155, Y167, M168, K169, D170, K178, D179, K180, A181, F184, Y186, D187, Q188, Y189, Q190, E191, N192, N193, V194, D195, H204, E206, V207, G208, Q210, V215, Y216, G217, E218, G219, E220, R221, N222, G223, L224, Y225, A226, D227, N228, D229, N230, H241, D242, T243, or N244 relative to human adiponectin, preferably any one of A108, Y109, V110, Y111, R112, E120, T121, Y122, V123, T124, I125, P126, N127, M128, R131, T133, K134, I135, Q139, N141, D144, G145, S146, T147, K149, H151, N153, P155, Y167, M168, K169, D170, K178, D179, K180, A181, F184, Y186, Q188, Y189,.Q190, E191, N192, N193, V194, H204, E206, V207, G208, Q210, V215, Y216, G217, E218, G219, E220, R221, N222, L224, Y225, D227, N228, D229, N230, H241, D242, T243, or N244. However, particular a preferred amino acid residue having an attachment group for said first non-polypeptide moiety is selected from a lysine, aspartic acid, or glutamic acid. In this respect the surface exposed amino acid residue may be selected from any one of E120, K134, D 144, K149, K169, D170, K172, K177, K178, D179, K180, E191, E206, D209, E218, E220, D227, D229, D231, or D242, such as from any one of E120, E191, E206, E218, or E220, or from any one of K134, K149, K169, K172, K177, K178, or K180, or from any one of D144, D170, D179, D209, D227, D229, D231, or D242.

It should be clear that the surface exposed amino acid residue having an attachment group for the first non-polypeptide moiety may either be located in the globular domain or in the collagen domain, or in case of more than one non-polypeptide moiety being attached they may be located in the globular domain or in the collagen domain, or in both the globular domain and the collagen domain.

Accordingly in a further embodiment the attachment group is located in the globular domain. In a further embodiment the adiponectin polypeptide further comprises a collagen domain. In one embodiment the attachment group is located in the collagen domain. If only one non-polypeptide is attached then it may be in the globular domain or in the collagen domain. If more than one, such as two non-polypeptides, are attached then one may be located in the collagen domain and one in the globular domain, or both may be in the collagen domain, or both may be in the globular domain.

In a further embodiment the adiponectin polypeptide comprises a non-homologous domain.

In a further embodiment the adiponectin polypeptide comprises a signal peptide.

In a further embodiment the adiponectin polypeptide is isolated.

In a further embodiment only one first non-polypeptide moiety is attached to the adiponectin polypeptide.

In a further embodiment the conjugate of the invention is mono pegylated.

In a further embodiment the first non-polypeptide moiety is selected from a polymer molecule, a lipophilic compound, and an organic derivatizing agent.

In a further embodiment the first non-polypeptide moiety is selected from a polymer molecule.

In a further embodiment the amino acid residue having the attachment group for said first non-polypeptide moiety is selected from a lysine, aspartic acid, or glutamic acid. In this respect the surface exposed amino acid residue may be selected from any one of K65, K68, K77, K101, E120, K134, D144, K149, K169, D170, K172, K177, K178, D179, K180, D187, E191, D195, E206, D209, E218, E220, D227, D229, D231, or D242. Typically, the surface exposed amino acid residue may be selected from any one of E120, K134, D144, K149, K169, D170, K172, K177, K178, D179, K180, E191, E206, D209, E218, E220, D227, D229, D231, or D242, preferably from any one of E120, K134, D144, K149, K169, D170, K178, D179, K180, E191, E206, E218, E220, D227, D229, or D242, more preferably from any one of E120, D144, K169, K178, D179, K180, E191, E206, E218, E220, D227, or D229, in particular from any one of E120, K169, E191, E218, E220, D227, or D229.

In a further embodiment the first non-polypeptide moiety is a polymer, typically a linear or branched polyethylene glycol. Such polymers are available from Shearwater, SunBio, Pierce, or Enzon.

In a further embodiment the polymer has a molecular weight of from lkDa to 200 kDa (kDa is a well known abbreviation and means kilo Dalton). In a still further embodiment the polymer has a molecular weight of from 2 kDa to 95 kDa. In a still further embodiment the polymer has a molecular weight of from 5 kDa to 80 kDa. In a still further embodiment the polymer has a molecular weight of from 12 kDa to 60 kDa, such as 5-20 kDa, 1240 kDa, 2040 kDa, 5 kDa, 10 kDa, 12 kDa, or 20 kDa.

In a further embodiment the amino acid residue having the attachment group is a lysine residue. Such lysine residue may be present in the non-homologous, collagen or globular domain, depending on the length of the adiponectin polypeptide. Typically, a part of the collagen domain linked to the globular domain will contain one to four lysine residues, that is positions K65, K68, K77, or K101. For instance, the sequence of SEQ ID NO:3 has four lysines in the collagen domain, the sequence of SEQ ID NO:4 has one lysine in the collagen domain, the sequence of SEQ ID NO:5 has one lysine in the collagen domain, the sequence of SEQ ID NO:10 has one lysine in the collagen domain, the sequence of SEQ ID NO:11 has one lysine in the collagen domain, the sequence of SEQ ID NO:12 has four lysines in the collagen domain, and the sequence of SEQ ID NO:13 has four lysines in the collagen domain.

When a lysine intended as the amino acid residue having the attachment group is located in the collagen domain of the adiponectin polypeptide then the lysine may be hydroxylated and glycosylated if produced in eg. a mammalian cell or may be free of any such glyco-hydroxy groups. If the lysine is hydroxylated and glycosylated then it is not preferred as an attachment group, although such glyco-hydroxy group could be attached to a polymer such as a PEG, eg. by using a mPEG-AMINE, cf. also the section “Conjugate of the invention comprising a second non-polypeptide moiety”. Thus, if it is intended that a lysine located in the collagen domain of the adiponectin polypeptide should be conjugated to a non-polypeptide, then such adiponectin polypeptide should be expressed in a bacterial cell, such as E. Coli.

In a further embodiment the lysine is selected from any one of the positions K65, K68, K77, or K101 of the collagen domain of human adiponectin.

In a further embodiment the lysine is selected from any one of the positions K134, K149, K169, K172, K177, K178, or K180 of the globular domain of human adiponectin, preferably any one of the positions K134, K149, K169, K178, or K180.

In a further embodiment the lysine is selected from any one of the positions K65, K68, K77, K101, K134, K149, K169, K172, K177, K178, or K180 of human adiponectin, preferably any one of the positions K134, K149, K169, K178, or K180.

Typically, the lysine is selected from any one of the positions K68, K77, K101, K134, K149, K169, K172, K177, K178, or K180 of human adiponectin, however, depending on the length of the adiponectin polypeptide, the skilled person will recognize that the lysine residues may also be selected from any one of the positions K77, K101, K134, K149, K169, K172, K177, K178, or K180 of human adiponectin, in particular from any one of the positions K101, K134, K149, K169, K172, K177, K178, or K180 of human adiponectin, preferably any one of the positions K134, K149, K169, K178, or K180.

In a further embodiment the polymer molecule is selected from the group consisting of SS-PEG, NPC-PEG, aldehyd-PEG, mPEG-SPA, mPEG-SBA, PEG-SCM, mPEG-BTC (All available from Shearwater), and SC-PEG (available from Enzon).

In a further embodiment the polymer molecule is selected from the group consisting of 5k-PEG-SCM, 12k-PEG-SCM, 20k-PEG-SCM, 5k-PEG-SPA, 12k-PEG-SPA, 20k-PEG-SPA. (All available from Shearwater).

In a further embodiment the conjugate further comprises a second non-polypeptide moiety selected from the group consisting of a polymer molecule, a lipophilic compound, a sugar moiety and an organic derivatizing agent. The second non-polypeptide moiety is different from the first non-polypeptide.

In a further embodiment the second non-polypeptide moiety is selected from a polymer molecule.

In a further embodiment the amino acid residue having the attachment group for said second non-polypeptide moiety is selected from a lysine, aspartic acid, glutamic acid or cysteine residue. In this respect the surface exposed amino acid residue may be selected from any one of K65, K68, K77, K101, E120, K134, D144, K149, K169, D170, K172, K177, K178, D179, K180, D187, E191, D195, E206, D209, E218, E220, D227, D229, D231, or D242. Typically, the surface exposed amino acid residue may be selected from any one of E120, K134, D144, K149, K169, D170, K172, K177, K178, D179, K180, E191, E206, D209, E218, E220, D227, D229, D231, or D242, preferably from any one of E120, K134, D144, K149, K169, D170, K178, D179, K180, E191, E206, E218, E220, D227, D229, or D242, more preferably from any one of E120, D144, K169, K178, D179, K180, E191, E206, E218, E220, D227, or D229, in particular from any one of E120, K169, E191, E218, E220, D227, or D229.

In a further embodiment the second non-polypeptide moiety is a polymer, typically a linear or branched polyethylene glycol.

In a further embodiment the amino acid sequence of the adiponectin polypeptide further comprises at least one removed lysine residue.

In a further embodiment one to four lysine residues selected from any one of the positions K65, K68, K77, or K101 of the collagen domain of human adiponectin is/are removed.

In a further embodiment one to six lysine residues selected from any one of the positions K134, K149, K169, K172, K177, K178, or K180 of the globular domain of wild-type human adiponectin is/are removed.

Such lysine residues may be removed from the collagen and/or globular domain, depending on the length of the adiponectin polypeptide. The skilled person will understand that the group of lysines to select from will depend on whether the full collagen domain or only a fragment thereof is present in the adiponectin polypeptide, and thus whether the group of lysine residues are the positions K65, K68, K77, or K101 of the collagen domain of human adiponectin and positions K134, K149, K169, K172, K177, K178, or K180 of the globular domain of human adiponectin, or a smaller group, such as K77, or K101 of the collagen domain and positions K134, K149, K169, K172, K177, K178, or K180 of the globular domain, or even a smaller group, such as K101 of the collagen domain and positions K134, K149, K169, K172, K177, K178, or K180 of the globular domain. Obviously, at least one lysine should be present in the adiponectin polypeptide in order to make possible the conjugation to a lysine.

Second Group of Conjugate(s) of the Invention

In a further aspect the invention relates to a conjugate comprising an adiponectin polypeptide, and a first non-polypeptide moiety covalently attached to the adiponectin polypeptide, wherein the adiponectin polypeptide comprises an amino acid residue having an attachment group for said first non-polypeptide moiety, wherein said amino acid residue is a cysteine residue.

In a further aspect the invention relates to a conjugate consisting essentially of an adiponectin polypeptide, and a first non-polypeptide moiety covalently attached to the adiponectin polypeptide, wherein the adiponectin polypeptide comprises an amino acid residue having an attachment group for said first non-polypeptide moiety, wherein said amino acid residue is a cysteine residue.

In a further aspect the invention relates to a conjugate comprising an adiponectin polypeptide selected from SEQ ID NO:5 or 6, and one first non-polypeptide moiety covalently attached to the adiponectin polypeptide, wherein the adiponectin polypeptide comprises an amino acid residue having an attachment group for said first non-polypeptide moiety, wherein said amino acid residue is a cysteine residue.

By using a cysteine residue which is already present in the wildtype molecule having a sulfhydryl attachment group for a non-polypeptide moiety it will not be necessary to make mutations, however, this does not exclude that mutations can be made, provided that the conjugate maintain biological activity, and thereby its usefulness for treating eg. impaired glucose tolerance, type 2 diabetes, syndrome X, obesity, a cardiovascular disease, such as atherosclerosis, or dyslipidemia, such activity could be tested in a relevant animal model, such as mouse models of insulin resistance and diabetes, such as db/db or ob/ob mice, or rat models such as zucker rats, or could be tested in a relevant in vitro assay, such as any one of the Test Assays A, B, or C described in the experimental section. The wildtype adiponectin polypeptide has two cysteine residues, that is, position C36 and C152 relative to SEQ ID NO:1.

The use of C152 relative to SEQ ID NO:1 in the globular domain of human adiponectin for conjugation to a non-polypeptide moiety is not an obvious choice, since this cysteine does not have its sulfhydryl group (—SH) exposed to the surface of human adiponectin, cf. the experimental section under “Surface exposure”. Such a non-polypeptide moiety is typically selected from a polymer molecule, a lipophilic compound, or an organic derivatizing agent. Suitable methods for attaching a non-polypeptide moiety to a cysteine residue in the globular domain of human adiponectin are well known to the skilled person. The preferred methods of attaching a non-polypeptide moiety selected from a polymer molecule, a lipophilic compound, or an organic derivatizing agent are described in more detail in the section “Methods of preparing a conjugate of the invention” hereinafter.

The adiponectin polypeptide should have a globular domain, such as indicated in the sequence of human adiponectin (108-244) (shown in SEQ ID NO:6). The adiponectin polypeptide part of the conjugate comprises the globular domain having the amino acid sequence shown in SEQ ID NO:6 as well as analogues thereof, including fragments. As mentioned also analogues are comprised, in particular analogues that differs in 1, 2, 3, 4, 5, 6, 7, 8, 9, 10, 11, 12, 13, 14, or 15 amino acid residues relative to the amino acid sequence shown in SEQ ID NO:6.

Thus, in a further embodiment the adiponectin polypeptide part of the conjugate comprises a globular domain, preferably a collagen and a globular domain. In a still further embodiment the adiponectin polypeptide comprises the amino acid sequence of SEQ ID NO:10. In a further embodiment the adiponectin polypeptide comprises the amino acid sequence of SEQ ID NO:1. In a further embodiment the adiponectin polypeptide comprises the amino acid sequence of SEQ ID NO:12. In a further embodiment the adiponectin polypeptide comprises the amino acid sequence of SEQ ID NO:13. In a further embodiment the adiponectin polypeptide comprises the amino acid sequence of SEQ ID NO:6. In a further embodiment the adiponectin polypeptide comprises the amino acid sequence of SEQ ID NO:5. In a further embodiment the adiponectin polypeptide comprises the amino acid sequence of SEQ ID NO:4. In a further embodiment the adiponectin polypeptide comprises the amino acid sequence of SEQ ID NO:3. In a further embodiment the adiponectin polypeptide comprises the amino acid sequence of SEQ ID NO:2. In a further embodiment the adiponectin polypeptide is consisting essentially of a globular domain. In a further embodiment the adiponectin polypeptide is consisting essentially of a collagen and a globular domain. In a further embodiment the adiponectin polypeptide is consisting essentially of the amino acid sequence of SEQ ID NO:10. In a further embodiment the adiponectin polypeptide is consisting essentially of the amino acid sequence of SEQ ID NO:1. In a further embodiment the adiponectin polypeptide is consisting essentially of the amino acid sequence of SEQ ID NO:12. In a further embodiment the adiponectin polypeptide is consisting essentially of the amino acid sequence of SEQ ID NO:13. In a further embodiment the adiponectin polypeptide is consisting essentially of the amino acid sequence of SEQ ID NO:6. In a further embodiment the adiponectin polypeptide is consisting essentially of the amino acid sequence of SEQ ID NO:5. In a further embodiment the adiponectin polypeptide is consisting essentially of the amino acid sequence of SEQ ID NO:4. In a further embodiment the adiponectin polypeptide is consisting essentially of the amino acid sequence of SEQ ID NO:3. In a further embodiment the adiponectin polypeptide is consisting essentially of the amino acid sequence of SEQ ID NO:2.

Typically, the adiponectin polypeptide is selected from any one of SEQ ID NO:3, 4, 5, 10, 11, 12, or 13, as well as sequences that differs from any one of the specified sequences, in one or more substitution(s), preferably from one to eleven, such as from one to eight. In one embodiment the adiponectin polypeptide is selected from any one of SEQ ID NO:3, 10, 12, or 13, as well as sequences that differs from any one of the specified sequences in one to eleven substitutions, such as one to eight substitutions, eg. 1-6 substitutions.

In a particular embodiment the adiponectin polypeptide is selected from any one of SEQ ID NO:3, 4, 5, 10, 11, 12, or 13, as well as sequences that differs from any one of the specified sequences, in one or more substitutions, and comprises one to four lysine residues selected from any one of the positions K65, K68, K77, or K101. In a further embodiment the adiponectin polypeptide is selected from any one of SEQ ID NO:3, 4, 5, 10, 11, 12, or 13, preferably 3, 10, 12, or 13. In a alternative embodiment the adiponectin polypeptide is selected from sequences that differs from any one of the SEQ ID NO:3, 4, 5, 10, 11, 12, or 13, preferably 3, 10, 12, or 13, in one or more substitutions, preferably from one to eleven substitutions, such as one to eight substitutions, eg. 1-6 substitutions. In a further embodiment the adiponectin polypeptide comprises at least one lysine residue selected from any one of the positions K65, K68, K77, or K101. As mentioned above when produced in a eucaryotic cell, such as a mammalian cell, lysine residues in the collagen domain are hydroxylated and glycosylated. Typically, the lysine residues are hydroxylated and glycosylated. In a further embodiment the adiponectin polypeptide comprises one lysine residue selected from any one of the positions K65, K68, K77, or K101, preferably K101, and preferably the position is hydroxylated and glycosylated, such as glyco-hydroxy-K101. In a further embodiment the adiponectin polypeptide comprises two lysine residues selected from any one of the positions K65, K68, K77, or K101, preferably K77 and K101, and preferably both of the positions are hydroxylated and glycosylated, such as glyco-hydroxy-K77 and glyco-hydroxy-K101. In a further embodiment the adiponectin polypeptide comprises three lysine residues selected from any one of the positions K65, K68, K77, or K101, preferably K68, K77 and K101, and preferably all three of the positions are hydroxylated and glycosylated, such as glyco-hydroxy-K68, glyco-hydroxy-K77 and glyco-hydroxy-K101. In a further embodiment the adiponectin polypeptide comprises four lysine residues selected from positions K65, K68, K77, and K101, and preferably all four of the positions are hydroxylated and glycosylated.

In a still further embodiment the adiponectin polypeptide is selected from any one of the adiponectin polypeptide fragments described in the above section “Adiponectin polypeptide fragment(s) of the invention”. Each of the described adiponectin polypeptide fragments is considered an embodiment suitable as the adiponectin polypeptide part of the conjugate.

Accordingly, one example of a preferred aspect of the conjugate is a conjugate comprising

an adiponectin polypeptide fragment comprising a globular domain and a collagen domain, wherein the globular domain comprises an amino acid sequence from position A108 to N244 as indicated in SEQ ID NO:1 as well as sequences that differs from the amino acid sequence in one or more substitution(s), and wherein the collagen domain comprises from 7 amino acids corresponding to position K101 as indicated in SEQ ID NO:1 to 56 amino acids corresponding to position A52 as indicated in SEQ 11) NO:1, and wherein the collagen domain comprises a lysine which is hydroxylated and glycosylated, and

a first non-polypeptide moiety covalently attached to the adiponectin polypeptide fragment,

wherein the adiponectin polypeptide fragment comprises an amino acid residue having an attachment group for said first non-polypeptide moiety, wherein said amino acid residue is a cysteine residue.

In a further embodiment the cysteine is Cys152 in the globular domain of human adiponectin.

In a further embodiment the adiponectin polypeptide comprises a collagen domain.

In a further embodiment the adiponectin polypeptide comprises a non-homologous domain. In a further embodiment the cysteine is Cys36 in the non-homologous domain of human adiponectin.

In a further embodiment the adiponectin polypeptide comprises a signal peptide.

In a further embodiment the adiponectin polypeptide is isolated.

In a further embodiment only one first non-polypeptide moiety is attached to the adiponectin polypeptide.

In a further embodiment the conjugate of the invention is mono pegylated.

In a further embodiment the first non-polypeptide moiety is selected from a polymer molecule, a lipophilic compound, and an organic derivatizing agent.

In a further embodiment the first non-polypeptide moiety is a polymer, typically a linear or branched polyethylene glycol.

In a further embodiment the polymer has a molecular weight of from lkDa to 200 kDa (kDa is a well known abbreviation and means kilo Dalton). In a still further embodiment the polymer has a molecular weight of from 2 kDa to 95 kDa. In a still further embodiment the polymer has a molecular weight of from 5 kDa to 80 kDa. In a still further embodiment the polymer has a molecular weight of from 12 kDa to 60 kDa, such as 1240 kDa, 2040 kDa, 5 kDa, 12 kDa, or 20 kDa.

In a further embodiment the polymer molecule is selected from the group consisting of mPEG(MAL), mPEG2(MAL), mPEG-OPSS, PEG-vinylsulphone, OPSS-PEG-hydrazide in combination with mPEG-ALD. In a further embodiment the polymer molecule is selected from the group consisting of 5k-mPEG(MAL), 20k-mPEG(MAL), 40k-mPEG2(MAL), 5k-mPEG-OPSS, 10k-mPEG-OPSS, 20k-mPEG-OPSS, OPSS-PEG₂k-hydrazide in combination with mPEG₃₀kD-ALD.

In a further embodiment the conjugate further comprises a second non-polypeptide moiety selected from the group consisting of a polymer molecule, a lipophilic compound, and an organic derivatizing agent. The second non-polypeptide moiety is different from the first non-polypeptide.

In a further embodiment the second non-polypeptide moiety is selected from a polymer molecule.

In a further embodiment the amino acid residue having the attachment group for said second non-polypeptide moiety is selected from a lysine, aspartic acid, glutamic acid or cysteine residue.

In a further embodiment the second non-polypeptide moiety is a polymer, typically a linear or branched polyethylene glycol.

In a further embodiment the amino acid sequence of the adiponectin polypeptide further comprises at least one removed lysine residue.

In a further embodiment one to four lysine residues selected from any one of the positions K65, K68, K77, or KIO of the collagen domain of human adiponectin is/are removed.

In a further embodiment one to six lysine residues selected from any one of the positions K134, K149, K169, K172, K177, K178, or K180 of the globular domain of wild-type human adiponectin is/are removed.

Such lysine residues may be removed from the collagen and/or globular domain, depending on the length of the adiponectin polypeptide. The skilled person will understand that the group of lysines to select from will depend on whether the full collagen domain or only a fragment thereof is present in the adiponectin polypeptide, and thus whether the group of lysine residues are the positions K65, K68, K77, or K101 of the collagen domain of human adiponectin and positions K134, K149, K169, K172, K177, K178, or K180 of the globular domain of human adiponectin, or a smaller group, such as K77, or K101 of the collagen domain and positions K134, K149, K169, K172, K177, K178, or K180 of the globular domain, or even a smaller group, such as K101 of the collagen domain and positions K134, K149, K169, K172, K177, K178, or K180 of the globular domain. If desired to introduce a second non-polypeptide moiety by conjugating it to a lysine, then obviously, at least one lysine should be present in the adiponectin polypeptide in order to make possible the conjugation to a lysine.

Third Group of Conjugate(s) of the Invention

In a further aspect the invention relates to a conjugate comprising an adiponectin polypeptide, and a first non-polypeptide moiety covalently attached to the adiponectin polypeptide, wherein the adiponectin polypeptide comprises an amino acid residue having an attachment group for said first non-polypeptide moiety, wherein the amino acid residue is the N-terminal amino acid residue.

In a further aspect the invention relates to a conjugate consisting essentially of an adiponectin polypeptide, and a first non-polypeptide moiety covalently attached to the adiponectin polypeptide, wherein the adiponectin polypeptide comprises an amino acid residue having an attachment group for said first non-polypeptide moiety, wherein the amino acid residue is the N-terminal amino acid residue.

By using the N-terminal amino acid residue which is already present in the wildtype molecule having an attachment group for a non-polypeptide moiety it will not be necessary to make mutations, however, this does not exclude that mutations can be made, provided that the conjugate maintain biological activity, and thereby its usefulness for treating eg. impaired glucose tolerance, type 2 diabetes, syndrome X, obesity, a cardiovascular disease, such as atherosclerosis, or dyslipidemia, such activity could be tested in a relevant animal model, such as mouse models of insulin resistance and diabetes, such as db/db or ob/ob mice, or rat models such as zucker rats, or could be tested in a relevant in vitro assay, such as any one of the Test Assays A, B, or C described in the experimental section.

Such a non-polypeptide moiety is typically selected from a polymer molecule, a lipophilic compound, or an organic derivatizing agent. Suitable methods for attaching a non-polypeptide moiety to the N-terminal amino acid residue in the adiponectin polypeptide are well known to the skilled person. The preferred methods of attaching a non-polypeptide moiety selected from a polymer molecule, a lipophilic compound, or an organic derivatizing agent are described in more detail in the section “Methods of preparing a conjugate of the invention” hereinafter.

The adiponectin polypeptide should have a globular domain, such as indicated in the sequence of human adiponectin (108-244) (shown in SEQ ID NO:6). The adiponectin polypeptide part of the conjugate comprises the globular domain having the amino acid sequence shown in SEQ ID NO:6 as well as analogues thereof, including fragments. As mentioned also analogues are comprised, in particular analogues that differs in 1, 2, 3, 4, 5, 6, 7, 8, 9, 10, 11, 12, 13, 14, or 15 amino acid residues relative to the amino acid sequence shown in SEQ ID NO:6.

Thus, in a further embodiment the adiponectin polypeptide comprises a globular domain, preferably a collagen and a globular domain. In a still further embodiment the adiponectin polypeptide comprises the amino acid sequence of SEQ ID NO:10. In a further embodiment the adiponectin polypeptide comprises the amino acid sequence of SEQ ID NO:1. In a further embodiment the adiponectin polypeptide comprises the amino acid sequence of SEQ ID NO:12. In a further embodiment the adiponectin polypeptide comprises the amino acid sequence of SEQ ID NO:13. In a further embodiment the adiponectin polypeptide comprises the amino acid sequence of SEQ ID NO:6. In a further embodiment the adiponectin polypeptide comprises the amino acid sequence of SEQ ID NO:5. In a further embodiment the adiponectin polypeptide comprises the amino acid sequence of SEQ ID NO:4. In a further embodiment the adiponectin polypeptide comprises the amino acid sequence of SEQ ID NO:3. In a further embodiment the adiponectin polypeptide comprises the amino acid sequence of SEQ ID NO:2. In a further embodiment the adiponectin polypeptide is consisting essentially of a globular domain. In a further embodiment the adiponectin polypeptide is consisting essentially of a collagen and a globular domain. In a further embodiment the adiponectin polypeptide is consisting essentially of the amino acid sequence of SEQ ID NO:10. In a further embodiment the adiponectin polypeptide is consisting essentially of the amino acid sequence of SEQ ID NO:1. In a further embodiment the adiponectin polypeptide is consisting essentially of the amino acid sequence of SEQ ID NO:12. In a further embodiment the adiponectin polypeptide is consisting essentially of the amino acid sequence of SEQ ID NO:13. In a further embodiment the adiponectin polypeptide is consisting essentially of the amino acid sequence of SEQ ID NO:6. In a further embodiment the adiponectin polypeptide is consisting essentially of the amino acid sequence of SEQ f) NO:5. In a further embodiment the adiponectin polypeptide is consisting essentially of the amino acid sequence of SEQ ID NO:4. In a further embodiment the adiponectin polypeptide is consisting essentially of the amino acid sequence of SEQ ID NO:3. In a further embodiment the adiponectin polypeptide is consisting essentially of the amino acid sequence of SEQ ID NO:2.

Typically, the adiponectin polypeptide is selected from any one of SEQ ID NO:2, 3, 4, 5, 10, 11, 12, or 13, as well as sequences that differs from any one of the specified sequences, in one or more substitution(s), preferably from one to eleven, such as one to eight. In one embodiment the adiponectin polypeptide is selected from any one of SEQ ID NO: 3, 10, 12, or 13, as well as sequences that differs from any one of the specified sequences in one to eleven, such as one to eight substitutions, eg. 1-6 substitutions.

In a particular embodiment the adiponectin polypeptide is selected from any one of SEQ ID NO:3, 4, 5, 10, 11, 12, or 13, as well as sequences that differs from any one of the specified sequences, in one or more substitutions, and comprises one to four lysine residues selected from any one of the positions K65, K68, K77, or K101. In a further embodiment the adiponectin polypeptide is selected from any one of SEQ ID NO:3, 4, 5, 10, 11, 12, or 13, preferably 3, 10, 12, or 13. In a alternative embodiment the adiponectin polypeptide is selected from sequences that differs from any one of the SEQ ID NO:3, 4, 5, 10, 11, 12, or 13, preferably 3, 10, 12, or 13, in one or more substitutions, preferably from one to eleven, such as one to eight, eg. 1-6. In a further embodiment the adiponectin polypeptide comprises at least one lysine residue selected from any one of the positions K65, K68, K77, or K101. As mentioned above when produced in a eucaryotic cell, such as a mammalian cell, lysine residues in the collagen domain are hydroxylated and glycosylated. Typically the lysine residues are hydroxylated and glycosylated. In a further embodiment the adiponectin polypeptide comprises one lysine residue selected from any one of the positions K65, K68, K77, or K101, preferably K101, and preferably the position is hydroxylated and glycosylated, such as glyco-hydroxy-K101. In a further embodiment the adiponectin polypeptide comprises two lysine residues selected from any one of the positions K65, K68, K77, or K101, preferably K77 and K101, and preferably both of the positions are hydroxylated and glycosylated, such as glyco-hydroxy-K77 and glyco-hydroxy-K101. In a further embodiment the adiponectin polypeptide comprises three lysine residues selected from any one of the positions K65, K68, K77, or K101, preferably K68, K77 and K101, and preferably all three of the positions are hydroxylated and glycosylated, such as glyco-hydroxy-K68, glyco-hydroxy-K77 and glyco-hydroxy-K101. In a further embodiment the adiponectin polypeptide comprises four lysine residues selected from positions K65, K68, K77, and K101, and preferably all four of the positions are hydroxylated and glycosylated.

In a still further embodiment the adiponectin polypeptide is selected from any one of the adiponectin polypeptide fragments described in the above section “Adiponectin polypeptide fragment(s) of the invention”. Each of the described adiponectin polypeptide fragments is considered an embodiment suitable as the adiponectin polypeptide part of the conjugate.

Accordingly, one example of a preferred aspect of the conjugate is a conjugate comprising

an adiponectin polypeptide fragment comprising a globular domain and a collagen domain, wherein the globular domain comprises an amino acid sequence from position A108 to N244 as indicated in SEQ ID NO:1 as well as sequences that differs from the amino acid sequence in one or more substitution(s), and wherein the collagen domain comprises from 7 amino acids corresponding to position K101 as indicated in SEQ ID NO:1 to 56 amino acids corresponding to position A52 as indicated in SEQ ID NO:1, and wherein the collagen domain comprises a lysine which is hydroxylated and glycosylated, and

a first non-polypeptide moiety covalently attached to the adiponectin polypeptide fragment,

wherein the adiponectin polypeptide fragment comprises an amino acid residue having an attachment group for said first non-polypeptide moiety, wherein the amino acid residue is the N-terminal amino acid residue.

In a further embodiment the adiponectin polypeptide comprises a collagen domain.

In a further embodiment the adiponectin polypeptide comprises a non-homologous domain.

In a further embodiment the adiponectin polypeptide comprises a signal peptide.

In a further embodiment the adiponectin polypeptide is isolated.

In a further embodiment only one first non-polypeptide moiety is attached to the adiponectin polypeptide.

In a further embodiment the conjugate of the invention is mono pegylated.

In a further embodiment the first non-polypeptide moiety is selected from a polymer molecule, a lipophilic compound, and an organic derivatizing agent.

In a further embodiment the first non-polypeptide moiety is a polymer, typically a linear or branched polyethylene glycol.

In a further embodiment the polymer has a molecular weight of from 1 kDa to 200 kDa (kDa is a well known abbreviation and means kilo Dalton). In a still further embodiment the polymer has a molecular weight of from 2 kDa to 95 kDa. In a still further embodiment the polymer has a molecular weight of from 5 kDa to 80 kDa. In a still further embodiment the polymer has a molecular weight of from 12 kDa to 60 kDa, such as 5-20 kDa, 1240 kDa, 2040 kDa, 5 kDa, 12 kDa, or 20 kDa.

In a further embodiment the polymer molecule is selected from the group consisting of SS-PEG, NPC-PEG, aldehyd-PEG, mPEG-SPA, mPEG-SBA, PEG-SCM, mPEG-OPSS, mPEG-BTC (All available from Shearwater), and SC-PEG.

In a further embodiment the polymer molecule is selected from the group consisting of 5k-PEG-SCM, 5k-mPEG-OPSS, 10k-mPEG-OPSS, 20k-mPEG-OPSS, 12k-PEG-SCM, 20k-PEG-SCM, 5k-mPEG-ALD, 20k-mPEG-ALD, 30k-mPEG-ALD, and 40k-mPEG2-ALD. (All available from Shearwater)

In a further embodiment the conjugate further comprises a second non-polypeptide moiety selected from the group consisting of a polymer molecule, a lipophilic compound, and an organic derivatizing agent. The second non-polypeptide moiety is different from the first non-polypeptide.

In a further embodiment the second non-polypeptide moiety is selected from a polymer molecule.

In a further embodiment the amino acid residue having the attachment group for said second non-polypeptide moiety is selected from a lysine, aspartic acid, glutamic acid or cysteine residue.

In a further embodiment the second non-polypeptide moiety is a polymer, typically a linear or branched polyethylene glycol.

In a further embodiment the amino acid sequence of the adiponectin polypeptide further comprises at least one removed lysine residue.

In a further embodiment one to four lysine residues selected from any one of the positions K65, K68, K77, or K101 of the collagen domain of human adiponectin is/are removed.

In a further embodiment one to six lysine residues selected from any one of the positions K134, K149, K169, K172, K177, K178, or K180 of the globular domain of wild-type human adiponectin is/are removed.

Such lysine residues may be removed from the collagen and/or globular domain, depending on the length of the adiponectin polypeptide. The skilled person will understand that the group of lysines to select from will depend on whether the full collagen domain or only a fragment thereof is present in the adiponectin polypeptide, and thus whether the group of lysine residues are the positions K65, K68, K77, or K101 of the collagen domain of human adiponectin and positions K134, K149, K169, K172, K177, K178, or K180 of the globular domain of human adiponectin, or a smaller group, such as K77, or K101 of the collagen domain and positions K134, K149, K169, K172, K177, K178, or K180 of the globular domain, or even a smaller group, such as K101 of the collagen domain and positions K134, K149, K169, K172, K177, K178, or K180 of the globular domain. If desired to introduce a second non-polypeptide moiety by conjugating it to a lysine, then obviously, at least one lysine should be present in the adiponectin polypeptide in order to make possible the conjugation to a lysine.

Fourth Group of Conjugate(s) of the Invention

In a further aspect the invention relates to a conjugate comprising an adiponectin polypeptide, and a first non-polypeptide moiety covalently attached to the adiponectin polypeptide, wherein the adiponectin polypeptide comprises an amino acid residue having an attachment group for said first non-polypeptide moiety, wherein said amino acid residue has been introduced in a position that in the parent adiponectin is occupied by a surface exposed amino acid residue.

In a further aspect the invention relates to a conjugate consisting essentially of an adiponectin polypeptide, and a first non-polypeptide moiety covalently attached to the adiponectin polypeptide, wherein the adiponectin polypeptide comprises an amino acid residue having an attachment group for said first non-polypeptide moiety, wherein said amino acid residue has been introduced in a position that in the parent adiponectin is occupied by a surface exposed amino acid residue.

In a further aspect the invention relates to a conjugate comprising an adiponectin polypeptide, and one first non-polypeptide moiety covalently attached to the adiponectin polypeptide, wherein the adiponectin polypeptide comprises an amino acid residue having an attachment group for said first non-polypeptide moiety, wherein said amino acid residue has been introduced in a position that in the parent adiponectin selected from SEQ ID NO:5 or 6 is occupied by a surface exposed amino acid residue.

It is clear that the introduction of an amino acid residue in a position that in the parent adiponectin is occupied by a surface exposed amino acid residue will lead to a novel adiponectin polypeptide. Such novel adiponectin polypeptide is also intended to be comprised within the scope of the present invention.

Thus, in a further aspect the invention relates to an adiponectin polypeptide comprising an amino acid residue having an attachment group for a first non-polypeptide moiety, wherein said amino acid residue has been introduced in a position that in the parent adiponectin is occupied by a surface exposed amino acid residue.

The amino acid residue having the attachment group for the first non-polypeptide moiety is located at the surface of the adiponectin polypeptide, and typically has more than 25% of its side chain exposed to the solvent, such as more than 50% of its side chain exposed to the solvent. We believe that such positions in the globular domain may be identified on the basis of an analysis of the 3D structure of the crystal structure of the globular domain of mouse ACRP30, cf Brief Communication, “The crystal structure of a complement-1q family protein suggets an evolutionary link to tumor necrosis factor”, Shapiro et al, pp 335-338. Typically, in the globular and collagen domains all lysine residues are surface exposed. The surface exposed amino acid residues have been identified as outlined in the experimental section herein.

By introducing an amino acid residue having an attachment group for a non-polypeptide moiety in a position that in the parent adiponectin polypeptide is occupied by a surface exposed amino acid residue a novel molecule is created. Such novel adiponectin polypeptide may or may not comprise further mutations, however, this does not exclude that mutations can be made, provided that the adiponectin polypeptide or the conjugate maintain biological activity, and thereby its usefulness for treating eg. impaired glucose tolerance, type 2 diabetes, syndrome X, obesity, a cardiovascular disease, such as atherosclerosis, or dyslipidemia, such activity could be tested in a relevant animal model, such as mouse models of insulin resistance and diabetes, such as db/db or ob/ob mice, or rat models such as zucker rats, or could be tested in a relevant in vitro assay, such as any one of the Test Assays A, B, or C described in the experimental section.

In one embodiment the surface exposed amino acid residue is an amino acid residue having at least 25%, such as at least 50% of its side chain exposed to the surface. In a particular embodiment the surface exposed amino acid residue is an amino acid residue having 100% of its side chain exposed to the surface.

In a further embodiment the surface exposed amino acid residue is selected from A108, Y109, V110, Y111, R112, L119, E120, T121, Y122, V123, T124, I125, P126, N127, M128, I130, R131, T133, K134, I135, F136, Y137, N138, Q139, Q140, N141, H142, D144, G145, S146, T147, K149, H151, N153, I154, P155, Y159, A161, H163, I164, T165, Y167, M168, K169, D170, V171, K172, F176, K177, K178, D179, K180, A181, M182, F184, T185, Y186, D187, Q188, Y189, Q190, E191, N192, N193, V194, D195, Q196, S198, G199, S200, H204, E206, V207, G208, D209, Q210, W212, Q214, V215, Y216, G217, E218, G219, E220, R221, N222, G223, L224, Y225, A226, D227, N228, D229, N230, D231, T233, F234, F237, H241, D242, T243, or N244 of human adiponectin.

In a further embodiment the surface exposed amino acid residue is selected from A108, Y109, V110, Y111, R112, E120, T121, Y122, V123, T124, I125, P126, N127, M128, R131, T133, K134, I135, Q139, N141, D144, G145, S146, T147, K149, H151, N153, P155, Y167, M168, K169, D170, K178, D179, K180, A181, F184, Y186, D187, Q188, Y189, Q190, E191, N192, N193, V194, D195, H204, E206, V207, G208, Q210, V215, Y216, G217, E218, G219, E220, R221, N222, G223, L224, Y225, A226, D227, N228, D229, N230, H241, D242, T243, or N244 of human adiponectin.

In a further embodiment the surface exposed amino acid residue is selected from A108, Y109, V110, Y111, E120, T121, Y122, V123, T124, I125, P126, N127, M128, R131, Q139, N141, D144, G145, S146, N153, Y167, M168, K169, K178, D179, K180, A181, Y186, D187, Q188, Y189, Q190, E191, N192, N193, V194, D195, E206, V207, G208, V215, Y216, G217, E218, G219, E220, R221, N222, G223, L224, Y225, A226, D227, N228, D229, N230, H241, T243, or N244 of human adiponectin.

In a further embodiment the surface exposed amino acid residue is selected from A108, Y109, E120, T121, Y122, V123, T124, I125, P126, N127, Y167, M168, K169, A181, Y186, D187, Q188, Y189, Q190, E191, N192, N193, V194, D195, V215, Y216, G217, E218, G219, E220, R221, N222, G223, L224, Y225, A226, D227, N228, D229, N230, T243, or N244 of human adiponectin.

Any one of the above positions which have been identified as surface exposed amino acid residues may be substituted with an amino acid residue having an attachment group for the first non-polypeptide moiety, and such amino acid residue is typically selected from a lysine, aspartic acid, glutamic acid or cysteine residue. Each of these positions is considered an embodiment and may be made the subject of a claim, moreover, any one of these positions may be combined with any one of the embodiments hereinafter.

The identification of surface exposed amino acids in the globular domain of human adiponectin has made it possible to select the desired target for introducing an amino acid residue having an attachment group for a first non-polypeptide moiety and subsequently attaching the first non-polypeptide moiety. Such a non-polypeptide moiety is typically selected from a polymer molecule, a lipophilic compound, or an organic derivatizing agent. Suitable methods for attaching a non-polypeptide moiety to any one of the surface exposed amino acids in the globular domain of human adiponectin are well known to the skilled person. The preferred methods of attaching a non-polypeptide moiety selected from a polymer molecule, a lipophilic compound, or an organic derivatizing agent are described in more detail in the section “Methods of preparing a conjugate of the invention” hereinafter.

The adiponectin polypeptide should have a globular domain, such as indicated in the sequence of human adiponectin (108-244) (shown in SEQ ID NO:6). The adiponectin polypeptide part of the conjugate comprises the globular domain having the amino acid sequence shown in SEQ ID NO:6 as well as analogues thereof, including fragments. As mentioned also analogues are comprised, in particular analogues that differs in 1, 2, 3, 4, 5, 6, 7, 8, 9, 10, 11, 12, 13, 14, or 15 amino acid residues relative to the amino acid sequence shown in SEQ ID NO:6.

Thus, in a further embodiment the parent adiponectin polypeptide comprises a globular domain, preferably a collagen and a globular domain. In a still further embodiment the parent adiponectin comprises the amino acid sequence of SEQ ID NO:10. In a further embodiment the parent adiponectin comprises the amino acid sequence of SEQ ID NO:11. In a further embodiment the adiponectin polypeptide comprises the amino acid sequence of SEQ ID NO:12. In a further embodiment the adiponectin polypeptide comprises the amino acid sequence of SEQ ID NO:13. In a further embodiment the parent adiponectin comprises the amino acid sequence of SEQ ID NO:6. In a further embodiment the parent adiponectin comprises the amino acid sequence of SEQ ID NO:5. In a further embodiment the parent adiponectin comprises the amino acid sequence of SEQ ID NO:4. In a further embodiment the parent adiponectin comprises the amino acid sequence of SEQ ID NO:3. In a further embodiment the parent adiponectin comprises the amino acid sequence of SEQ ID NO:2. In a further embodiment the parent adiponectin consist essentially of a globular domain. In a further embodiment the parent adiponectin consist essentially of a collagen and a globular domain. In a further embodiment the parent adiponectin consist essentially of the amino acid sequence of SEQ ID NO:10. In a further embodiment the parent adiponectin consist essentially of the amino acid sequence of SEQ ID NO:11. In a further embodiment the parent adiponectin consist essentially of the amino acid sequence of SEQ ID NO:12. In a further embodiment the parent adiponectin consist essentially of the amino acid sequence of SEQ ID NO:13. In a further embodiment the parent adiponectin consist essentially of the amino acid sequence of SEQ ID NO:6. In a further embodiment the parent adiponectin consist essentially of the amino acid sequence of SEQ ID NO:5. In a further embodiment the parent adiponectin consist essentially of the amino acid sequence of SEQ ID NO:4. In a further embodiment the parent adiponectin consist essentially of the amino acid sequence of SEQ ID NO:3. In a further embodiment the parent adiponectin consist essentially of the amino acid sequence of SEQ ID NO:2.

Typically, the parent adiponectin is selected from any one of SEQ ID NO:2, 3, 4, 5, 10, 11, 12, or 13, as well as sequences that differs from any one of the specified sequences, in one or more substitution(s), preferably from one to eleven, such as one to eight. In one embodiment the adiponectin polypeptide is selected from any one of SEQ ID NO:3, 10, 12, or 13, as well as sequences that differs from any one of the specified sequences in one to eleven substitutions, such as one to eight substitutions, eg. 1-6 substitutions.

In a particular embodiment the parent adiponectin is selected from any one of SEQ ID NO:3, 4, 5, 10, 11, 12, or 13, as well as sequences that differs from any one of the specified sequences, in one or more substitutions, and comprises one to four lysine residues selected from any one of the positions K65, K68, K77, or K101. In a further embodiment the parent adiponectin is selected from any one of SEQ ID NO:3, 4, 5, 10, 11, 12, or 13, preferably 3, 10, 12, or 13. In a alternative embodiment the parent adiponectin is selected from sequences that differs from any one of the SEQ ID NO:3, 4, 5, 10, 11, 12, or 13, preferably 3, 10, 12, or 13, in one or more substitutions, preferably from one to eleven substitutions, such as one to eight substitutions, eg. 1-6 substitutions. In a further embodiment the parent adiponectin comprises at least one lysine residue selected from any one of the positions K65, K68, K77, or K101. As mentioned above when produced in a eucaryotic cell, such as a mammalian cell, lysine residues in the collagen domain are hydroxylated and glycosylated. Typically the lysine residues are hydroxylated and glycosylated. In a further embodiment the parent adiponectin comprises one lysine residue selected from any one of the positions K65, K68, K77, or K101, preferably K101, and preferably the position is hydroxylated and glycosylated, such as glyco-hydroxy-K101. In a further embodiment the parent adiponectin comprises two lysine residues selected from any one of the positions K65, K68, K77, or K101, preferably K77 and K101, and preferably both of the positions are hydroxylated and glycosylated, such as glyco-hydroxy-K77 and glyco-hydroxy-K101. In a further embodiment the parent adiponectin comprises three lysine residues selected from any one of the positions K65, K68, K77, or K101, preferably K68, K77 and K101, and preferably all three of the positions are hydroxylated and glycosylated, such as glyco-hydroxy-K68, glyco-hydroxy-K77 and glyco-hydroxy-K101. In a further embodiment the parent adiponectin comprises four lysine residues selected from positions K65, K68, K77, and K101, and preferably all four of the positions are hydroxylated and glycosylated.

In a still further embodiment the parent adiponectin polypeptide is selected from any one of the adiponectin polypeptide fragments described in the above section “Adiponectin polypeptide fragment(s) of the invention”. Each of the described adiponectin polypeptide fragments is considered an embodiment suitable as the parent adiponectin polypeptide in either the adiponectin polypeptide or the conjugate.

Accordingly, one example of a preferred aspect of the adiponectin polypeptide relates to an adiponectin polypeptide fragment comprising an amino acid residue having an attachment group for a first non-polypeptide moiety, wherein said amino acid residue has been introduced in a position that in the parent adiponectin is occupied by a surface exposed amino acid residue. In one embodiment the parent adiponectin is selected from an adiponectin polypeptide fragment comprising a globular domain and a collagen domain, wherein the globular domain comprises an amino acid sequence from position A108 to N244 as indicated in SEQ ID NO:1 as well as sequences that differs from the amino acid sequence in one or more substitution(s), and wherein the collagen domain comprises from 7 amino acids corresponding to position K101 as indicated in SEQ ID NO:1 to 56 amino acids corresponding to position A52 as indicated in SEQ ID NO:1, and wherein the collagen domain comprises a lysine which is hydroxylated and glycosylated.

Furthermore, one example of a preferred aspect of the conjugate relates to a conjugate comprising an adiponectin polypeptide fragment, and a first non-polypeptide moiety covalently attached to the adiponectin polypeptide fragment, wherein the adiponectin polypeptide fragment comprises an amino acid residue having an attachment group for said first non-polypeptide moiety, wherein said amino acid residue has been introduced in a position that in the parent adiponectin is occupied by a surface exposed amino acid residue. In one embodiment the parent adiponectin is selected from an adiponectin polypeptide fragment comprising a globular domain and a collagen domain, wherein the globular domain comprises an amino acid sequence from position A108 to N244 as indicated in SEQ ID NO:1 as well as sequences that differs from the amino acid sequence in one or more substitution(s), and wherein the collagen domain comprises from 7 amino acids corresponding to position K101 as indicated in SEQ ID NO:1 to 56 amino acids corresponding to position A52 as indicated in SEQ ID NO:1, and wherein the collagen domain comprises a lysine which is hydroxylated and glycosylated.

It should be clear that the surface exposed amino acid residue having an attachment group for the first non-polypeptide moiety may either be introduced in the globular domain or in the collagen domain, or in case of more than one non-polypeptide moiety being attached they may be introduced in the globular domain or in the collagen domain, or in both the globular domain and the collagen domain.

Accordingly, in a further embodiment of the conjugate or the adiponectin polypeptide or the adiponectin polypeptide fragment the surface exposed amino acid residue having the attachment group is introduced in the globular domain.

In a further embodiment the adiponectin polypeptide comprises a collagen domain.

In a further embodiment the surface exposed amino acid residue having the attachment group is introduced in the collagen domain. If only one first non-polypeptide is attached then it may be in the globular domain or in the collagen domain. If more than one, such as two non-polypeptides, are attached then one may be located in the collagen domain and one in the globular domain, or both may be in the collagen domain, or both may be in the globular domain.

In a further embodiment the adiponectin polypeptide comprises a non-homologous domain.

In a further embodiment the adiponectin polypeptide comprises a signal peptide.

In a further embodiment the adiponectin polypeptide is isolated.

In a further embodiment only one first non-polypeptide moiety is attached to the adiponectin polypeptide.

In a further embodiment the conjugate of the invention is mono pegylated.

In a further embodiment the amino acid residue having the attachment group for said first non-polypeptide moiety is selected from a lysine, aspartic acid, glutamic acid or cysteine residue.

In a further embodiment the amino acid residue having the attachment group for said first non-polypeptide moiety is an glutamic acid residue. Preferred Glu mutations are made in the globular domain and may be selected from any one of A108E, Y109E, V110E, Y111E, R112E, T121E, Y122E, V123E, T124E, I125E, P126E, N127E, M128E, R131E, T133E, K134E, I135E, Q139E, N141E, D144E, G145E, S146E, T147E, K149E, H151E, N153E, P1SSE, Y167E, M168E, K169E, D170E, K178E, D179E, K180E, A181E, F184E, Y186E, Q188E, Y189E, Q190E, N192E, N193E, V194E, H204E, E206E, V207E, G208E, Q210E, V215E, Y216E, G217E, G219E, R221E, N222E, L224E, Y225E, D227E, N228E, D229E, N230E, H241E, D242E, T243E, or N244E.

In a further embodiment the amino acid residue having the attachment group for said first non-polypeptide moiety is an aspartic acid residue. Preferred Asp mutations are made in the globular domain and may be selected from any one of A108D, Y109D, V110D, Y111D, R112D, E120D, T121D, Y122D, V123D, T124D, I125D, P126D, N127D, M128D, R131D, T133D, K134D, I135D, Q139D, N141D, G145D, S146D, T147D, K149D, H151D, N153D, P155D, Y167D, M168D, K169D, K178D, K180D, A181D, F184D, Y186D, Q188D, Y189D, Q190D, E191D, N192D, N193D, V194D, H204D, E206D, V207D, G208D, Q210D, V215D, Y216D, G217D, E218D, G219D, E220D, R221D, N222D, L224D, Y225D, N228D, N230D, H241D, T243D, or N244D.

In a further embodiment the amino acid residue having the attachment group for said first non-polypeptide moiety is a lysine residue. Preferred Lys mutations are made in the globular domain and may be selected from any one of A108K, Y109K, V110K, Y111K, R112K, E120K, T121K, Y122K, V123K, T124K, I125K, P126K, N127K, M128K, R131K, T133K, I135K, Q139K, N141K, D144K, G145K, S146K, T147K, H151K, N153K, P155K, Y167K, M168K, D170K, D179K, A181K, F184K, Y186K, Q188K, Y189K, Q190K, E191K, N192K, N193K, V194K, H204K, E206K, V207K, G208K, Q210K, V215K, Y216K, G217K, E218K, G219K, E220K, R221K, N222K, L224K, Y225K, D227K, N228K, D229K, N230K, H241K, D242K, T243K, or N244K.

In a further embodiment the amino acid residue having the attachment group for said first non-polypeptide moiety is a cysteine residue. Preferred Cys mutations are made in the globular domain and may be selected from any one of A108C, Y109C, V110C, Y111C, R112C, E120C, T121C, Y122C, V123C, T124C, I125C, P126C, N127C, M128C, R131C, T133C, K134C, I135C, Q139C, N141C, D144C, G145C, S146C, T147C, K149C, H151C, N153C, P155C, Y167C, M168C, K169C, D170C, K178C, D179C, K180C, A181C, F184C, Y186C, Q188C, Y189C, Q190C, E191C, N192C, N193C, V194C, H204C, E206C, V207C, G208C, Q210C, V215C, Y216C, G217C, E218C, G219C, E220C, R221C, N222C, L224C, Y225C, D227C, N228C, D229C, N230C, H241C, D242C, T243C, or N244C, such as T121C, S146C, or T243C. Cys152 (which is not surface exposed) relative to human adiponectin is preferably maintained so that the adiponectin polypeptide contains two cysteins in the globular domain.

The above Lys, Glu, Asp, or Cys mutations may be introduced in any one of the parent adiponectin polypeptides as part of the conjugate or as the adiponectin polypeptide, including fragments thereof, such as any one of the sequences SEQ ID NO:3, 4, 5, 6, 10, 11, 12, or 13, or the adiponectin polypeptide fragments selected from any one of the adiponectin polypeptide fragments described in the above section “Adiponectin polypeptide fragment(s) of the invention”.

To illustrate the non-conjugated polypeptide part of the invention some embodiments have been outlined hereafter.

Typical, embodiments of the invention relates to an adiponectin polypeptide comprising a mutation selected from any one of A108C, Y109C, V110C, Y111C, R112C, E120C, T121C, Y122C, V123C, T124C, I125C, P126C, N127C, M128C, R131C, T133C, K134C, I135C, Q139C, N141C, D144C, G145C, S146C, T147C, K149C, H151C, N153C, P155C, Y167C, M168C, K169C, D170C, K178C, D179C, K180C, A181C, F184C, Y186C, Q188C, Y189C, Q190C, E191C, N192C, N193C, V194C, H204C, E206C, V207C, G208C, Q210C, V215C, Y216C, G217C, E218C, G219C, E220C, R221C, N222C, L224C, Y225C, D227C, N228C, D229C, N230C, H241C, D242C, T243C, or N244C, such as T121C, S146C, or T243C. Preferably, the adiponectin polypeptide contains only one of these cysteine mutations, since two or more may lead to loss of product upon expression, for instance, due to inter and/or intra molecular sulphurbridges being formed. Cys152 (which is not surface exposed) relative to human adiponectin is preferably maintained so that the adiponectin polypeptide contains two or more cysteins in the globular domain, that is Cys152 and one or more introduced cysteins, preferably one introduced cystein and the conserved Cys152.

Typically, the adiponectin polypeptide is selected from any one of the sequences SEQ ID NO:3, 4, 5, 6, 10, 11, 12, or 13, or any one of the adiponectin polypeptide fragments described in the above section “Adiponectin polypeptide fragment(s) of the invention”. Typical embodiments of the adiponectin polypeptide are selected from any one of the sequences SEQ ID NO:17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, or 52.

Accordingly, the present invention relates to an adiponectin polypeptide having an amino acid sequence selected from any one of the SEQ ID NO:3, 4, 5, 6, 10, 11, 12, or 13, wherein the adiponectin polypeptide comprises a mutation selected from any one of A108C, Y109C, V110C, Y111C, R112C, E120C, T121C, Y122C, V123C, T124C, I125C, P126C, N127C, M128C, R131C, T133C, K134C, 1135C, Q139C, N141C, D144C, G145C, S146C, T147C, K149C, H151C, N153C, P155C, Y167C, M168C, K169C, D170C, K178C, D179C, K180C, A181C, F184C, Y186C, Q188C, Y189C, Q190C, E191C, N192C, N193C, V194C, H204C, E206C, V207C, G208C, Q210C, V215C, Y216C, G217C, E218C, G219C, E220C, R221C, N222C, L224C, Y225C, D227C, N228C, D229C, N230C, H241C, D242C, T243C, or N244C. Thus, each of these amino acid sequences in combination with one of the specified mutations constitutes embodiments of the invention and may be made the subject of one or more claims. Typically, the adiponectin polypeptide comprising a mutation is produced in a eucaryotic cell, such as a mammalian cell, and thus, any one of the sequences SEQ ID NO:3, 4, 5, 10, 11, 12, or 13 comprises a lysine in the collagen domain which is hydroxylated and glycosylated. Alternatively, any one of the sequences SEQ ID NO:3, 4, 5, 10, 11, 12, or 13 is produced in a bacterial cell, such as E. Coli, and thus, is not hydroxylated and glycosylated. For instance, in one example the present invention relates to an adiponectin polypeptide having an amino acid sequence selected from the SEQ ID NO:10, wherein the adiponectin polypeptide comprises the mutation T121C, such as the adiponectin polypeptide having the amino acid sequence of SEQ ID NO:17; in another example the present invention relates to an adiponectin polypeptide having an amino acid sequence selected from the SEQ ID NO:10, wherein the adiponectin polypeptide comprises the mutation S146C, such as the adiponectin polypeptide having the amino acid sequence of SEQ ID NO:18; in a further example the present invention relates to an adiponectin polypeptide having an amino acid sequence selected from the SEQ ID NO:10, wherein the adiponectin polypeptide comprises the mutation T243C, such as the adiponectin polypeptide having the amino acid sequence of SEQ ID NO:19; in a further example the present invention relates to an adiponectin polypeptide having an amino acid sequence selected from the SEQ ID NO:10, wherein the adiponectin polypeptide comprises the mutation N127C, such as the adiponectin polypeptide having the amino acid sequence of SEQ ID NO:35; in a further example the present invention relates to an adiponectin polypeptide having an amino acid sequence selected from the SEQ ID NO:10, wherein the adiponectin polypeptide comprises the mutation N141C, such as the adiponectin polypeptide having the amino acid sequence of SEQ ID NO:36; in a further example the present invention relates to an adiponectin polypeptide having an amino acid sequence selected from the SEQ ID NO:10, wherein the adiponectin polypeptide comprises the mutation N228C, such as the adiponectin polypeptide having the amino acid sequence of SEQ ID NO:37;in a further example the present invention relates to an adiponectin polypeptide having an amino acid sequence selected from the SEQ ID NO:5, wherein the adiponectin polypeptide comprises the mutation T121C, such as the adiponectin polypeptide having the amino acid sequence of SEQ ID NO:23; in a further example the present invention relates to an adiponectin polypeptide having an amino acid sequence selected from the SEQ ID NO:5, wherein the adiponectin polypeptide comprises the mutation S 146C, such as the adiponectin polypeptide having the amino acid sequence of SEQ ID NO:24; in a further example the present invention relates to an adiponectin polypeptide having an amino acid sequence selected from the SEQ ID NO:5, wherein the adiponectin polypeptide comprises the mutation T243C, such as the adiponectin polypeptide having the amino acid sequence of SEQ ID NO:25; in a further example the present invention relates to an adiponectin polypeptide having an amino acid sequence selected from the SEQ ID NO:5, wherein the adiponectin polypeptide comprises the mutation N127C, such as the adiponectin polypeptide having the amino acid sequence of SEQ ID NO:41; in a further example the present invention relates to an adiponectin polypeptide having an amino acid sequence selected from the SEQ ID NO:5, wherein the adiponectin polypeptide comprises the mutation N141C, such as the adiponectin polypeptide having the amino acid sequence of SEQ ID NO:42; in a further example the present invention relates to an adiponectin polypeptide having an amino acid sequence selected from the SEQ ID NO:5, wherein the adiponectin polypeptide comprises the mutation N228C, such as the adiponectin polypeptide having the amino acid sequence of SEQ ID NO:43; in a further example the present invention relates to an adiponectin polypeptide having an amino acid sequence selected from the SEQ ID NO:13, wherein the adiponectin polypeptide comprises the mutation T121C, such as the adiponectin polypeptide having the amino acid sequence of SEQ ID NO:32; in a further example the present invention relates to an adiponectin polypeptide having an amino acid sequence selected from the SEQ ID NO:13, wherein the adiponectin polypeptide comprises the mutation S 146C, such as the adiponectin polypeptide having the amino acid sequence of SEQ ID NO:33; in a further example the present invention relates to an adiponectin polypeptide having an amino acid sequence selected from the SEQ ID NO:13, wherein the adiponectin polypeptide comprises the mutation T243C, such as the adiponectin polypeptide having the amino acid sequence of SEQ ID NO:34; in a further example the present invention relates to an adiponectin polypeptide having an amino acid sequence selected from the SEQ ID NO:13, wherein the adiponectin polypeptide comprises the mutation N127C, such as the adiponectin polypeptide having the amino acid sequence of SEQ ID NO:50; in a further example the present invention relates to an adiponectin polypeptide having an amino acid sequence selected from the SEQ ID NO:13, wherein the adiponectin polypeptide comprises the mutation N141C, such as the adiponectin polypeptide having the amino acid sequence of SEQ ID NO:51; in a further example the present invention relates to an adiponectin polypeptide having an amino acid sequence selected from the SEQ ID NO:13, wherein the adiponectin polypeptide comprises the mutation N228C, such as the adiponectin polypeptide having the amino acid sequence of SEQ ID NO:52; and so forth. Preferably, any one of the above adiponectin polypeptides comprising a cystein introduced in the globular domain is produced in a eucaryotic cell, such as a mammalian cell.

Furthermore, the present invention relates to an adiponectin polypeptide fragment comprising a globular domain and a collagen domain,

wherein the globular domain comprises an amino acid sequence as indicated in SEQ ID NO:1 from position A108 to N244, and wherein the collagen domain comprises from 7 amino acids corresponding to position K101 as indicated in SEQ ID NO:1 to 66 amino acids corresponding to position G42 as indicated in SEQ ID NO:1, and wherein the collagen domain comprises a lysine which is hydroxylated and glycosylated,

wherein the adiponectin polypeptide fragment comprises a mutation selected from any one of A108C, Y109C, V110C, Y111C, R112C, E120C, T121C, Y122C, V123C, T124C, I125C, P126C, N127C, M128C, R131C, T133C, K134C, I135C, Q139C, N141C, D144C, G145C, S146C, T147C, K149C, H151C, N153C, P155C, Y167C, M168C, K169C, D170C, K178C, D179C, K180C, A181C, F184C, Y186C, Q188C, Y189C, Q190C, E191C, N192C, N193C, V194C, H204C, E206C, V207C, G208C, Q210C, V215C, Y216C, G217C, E218C, G219C, E220C, R221C, N222C, L224C, Y225C, D227C, N228C, D229C, N230C, H241C, D242C, T243C, or N244C. Thus, each of these amino acid sequences, including the embodiments described in the above section “Adiponectin polypeptide fragment(s) of the invention”, in combination with one of the specified mutations constitutes embodiments of the invention and may be made the subject of one or more claims. As mentioned above it is preferred that the adiponectin polypeptide fragment only contains one introduced Cys. However, the adiponectin polypeptide fragment may contain other mutations as long as the biological activity is maintained (as mentioned above), which means that preferably up to eleven (11) substitutions may be made in the globular domain. For instance, in one example the present invention relates to an adiponectin polypeptide fragment comprising a globular domain and a collagen domain,

wherein the globular domain comprises an amino acid sequence from position A108 to N244 as indicated in SEQ ID NO:1, and wherein the collagen domain comprises from 7 amino acids corresponding to position K101 as indicated in SEQ ID NO:1 to 56 amino acids corresponding to position A52 as indicated in SEQ ID NO:1, and wherein the collagen domain comprises a lysine which is hydroxylated and glycosylated, wherein the adiponectin polypeptide fragment comprises the mutation T121 C. Examples of such adiponectin polypeptide fragments are any one of the sequences SEQ ID NO:17, 23, 26, 29, or 32. In another example the present invention relates to an adiponectin polypeptide fragment comprising a globular domain and a collagen domain,

wherein the globular domain comprises an amino acid sequence from position A108 to N244 as indicated in SEQ ID NO:1, and wherein the collagen domain comprises from 8 amino acids corresponding to position R100 as indicated in SEQ ID NO:1 to 50 amino acids corresponding to position R58 as indicated in SEQ ID NO:1, and wherein the collagen domain comprises a lysine which is hydroxylated and glycosylated, wherein the adiponectin polypeptide comprises the mutation S146C. Examples of such adiponectin polypeptide fragments are any one of the sequences SEQ ID NO:18, 27, or 33. In a further example the present invention relates to an adiponectin polypeptide fragment comprising a globular domain and a collagen domain,

wherein the globular domain comprises an amino acid sequence from position A108 to N244 as indicated in SEQ ID NO:1, and wherein the collagen domain comprises from 8 amino acids corresponding to position R100 as indicated in SEQ ID NO:1 to 29 amino acids corresponding to position D79 as indicated in SEQ ID NO:1, and wherein the collagen domain comprises a lysine which is hydroxylated and glycosylated, wherein the adiponectin polypeptide comprises the mutation T243C. Examples of such adiponectin polypeptide fragments are any one of the sequences SEQ ID NO:19, or 28. In a further example the present invention relates to an adiponectin polypeptide fragment comprising a globular domain and a collagen domain,

wherein the globular domain comprises an amino acid sequence from position A108 to N244 as indicated in SEQ ID NO:1, and wherein the collagen domain comprises from 7 amino acids corresponding to position K101 as indicated in SEQ ID NO:1 to 56 amino acids corresponding to position A52 as indicated in SEQ ID NO:1, and wherein the collagen domain comprises a lysine which is hydroxylated and glycosylated, wherein the adiponectin polypeptide fragment comprises the mutation N127C. Examples of such adiponectin polypeptide fragments are any one of the sequences SEQ ID NO:35, 41, 44, 47, or 50. In a further example the present invention relates to an adiponectin polypeptide fragment comprising a globular domain and a collagen domain,

wherein the globular domain comprises an amino acid sequence from position A108 to N244 as indicated in SEQ ID NO:1, and wherein the collagen domain comprises from 8 amino acids corresponding to position R100 as indicated in SEQ ID NO:1 to 50 amino acids corresponding to position R58 as indicated in SEQ ID NO:1, and wherein the collagen domain comprises a lysine which is hydroxylated and glycosylated, wherein the adiponectin polypeptide comprises the mutation N141C. Examples of such adiponectin polypeptide fragments are any one of the sequences SEQ ID NO:36, 45, or 51. In a further example the present invention relates to an adiponectin polypeptide fragment comprising a globular domain and a collagen domain,

wherein the globular domain comprises an amino acid sequence from position A108 to N244 as indicated in SEQ ID NO:1, and wherein the collagen domain comprises from 8 amino acids corresponding to position R100 as indicated in SEQ ID NO:1 to 29 amino acids corresponding to position D79 as indicated in SEQ ID NO:1, and wherein the collagen domain comprises a lysine which is hydroxylated and glycosylated, wherein the adiponectin polypeptide comprises the mutation N228C. Examples of such adiponectin polypeptide fragments are any one of the sequences SEQ ID NO:37, or 46. And so forth.

Also, as described above the above Lys, Glu, Asp, or Cys mutations may be introduced in any one of the parent adiponectin polypeptides as part of the conjugate, including fragments thereof, such as any one of the sequences SEQ ID NO:3, 4, 5, 6, 10, 11, 12, or 13, or the adiponectin polypeptide fragments selected from any one of the adiponectin polypeptide fragments described in the above section “Adiponectin polypeptide fragment(s) of the invention”, in which respect a first non-polypeptide moiety is attached to the introduced amino acid residue having an attachment group for said first non-polypeptide moiety.

To illustrate this conjugate part of the invention some embodiments have been outlined hereafter. Typical, embodiments of the invention relates to a conjugate comprising an adiponectin polypeptide comprising a mutation selected from any one of A108C, Y109C, V110C, Y111C, R112C, E120C, T121C, Y122C, V123C, T124C, I125C, P126C, N127C, M128C, R131C, T133C, K134C, 1135C, Q139C, N141C, D144C, G145C, S146C, T147C, K149C, H151C, N153C, P155C, Y167C, M168C, K169C, D170C, K178C, D179C, K180C, A181C, F184C, Y186C, Q188C, Y189C, Q190C, E191C, N192C, N193C, V194C, H204C, E206C, V207C, G208C, Q210C, V215C, Y216C, G217C, E218C, 6219C, E220C, R221C, N222C, L224C, Y225C, D227C, N228C, D229C, N230C, H241C, D242C, T243C, or N244C, such as T121C, S146C, or T243C, and a first non-polypeptide moiety covalently attached to the introduced cystein residue. Preferably, the adiponectin polypeptide of the conjugate contains only one of these cysteine mutations, since two or more may lead to loss of product upon expression, for instance, due to inter and/or intra molecular sulphurbridges being formed. Typically, the adiponectin polypeptide of the conjugate is selected from any one of the sequences SEQ ID NO:3, 4, 5, 6, 10, 11, 12, or 13, or any one of the adiponectin polypeptide fragments described in the above section “Adiponectin polypeptide fragment(s) of the invention”.

Accordingly, the present invention relates to a conjugate comprising an adiponectin polypeptide having an amino acid sequence selected from any one of the SEQ ID NO:3, 4, 5, 6, 10, 11, 12, or 13, wherein the adiponectin polypeptide comprises a mutation selected from any one of A108E, Y109E, V110E, Y111E, R112E, T121E, Y122E, V123E, T124E, I125E, P126E, N127E, M128E, R131E, T133E, K134E, I135E, Q139E, N141E, D144E, G145E, S146E, T147E, K149E, H151E, N153E, P155E, Y167E, M168E, K169E, D170E, K178E, D179E, K180E, A181E, F184E, Y186E, Q188E, Y189E, Q190E, N192E, N193E, V194E, H204E, E206E, V207E, G208E, Q210E, V215E, Y216E, G217E, G219E, R221E, N222E, L224E, Y225E, D227E, N228E, D229E, N230E, H241E, D242E, T243E, or N244E, and a first non-polypeptide moiety covalently attached to the introduced Glu residue. Thus, each of these amino acid sequences in combination with one of the specified mutations constitutes embodiments of the invention and may be made the subject of one or more claims.

Furthermore, the present invention relates to a conjugate comprising an adiponectin polypeptide having an amino acid sequence selected from any one of the SEQ ID NO:3, 4, 5, 6, 10, 11, 12, or 13, wherein the adiponectin polypeptide comprises a mutation selected from any one of A108D, Y109D, V110D, Y111D, R112D, E120D, T121D, Y122D, V123D, T124D, I125D, P126D, N127D, M128D, R131D, T133D, K134D, I135D, Q139D, N141D, G145D, S146D, T147D, K149D, H151D, N153D, P155D, Y167D, M168D, K169D, K178D, K180D, A181D, F184D, Y186D, Q188D, Y189D, Q190D, E191D, N192D, N193D, V194D, H204D, E206D, V207D, G208D, Q210D, V215D, Y216D, G217D, E218D, G219D, E220D, R221D, N222D, L224D, Y225D, N228D, N230D, H241D, T243D, or N244D, and a first non-polypeptide moiety covalently attached to the introduced Asp residue. Thus, each of these amino acid sequences in combination with one of the specified mutations constitutes embodiments of the invention and may be made the subject of one or more claims.

Furthermore, the present invention relates to a conjugate comprising an adiponectin polypeptide having an amino acid sequence selected from any one of the SEQ ID NO:3, 4, 5, 6, 10, 11, 12, or 13, wherein the adiponectin polypeptide comprises a mutation selected from any one of A108K, Y109K, V110K, Y111K, R112K, E120K, T121K, Y122K, V123K, T124K, I125K, P126K, N127K, M128K, R131K, T133K, I135K, Q139K, N141K, D144K, G145K, S146K, T147K, H151K, N153K, P155K, Y167K, M168K, D170K, D179K, A181K, F184K, Y186K, Q188K, Y189K, Q190K, E191K, N192K, N193K, V194K, H204K, E206K, V207K, G208K, Q210K, V215K, Y216K, G217K, E218K, G219K, E220K, R221K, N222K, L224K, Y225K, D227K, N228K, D229K, N230K, H241K, D242K, T243K, or N244K, and a first non-polypeptide moiety covalently attached to the introduced Lys residue. Thus, each of these amino acid sequences in combination with one of the specified mutations constitutes embodiments of the invention and may be made the subject of one or more claims.

Furthermore, the present invention relates to a conjugate comprising an adiponectin polypeptide having an amino acid sequence selected from any one of the SEQ ID NO:3, 4, 5, 6, 10, 11, 12, or 13, wherein the adiponectin polypeptide comprises a mutation selected from any one of A108C, Y109C, V110C, Y111C, R112C, E120C, T121C, Y122C, V123C, T124C, I125C, P126C, N127C, M128C, R131C, T133C, K134C, I135C, Q139C, N141C, D144C, G145C, S146C, T147C, K149C, H151C, N153C, P155C, Y167C, M168C, K169C, D170C, K178C, D179C, K180C, A181C, F184C, Y186C, Q188C, Y189C, Q190C, E191C, N192C, N193C, V194C, H204C, E206C, V207C, G208C, Q210C, V215C, Y216C, G217C, E218C, G219C, E220C, R221C, N222C, L224C, Y225C, D227C, N228C, D229C, N230C, H241C, D242C, T243C, or N244C, and a first non-polypeptide moiety covalently attached to the introduced cystein residue. Thus, each of these amino acid sequence sequences in combination with one of the specified mutations constitutes embodiments of the invention and may be made the subject of one or more claims. Typical embodiments of the adiponectin polypeptide part of the conjugate are any one of the sequences SEQ ID NO:17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, or 52. Preferably, any one of the above adiponectin polypeptides comprising a cystein introduced in the globular domain is produced in a eucaryotic cell, such as a mammalian cell, e.g. a CHO, BHK, HEK293 cell or an SF9 cell.

For instance, in one example the present invention relates to a conjugate comprising an adiponectin polypeptide having an amino acid sequence selected from the SEQ ID NO:10, wherein the adiponectin polypeptide comprises the mutation T121C, and a first non-polypeptide moiety covalently attached to the introduced cystein residue. Such as a conjugate comprising an adiponectin polypeptide having the sequence SEQ ID NO:17, and a first non-polypeptide moiety covalently attached to the introduced cystein residue T121C. In another example the present invention relates to a conjugate comprising an adiponectin polypeptide having an amino acid sequence selected from the SEQ ID NO:10, wherein the adiponectin polypeptide comprises the mutation S 146C, and a first non-polypeptide moiety covalently attached to the introduced cystein residue. Such as a conjugate comprising an adiponectin polypeptide having the sequence SEQ ID NO:18, and a first non-polypeptide moiety covalently attached to the introduced cystein residue S146C. In a further example the present invention relates to a conjugate comprising an adiponectin polypeptide having an amino acid sequence selected from the SEQ ID NO:10, wherein the adiponectin polypeptide comprises the mutation T243C, and a first non-polypeptide moiety covalently attached to the introduced cystein residue. Such as a conjugate comprising an adiponectin polypeptide having the sequence SEQ ID NO:19, and a first non-polypeptide moiety covalently attached to the introduced cystein residue T243C. In a further example the present invention relates to a conjugate comprising an adiponectin polypeptide having an amino acid sequence selected from the SEQ ID NO:10, wherein the adiponectin polypeptide comprises the mutation N127C, and a first non-polypeptide moiety covalently attached to the introduced cystein residue. Such as a conjugate comprising an adiponectin polypeptide having the sequence SEQ ID NO:35, and a first non-polypeptide moiety covalently attached to the introduced cystein residue N127C. In a further example the present invention relates to a conjugate comprising an adiponectin polypeptide having an amino acid sequence selected from the SEQ ID NO:10, wherein the adiponectin polypeptide comprises the mutation N141C, and a first non-polypeptide moiety covalently attached to the introduced cystein residue. Such as a conjugate comprising an adiponectin polypeptide having the sequence SEQ ID NO:36, and a first non-polypeptide moiety covalently attached to the introduced cystein residue N141C. In a further example the present invention relates to a conjugate comprising an adiponectin polypeptide having an amino acid sequence selected from the SEQ ID NO:10, wherein the adiponectin polypeptide comprises the mutation N228C, and a first non-polypeptide moiety covalently attached to the introduced cystein residue. Such as a conjugate comprising an adiponectin polypeptide having the sequence SEQ ID NO:37, and a first non-polypeptide moiety covalently attached to the introduced cystein residue N228C. In a further example the present invention relates to a conjugate comprising an adiponectin polypeptide having an amino acid sequence selected from the SEQ ID NO:5, wherein the adiponectin polypeptide comprises the mutation T121C, and a first non-polypeptide moiety covalently attached to the introduced cystein residue. Such as a conjugate comprising an adiponectin polypeptide having the sequence SEQ ID NO:23, and a first non-polypeptide moiety covalently attached to the introduced cystein residue T121C. In a further example the present invention relates to a conjugate comprising an adiponectin polypeptide having an amino acid sequence selected from the SEQ ID NO:5, wherein the adiponectin polypeptide comprises the mutation S146C, and a first non-polypeptide moiety covalently attached to the introduced cystein residue. Such as a conjugate comprising an adiponectin polypeptide having the sequence SEQ ID NO:24, and a first non-polypeptide moiety covalently attached to the introduced cystein residue S146C. In a further example the present invention relates to a conjugate comprising an adiponectin polypeptide having an amino acid sequence selected from the SEQ ID NO:5, wherein the adiponectin polypeptide comprises the mutation T243C, and a first non-polypeptide moiety covalently attached to the introduced cystein residue. Such as a conjugate comprising an adiponectin polypeptide having the sequence SEQ ID NO:25, and a first non-polypeptide moiety covalently attached to the introduced cystein residue T243C. In a further example the present invention relates to a conjugate comprising an adiponectin polypeptide having an amino acid sequence selected from the SEQ ID NO:5, wherein the adiponectin polypeptide comprises the mutation N127C, and a first non-polypeptide moiety covalently attached to the introduced cystein residue. Such as a conjugate comprising an adiponectin polypeptide having the sequence SEQ ID NO:41, and a first non-polypeptide moiety covalently attached to the introduced cystein residue N127C. In a further example the present invention relates to a conjugate comprising an adiponectin polypeptide having an amino acid sequence selected from the SEQ ID NO:5, wherein the adiponectin polypeptide comprises the mutation N141C, and a first non-polypeptide moiety covalently attached to the introduced cystein residue. Such as a conjugate comprising an adiponectin polypeptide having the sequence SEQ ID NO:42, and a first non-polypeptide moiety covalently attached to the introduced cystein residue N141C. In a further example the present invention relates to a conjugate comprising an adiponectin polypeptide having an amino acid sequence selected from the SEQ ID NO:5, wherein the adiponectin polypeptide comprises the mutation N228C, and a first non-polypeptide moiety covalently attached to the introduced cystein residue. Such as a conjugate comprising an adiponectin polypeptide having the sequence SEQ ID NO:43, and a first non-polypeptide moiety covalently attached to the introduced cystein residue N228C. In a further example the present invention relates to a conjugate comprising an adiponectin polypeptide having an amino acid sequence selected from the SEQ ID NO:13, wherein the adiponectin polypeptide comprises the mutation T121C, and a first non-polypeptide moiety covalently attached to the introduced cystein residue. Such as a conjugate comprising an adiponectin polypeptide having the sequence SEQ ID NO:32, and a first non-polypeptide moiety covalently attached to the introduced cystein residue T121C. In a further example the present invention relates to a conjugate comprising an adiponectin polypeptide having an amino acid sequence selected from the SEQ ID NO:13, wherein the adiponectin polypeptide comprises the mutation S146C, and a first non-polypeptide moiety covalently attached to the introduced cystein residue. Such as a conjugate comprising an adiponectin polypeptide having the sequence SEQ ID NO:33, and a first non-polypeptide moiety covalently attached to the introduced cystein residue S146C. In a further example the present invention relates to a conjugate comprising an adiponectin polypeptide having an amino acid sequence selected from the SEQ ID NO:13, wherein the adiponectin polypeptide comprises the mutation T243C, and a first non-polypeptide moiety covalently attached to the introduced cystein residue. Such as a conjugate comprising an adiponectin polypeptide having the sequence SEQ ID NO:34, and a first non-polypeptide moiety covalently attached to the introduced cystein residue T243C. In a further example the present invention relates to a conjugate comprising an adiponectin polypeptide having an amino acid sequence selected from the SEQ ID NO:13, wherein the adiponectin polypeptide comprises the mutation N127C, and a first non-polypeptide moiety covalently attached to the introduced cystein residue. Such as a conjugate comprising an adiponectin polypeptide having the sequence SEQ ID NO:50, and a first non-polypeptide moiety covalently attached to the introduced cystein residue N127C. In a further example the present invention relates to a conjugate comprising an adiponectin polypeptide having an amino acid sequence selected from the SEQ ID NO:13, wherein the adiponectin polypeptide comprises the mutation N141C, and a first non-polypeptide moiety covalently attached to the introduced cystein residue. Such as a conjugate comprising an adiponectin polypeptide having the sequence SEQ ID NO:51, and a first non-polypeptide moiety covalently attached to the introduced cystein residue N141C. In a further example the present invention relates to a conjugate comprising an adiponectin polypeptide having an amino acid sequence selected from the SEQ ID NO:13, wherein the adiponectin polypeptide comprises the mutation N228C, and a first non-polypeptide moiety covalently attached to the introduced cystein residue. Such as a conjugate comprising an adiponectin polypeptide having the sequence SEQ ID NO:52, and a first non-polypeptide moiety covalently attached to the introduced cystein residue N228C. And so forth. Preferably, any one of the above adiponectin polypeptides comprising a mutation, such as cystein, introduced in the globular domain is produced in a eucaryotic cell, such as a mammalian cell, e.g. a CHO, BHK, HEK293 cell or an SF9 cell.

Furthermore, the present invention relates to a conjugate comprising

an adiponectin polypeptide fragment comprising a globular domain and a collagen domain, wherein the globular domain comprises an amino acid sequence as indicated in SEQ ID NO:1 from position A108 to N244, and wherein the collagen domain comprises from 7 amino acids corresponding to position K101 as indicated in SEQ ID NO:1 to 66 amino acids corresponding to position G42 as indicated in SEQ ID NO:1, and wherein the collagen domain comprises a lysine which is hydroxylated and glycosylated,

wherein the adiponectin polypeptide fragment comprises a mutation selected from any one of A108C, Y109C, V110C, Y111C, R112C, E120C, T121C, Y122C, V123C, T124C, I125C, P126C, N127C, M128C, R131C, T133C, K134C, I135C, Q139C, N141C, D144C, G145C, S146C, T147C, K149C, H151C, N153C, P155C, Y167C, M168C, K169C, D170C, K178C, D179C, K180C, A181C, F184C, Y186C, Q188C, Y189C, Q190C, E191C, N192C, N193C, V194C, H204C, E206C, V207C, G208C, Q210C, V215C, Y216C, G217C, E218C, G219C, E220C, R221C, N222C, L224C, Y225C, D227C, N228C, D229C, N230C, H241C, D242C, T243C, or N244C; and

a first non-polypeptide moiety covalently attached to the introduced cystein residue. Thus, each of these amino acid sequences as part of the conjugate, including the embodiments described in the above section “Adiponectin polypeptide fragment(s) of the invention”, in combination with one of the specified mutations constitutes embodiments of the invention and may be made the subject of one or more claims. As mentioned above it is preferred that the adiponectin polypeptide fragment only contains one introduced Cys, and that Cys 152 is maintained. However, the adiponectin polypeptide fragment may contain other mutations as long as the biological activity is maintained (as mentioned above), which means that preferably up to eleven (11) substitutions may be made in the globular domain. Typical embodiments of the adiponectin polypeptide fragment part of the conjugate are any one of the sequences SEQ ID NO:17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, or 52.

For instance, in one example the present invention relates to a conjugate comprising

an adiponectin polypeptide fragment comprising a globular domain and a collagen domain, wherein the globular domain comprises an amino acid sequence from position A108 to N244 as indicated in SEQ ID NO:1, and wherein the collagen domain comprises from 7 amino acids corresponding to position K101 as indicated in SEQ ID NO:1 to 56 amino acids corresponding to position A52 as indicated in SEQ ID NO:1, and wherein the collagen domain comprises a lysine which is hydroxylated and glycosylated, wherein the adiponectin polypeptide fragment comprises the mutation T121C; and

a first non-polypeptide moiety covalently attached to the introduced cystein residue.

In another example the present invention relates to a conjugate comprising an adiponectin polypeptide fragment comprising a globular domain and a collagen domain,

wherein the globular domain comprises an amino acid sequence from position A108 to N244 as indicated in SEQ ID NO:1, and wherein the collagen domain comprises from 8 amino acids corresponding to position R100 as indicated in SEQ ID NO:1 to 50 amino acids corresponding to position R58 as indicated in SEQ ID NO:1, and wherein the collagen domain comprises a lysine which is hydroxylated and glycosylated, wherein the adiponectin polypeptide comprises the mutation T121C; and

a first non-polypeptide moiety covalently attached to the introduced cystein residue. Such as a conjugate comprising

an adiponectin polypeptide fragment having a sequence selected from any one of SEQ ID NO:17, 23, 26, 29, or 32, and wherein the collagen domain comprises a lysine which is hydroxylated and glycosylated; and

a first non-polypeptide moiety covalently attached to the introduced cystein residue T 12 IC.

In a further example the present invention relates to a conjugate comprising an adiponectin polypeptide fragment comprising a globular domain and a collagen domain,

wherein the globular domain comprises an amino acid sequence from position A108 to N244 as indicated in SEQ ID NO:1, and wherein the collagen domain comprises from 8 amino acids corresponding to position R100 as indicated in SEQ ID NO:1 to 29 amino acids corresponding to position D79 as indicated in SEQ ID NO:1, and wherein the collagen domain comprises a lysine which is hydroxylated and glycosylated, wherein the adiponectin polypeptide comprises the mutation T121C; and

a first non-polypeptide moiety covalently attached to the introduced cystein residue. Such as a conjugate comprising

an adiponectin polypeptide fragment having a sequence selected from any one of SEQ ID NO:17, or 26, and wherein the collagen domain comprises a lysine which is hydroxylated and glycosylated; and

a first non-polypeptide moiety covalently attached to the introduced cystein residue T121C.

In a further example the present invention relates to a conjugate comprising

an adiponectin polypeptide fragment comprising a globular domain and a collagen domain, wherein the globular domain comprises an amino acid sequence from position A108 to N244 as indicated in SEQ ID NO:1, and wherein the collagen domain comprises from 7 amino acids corresponding to position K101 as indicated in SEQ ID NO:1 to 56 amino acids corresponding to position A52 as indicated in SEQ ID NO:1, and wherein the collagen domain comprises a lysine which is hydroxylated and glycosylated, wherein the adiponectin polypeptide fragment comprises the mutation N127C; and

a first non-polypeptide moiety covalently attached to the introduced cystein residue.

In a further example the present invention relates to a conjugate comprising an adiponectin polypeptide fragment comprising a globular domain and a collagen domain,

wherein the globular domain comprises an amino acid sequence from position A108 to N244 as indicated in SEQ ID NO:1, and wherein the collagen domain comprises from 8 amino acids corresponding to position R100 as indicated in SEQ ID NO:1 to 50 amino acids corresponding to position R58 as indicated in SEQ ID NO:1, and wherein the collagen domain comprises a lysine which is hydroxylated and glycosylated, wherein the adiponectin polypeptide comprises the mutation N127C; and

a first non-polypeptide moiety covalently attached to the introduced cystein residue. Such as a conjugate comprising

an adiponectin polypeptide fragment having a sequence selected from any one of SEQ ID NO:35, 41, 44, 47, or 50, and wherein the collagen domain comprises a lysine which is hydroxylated and glycosylated; and

a first non-polypeptide moiety covalently attached to the introduced cystein residue N127C.

In a further example the present invention relates to a conjugate comprising an adiponectin polypeptide fragment comprising a globular domain and a collagen domain,

wherein the globular domain comprises an amino acid sequence from position A108 to N244 as indicated in SEQ ID NO:1, and wherein the collagen domain comprises from 8 amino acids corresponding to position R100 as indicated in SEQ ID NO:1 to 29 amino acids corresponding to position D79 as indicated in SEQ ID NO:1, and wherein the collagen domain comprises a lysine which is hydroxylated and glycosylated, wherein the adiponectin polypeptide comprises the mutation N127C; and

a first non-polypeptide moiety covalently attached to the introduced cystein residue. Such as a conjugate comprising

an adiponectin polypeptide fragment having a sequence selected from any one of SEQ ID NO:35, or 44, and wherein the collagen domain comprises a lysine which is hydroxylated and glycosylated; and

a first non-polypeptide moiety covalently attached to the introduced cystein residue N127C.

In a further example the present invention relates to a conjugate comprising an adiponectin polypeptide fragment comprising a globular domain and a collagen domain, wherein the globular domain comprises an amino acid sequence from position A108 to N244 as indicated in SEQ ID NO:1, and wherein the collagen domain comprises from 7 amino acids corresponding to position K101 as indicated in SEQ ID NO:1 to 56 amino acids corresponding to position A52 as indicated in SEQ ID NO:1, and wherein the collagen domain comprises a lysine which is hydroxylated and glycosylated, wherein the adiponectin polypeptide fragment comprises the mutation S146C; and a first non-polypeptide moiety covalently attached to the introduced cystein residue.

In a further example the present invention relates to a conjugate comprising an adiponectin polypeptide fragment comprising a globular domain and a collagen domain,

wherein the globular domain comprises an amino acid sequence from position A108 to N244 as indicated in SEQ ID NO:1, and wherein the collagen domain comprises from 8 amino acids corresponding to position R100 as indicated in SEQ ID NO:1 to 50 amino acids corresponding to position R58 as indicated in SEQ ID NO:1, and wherein the collagen domain comprises a lysine which is hydroxylated and glycosylated, wherein the adiponectin polypeptide comprises the mutation S146C; and

a first non-polypeptide moiety covalently attached to the introduced cystein residue. Such as a conjugate comprising

an adiponectin polypeptide fragment having a sequence selected from any one of SEQ ID NO:18, 27, or 33, and wherein the collagen domain comprises a lysine which is hydroxylated and glycosylated; and

a first non-polypeptide moiety covalently attached to the introduced cystein residue S146C.

In a further example the present invention relates to a conjugate comprising an adiponectin polypeptide fragment comprising a globular domain and a collagen domain,

wherein the globular domain comprises an amino acid sequence from position A108 to N244 as indicated in SEQ ID NO:1, and wherein the collagen domain comprises from 8 amino acids corresponding to position R100 as indicated in SEQ ID NO:1 to 29 amino acids corresponding to position D79 as indicated in SEQ ID NO:1, and wherein the collagen domain comprises a lysine which is hydroxylated and glycosylated, wherein the adiponectin polypeptide comprises the mutation S146C; and

a first non-polypeptide moiety covalently attached to the introduced cystein residue. Such as a conjugate comprising

an adiponectin polypeptide fragment having a sequence selected from any one of SEQ ID NO:18, or 27, and wherein the collagen domain comprises a lysine which is hydroxylated and glycosylated; and

a first non-polypeptide moiety covalently attached to the introduced cystein residue S146C.

In a further example the present invention relates to a conjugate comprising an adiponectin polypeptide fragment comprising a globular domain and a collagen domain, wherein the globular domain comprises an amino acid sequence from position A108 to N244 as indicated in SEQ ID NO:1, and wherein the collagen domain comprises from 7 amino acids corresponding to position K101 as indicated in SEQ ID NO:1 to 56 amino acids corresponding to position A52 as indicated in SEQ ID NO:1, and wherein the collagen domain comprises a lysine which is hydroxylated and glycosylated, wherein the adiponectin polypeptide fragment comprises the mutation N141C; and a first non-polypeptide moiety covalently attached to the introduced cystein residue.

In a further example the present invention relates to a conjugate comprising an adiponectin polypeptide fragment comprising a globular domain and a collagen domain,

wherein the globular domain comprises an amino acid sequence from position A108 to N244 as indicated in SEQ ID NO:1, and wherein the collagen domain comprises from 8 amino acids corresponding to position R100 as indicated in SEQ ID NO:1 to 50 amino acids corresponding to position R58 as indicated in SEQ ID NO:1, and wherein the collagen domain comprises a lysine which is hydroxylated and glycosylated, wherein the adiponectin polypeptide comprises the mutation N141C; and

a first non-polypeptide moiety covalently attached to the introduced cystein residue. Such as a conjugate comprising

an adiponectin polypeptide fragment having a sequence selected from any one of SEQ ID NO:36, 45, or 51, and wherein the collagen domain comprises a lysine which is hydroxylated and glycosylated; and

a first non-polypeptide moiety covalently attached to the introduced cystein residue N141C.

In a further example the present invention relates to a conjugate comprising an adiponectin polypeptide fragment comprising a globular domain and a collagen domain,

wherein the globular domain comprises an amino acid sequence from position A108 to N244 as indicated in SEQ ID NO:1, and wherein the collagen domain comprises from 8 amino acids corresponding to position R100 as indicated in SEQ ID NO:1 to 29 amino acids corresponding to position D79 as indicated in SEQ ID NO:1, and wherein the collagen domain comprises a lysine which is hydroxylated and glycosylated, wherein the adiponectin polypeptide comprises the mutation N141C; and

a first non-polypeptide moiety covalently attached to the introduced cystein residue. Such as a conjugate comprising

an adiponectin polypeptide fragment having a sequence selected from any one of SEQ ID NO:36, or 45, and wherein the collagen domain comprises a lysine which is hydroxylated and glycosylated; and

a first non-polypeptide moiety covalently attached to the introduced cystein residue N141 C.

In a further example the present invention relates to a conjugate comprising

an adiponectin polypeptide fragment comprising a globular domain and a collagen domain, wherein the globular domain comprises an amino acid sequence from position A 108 to N244 as indicated in SEQ ID NO:1, and wherein the collagen domain comprises from 7 amino acids corresponding to position K101 as indicated in SEQ ID NO:1 to 56 amino acids corresponding to position A52 as indicated in SEQ ID NO:1, and wherein the collagen domain comprises a lysine which is hydroxylated and glycosylated, wherein the adiponectin polypeptide fragment comprises the mutation T243C; and

a first non-polypeptide moiety covalently attached to the introduced cystein residue.

In a further example the present invention relates to a conjugate comprising an adiponectin polypeptide fragment comprising a globular domain and a collagen domain,

wherein the globular domain comprises an amino acid sequence from position A108 to N244 as indicated in SEQ ID NO:1, and wherein the collagen domain comprises from 8 amino acids corresponding to position R100 as indicated in SEQ ID NO:1 to 50 amino acids corresponding to position R58 as indicated in SEQ ID NO:1, and wherein the collagen domain comprises a lysine which is hydroxylated and glycosylated, wherein the adiponectin polypeptide comprises the mutation T243C; and

a first non-polypeptide moiety covalently attached to the introduced cystein residue.

In a further example the present invention relates to a conjugate comprising an adiponectin polypeptide fragment comprising a globular domain and a collagen domain,

wherein the globular domain comprises an amino acid sequence from position A108 to N244 as indicated in SEQ ID NO:1, and wherein the collagen domain comprises from 8 amino acids corresponding to position R100 as indicated in SEQ ID NO:1 to 29 amino acids corresponding to position D79 as indicated in SEQ ID NO:1, and wherein the collagen domain comprises a lysine which is hydroxylated and glycosylated, wherein the adiponectin polypeptide comprises the mutation T243C; and

a first non-polypeptide moiety covalently attached to the introduced cystein residue. Such as a conjugate comprising

an adiponectin polypeptide fragment having a sequence selected from any one of SEQ ID NO:19, or 28, and wherein the collagen domain comprises a lysine which is hydroxylated and glycosylated; and

a first non-polypeptide moiety covalently attached to the introduced cystein residue T243C.

In a further example the present invention relates to a conjugate comprising

an adiponectin polypeptide fragment comprising a globular domain and a collagen domain, wherein the globular domain comprises an amino acid sequence from position A108 to N244 as indicated in SEQ ID NO:1, and wherein the collagen domain comprises from 7 amino acids corresponding to position K101 as indicated in SEQ ID NO:1 to 56 amino acids corresponding to position A52 as indicated in SEQ ID NO:1, and wherein the collagen domain comprises a lysine which is hydroxylated and glycosylated, wherein the adiponectin polypeptide fragment comprises the mutation N228C; and

a first non-polypeptide moiety covalently attached to the introduced cystein residue.

In a further example the present invention relates to a conjugate comprising an adiponectin polypeptide fragment comprising a globular domain and a collagen domain,

wherein the globular domain comprises an amino acid sequence from position A108 to N244 as indicated in SEQ ID NO:1, and wherein the collagen domain comprises from 8 amino acids corresponding to position R100 as indicated in SEQ ID NO:1 to 50 amino acids corresponding to position R58 as indicated in SEQ ID NO:1, and wherein the collagen domain comprises a lysine which is hydroxylated and glycosylated, wherein the adiponectin polypeptide comprises the mutation N228C; and

a first non-polypeptide moiety covalently attached to the introduced cystein residue.

In a further example the present invention relates to a conjugate comprising an adiponectin polypeptide fragment comprising a globular domain and a collagen domain,

wherein the globular domain comprises an amino acid sequence from position A108 to N244 as indicated in SEQ ID NO:1, and wherein the collagen domain comprises from 8 amino acids corresponding to position R100 as indicated in SEQ ID NO:1 to 29 amino acids corresponding to position D79 as indicated in SEQ ID NO:1, and wherein the collagen domain comprises a lysine which is hydroxylated and glycosylated, wherein the adiponectin polypeptide comprises the mutation N228C; and

a first non-polypeptide moiety covalently attached to the introduced cystein residue. Such as a conjugate comprising

an adiponectin polypeptide fragment having a sequence selected from any one of SEQ ID NO:37, or 46, and wherein the collagen domain comprises a lysine which is hydroxylated and glycosylated; and

a first non-polypeptide moiety covalently attached to the introduced cystein residue N228C. And so forth.

The first non-polypeptide moiety to be attached to the introduced amino acid residue having the attachment group for said first non-polypeptide moiety, such as a cysteine, lysine, aspartic acid, or glutamic acid may be introduced by methods known to the person skilled in the art, or as suggested in the section “Methods of preparing a conjugate of the invention” herein. Thus, when a conjugate is to be prepared, a further embodiment of the first non-polypeptide moiety is selected from a polymer, a lipophilic compound, and an organic derivatizing agent.

In a further embodiment the first non-polypeptide moiety is a polymer, typically a linear or branched polyethylene glycol. Such polymers are available from Shearwater.

In a specific aspect the present invention relates to a conjugate comprising

an adiponectin polypeptide fragment comprising a globular domain and a collagen domain, wherein the globular domain comprises an amino acid sequence from position A108 to N244 as indicated in SEQ ID NO:1, and wherein the collagen domain comprises from 7 amino acids corresponding to position K101 as indicated in SEQ ID NO:1 to 56 amino acids corresponding to position A52 as indicated in SEQ ID NO:1, and wherein the collagen domain comprises a lysine which is hydroxylated and glycosylated, wherein the adiponectin polypeptide fragment comprises the mutation T121C; and

a first polymer covalently attached to the introduced cystein residue.

In another example the present invention relates to a conjugate comprising an adiponectin polypeptide fragment comprising a globular domain and a collagen domain,

wherein the globular domain comprises an amino acid sequence from position A108 to N244 as indicated in SEQ ID NO:1, and wherein the collagen domain comprises from 8 amino acids corresponding to position R100 as indicated in SEQ ID NO:1 to 50 amino acids corresponding to position R58 as indicated in SEQ ID NO:1, and wherein the collagen domain comprises a lysine which is hydroxylated and glycosylated, wherein the adiponectin polypeptide comprises the mutation T121C; and

a first polymer covalently attached to the introduced cystein residue. Such as a conjugate comprising

an adiponectin polypeptide fragment having a sequence selected from any one of SEQ ID NO:17, 23, 26, 29, or 32, and wherein the collagen domain comprises a lysine which is hydroxylated and glycosylated; and

a first polymer covalently attached to the introduced cystein residue T121C.

In a further example the present invention relates to a conjugate comprising an adiponectin polypeptide fragment comprising a globular domain and a collagen domain,

wherein the globular domain comprises an amino acid sequence from position A108 to N244 as indicated in SEQ ID NO:1, and wherein the collagen domain comprises from 8 amino acids corresponding to position R100 as indicated in SEQ ID NO:1 to 29 amino acids corresponding to position D79 as indicated in SEQ ID NO:1, and wherein the collagen domain comprises a lysine which is hydroxylated and glycosylated, wherein the adiponectin polypeptide comprises the mutation T121C; and

a first polymer covalently attached to the introduced cystein residue. Such as a conjugate comprising

an adiponectin polypeptide fragment having a sequence selected from any one of SEQ ID NO:17, or 26, and wherein the collagen domain comprises a lysine which is hydroxylated and glycosylated; and

a first polymer covalently attached to the introduced cystein residue T121C.

In a further example the present invention relates to a conjugate comprising

an adiponectin polypeptide fragment comprising a globular domain and a collagen domain, wherein the globular domain comprises an amino acid sequence from position A 108 to N244 as indicated in SEQ ID NO:1, and wherein the collagen domain comprises from 7 amino acids corresponding to position K101 as indicated in SEQ ID NO:1 to 56 amino acids corresponding to position A52 as indicated in SEQ ID NO:1, and wherein the collagen domain comprises a lysine which is hydroxylated and glycosylated, wherein the adiponectin polypeptide fragment comprises the mutation N127C; and

a first polymer covalently attached to the introduced cystein residue.

In a further example the present invention relates to a conjugate comprising an adiponectin polypeptide fragment comprising a globular domain and a collagen domain,

wherein the globular domain comprises an amino acid sequence from position A108 to N244 as indicated in SEQ ID NO:1, and wherein the collagen domain comprises from 8 amino acids corresponding to position R100 as indicated in SEQ ID NO:1 to 50 amino acids corresponding to position R58 as indicated in SEQ ID NO:1, and wherein the collagen domain comprises a lysine which is hydroxylated and glycosylated, wherein the adiponectin polypeptide comprises the mutation N127C; and

a first polymer covalently attached to the introduced cystein residue. Such as a conjugate comprising

an adiponectin polypeptide fragment having a sequence selected from any one of SEQ ID NO:35, 41, 44, 47, or 50, and wherein the collagen domain comprises a lysine which is hydroxylated and glycosylated; and

a first polymer covalently attached to the introduced cystein residue N127C.

In a further example the present invention relates to a conjugate comprising an adiponectin polypeptide fragment comprising a globular domain and a collagen domain,

wherein the globular domain comprises an amino acid sequence from position A108 to N244 as indicated in SEQ ID NO:1, and wherein the collagen domain comprises from 8 amino acids corresponding to position R100 as indicated in SEQ ID NO:1 to 29 amino acids corresponding to position D79 as indicated in SEQ ID NO:1, and wherein the collagen domain comprises a lysine which is hydroxylated and glycosylated, wherein the adiponectin polypeptide comprises the mutation N127C; and

a first polymer covalently attached to the introduced cystein residue. Such as a conjugate comprising

an adiponectin polypeptide fragment having a sequence selected from any one of SEQ ID NO:35, or 44, and wherein the collagen domain comprises a lysine which is hydroxylated and glycosylated; and

a first polymer covalently attached to the introduced cystein residue N127C.

In a further example the present invention relates to a conjugate comprising an adiponectin polypeptide fragment comprising a globular domain and a collagen domain, wherein the globular domain comprises an amino acid sequence from position A108 to N244 as indicated in SEQ ID NO:1, and wherein the collagen domain comprises from 7 amino acids corresponding to position K101 as indicated in SEQ ID NO:1 to 56 amino acids corresponding to position A52 as indicated in SEQ ID NO:1, and wherein the collagen domain comprises a lysine which is hydroxylated and glycosylated, wherein the adiponectin polypeptide fragment comprises the mutation S146C; and

a first polymer covalently attached to the introduced cystein residue.

In a further example the present invention relates to a conjugate comprising an adiponectin polypeptide fragment comprising a globular domain and a collagen domain,

wherein the globular domain comprises an amino acid sequence from position A108 to N244 as indicated in SEQ ID NO:1, and wherein the collagen domain comprises from 8 amino acids corresponding to position R100 as indicated in SEQ ID NO:1 to 50 amino acids corresponding to position R58 as indicated in SEQ ID NO:1, and wherein the collagen domain comprises a lysine which is hydroxylated and glycosylated, wherein the adiponectin polypeptide comprises the mutation S146C; and

a first polymer covalently attached to the introduced cystein residue. Such as a conjugate comprising

an adiponectin polypeptide fragment having a sequence selected from any one of SEQ ID NO:18, 27, or 33, and wherein the collagen domain comprises a lysine which is hydroxylated and glycosylated; and

a first polymer covalently attached to the introduced cystein residue S146C.

In a further example the present invention relates to a conjugate comprising an adiponectin polypeptide fragment comprising a globular domain and a collagen domain,

wherein the globular domain comprises an amino acid sequence from position A108 to N244 as indicated in SEQ ID NO:1, and wherein the collagen domain comprises from 8 amino acids corresponding to position R100 as indicated in SEQ ID NO:1 to 29 amino acids corresponding to position D79 as indicated in SEQ ID NO:1, and wherein the collagen domain comprises a lysine which is hydroxylated and glycosylated, wherein the adiponectin polypeptide comprises the mutation S146C; and

a first polymer covalently attached to the introduced cystein residue. Such as a conjugate comprising

an adiponectin polypeptide fragment having a sequence selected from any one of SEQ ID NO:18, or 27, and wherein the collagen domain comprises a lysine which is hydroxylated and glycosylated; and

a first polymer covalently attached to the introduced cystein residue S146C.

In a further example the present invention relates to a conjugate comprising an adiponectin polypeptide fragment comprising a globular domain and a collagen domain, wherein the globular domain comprises an amino acid sequence from position A108 to N244 as indicated in SEQ ID NO:1, and wherein the collagen domain comprises from 7 amino acids corresponding to position K101 as indicated in SEQ ID NO:1 to 56 amino acids corresponding to position A52 as indicated in SEQ ID NO:1, and wherein the collagen domain comprises a lysine which is hydroxylated and glycosylated, wherein the adiponectin polypeptide fragment comprises the mutation N141C; and

a first polymer covalently attached to the introduced cystein residue.

In a further example the present invention relates to a conjugate comprising an adiponectin polypeptide fragment comprising a globular domain and a collagen domain,

wherein the globular domain comprises an amino acid sequence from position A108 to N244 as indicated in SEQ ID NO:1, and wherein the collagen domain comprises from 8 amino acids corresponding to position R100 as indicated in SEQ ID NO:1 to 50 amino acids corresponding to position R58 as indicated in SEQ ID NO:1, and wherein the collagen domain comprises a lysine which is hydroxylated and glycosylated, wherein the adiponectin polypeptide comprises the mutation N141C; and

a first polymer covalently attached to the introduced cystein residue. Such as a conjugate comprising

an adiponectin polypeptide fragment having a sequence selected from any one of SEQ ID NO:36, 45, or 51, and wherein the collagen domain comprises a lysine which is hydroxylated and glycosylated; and

a first polymer covalently attached to the introduced cystein residue N141C.

In a further example the present invention relates to a conjugate comprising an adiponectin polypeptide fragment comprising a globular domain and a collagen domain,

wherein the globular domain comprises an amino acid sequence from position A108 to N244 as indicated in SEQ ID NO:1, and wherein the collagen domain comprises from 8 amino acids corresponding to position R100 as indicated in SEQ ID NO:1 to 29 amino acids corresponding to position D79 as indicated in SEQ ID NO:1, and wherein the collagen domain comprises a lysine which is hydroxylated and glycosylated, wherein the adiponectin polypeptide comprises the mutation N141C; and

a first polymer covalently attached to the introduced cystein residue. Such as a conjugate comprising

an adiponectin polypeptide fragment having a sequence selected from any one of SEQ ID NO:36, or 45, and wherein the collagen domain comprises a lysine which is hydroxylated and glycosylated; and

a first polymer covalently attached to the introduced cystein residue N141C.

In a further example the present invention relates to a conjugate comprising

an adiponectin polypeptide fragment comprising a globular domain and a collagen domain, wherein the globular domain comprises an amino acid sequence from position A108 to N244 as indicated in SEQ ID NO:1, and wherein the collagen domain comprises from 7 amino acids corresponding to position K101 as indicated in SEQ ID NO:1 to 56 amino acids corresponding to position A52 as indicated in SEQ ID NO:1, and wherein the collagen domain comprises a lysine which is hydroxylated and glycosylated, wherein the adiponectin polypeptide fragment comprises the mutation T243C; and

a first polymer covalently attached to the introduced cystein residue.

In a further example the present invention relates to a conjugate comprising an adiponectin polypeptide fragment comprising a globular domain and a collagen domain,

wherein the globular domain comprises an amino acid sequence from position A108 to N244 as indicated in SEQ ID NO:1, and wherein the collagen domain comprises from 8 amino acids corresponding to position R100 as indicated in SEQ ID NO:1 to 50 amino acids corresponding to position R58 as indicated in SEQ ID NO:1, and wherein the collagen domain comprises a lysine which is hydroxylated and glycosylated, wherein the adiponectin polypeptide comprises the mutation T243C; and

a first polymer covalently attached to the introduced cystein residue.

In a further example the present invention relates to a conjugate comprising an adiponectin polypeptide fragment comprising a globular domain and a collagen domain,

wherein the globular domain comprises an amino acid sequence from position A108 to N244 as indicated in SEQ ID NO:1, and wherein the collagen domain comprises from 8 amino acids corresponding to position R100 as indicated in SEQ ID NO:1 to 29 amino acids corresponding to position D79 as indicated in SEQ ID NO:1, and wherein the collagen domain comprises a lysine which is hydroxylated and glycosylated, wherein the adiponectin polypeptide comprises the mutation T243C; and

a first polymer covalently attached to the introduced cystein residue. Such as a conjugate comprising

an adiponectin polypeptide fragment having a sequence selected from any one of SEQ ID NO:19, or 28, and wherein the collagen domain comprises a lysine which is hydroxylated and glycosylated; and

a first polymer covalently attached to the introduced cystein residue T243C.

In a further example the present invention relates to a conjugate comprising

an adiponectin polypeptide fragment comprising a globular domain and a collagen domain, wherein the globular domain comprises an amino acid sequence from position A108 to N244 as indicated in SEQ ID NO:1, and wherein the collagen domain comprises from 7 amino acids corresponding to position K101 as indicated in SEQ ID NO:1 to 56 amino acids corresponding to position A52 as indicated in SEQ ID NO:1, and wherein the collagen domain comprises a lysine which is hydroxylated and glycosylated, wherein the adiponectin polypeptide fragment comprises the mutation N228C; and

a first polymer covalently attached to the introduced cystein residue.

In a further example the present invention relates to a conjugate comprising an adiponectin polypeptide fragment comprising a globular domain and a collagen domain,

wherein the globular domain comprises an amino acid sequence from position A108 to N244 as indicated in SEQ ID NO:1, and wherein the collagen domain comprises from 8 amino acids corresponding to position R100 as indicated in SEQ ID NO:1 to 50 amino acids corresponding to position R58 as indicated in SEQ ID NO:1, and wherein the collagen domain comprises a lysine which is hydroxylated and glycosylated, wherein the adiponectin polypeptide comprises the mutation N228C; and

a first polymer covalently attached to the introduced cystein residue.

In a further example the present invention relates to a conjugate comprising an adiponectin polypeptide fragment comprising a globular domain and a collagen domain,

wherein the globular domain comprises an amino acid sequence from position A108 to N244 as indicated in SEQ ID NO:1, and wherein the collagen domain comprises from 8 amino acids corresponding to position R100 as indicated in SEQ ID NO:1 to 29 amino acids corresponding to position D79 as indicated in SEQ ID NO:1, and wherein the collagen domain comprises a lysine which is hydroxylated and glycosylated, wherein the adiponectin polypeptide comprises the mutation N228C; and

a first polymer covalently attached to the introduced cystein residue. Such as a conjugate comprising

an adiponectin polypeptide fragment having a sequence selected from any one of SEQ ID NO:37, or 46, and wherein the collagen domain comprises a lysine which is hydroxylated and glycosylated; and

a first polymer covalently attached to the introduced cystein residue N228C.

In a further embodiment the polymer or first polymer has a molecular weight of from 1 kDa to 200 kDa (kDa is a well known abbreviation and means kilo Dalton). In a still further embodiment the polymer has a molecular weight of from 2 kDa to 95 kDa. In a still further embodiment the polymer has a molecular weight of from 5 kDa to 80 kDa. In a still further embodiment the polymer has a molecular weight of from 5 or 12 kDa to 60 kDa, such as 5-20 kDa, 1240 kDa, 2040 kDa, 5 kDa, 12 kDa, or 20 kDa. In a further embodiment the polymer or first polymer molecule is selected from the group consisting of mPEG(MAL), mPEG2(MAL), PEG-vinylsulphone, mPEG-OPSS, OPSS-PEG-hydrazide in combination with mPEG-ALD.

To attach for instance, a polymer, such as a PEG to an introduced Cys the OPSS and VS chemistries, e.g. as described in the examples, are suitable. Suitable methods of preparing the conjugate, such as attaching a polymer, is described in the section “Methods of preparing a conjugate of the invention”.

In a further embodiment the polymer or first polymer molecule is selected from the group consisting of 5k-mPEG(MAL), 5k-mPEG-OPSS, 10k-mPEG-OPSS, 20k-mPEG-OPSS, 20k-mPEG(MAL), 40k-mPEG2(MAL), OPSS-PEG₂k-hydrazide in combination with mPEG₃₀ kD-ALD.

In a further embodiment the polymer molecule is selected from the group consisting of SS-PEG, NPC-PEG, aldehyd-PEG, MPEG-SPA, mPEG-SBA, PEG-SCM, mPEG-BTC (All available from Shearwater), and SC-PEG.

In a further embodiment the polymer molecule is selected from the group consisting of 5k-PEG-SCM, 12k-PEG-SCM, 20k-PEG-SCM, 5k-PEG-SPA, 12k-PEG-SPA, 20k-PEG-SPA. (All available from Shearwater).

In the situation where it is decided to introduce a glycosylation site in the adiponectin polypeptide, in order to attach a sugar moiety, such sugar moiety is comprised within the term first non-polypeptide moiety. Accordingly, such particular aspect of the invention relates to a conjugate comprising an adiponectin polypeptide, and a sugar moiety covalently attached to the adiponectin polypeptide, wherein the adiponectin polypeptide comprises an amino acid residue having an attachment group for said sugar moiety, wherein said amino acid residue has been introduced in a position that in the parent adiponectin is occupied by a surface exposed amino acid residue. Moreover, since the introduction of the amino acid residue in a position that in the parent adiponectin is occupied by a surface exposed amino acid residue will lead to a novel polypeptide, then a still further aspect of the invention relates to an adiponectin polypeptide comprising an amino acid residue having an attachment group for a sugar moiety, wherein said amino acid residue has been introduced in a position that in the parent adiponectin is occupied by a surface exposed amino acid residue. Moreover, the introduction of a glycosylation site is preferably done in the globular domain in order not to disturb the collagen structure. Typically, the surface exposed amino acid residue is selected from A108, Y109, V110, Y111, R112, L119, E120, T121, Y122, V123, T124, I125, P126, N127, M128, I130, R131, T133, K134, I135, F136, Y137, N138, Q139, Q140, N141, H142, D144, G145, S146, T147, K149, H151, N153, I154, P155, Y159, A161, I164, T165, Y167, M168, K169, D170, V171, K172, F176, K177, K178, D179, K180, A181, M182, F184, T185, Y186, Q188, Y189, Q190, E191, N192, N193, V194, Q196, S198, G199, S200, H204, E206, V207, G208, D209, Q210, W212, Q214, V215, Y216, G217, E218, G219, E220, R221, N222, L224, Y225, D227, N228, D229, N230, D231, T233, H241, D242, T243, or N244 relative to SEQ ID NO:1.

Any one of the above positions which have been identified as surface exposed amino acid residues may be substituted with an amino acid residue having an attachment group for the sugar moiety. The attachment group for the sugar moiety is selected from an N— or O-glycosylation site. As described above the N-glycosylation site must have the pattern N-X′-S/T/C-X″, wherein X′ and X″ are as defined above.

Thus, in one embodiment the attachment group is selected from an O-glycosylation site. In particular, the adiponectin polypeptide comprises a mutation selected from any one of A108T/S, Y109T/S, V110T/S, Y111T/S, R112T/S, L119T/S, E120T/S, T121S, Y122T/S, V123T/S, T124S, 1125T/S, P126T/S, N127T/S, M128T/S, I130T/S, R131T/S, T133S, K134T/S, I135T/S, F136T/S, Y137T/S, N138T/S, Q139T/S, Q140T/S, N141T/S, H142T/S, D144T/S, G145T/S, S146T, T147S, K149T/S, H151T/S, N153T/S, I154T/S, P155T/S, Y159T/S, A161T/S, H163T/S, I164T/S, T165S, Y167T/S, M168T/S, K169T/S, D170T/S, V171T/S, K172T/S, F176T/S, K177T/S, K178T/S, D179T/S, K180T/S, A181T/S, M182T/S, F184T/S, T185S, Y186T/S, D187T/S, Q188T/S, Y189T/S, Q190T/S, E191T/S, N192T/S, N193T/S, V194T/S, D195T/S, Q196T/S, S198T, G199T/S, S200T, H204T/S, E206T/S, V207T/S, G208T/S, D209T/S, Q210T/S, W212T/S, Q214T/S, V215T/S, Y216T/S, G217T/S, E218T/S, G219T/S, E220T/S, R221T/S, N222T/S, G223T/S, L224T/S, Y225T/S, A226T/S, D227T/S, N228T/S, D229T/S, N230T/S, D231T/S, T233S, F234T/S, F237T/S, H241T/S, D242T/S, T243S, or N244T/S relative to SEQ ID NO:1, preferably A108T/S, Y109T/S, V110T/S, Y111T/S, R112T/S, L119T/S, E120T/S, T121S, Y 122T/S, V 123T/S, T 124S, I125T/S, P126T/S, N127T/S, M 128T/S, 1130T/S, R131T/S, T133S, K134T/S, I135T/S, F136T/S, Y137T/S, N138T/S, Q139T/S, Q140T/S, N141T/S, H142T/S, D144T/S, G145T/S, S146T, T147S, K149T/S, H151T/S, N153T/S, I154T/S, P155T/S, Y159T/S, A161T/S, I164T/S, T165S, Y167T/S, M168T/S, K169T/S, D170T/S, V171T/S, K172T/S, F176T/S, K177T/S, K178T/S, D179T/S, K180T/S, A181T/S, M182T/S, F184T/S, T185S, Y186T/S, Q188T/S, Y189T/S, Q190T/S, E191T/S, N192T/S, N193T/S, V194T/S, Q196T/S, G199T/S, S200T, H204T/S, E206T/S, V207T/S, G208T/S, D209T/S, Q210T/S, W212T/S, Q214T/S, V215T/S, Y216T/S, G217T/S, E218T/S, G219T/S, E220T/S, R221T/S, N222T/S, L224T/S, Y225T/S, D227T/S, N228T/S, D229T/S, N230T/S, D23 IT/S, T233S, H241T/S, D242T/S, T243S, or N244T/S. T/S means either T or S, T is preferred, eg. D242T/S means D242T or D242S, where D242T is preferred. Each of these mutations constitutes an individual embodiment and may be the subject of a claim in combination with any one of the above adiponectin polypeptides, such as any one of the sequences SEQ ID NO:3, 4, 5, 6, 10, 11, 12, or 13, or any one of the adiponectin polypeptide fragments described in the above section “Adiponectin polypeptide fragment(s) of the invention”.

Further, in a particular embodiment the invention relates to a conjugate comprising an adiponectin polypeptide, wherein the adiponectin polypeptide comprises a mutation selected from any one of A108T/S, Y109T/S, V110T/S, Y111T/S, R112T/S, L119T/S, E120T/S, T121S, Y122T/S, V123T/S, T124S, I125T/S, P126T/S, N127T/S, M128T/S, I130T/S, R131T/S, T133S, K134T/S, 1135T/S, F136T/S, Y137T/S, N138T/S, Q139T/S, Q140T/S, N141T/S, H142T/S, D144T/S, G145T/S, S146T, T147S, K149T/S, H151T/S, N153T/S, I154T/S, P155T/S, Y159T/S, A161T/S, I164T/S, T165S, Y167T/S, M168T/S, K169T/S, D170T/S, V171T/S, K172T/S, F176T/S, K177T/S, K178T/S, D179T/S, K180T/S, A181T/S, M182T/S, F184T/S, T185S, Y186T/S, Q188T/S, Y189T/S, Q190T/S, E191T/S, N192T/S, N193T/S, V194T/S, Q196T/S, G199T/S, S200T, H204T/S, E206T/S, V207T/S, G208T/S, D209T/S, Q210T/S, W212T/S, Q214T/S, V215T/S, Y216T/S, G217T/S, E218T/S, G219T/S, E220T/S, R221T/S, N222T/S, L224T/S, Y225T/S, D227T/S, N228T/S, D229T/S, N230T/S, D231T/S, T233S, H241T/S, D242T/S, T243S, or N244T/S relative to SEQ ID NO:1; and a sugar moiety covalently attached to the introduced O-glycosylation site. Moreover, since the introduction of the amino acid residue in a position that in the parent adiponectin is occupied by a surface exposed amino acid residue will lead to a novel polypeptide, then in a still further embodiment the invention relates to an adiponectin polypeptide comprising a mutation selected from any one of A108T/S, Y109T/S, V110T/S, Y110T/S, R112T/S, L119T/S, E120T/S, T121S, Y122T/S, V123T/S, T124S, I125T/S, P126T/S, N127T/S, M128T/S, I130T/S, R131T/S, T133S, K134T/S, I135T/S, F136T/S, Y137T/S, N138T/S, Q139T/S, Q140T/S, N141T/S, H142T/S, D144T/S, G145T/S, S146T, T147S, K149T/S, H151T/S, N153T/S, I154T/S, P155T/S, Y159T/S, A161T/S, I164T/S, T165S, Y167T/S, M168T/S, K169T/S, D170T/S, V171T/S, K172T/S, F176T/S, K177T/S, K178T/S, D179T/S, K180T/S, A181T/S, M182T/S, F184T/S, T185S, Y186T/S, Q188T/S, Y189T/S, Q190T/S, E191T/S, N192T/S, N193T/S, V194T/S, Q196T/S, G199T/S, S200T, H204T/S, E206T/S, V207T/S, G208T/S, D209T/S, Q210T/S, W212T/S, Q214T/S, V215T/S, Y216T/S, G217T/S, E218T/S, G219T/S, E220T/S, R221T/S, N222T/S, L224T/S, Y225T/S, D227T/S, N228T/S, D229T/S, N230T/S, D23 IT/S, T233S, H241T/S, D242T/S, T243S, or N244T/S relative to SEQ ID NO:1, T is preferred.

Thus, in another embodiment the attachment group is selected from an N-glycosylation site. In particular, the adiponectin polypeptide comprises a mutation selected from any one of A108N+V110T/S, Y109N+Y111T/S, V110N+R112T/S, Y111N, Y111N+S113T, R112N+A114T/S, L119N+T121S, L119N, E120N+Y122T/S, T121N+V 123T/S, Y122N, Y122N+T124S, V123N+I125T/S, T124N+P126T/S, P126N+M128T/S, P129T/S, M128N+I130T/S, I130N+F132T/S, R131N, R131N+T133S, T133N+I135T/S, K134N+F136T/S, I135N+Y137T/S, F136N+N138T/S, Y137N+Q139T/S, Q140T/S, Q139N+N141T/S, Q140N+H142T/S, Y143T/S, H142N+D144T/S, D144N, D144N+S146T, G145N, G145N+T147S, S146N+G148T/S, T147N+K149T/S, K149N+H111T/S, H151N+N153T/S, P155T/S, P155N+L157T/S, Y159N+A161T/S, A161N+H163T/S, H163N, H163N+T165S, I164N+V166T/S, T165N+Y167T/S, Y167N+K169T/S, M168N+D170T/S, K169N+V171T/S, D170N+K172T/S, V171N+V173T/S, K172N, K172N+S174T, F176N+K178T/S, K177N+D179T/S, K178N+K180T/S, D179N+A181T/S, K180N+M182T/S, A181N+L183T/S, M182N+F184T/S, F184N+Y186T/S, T185N+D187T/S, Y186N+Q188T/S, D187N+Y189T/S, Q188N+Q190T/S, Y189N+E191T/S, Q190N+N192T/S, E191N+N193T/S, V194T/S, D195T/S, V194N+Q196T/S, D195N+A197T/S, Q196N, Q196N+S198T, S198N, S198N+S200T, G199N+V201T/S, S200N+L202T/S, H204N+E206T/S, E206N+G208T/S, V207N+D209T/S, G208N+Q210T/S, D209N+V211T/S, Q210N+W212T/S, W212N+Q214T/S, Q214N+Y216T/S, V215N+G217T/S, Y216N+E218T/S, G217N+G219T/S, E218N+E220T/S, G219N+R221T/S, E220N+N222T/S, R221N+G223T/S, L224T/S, G223N+Y225T/S, L224N+A226T/S, Y225N+D227T/S, A226N+N228T/S, D227N+D229T/S, N230T/S, D229N+D231T/S, S232T, D231N, D231N+T233S, T233N, T233N+T235S, F234N+G236T/S, F237N+L239T/S, H241N, H241N+T243S, or D242N+N244T/S relative to SEQ ID NO:1, such as A108N+V110T/S, Y109N+Y111T/S, V110N+R112T/S, Y111N, Y111N+S113T, R112N+A114T/S, L119N+T121S, L119N, E120N+Y122T/S, T121N+V123T/S, Y122N, Y122N+T124S, T124N+P126T/S, P126N+M128T/S, P129T/S, M128N+I130T/S, I130N+F132T/S, R131N, R131N+T133S, T133N+I135T/S, K134N+F136T/S, I135N+Y137T/S, F136N+N138T/S, Y137N+Q139T/S, Q140T/S, Q139N+N141T/S, Q140N+H142T/S, Y143T/S, H142N+D144T/S, D144N, D144N+S146T, G145N, G145N+T147S, S146N+G148T/S, T147N+K149T/S, K149N+H151T/S, H151N+N153T/S, P155T/S, P155N+L157T/S, Y159N+A161T/S, I164N+V166T/S, T165N+Y167T/S, Y167N+K169T/S, M168N+D170T/S, K169N+V171T/S, D170N+K172T/S, V171N+V173T/S, K172N, K172N+S174T, F176N+K178T/S, K177N+D179T/S, K178N+K180T/S, D179N+A181T/S, K180N+M182T/S, A181N+L183T/S, M182N+F184T/S, F184N+Y186T/S, Y186N+Q188T/S, Q188N+Q190T/S, Y189N+E191T/S, Q190N+N192T/S, E191N+N193T/S, V194T/S, V194N+Q196T/S, Q196N, Q196N+S198T, G199N+V201T/S, S200N+L202T/S, H204N+E206T/S, E206N+G208T/S, V207N+D209T/S, G208N+Q210T/S, D209N+V211T/S, Q210N+W212T/S, W212N+Q214T/S, Q214N+Y216T/S, V215N+G217T/S, Y216N+E218T/S, G217N+G219T/S, E218N+E220T/S, G219N+R221T/S, or E220N+N222T/S, e.g. A108N+V110T, Y109N+Y111T, V110N+R112T, Y111N, Y111N+S113T, R112N+A114T, L119N+T121S, L119N, E120N+Y122T, T121N+V123T, Y122N, Y122N+T124S, T124N+P126T, P126N+M128T, P129T, M128N+I130T, I130N+F132T, R131N, R131N+T133S, T133N+I135T, K134N+F136T, I135N+Y137T, F136N+N138T, Y137N+Q139T, Q140T, Q139N+N141T, Q140N+H142T, Y143T, H142N+D144T, D144N, D144N+S146T, G145N, G145N+T147S, S146N+G148T, T147N+K149T, K149N+H151T, H151N+N153T, P155T, P155N+L157T, Y159N+A161T, I164N+V166T, T165N+Y167T, Y167N+K169T, M168N+D170T, K169N+V171T, D170N+K172T, V171N+V173T, K172N, K172N+S174T, F176N+K178T, K177N+D179T, K178N+K180T, D179N+A181T, K180N+M182T, A181N+L183T, M182N+F184T, F184N+Y186T, Y186N+Q188T, Q188N+Q190T, Y189N+E191T, Q190N+N192T, E191N+N193T, V194T, V194N+Q196T, Q196N, Q196N+S198T, G199N+V201T, S200N+L202T, H204N+E206T, E206N+G208T, V207N+D209T, G208N+Q210T, D209N+V211T, Q210N+W212T, W212N+Q214T, Q214N+Y216T, V215N+G217T, Y216N+E218T, G217N+G219T, E218N+E220T, G219N+R221T, or E220N+N222T. Each of these mutations constitutes an individual embodiment and may be the subject of a claim in combination with any one of the above adiponectin polypeptides, such as any one of the sequences SEQ ID NO:3, 4, 5, 6, 10, 11, 12, or 13, or any one of the adiponectin polypeptide fragments described in the above section “Adiponectin polypeptide fragment(s) of the invention”.

Further, in a particular embodiment the invention relates to a conjugate comprising an adiponectin polypeptide, wherein the adiponectin polypeptide comprises a mutation selected from any one of A108N+V110T/S, Y109N+Y111T/S, V110N+R112T/S, Y111N, Y111N+S113T, R112N+A114T/S, L119N+T121S, L119N, E120N+Y122T/S, T121N+V123T/S, Y122N, Y122N+T124S, T124N+P126T/S, P126N+M128T/S, P129T/S, M128N+I130T/S, I130N+F132T/S, R131N, R131N+T133S, T133N+I135T/S, K134N+F136T/S, I135N+Y137T/S, F136N+N138T/S, Y137N+Q139T/S, Q140T/S, Q139N+N141T/S, Q140N+H142T/S, Y143T/S, H142N+D144T/S, D144N, D144N+S146T, G145N, G145N+T147S, S146N+G148T/S, T147N+K149T/S, K149N+H151T/S, H151N+N153T/S, P155T/S, P155N+L157T/S, Y159N+A161T/S, I164N+V166T/S, T165N+Y167T/S, Y167N+K169T/S, M168N+D 170T/S, K169N+V171T/S, D170N+K172T/S, V171N+V173T/S, K172N, K172N+S174T, F176N+K178T/S, K177N+D179T/S, K178N+K180T/S, D179N+A181T/S, K180N+M182T/S, A181N+L183T/S, M182N+F184T/S, F184N+Y186T/S, Y186N+Q188T/S, Q188N+Q190T/S, Y189N+E191T/S, Q190N+N192T/S, E191N+N193T/S, V194T/S, V194N+Q196T/S, Q196N, Q196N+S198T, G199N+V201T/S, S200N+L202T/S, H204N+E206T/S, E206N+G208T/S, V207N+D209T/S, G208N+Q210T/S, D209N+V211T/S, Q210N+W212T/S, W212N+Q214T/S, Q214N+Y216T/S, V215N+G217T/S, Y216N+E218T/S, G217N+G219T/S, E218N+E220T/S, G219N+R221T/S, or E220N+N222T/S relative to SEQ ID NO:1; and a sugar moiety covalently attached to the introduced N-glycosylation site. Moreover, since the introduction of the N-glycosylation site in a position that in the parent adiponectin is occupied by a surface exposed amino acid residue will lead to a novel polypeptide, then in a still further embodiment the invention relates to an adiponectin polypeptide comprising a mutation selected from any one of A108N+V110T/S, Y109N+Y111T/S, V110N+R112T/S, Y111N, Y111N+S113T, R112N+A114T/S, L119N+T121S, L119N, E120N+Y122T/S, T121N+V123T/S, Y122N, Y122N+T124S, T124N+P126T/S, P126N+M128T/S, P129T/S, M128N+I130T/S, I130N+F132T/S, R131N, R131N+T133S, T133N+I135T/S, K134N+F136T/S, I135N+Y137T/S, F136N+N138T/S, Y137N+Q139T/S, Q140T/S, Q139N+N141T/S, Q140N+H142T/S, Y143T/S, H142N+D144T/S, D144N, D144N+S146T, G145N, G145N+T147S, S146N+G148T/S, T147N+K149T/S, K149N+H151T/S, H151N+N153T/S, P155T/S, P155N+L157T/S, Y159N+A161T/S, I164N+V166T/S, T165N+Y167T/S, Y167N+K169T/S, M168N+D170T/S, K169N+V171T/S, D170N+K172T/S, V171N+V173T/S, K172N, K172N+S174T, F176N+K178T/S, K177N+D179T/S, K178N+K180T/S, D179N+A181T/S, K180N+M182T/S, A181N+L183T/S, M182N+F184T/S, F184N+Y186T/S, Y186N+Q188T/S, Q188N+Q190T/S, Y189N+E191T/S, Q190N+N192T/S, E191N+N193T/S, V194T/S, V194N+Q196T/S, Q196N, Q196N+S198T, G199N+V201T/S, S200N+L202T/S, H204N+E206T/S, E206N+G208T/S, V207N+D209T/S, G208N+Q210T/S, D209N+V211T/S, Q210N+W212T/S, W212N+Q214T/S, Q214N+Y216T/S, V215N+G217T/S, Y216N+E218T/S, G217N+G219T/S, E218N+E220T/S, G219N+R221T/S, or E220N+N222T/S relative to SEQ ID NO:1.

Typically, the adiponectin polypeptide wherein a glycosylation site is introduced is selected from any one of the sequences SEQ ID NO:3, 4, 5, 6, 10, 11, 12, or 13, or any one of the adiponectin polypeptide fragments described in the above section “Adiponectin polypeptide fragment(s) of the invention”, such adiponectin polypeptide is preferably expressed in a eucaryotic cell, such as a mammalian cell, and in this respect will be conjugated to a sugar moiety. To illustrate this a few embodiments are outlined hereafter.

Accordingly, the invention relates to an adiponectin polypeptide selected from any one of the sequences SEQ ID NO:3, 4, 5, 6, 10, 11, 12, or 13, such as SEQ ID NO:10, or SEQ ID NO:11, comprising a mutation selected from any one of A108N+V110T/S, Y109N+Y111T/S, V110N+R112T/S, Y111N, Y111N+S113T, R112N+A114T/S, L119N+T121S, L119N, E120N+Y122T/S, T121N+V123T/S, Y122N, Y122N+T124S, T124N+P126T/S, P126N+M128T/S, P129T/S, M128N+I130T/S, I130N+F132T/S, R131N, R131N+T133S, T133N+I135T/S, K134N+F136T/S, I135N+Y137T/S, F136N+N138T/S, Y137N+Q139T/S, Q140T/S, Q139N+N141T/S, Q140N+H142T/S, Y143T/S, H142N+D144T/S, D144N, D144N+S146T, G145N, G145N+T147S, S146N+G148T/S, T147N+K149T/S, K149N+H151T/S, H151N+N153T/S, P155T/S, P155N+L157T/S, Y159N+A161T/S, I164N+V166T/S, T165N+Y167T/S, Y167N+K169T/S, M168N+D170T/S, K169N+V171T/S, D170N+K172T/S, V171N+V173T/S, K172N, K172N+S174T, F176N+K178T/S, K177N+D179T/S, K178N+K180T/S, D179N+A181T/S, K180N+M182T/S, A181N+L183T/S, M182N+F184T/S, F184N+Y186T/S, Y 186N+Q188T/S, Q 188N+Q190T/S, Y189N+E191T/S, Q190N+N192T/S, E191N+N193T/S, V194T/S, V194N+Q196T/S, Q196N, Q196N+S198T, G199N+V201T/S, S200N+L202T/S, H204N+E206T/S, E206N+G208T/S, V207N+D209T/S, G208N+Q210T/S, D209N+V211T/S, Q210N+W212T/S, W212N+Q214T/S, Q214N+Y216T/S, V215N+G217T/S, Y216N+E218T/S, G217N+G219T/S, E218N+E220T/S, G219N+R221T/S, or E220N+N222T/S relative to SEQ ID NO:1, such as a mutation selected from any one of A108N+V110T/S, Y109N+Y111T/S, V110N+R112T/S, Y111N, Y111N+S113T, R112N+A114T/S, L119N+T121S, L119N, E120N+Y122T/S, T121N+V123T/S, Y122N, Y122N+T124S, T124N+P126T/S, P126N+M128T/S, P129T/S, M128N+I130T/S, I130N+F132T/S, R131N, R131N+T133S, T133N+I135T/S, K134N+F136T/S, I135N+Y137T/S, F136N+N138T/S, Y137N+Q139T/S, Q140T/S, Q139N+N141T/S, Q140N+H142T/S, Y143T/S, H142N+D144T/S, D144N, D144N+S146T, G145N, G145N+T147S, S146N+G148T/S, T147N+K149T/S, K149N+H111T/S, H151N+N153T/S, P155T/S, P155N+L157T/S, Y159N+A161T/S, I164N+V166T/S, T165N+Y167T/S, Y167N+K169T/S, M168N+D170T/S, K169N+V171T/S, D170N+K172T/S, V171N+V173T/S, K172N, K172N+S174T, F176N+K178T/S, K177N+D179T/S, K178N+K180T/S, D179N+A181T/S, K180N+M182T/S, A181N+L183T/S, M182N+F184T/S, F184N+Y186T/S, Y186N+Q188T/S, Q188N+Q190T/S, Y189N+E191T/S, Q190N+N192T/S, E191N+N193T/S, V194T/S, V194N+Q196T/S, Q196N, Q196N+S198T, G199N+V201T/S, S200N+L202T/S, or H204N+E206T/S, in particular a mutation selected from any one of A108N+V110T/S, Y109N+Y111T/S, V110N+R112T/S, Y111N, Y111N+S113T, R112N+A114T/S, L119N+T121S, L119N, E120N+Y122T/S, T121N+V123T/S, Y122N, Y122N+T124S, T124N+P126T/S, P126N+M128T/S, P129T/S, M128N+I130T/S, I130N+F132T/S, R131N, R131N+T133S, T133N+I135T/S, K134N+F136T/S, I135N+Y137T/S, F136N+N138T/S, Y137N+Q139T/S, Q140T/S, Q139N+N141T/S, Q140N+H142T/S, Y143T/S, H142N+D144T/S, D144N, D144N+S146T, G145N, G145N+T147S, S146N+G148T/S, T147N+K149T/S, K149N+H111T/S, H151N+N153T/S, P155T/S, P155N+L157T/S, Y159N+A161T/S, I164N+V 166T/S, T165N+Y167T/S, Y 167N+K169T/S, M168N+D170T/S, K169N+V171T/S, D170N+K172T/S, V171N+V173T/S, K172N, K172N+S174T, F176N+K178T/S, K177N+D179T/S, K178N+K180T/S, D179N+A181T/S, K180N+M182T/S, A181N+L183T/S, M182N+F184T/S, F184N+Y186T/S, Y186N+Q188T/S, Q188N+Q190T/S, Y189N+E191T/S, Q190N+N192T/S, E191N+N193T/S, V194T/S, or V194N+Q196T/S, preferably a mutation selected from any one of A108N+V110T, Y109N+Y111T, V110N+R112T, Y111N, Y111N+S113T, R112N+A114T, L119N+T121S, L119N, E120N+Y122T, T121N+V123T, Y122N, Y122N+T124S, T124N+P126T, P126N+M128T, P129T, M128N+I130T, I130N+F132T, R131N, R131N+T133S, T133N+I135T, K134N+F136T, I135N+Y137T, F136N+N138T, Y137N+Q139T, Q140T, Q139N+N141T, Q140N+H142T, Y143T, H142N+D144T, D144N, D144N+S146T, G145N, G145N+T147S, S146N+G148T, T147N+K149T, K149N+H151T, H151N+N153T, P155T, P155N+L157T, Y159N+A161T, I164N+V166T, T165N+Y167T, Y167N+K169T, M168N+D170T, K169N+V171T, D170N+K172T, V171N+V173T, K172N, K172N+S174T, F176N+K178T, K177N+D179T, K178N+K180T, D179N+A181T, K180N+M182T, A181N+L183T, M182N+F184T, F184N+Y186T, Y186N+Q188T, Q188N+Q190T, Y189N+E191T, Q190N+N192T, E191N+N193T, V194T, or V194N+Q196T. In a further embodiment the invention relates to an adiponectin polypeptide selected from the sequence SEQ ID NO:10 comprising a mutation selected from any one of Y111N, Y122N, P129T, R131N, D144N+S146T, G145N, H₁₅N+N153T, P155T, K178N+K180T, such as Y111N, Y122N, R131N, D144N+S146T, H511N+N153T, K178N+K180T. In a further embodiment the invention relates to an adiponectin polypeptide selected from the sequence SEQ ID NO:10 comprising a mutation selected from Y111N, such as the adiponectin polypeptide having the SEQ ID NO:53. In a further embodiment the invention relates to an adiponectin polypeptide selected from the sequence SEQ ID NO:10 comprising a mutation selected from Y122N, such as the adiponectin polypeptide having the SEQ ID NO:54. In a further embodiment the invention relates to an adiponectin polypeptide selected from the sequence SEQ ID NO:10 comprising a mutation selected from R13 iN, such as the adiponectin polypeptide having the SEQ ID NO:55. In a further embodiment the invention relates to an adiponectin polypeptide selected from the sequence SEQ ID NO:10 comprising a mutation selected from D144N+S146T, such as the adiponectin polypeptide having the SEQ ID NO:56. In a further embodiment the invention relates to an adiponectin polypeptide selected from the sequence SEQ ID NO:10 comprising a mutation selected from H151N+N153T, such as the adiponectin polypeptide having the SEQ ID NO:57. In a further embodiment the invention relates to an adiponectin polypeptide selected from the sequence SEQ ID NO:10 comprising a mutation selected from K178N+K180T, such as the adiponectin polypeptide having the SEQ ID NO:58. In a further embodiment the invention relates to an adiponectin polypeptide selected from the sequence SEQ ID NO:10 comprising a mutation selected from P129T, such as the adiponectin polypeptide having the SEQ ID NO:59. In a further embodiment the invention relates to an adiponectin polypeptide selected from the sequence SEQ ID NO:10 comprising a mutation selected from G145N, such as the adiponectin polypeptide having the SEQ ID NO:60. In a further embodiment the invention relates to an adiponectin polypeptide selected from the sequence SEQ ID NO:10 comprising a mutation selected from P155T, such as the adiponectin polypeptide having the SEQ ID NO:61. In a further embodiment the invention relates to an adiponectin polypeptide selected from the sequence SEQ ID NO:11 comprising a mutation selected from any one of Y111N, Y122N, P129T, R131N, D144N+S146T, G145N, H₁₅₁N+N153T, P155T, K178N+K180T, such as Y111N, Y122N, R131N, D144N+S146T, H151N+N153T, K178N+K180T. In a further embodiment the invention relates to an adiponectin polypeptide selected from the sequence SEQ ID NO:11 comprising a mutation selected from Y111N. In a further embodiment the invention relates to an adiponectin polypeptide selected from the sequence SEQ ID NO:11 comprising a mutation selected from Y122N. In a further embodiment the invention relates to an adiponectin polypeptide selected from the sequence SEQ ID NO:11 comprising a mutation selected from R131N. In a further embodiment the invention relates to an adiponectin polypeptide selected from the sequence SEQ ID NO:11 comprising a mutation selected from D144N+S146T. In a further embodiment the invention relates to an adiponectin polypeptide selected from the sequence SEQ ID NO:11 comprising a mutation selected from H151N+N153T. In a further embodiment the invention relates to an adiponectin polypeptide selected from the sequence SEQ ID NO:11 comprising a mutation selected from K178N+K180T.

Hereafter, in connection with the introduction of an N-glycosylation site in the adiponectin polypeptide either conjugated to a sugar moiety or non-conjugated, the mutation may be selected from any one of the above mentioned N-glycosylation sites, and the adiponectin polypeptide may be selected from any one of the above mentioned adiponectin polypeptide fragments, however, for illustrative purposes only a small group of adiponectin polypeptide fragments and of N-glycosylation sites will be indicated.

Thus, the invention relates to an adiponectin polypeptide selected from an adiponectin polypeptide fragment comprising a globular domain and a collagen domain, wherein the globular domain comprises an amino acid sequence from position A108 to N244 as indicated in SEQ ID NO:1, and wherein the collagen domain comprises from 7 amino acids corresponding to position K101 as indicated in SEQ ID NO:1 to 56 amino acids corresponding to position A52 as indicated in SEQ ID NO:1, and wherein the collagen domain comprises a lysine which is hydroxylated and glycosylated, the adiponectin polypeptide fragment comprising a mutation selected from any one of Y111N, Y122N, P129T, R131N, D144N+S146T, G145N, H151N+N153T, P155T, K178N+K180T. In a further embodiment the adiponectin polypeptide fragment comprises a globular domain and a collagen domain, wherein the globular domain comprises an amino acid sequence from position A 108 to N244 as indicated in SEQ ID NO:1, and wherein the collagen domain comprises from 8 amino acids corresponding to position R100 as indicated in SEQ ID NO:1 to 50 amino acids corresponding to position R58 as indicated in SEQ ID NO:1, and wherein the collagen domain comprises a lysine which is hydroxylated and glycosylated.

Furthermore, the invention relates to a conjugate comprising an adiponectin polypeptide, wherein the adiponectin polypeptide has the sequence SEQ ID NO:10 comprising a mutation selected from any one of Y111N, Y122N, P129T, R131N, D144N+S146T, G145N, H151N+N153T, P155T, or K178N+K180T relative to SEQ ID NO:1; and a sugar moiety covalently attached to the introduced N-glycosylation site. In a further embodiment the invention relates to a conjugate comprising an adiponectin polypeptide, wherein the adiponectin polypeptide has the sequence SEQ ID NO: II comprising a mutation selected from any one of Y111N, Y122N, P129T, R131N, D144N+S146T, G145N, H151N+N153T, P155T, or K178N+K180T relative to SEQ ID NO:1; and a sugar moiety covalently attached to the introduced N-glycosylation site.

Moreover, the invention relates to a conjugate comprising an adiponectin polypeptide fragment which comprises a globular domain and a collagen domain, wherein the globular domain comprises an amino acid sequence from position A108 to N244 as indicated in SEQ ID NO:1, and wherein the collagen domain comprises from 8 amino acids corresponding to position R100 as indicated in SEQ ID NO:1 to 50 amino acids corresponding to position R58 as indicated in SEQ ID NO:1, and wherein the collagen domain comprises a lysine which is hydroxylated and glycosylated, the adiponectin polypeptide fragment comprising a mutation selected from any one of Y111N, Y122N, P129T, R131N, D144N+S146T, G145N, H151N+N153T, P155T, K178N+K180T;

and a sugar moiety covalently attached to the introduced N-glycosylation site.

The sugar moiety may be introduced by methods known to the person skilled in the art, or as suggested in the section “Methods of preparing a conjugate of the invention” herein. Preferably, a mammalian cell line is used to express the adiponectin polypeptide, such as a CHO cell, BHK cell, or HEK cell.

When the collagen domain comprises a lysine which is hydroxylated and glycosylated, it may comprise 1, 2, 3, or 4 lysine(s), as explained in detail above.

Although, the adiponectin polypeptide may be modified to comprise more than one introduced glycosylation site in the globular domain it is preferred that no more than four glycosylation sites are introduced, that is, one to four N-glycosylation site(s) or one to four O-glycosylation site(s) or mixtures thereof, provided that no more than four glycosylation sites are introduced, such as one N-glycosylation site, two N-glycosylation sites, three N-glycosylation sites, four N-glycosylation sites, three O-glycosylation sites, four O-glycosylation sites, or one N-glycosylation site and one O-glycosylation site. In a more preferred embodiment the adiponectin polypeptide comprises at least one introduced N-glycosylation site, such as one introduced N-glycosylation site. The adiponectin polypeptide may be non-conjugated, or preferably, conjugated to a sugar moiety attached to the introduced glycosylation site, such as an N-glycosylation site.

In addition to the first non-polypeptide moiety, which is typically selected from a polymer attached to an amino acid residue such as a lysine, aspartic acid, glutamic acid or cysteine residue, or a sugar moiety attached to an introduced N-glycosylation site, the adiponectin polypeptide may optionally also comprise a second non-polypeptide moiety which is different from the first non-polypeptide moiety. Thus, if for instance, the first non-polypeptide moiety is a polymer, then the second non-polypeptide moiety is typically a sugar moiety, or if the first non-polypeptide moiety is a sugar moiety, then the second non-polypeptide moiety is typically a polymer.

Accordingly, in a further embodiment the conjugate further comprises a second non-polypeptide moiety selected from the group consisting of a polymer molecule, a lipophilic compound, a sugar moiety and an organic derivatizing agent. The second non-polypeptide moiety is different from the first non-polypeptide moiety, however, the above embodiments described in connection with the first non-polypeptide moiety are also considered embodiments for the second non-polypeptide moiety.

In a further embodiment the second non-polypeptide moiety is selected from a polymer molecule.

In a further embodiment the amino acid residue having the attachment group for said second non-polypeptide moiety is selected from a lysine, aspartic acid, glutamic acid or cysteine residue, such as a cysteine residue.

In a further embodiment the second non-polypeptide moiety is a polymer, typically a linear or branched polyethylene glycol.

In a further embodiment the second non-polypeptide moiety is a polymer molecule having a sugar moiety as an attachment group.

In a further embodiment the polymer molecule is selected from the group consisting of mPEG-AMINE. (Available from Shearwater).

In a further embodiment the polymer molecule is selected from the group consisting of 5k-mPEG-AMINE. (Available from Shearwater)

In a further embodiment the amino acid sequence of the adiponectin polypeptide further comprises at least one removed lysine residue.

In a further embodiment one to four lysine residues selected from any one of the positions K65, K68, K77, or K101 of the collagen domain of human adiponectin is/are removed.

In a further embodiment one to six lysine residues selected from any one of the positions K134, K149, K169, K172, K177, K178, or K180 of the globular domain of wild-type human adiponectin is/are removed.

Such lysine residues may be removed from the collagen and/or globular domain, depending on the length of the adiponectin polypeptide. The skilled person will understand that the group of lysines to select from will depend on whether the full collagen domain or only a fragment thereof is present in the adiponectin polypeptide, and thus whether the group of lysine residues are the positions K65, K68, K77, or K101 of the collagen domain of human adiponectin and positions K134, K149, K169, K172, K177, K178, or K180 of the globular domain of human adiponectin, or a smaller group, such as K77, or K110 of the collagen domain and positions K134, K149, K169, K172, K177, K178, or K180 of the globular domain, or even a smaller group, such as K110 of the collagen domain and positions K134, K149, K169, K172, K177, K178, or K180 of the globular domain. If desired to introduce a second non-polypeptide moiety by conjugating it to a lysine, then obviously, at least one lysine should be present in the adiponectin polypeptide in order to make possible the conjugation to a lysine.

Calcium Composition Aspects

We have shown that calcium ions are crucial for the adiponectin polypeptide to form stable trimers and that removal of such calcium ions leads to destabilization of the trimer structure. No effect could be seen with other divalent cations such as magnesium and zinc ions. The destabilization of the trimer structure leads to a heterogenous composition as shown in native gels. The addition of calcium ions to a liquid solution of adiponectin which had a destabilized trimer structure lead to recovery of the stable trimer structure. In particular we have shown that lowering pH in the absence of calcium ions destabilize the trimer structure, and that adding calcium ions leads to a stable trimer. The stable trimer structure has biological activity which may be tested in various in vitro or in vivo models, such in vivo models may be one of the recognized mouse models for testing insulin sensitivity, or obesity. From our experimental analysis of the structure of a human adiponectin fragment (apM1(82-244) it has become clear that D187, and D195, releative to SEQ ID NO:1 in the globular domain of human adiponectin are involved in the binding of calcium ions, and that mutation in one or both of these positions results in reduced affinity to calcium ions. Furthermore H163 is also believed to be important for calcium binding. Thus, in order to maintain the calcium binding it is preferred to maintain D187, and D195 releative to SEQ ID NO:1, and more preferably D187, D195, and H163 should be maintained.

A typical way of testing biological activity is in the test Assay A, B, or C, described in the experimental section. The adiponectin polypeptide trimer will usually consist of three identical monomers, however, the trimer may also be heterogenous, for instance, two of the monomers may be the same and the third may be different, or all three monomers may be different. The difference being that one or two monomer(s) has/have an amino acid sequence that differs from that of the other monomer(s). Another difference could be in a sugar moiety, eg. in different hydroxy-glycosylations in the collagenous domain on each adiponectin polypeptide monomer. In case the individual monomers are identical but have different sugar moieties attached, this is intended to be comprised within the term “homotrimer”. In case the adiponectin polypeptide trimer consist of three identical monomers, that is three identical amino acid sequences, it is referred to as a homotrimer. In a further embodiment the trimer is a heterotrimer. In a further embodiment the trimer is a homotrimer.

Thus, calcium ions stabilize the adiponectin polypeptide trimer and this intimate assembly is referred to herein as a complex.

Accordingly, in a broad aspect the present invention relates to an isolated complex comprising a) an adiponectin polypeptide or a conjugate comprising an adiponectin polypeptide, and a first non-polypeptide moiety covalently attached to the adiponectin polypeptide, and b) calcium ions.

In a further aspect the present invention relates to an isolated complex comprising a) an adiponectin polypeptide, and b) calcium ions.

In a further aspect the present invention relates to an isolated complex comprising a) a conjugate comprising an adiponectin polypeptide, and a first non-polypeptide moiety covalently attached to the adiponectin polypeptide, and b) calcium ions.

In an embodiment of the above aspects the adiponectin polypeptide is expressed and recovered from mammalian host cells. Preferred host cells are CHO, BHK, or HEK cells, in particular CHO-K1 and HEK293 cells.

In a further embodiment of the above aspects the adiponectin polypeptide is expressed and recovered from yeast cells.

In an alternative embodiment of the above aspects the adiponectin polypeptide is expressed and recovered from bacterial cells. Examples of bacterial host cells include grampositive bacteria such as strains of Bacillus, e.g. B. brevis or B. subtilis, Pseudomonas or Streptomyces, or gramnegative bacteria, such as strains of E. coli. A typically, embodiment is an E. Coli host cell.

In a further aspect the present invention relates to an isolated complex comprising a) an adiponectin polypeptide or a conjugate comprising an adiponectin polypeptide, and a first non-polypeptide moiety covalently attached to the adiponectin polypeptide, and b) calcium ions, provided that the adiponectin polypeptide is expressed and recovered from mammalian host cells.

In a further aspect the present invention relates to an isolated complex comprising a) an adiponectin polypeptide or a conjugate comprising an adiponectin polypeptide, and a first non-polypeptide moiety covalently attached to the adiponectin polypeptide, and b) calcium ions, provided that the adiponectin polypeptide is expressed and recovered from bacterial host cells.

Thus, the group under a) may be selected from an adiponectin polypeptide in one embodiment or from a conjugate comprising an adiponectin polypeptide, and a first non-polypeptide moiety covalently attached to the adiponectin polypeptide in another embodiment.

In particular human adiponectin (apM1) and fragments thereof, as well as analogs thereof, such as an adiponectin polypeptide comprising a globular domain having at least 80% identity to the globular domain of apM1 (shown in sequence id no 6) and optionally comprising a collagen domain or fragment thereof, are preferred embodiments of the adiponectin polypeptide.

In one embodiment the adiponectin polypeptide is not full-length acrp30. In another embodiment the adiponectin polypeptide is not acrp30 fragment (104-247). In a further embodiment the adiponectin polypeptide is not acrp30 fragments. In a further embodiment the adiponectin polypeptide is not human full-length adiponectin. The human full-length adiponectin may be purified from human plasma or produced recombinantly from E. Coli cells.

The stable adiponectin polypeptide is a trimer wherein the trimer consists of three monomers. Thus, in a further embodiment the adiponectin polypeptide is a trimer (adiponectin polypeptide trimer). Typically, the trimer is a homotrimer. However, the trimer may also be a heterotrimer.

In a further aspect the present invention relates to a liquid composition comprising an isolated complex wherein the complex comprises a) an adiponectin polypeptide or a conjugate comprising an adiponectin polypeptide, and a first non-polypeptide moiety covalently attached to the adiponectin polypeptide, and b) calcium ions.

In a further aspect the present invention relates to a liquid composition comprising an isolated complex wherein the complex comprises a) an adiponectin polypeptide, and b) calcium ions.

In a further aspect the present invention relates to a liquid composition comprising an isolated complex wherein the complex comprises a) a conjugate comprising an adiponectin polypeptide, and a first non-polypeptide moiety covalently attached to the adiponectin polypeptide, and b) calcium ions.

In a further aspect the present invention relates to a liquid composition comprising an isolated complex wherein the complex comprises a) an adiponectin polypeptide or a conjugate comprising an adiponectin polypeptide, and a first non-polypeptide moiety covalently attached to the adiponectin polypeptide, and b) calcium ions, provided that the adiponectin polypeptide is expressed and recovered from mammalian host cells.

In a further aspect the present invention relates to a liquid composition comprising an isolated complex wherein the complex comprises a) an adiponectin polypeptide or a conjugate comprising an adiponectin polypeptide, and a first non-polypeptide moiety covalently attached to the adiponectin polypeptide, and b) calcium ions, provided that the adiponectin polypeptide is expressed and recovered from bacterial host cells.

The liquid composition may be a solution or suspension, and may comprise a buffer. However, liquid solutions are preferred. Thus, in an embodiment the liquid composition is a liquid solution, such as an aqueous solution. In a further embodiment the liquid composition, such as the liquid solution, further comprises a buffer. The buffer may be any suitable buffer such as any one of those mentioned below in the section “Pharmaceutical composition and uses of a conjugate or adiponectin polypeptide fragment of the invention”, however, care should be taken that if a phosphate buffer is used then pH should not be too low and preferably above 4, such as above 5, even more preferably above 6. However, if calcium ions are added to the composition then the trimer structure will be stable in a broad pH range, such as from pH 2-10, preferably from 3-9.

In a further aspect the present invention relates to a pharmaceutical composition comprising a) an adiponectin polypeptide or a conjugate comprising an adiponectin polypeptide, and a first non-polypeptide moiety covalently attached to the adiponectin polypeptide, b) calcium ions, and c) a pharmaceutically acceptable carrier. In one embodiment such pharmaceutical composition is a liquid composition, such as a liquid solution. In a further embodiment the pharmaceutical composition comprises a buffer and has a pH from 2-10, provided that the buffer is not a phosphate buffer. In another embodiment the pharmaceutical composition comprises a buffer and has a pH from 4-10, such as 5-10, preferably 6-9. In a further embodiment the pharmaceutical composition comprises a buffer and has a pH from 2-10, such as 3-9, and calcium ions. Typically, a molar surplus of calcium ions relative to the adiponectin polypeptide is present in the composition.

The use of calcium ions to prepare an isolated complex comprising an adiponectin polypeptide or a conjugate comprising an adiponectin polypeptide, and a first non-polypeptide moiety covalently attached to the adiponectin polypeptide, provides a stable trimer of the adiponectin polypeptide wherein the complex has biological activity. Typically, such biological activity can be measured in any one of the test assays described in the experimental section, that is, Test Assay: Determination of adiponectin's effect on glucose uptake in C2C12 cells; or Test Assay: Measurement of inhibition of LPS-induced TNF-alpha production. Moreover, the effect of calcium ions to stabilize the trimer structure of the adiponectin polypeptide or the conjugate may be tested by reducing pH in a phosphate containing buffer in the absence or presence of calcium ions.

Thus, in a further aspect the present invention relates to use of calcium ions to prepare an isolated complex comprising an adiponectin polypeptide or a conjugate comprising an adiponectin polypeptide, and a first non-polypeptide moiety covalently attached to the adiponectin polypeptide, wherein the complex is able to inhibit LPS-induced TNF-alpha production, or is able to enhance the glucose uptake in muscle cells. Preferably, the complex is able to enhance the glucose uptake in muscle cells, in particular as described in the experimental section.

The adiponectin polypeptide may be prepared as described in the section below: “Methods of preparing an adiponectin polypeptide for use in the invention”. Moreover, the conjugate comprising an adiponectin polypeptide, and a first non-polypeptide moiety covalently attached to the adiponectin polypeptide, may be prepared as described in the section below: “Methods of preparing a conjugate of the invention”.

In a further aspect the present invention relates. to a method of preparing an isolated complex comprising a) an adiponectin polypeptide or a conjugate comprising an adiponectin polypeptide, and a first non-polypeptide moiety covalently attached to the adiponectin polypeptide, and b) calcium ions, provided the adiponectin polypeptide is expressed and recovered from mammalian host cells, the method comprising bringing calcium ions in contact with the adiponectin polypeptide and optionally reacting the adiponectin polypeptide with the first non-polypeptide moiety.

In a further aspect the present invention relates to a method of preparing an isolated complex comprising a) an adiponectin polypeptide or a conjugate comprising an adiponectin polypeptide, and a first non-polypeptide moiety covalently attached to the adiponectin polypeptide, and b) calcium ions, provided the adiponectin polypeptide is expressed and recovered from bacterial host cells, the method comprising bringing calcium ions in contact with the adiponectin polypeptide and optionally reacting the adiponectin polypeptide with the first non-polypeptide moiety.

By making sure that calcium ions are present during the preparation of the adiponectin polypeptide or conjugate, such as in the culture medium, (such as DMEMIF-12(1:1) medium Cat no 21041(Invitrogen)) or suspension used, in the cells, or added to the preparation (such as in the form of calcium chloride (CaCl₂)), a stable trimer is obtained. In fact, during culturing of the host cells the presence of calcium ions will provide a stable trimer. Such stability may be verified on native gels. Preferably a medium containing calcium is used, such as DMEMIF-12(1:1) medium Cat no 21041(Invitrogen). However, media without calcium may also be used, such as DMEM Cat no 21068 (Invitrogen), in which case calcium is preferably added to the preparation.

Accordingly, in a further aspect the present invention relates to a culture comprising a) a mammalian host cell expressing an adiponectin polypeptide, and b) calcium ions.

In a further aspect the present invention relates to a culture comprising a) a bacterial host cell expressing an adiponectin polypeptide, and b) calcium ions.

In a further aspect the present invention relates to a method of preparing an isolated complex comprising a) an adiponectin polypeptide or a conjugate comprising an adiponectin polypeptide, and a first non-polypeptide moiety covalently attached to the adiponectin polypeptide, and b) calcium ions, comprising

a) preparing a nucleotide sequence encoding: a signal peptide and the adiponectin polypeptide,

b) inserting the nucleotide sequence into a vector,

c) transfecting the vector into a mammalian cell,

d) expressing and optionally secreting the adiponectin polypeptide,

e) recovering the complex, and optionally

f) reacting the adiponectin polypeptide with the molecule to which it is to be conjugated under conditions conducive for the conjugation to take place, and recovering the conjugate;

provided that any one of steps d), e), or f) is carried out in a calcium ion rich environment.

The term “calcium ion rich environment” is intended to mean that calcium ions (preferably in a molar surplus relative to the adiponectin polypeptide) are present during the preparation of the adiponectin polypeptide, and in particular are present during any one of steps d), e), or f), such as in the culture medium or suspension used, or added during steps d), e), or f), (such as in the form of calcium chloride (CaCl₂)).

It is intended that the particular embodiments of the adiponectin polypeptide and the conjugate comprising an adiponectin polypeptide, and a first non-polypeptide moiety covalently attached to the adiponectin polypeptide mentioned in the above sections “Adiponectin polypeptide fragment(s) of the invention”, “First group of conjugate(s) of the invention”, “Second group of conjugate(s) of the invention”, “Third group of conjugate(s) of the invention”, and “Fourth group of conjugate(s) of the invention”, also apply to this calcium composition aspect of the invention. Moreover, when the non-polypeptide is a polymer, then the embodiments mentioned in the above sections in connection with a polymer also applies to the polymer attached to the adiponectin polypeptide. Thus, the embodiments described below should not be seen as limiting this particular aspect of the invention in any way.

Thus, in any one of the aspects of the calcium composition aspects mentioned above the adiponectin polypeptide or conjugate may be selected from the below embodiments.

In an embodiment the adiponectin polypeptide is selected from any one of the SEQ ID NO:2-8, 10-12, or 13, and sequences having at least 80% identity to any one of SEQ ID NO:2-8, 10-12, or 13. Typically, the adiponectin polypeptide is selected from any one of the SEQ ID NO:5, 10, 11, 12, or 13, and sequences having at least 80% identity to any one of SEQ ID NO:5, 10, 11, 12, or 13.

In a further embodiment the adiponectin polypeptide is selected from any one of the SEQ ID NO:2-8, 10-12, or 13, and sequences having at least 90% identity to any one of SEQ ID NO:2-8, 10-12, or 13. Typically, the adiponectin polypeptide is selected from any one of the SEQ ID NO:3, 4, 5, 6, 10, 11, 12, or 13, and sequences having at least 90% identity to any one of SEQ ID NO:3, 4, 5, 6, 10, 11, 12, or 13.

In a further embodiment the adiponectin polypeptide is selected from any one of the SEQ iD NO:3, 4, 5, 6, 10, 11, 12, or 13, and sequences having at least 92% identity to any one of SEQ ID NO:3, 4, 5, 6, 10, 11, 12, or 13. The percent identity as stated above is determined using the CLUSTALW program.

In a further embodiment the adiponectin polypeptide is selected from any one of the SEQ ID NO:2-8, 10-12, or 13. In a further embodiment the adiponectin polypeptide is SEQ ID NO:5. In a further embodiment the adiponectin polypeptide is SEQ ID NO:10. In a further embodiment the adiponectin polypeptide is SEQ ID NO:11. In a further embodiment the adiponectin polypeptide is SEQ ID NO:12. In a further embodiment the adiponectin polypeptide is selected from any one of the SEQ ID NO:13.

In a further embodiment the adiponectin polypeptide is selected from an adiponectin polypeptide fragment comprising a globular domain and a collagen domain, wherein the globular domain comprises an amino acid sequence as indicated in SEQ ID NO:1 from position A108 to N244 as well as sequences that differs from the amino acid sequence in one or more substitution(s), and wherein the collagen domain comprises from 7 amino acids corresponding to position K101 as indicated in SEQ ID NO:1 to 66 amino acids corresponding to position G42 as indicated in SEQ ID NO:1, and wherein the collagen domain comprises a lysine which is hydroxylated and glycosylated.

In a further embodiment the adiponectin polypeptide is selected from an adiponectin polypeptide having an amino acid sequence selected from the SEQ ID NO:10, wherein the adiponectin polypeptide comprises the mutation T121C, such as the adiponectin polypeptide having the amino acid sequence of SEQ ID NO:17; in another embodiment the adiponectin polypeptide is selected from an adiponectin polypeptide having an amino acid sequence selected from the SEQ ID NO:10, wherein the adiponectin polypeptide comprises the mutation S146C, such as the adiponectin polypeptide having the amino acid sequence of SEQ ID NO:18; in a further embodiment the adiponectin polypeptide is selected from an adiponectin polypeptide having an amino acid sequence selected from the SEQ ID NO:10, wherein the adiponectin polypeptide comprises the mutation T243C, such as the adiponectin polypeptide having the amino acid sequence of SEQ ID NO:19; in a further embodiment the adiponectin polypeptide is selected from an adiponectin polypeptide having an amino acid sequence selected from the SEQ ID NO:10, wherein the adiponectin polypeptide comprises the mutation N127C, such as the adiponectin polypeptide having the amino acid sequence of SEQ ID NO:35; in a further embodiment the adiponectin polypeptide is selected from an adiponectin polypeptide having an amino acid sequence selected from the SEQ ID NO:10, wherein the adiponectin polypeptide comprises the mutation N141C, such as the adiponectin polypeptide having the amino acid sequence of SEQ ID NO:36; in a further embodiment the adiponectin polypeptide is selected from an adiponectin polypeptide having an amino acid sequence selected from the SEQ ID NO:10, wherein the adiponectin polypeptide comprises the mutation N228C, such as the adiponectin polypeptide having the amino acid sequence of SEQ ID NO:37; in a further embodiment the adiponectin polypeptide is selected from an adiponectin polypeptide having an amino acid sequence selected from the SEQ ID NO:5, wherein the adiponectin polypeptide comprises the mutation T121C, such as the adiponectin polypeptide having the amino acid sequence of SEQ ID NO:23; in a further embodiment the adiponectin polypeptide is selected from an adiponectin polypeptide having an amino acid sequence selected from the SEQ ID NO:5, wherein the adiponectin polypeptide comprises the mutation S146C, such as the adiponectin polypeptide having the amino acid sequence of SEQ ID NO:24; in a further embodiment the adiponectin polypeptide is selected from an adiponectin polypeptide having an amino acid sequence selected from the SEQ ID NO:5, wherein the adiponectin polypeptide comprises the mutation T243C, such as the adiponectin polypeptide having the amino acid sequence of SEQ ID NO:25; in a further embodiment the adiponectin polypeptide is selected from an adiponectin polypeptide having an amino acid sequence selected from the SEQ ID NO:5, wherein the adiponectin polypeptide comprises the mutation N127C, such as the adiponectin polypeptide having the amino acid sequence of SEQ ID NO:41; in a further embodiment the adiponectin polypeptide is selected from an adiponectin polypeptide having an amino acid sequence selected from the SEQ ID NO:5, wherein the adiponectin polypeptide comprises the mutation N141C, such as the adiponectin polypeptide having the amino acid sequence of SEQ ID NO:42; in a further embodiment the adiponectin polypeptide is selected from an adiponectin polypeptide having an amino acid sequence selected from the SEQ ID NO:5, wherein the adiponectin polypeptide comprises the mutation N228C, such as the adiponectin polypeptide having the amino acid sequence of SEQ ID NO:43; in a further embodiment the adiponectin polypeptide is selected from an adiponectin polypeptide having an amino acid sequence selected from the SEQ ID NO:13, wherein the adiponectin polypeptide comprises the mutation T121C, such as the adiponectin polypeptide having the amino acid sequence of SEQ ID NO:32; in a further embodiment the adiponectin polypeptide is selected from an adiponectin polypeptide having an amino acid sequence selected from the SEQ ID NO:13, wherein the adiponectin polypeptide comprises the mutation S146C, such as the adiponectin polypeptide having the amino acid sequence of SEQ ID NO:33; in a further embodiment the adiponectin polypeptide is selected from an adiponectin polypeptide having an amino acid sequence selected from the SEQ ID NO:13, wherein the adiponectin polypeptide comprises the mutation T243C, such as the adiponectin polypeptide having the amino acid sequence of SEQ ID NO:34; in a further embodiment the adiponectin polypeptide is selected from an adiponectin polypeptide having an amino acid sequence selected from the SEQ ID NO:13, wherein the adiponectin polypeptide comprises the mutation N127C, such as the adiponectin polypeptide having the amino acid sequence of SEQ ID NO:50; in a further embodiment the adiponectin polypeptide is selected from an adiponectin polypeptide having an amino acid sequence selected from the SEQ ID NO:13, wherein the adiponectin polypeptide comprises the mutation N141C, such as the adiponectin polypeptide having the amino acid sequence of SEQ ID NO:51; in a further embodiment the adiponectin polypeptide is selected from an adiponectin polypeptide having an amino acid sequence selected from the SEQ ID NO:13, wherein the adiponectin polypeptide comprises the mutation N228C, such as the adiponectin polypeptide having the amino acid sequence of SEQ ID NO:52.

In a further embodiment the adiponectin polypeptide is selected from an adiponectin polypeptide fragment comprising a globular domain and a collagen domain, wherein the globular domain comprises an amino acid sequence as indicated in SEQ ID NO:1 from position A108 to N244, and wherein the collagen domain comprises from 7 amino acids corresponding to position K101 as indicated in SEQ ID NO:1 to 66 amino acids corresponding to position G42 as indicated in SEQ ID NO:1, and wherein the collagen domain comprises a lysine which is hydroxylated and glycosylated, wherein the adiponectin polypeptide fragment comprises a mutation selected from any one of A108C, Y109C, V110C, Y111C, R112C, E120C, T121C, Y122C, V123C, T124C, I125C, P126C, N127C, M128C, R131C, T133C, K134C, I135C, Q139C, N141C, D144C, G145C, S146C, T147C, K149C, H151C, N153C, P155C, Y167C, M168C, K169C, D170C, K178C, D179C, K180C, A181C, F184C, Y186C, Q188C, Y189C, Q190C, E191C, N192C, N193C, V194C, H204C, E206C, V207C, G208C, Q210C, V215C, Y216C, G217C, E218C, G219C, E220C, R221C, N222C, L224C, Y225C, D227C, N228C, D229C, N230C, H241C, D242C, T243C, or N244C.

In a further embodiment the adiponectin polypeptide is selected from an adiponectin polypeptide fragment comprising a globular domain and a collagen domain,

wherein the globular domain comprises an amino acid sequence from position A108 to N244 as indicated in SEQ ID NO:1, and wherein the collagen domain comprises from 7 amino acids corresponding to position KIO as indicated in SEQ ID NO:1 to 56 amino acids corresponding to position A52 as indicated in SEQ ID NO:1, and wherein the collagen domain comprises a lysine which is hydroxylated and glycosylated, wherein the adiponectin polypeptide fragment comprises the mutation T121C. Examples of such adiponectin polypeptide fragments are any one of the sequences SEQ ID NO:17, 23, 26, 29, or 32. In another embodiment the adiponectin polypeptide is selected from an adiponectin polypeptide fragment comprising a globular domain and a collagen domain,

wherein the globular domain comprises an amino acid sequence from position A108 to N244 as indicated in SEQ ID NO:1, and wherein the collagen domain comprises from 8 amino acids corresponding to position R100 as indicated in SEQ ID NO:1 to 50 amino acids corresponding to position R58 as indicated in SEQ ID NO:1, and wherein the collagen domain comprises a lysine which is hydroxylated and glycosylated, wherein the adiponectin polypeptide comprises the mutation S146C. Examples of such adiponectin polypeptide fragments are any one of the sequences SEQ ID NO:18, 27, or 33. In a further embodiment the adiponectin polypeptide is selected from an adiponectin polypeptide fragment comprising a globular domain and a collagen domain,

wherein the globular domain comprises an amino acid sequence from position A108 to N244 as indicated in SEQ ID NO:1, and wherein the collagen domain comprises from 8 amino acids corresponding to position R100 as indicated in SEQ ID NO:1 to 29 amino acids corresponding to position D79 as indicated in SEQ ID NO:1, and wherein the collagen domain comprises a lysine which is hydroxylated and glycosylated, wherein the adiponectin polypeptide comprises the mutation T243C. Examples of such adiponectin polypeptide fragments are any one of the sequences SEQ ID NO:19, or 28. In a further embodiment the adiponectin polypeptide is selected from an adiponectin polypeptide fragment comprising a globular domain and a collagen domain, wherein the globular domain comprises an amino acid sequence from position A108 to N244 as indicated in SEQ ID NO:1, and wherein the collagen domain comprises from 7 amino acids corresponding to position K101 as indicated in SEQ ID NO:1 to 56 amino acids corresponding to position A52 as indicated in SEQ ID NO:1, and wherein the collagen domain comprises a lysine which is hydroxylated and glycosylated, wherein the adiponectin polypeptide fragment comprises the mutation N127C. Examples of such adiponectin polypeptide fragments are any one of the sequences SEQ Ip NO:35, 41, 44, 47, or 50. In a further embodiment the adiponectin polypeptide is selected from an adiponectin polypeptide fragment comprising a globular domain and a collagen domain, wherein the globular domain comprises an amino acid sequence from position A108 to N244 as indicated in SEQ ID NO:1, and wherein the collagen domain comprises from 8 amino acids corresponding to position R100 as indicated in SEQ ID NO:1 to 50 amino acids corresponding to position R58 as indicated in SEQ ID NO:1, and wherein the collagen domain comprises a lysine which is hydroxylated and glycosylated, wherein the adiponectin polypeptide comprises the mutation N141C. Examples of such adiponectin polypeptide fragments are any one of the sequences SEQ ID NO:36, 45, or 51. In a further embodiment the adiponectin polypeptide is selected from an adiponectin polypeptide fragment comprising a globular domain and a collagen domain, wherein the globular domain comprises an amino acid sequence from position A108 to N244 as indicated in SEQ ID NO:1, and wherein the collagen domain comprises from 8 amino acids corresponding to position R100 as indicated in SEQ ID NO:1 to 29 amino acids corresponding to position D79 as indicated in SEQ ID NO:1, and wherein the collagen domain comprises a lysine which is hydroxylated and glycosylated, wherein the adiponectin polypeptide comprises the mutation N228C. Examples of such adiponectin polypeptide fragments are any one of the sequences SEQ ID NO:37, or 46.

When the adiponectin polypeptide is part of a conjugate, that is, a conjugate comprising an adiponectin polypeptide, and a first non-polypeptide moiety covalently attached to the adiponectin polypeptide, the first non-polypeptide moiety is selected from a polymer molecule, a lipophilic compound, a sugar moiety and an organic derivatizing agent.

The first non-polypeptide moiety may be attached to an amino acid which is one of the naturally occurring present in the adiponectin polypeptide, as described in the above sections “First group of conjugate(s) of the invention”, “Second group of conjugate(s) of the invention”, and “Third group of conjugate(s) of the invention”, or to an introduced amino acid as described in the above section “Fourth group of conjugate(s) of the invention”.

To further illustrate the conjugate part of the complex comprising a conjugate comprising an adiponectin polypeptide, and a first non-polypeptide moiety covalently attached to the adiponectin polypeptide, and calcium ions, some non-exhaustive embodiments are disclosed below.

In one embodiment the conjugate comprises

an adiponectin polypeptide fragment comprising a globular domain and a collagen domain, wherein the globular domain comprises an amino acid sequence as indicated in SEQ ID NO:1 from position A108 to N244, and wherein the collagen domain comprises from 7 amino acids corresponding to position K101 as indicated in SEQ ID NO:1 to 66 amino acids corresponding to position G42 as indicated in SEQ ID NO:1, and wherein the collagen domain comprises a lysine which is hydroxylated and glycosylated, wherein the adiponectin polypeptide fragment comprises a mutation selected from any one of A108C, Y109C, VIlOC, YIIIC, R1 12C, E120C, T121C, Y122C, V123C, T124C, I125C, P126C, N127C, M128C, R131C, T133C, K134C, I135C, Q139C, N141C, D144C, G145C, S146C, T147C, K149C, H151C, N153C, P155C, Y167C, M168C, K169C, D170C, K178C, D179C, K180C, A181C, F184C, Y186C, Q188C, Y189C, Q190C, E191C, N192C, N193C, V194C, H204C, E206C, V207C, G208C, Q210C, V215C, Y216C, G217C, E218C, G219C, E220C, R221C, N222C, L224C, Y225C, D227C, N228C, D229C, N230C, H241C, D242C, T243C, or N244C; and

a first non-polypeptide moiety covalently attached to the iritroduced cystein residue. Preferably, the first non-polypeptide moiety is a polymer, typically a linear or branched polyethylene glycol.

In another embodiment the conjugate comprises

an adiponectin polypeptide fragment comprising a globular domain and a collagen domain, wherein the globular domain comprises an amino acid sequence from position A108 to N244 as indicated in SEQ ID NO:1, and wherein the collagen domain comprises from 7 amino acids corresponding to position K101 as indicated in SEQ ID NO:1 to 56 amino acids corresponding to position A52 as indicated in SEQ ID NO:1, and wherein the collagen domain comprises a lysine which is hydroxylated and glycosylated,

the adiponectin polypeptide fragment comprising a mutation selected from any one of A108N+V110T, Y109N+Y111T, V110N+R112T, Y111N, Y111N+S113T, R112N+A114T, L119N+T121S, L119N, E120N+Y122T, T121N+V123T, Y122N, Y122N+T124S, T124N+P126T, P126N+M128T, P129T, M128N+I130T, I130N+F132T, R131N, R131N+T133S, T133N+I135T, K134N+F136T, I135N+Y137T, F136N+N138T, Y137N+Q139T, Q140T, Q139N+N141T, Q140N+H142T, Y143T, H142N+D144T, D144N, D144N+S146T, G145N, G145N+T147S, S146N+G148T, T147N+K149T, K149N+H151T, H151N+N153T, P155T, P155N+L157T, Y159N+A161T, I164N+V166T, T165N+Y167T, Y167N+K169T, M168N+D170T, K169N+V171T, D170N+K172T, V171N+V173T, K172N, K172N+S174T, F176N+K178T, K177N+D179T, K178N+K180T, D179N+A181T, K180N+M182T, A181N+L183T, M182N+F184T, F184N+Y186T, Y186N+Q188T, Q188N+Q190T, Y189N+E191T, Q190N+N192T, E191N+N193T, V194T, or V194N+Q196T;

and a sugar moiety covalently attached to the introduced N-glycosylation site. A further specific conjugate comprises an adiponectin polypeptide selected from the sequence SEQ ID NO:10 comprising a mutation selected from Y111N, such as the adiponectin polypeptide having the SEQ ID NO:53, and a sugar moiety covalently attached to the introduced N-glycosylation site. In a further embodiment the conjugate comprises an adiponectin polypeptide selected from the sequence SEQ ID NO:10 comprising a mutation selected from Y122N, such as the adiponectin polypeptide having the SEQ ID NO:54, and a sugar moiety covalently attached to the introduced N-glycosylation site. In a further embodiment the conjugate comprises an adiponectin polypeptide selected from the sequence SEQ ID NO:10 comprising a mutation selected from R131N, such as the adiponectin polypeptide having the SEQ ID NO:55, and a sugar moiety covalently attached to the introduced N-glycosylation site. In a further embodiment the conjugate comprises an adiponectin polypeptide selected from the sequence SEQ ID NO:10 comprising a mutation selected from D 144N+S146T, such as the adiponectin polypeptide having the SEQ ID NO:56, and a sugar moiety covalently attached to the introduced N-glycosylation site. In a further embodiment the conjugate comprises an adiponectin polypeptide selected from the sequence SEQ ID NO:10 comprising a mutation selected from H151N+N153T, such as the adiponectin polypeptide having the SEQ ID NO:57, and a sugar moiety covalently attached to the introduced N-glycosylation site. In a further embodiment the conjugate comprises an adiponectin polypeptide selected from the sequence SEQ ID NO:10 comprising a mutation selected from K178N+K180T, such as the adiponectin polypeptide having the SEQ ID NO:58, and a sugar moiety covalently attached to the introduced N-glycosylation site. In a further embodiment the conjugate comprises an adiponectin polypeptide selected from the sequence SEQ ID NO:10 comprising a mutation selected from P 129T, such as the adiponectin polypeptide having the SEQ ID NO:59, and a sugar moiety covalently attached to the introduced N-glycosylation site. In a further embodiment the conjugate comprises an adiponectin polypeptide selected from the sequence SEQ ID NO:10 comprising a mutation selected from G145N, such as the adiponectin polypeptide having the SEQ ID NO:60, and a sugar moiety covalently attached to the introduced N-glycosylation site. In a further embodiment the conjugate comprises an adiponectin polypeptide selected from the sequence SEQ ID NO:10 comprising a mutation selected from P155T, such as the adiponectin polypeptide having the SEQ ID NO:61, and a sugar moiety covalently attached to the introduced N-glycosylation site.

In a further embodiment the conjugate comprises

an adiponectin polypeptide fragment comprising a globular domain and a collagen domain, wherein the globular domain comprises an amino acid sequence from position A108 to N244 as indicated in SEQ ID NO:1 as well as sequences that differs from the amino acid sequence in one or more substitution(s), and wherein the collagen domain comprises from 7 amino acids corresponding to position K101 as indicated in SEQ ID NO:1 to 56 amino acids corresponding to position A52 as indicated in SEQ ID NO:1, and wherein the collagen domain comprises a lysine which is hydroxylated and glycosylated, and

a first non-polypeptide moiety covalently attached to the adiponectin polypeptide fragment,

wherein the adiponectin polypeptide fragment comprises an amino acid residue having an attachment group for said first non-polypeptide moiety, wherein the amino acid residue is the N-terminal amino acid residue. Preferably, the first non-polypeptide moiety is a polymer, typically a linear or branched polyethylene glycol.

Further preferred embodiments of the adiponectin polypeptide fragment are any one of the sequences selected from SEQ ID NO:2, 3, 4, 5, 6, 10, 11, 12, 13, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, or 61. These sequences are also preferred embodiments when being the part of the conjugate.

During pegylation of the adiponectin polypeptide fragment of the present invention, in the presence of calcium ions, to produce a conjugate, we discovered that it was possible to introduce one PEG molecule into a trimer of the polypeptide without destroying the trimer, and the biological activity was maintained at least in part. The PEG molecule was attached to the N-terminal residue of one of the adiponectin polypeptide monomers, thus producing a trimer containing one PEG molecule. Moreover, we also succeded in introducing two and three PEG molecule into a trimer of the polypeptide, as shown in example 11.

Accordingly, in a further embodiment the conjugate comprises an adiponectin polypeptide trimer, and one polymer covalently attached to the adiponectin polypeptide trimer.

In a further embodiment the conjugate consists of

a) an adiponectin polypeptide trimer wherein the adiponectin polypeptide trimer contains three adiponectin polypeptide monomers, and

b) one polymer covalently attached to any one of the three monomers of the adiponectin polypeptide trimer in such a way that the adiponectin polypeptide trimer only contains one polymer.

In a still further embodiment the conjugate comprises an adiponectin polypeptide trimer, and two polymers covalently attached to the adiponectin polypeptide trimer.

In a further embodiment the conjugate consists of

a) an adiponectin polypeptide trimer wherein the adiponectin polypeptide trimer contains three adiponectin polypeptide monomers, and

b) two polymers covalently attached to any one of the three monomers of the adiponectin polypeptide trimer in such a way that the adiponectin polypeptide trimer only contains two polymers.

In a further embodiment the conjugate comprises an adiponectin polypeptide trimer, and three polymers covalently attached to the adiponectin polypeptide trimer.

In a further embodiment the conjugate consists of

a) an adiponectin polypeptide trimer wherein the adiponectin polypeptide trimer contains three adiponectin polypeptide monomers, and

b) three polymers covalently attached to any one of the three monomers of the adiponectin polypeptide trimer in such a way that the adiponectin polypeptide trimer contains three polymers.

Even though this embodiment relates to trimeric adiponectin polypeptides it is intended that the particular embodiments mentioned in the above sections “Adiponectin polypeptide fragment(s) of the invention”, “First group of conjugate(s) of the invention”, “Second group of conjugate(s) of the invention”, “Third group of conjugate(s) of the invention”, and “Fourth group of conjugate(s) of the invention”, also apply to this trimeric embodiment of the invention, for instance, the embodiments mentioned in connection with an adiponectin polypeptide also applies to the adiponectin polypeptide monomer as part of the trimer. Moreover, when the non-polypeptide is a polymer, then the embodiments mentioned in the above sections in connection with a polymer also applies to the one, two, or three polymer(s) attached to the adiponectin polypeptide trimer. Thus, the embodiments described below should not be seen as limiting this particular aspect of the invention in any way.

As described in the above sections “First group of conjugate(s) of the invention”, “Second group of conjugate(s) of the invention”, and “Third group of conjugate(s) of the invention”, then in a further embodiment the adiponectin polypeptide monomer comprises an amino acid residue having an attachment group for the polymer. Such amino acid residue may be any amino acid residue suitable for polymer conjugation, preferably the amino acid residue is selected from a lysine, a cysteine, or an N-terminal residue. In a further embodiment the amino acid residue having an attachment group for the polymer is selected from an N-terminal residue.

In a further embodiment the adiponectin polypeptide monomer is selected from any one of the SEQ ID NO:1-8, 10-12, or 13, such as the SEQ ID NO:2, 3, 4, 5, 6, 10, 11, 12, or 13, in particular SEQ ID NO:10, 11, 12, or 13.

As described in the above section “Fourth group of conjugate(s) of the invention”, then in a further embodiment the adiponectin polypeptide monomer comprises an amino acid residue having an attachment group for a polymer, wherein said amino acid residue has been introduced in a position that in the parent adiponectin is occupied by a surface exposed amino acid residue, such introduced amino acid is typically, selected from C, K, D, or E, preferably C.

Thus, in a further embodiment the adiponectin polypeptide monomer is selected from any one of the SEQ ID NO:17-52, such as 17, 18, 19, 35, 36, or 37. In this respect, the polymer is attached to the introduced cysteine.

If one, two, or three polymers are attached to the trimer it is preferred that such polymer is selected from a polyethylene glycol. Thus, in a further embodiment the polymer comprises a polyethylene glycol, such as a linear or branched polyethylene glycol. In a further embodiment the polymer is a polyethylene glycol, such as a linear or branched polyethylene glycol.

In a still further embodiment the polymer, such as a polyethylene glycol, has a molecular weight of from lkDa to 200 kDa, such as from lkDa to 20 kDa, e.g. from 5 kDa to 20 kDa, such as 5 kDa, 10 kDa, or 20 kDa.

The composition comprising the conjugate may be any suitable composition such as any one of those mentioned below in the section “Pharmaceutical composition and uses of a conjugate or adiponectin polypeptide fragment of the invention”. Thus, the composition may be formulated in a variety of forms, such as liquid or solid compositions. The term “liquid” is intended to include aqueous.

In a further embodiment the composition is selected from a liquid composition.

In a further embodiment the liquid composition is a solution or suspension, such as an aqueous solution.

Further Embodiments of any one of the Above Conjugates of the Invention.

By removing and/or introducing amino acid residues comprising an attachment group for the non-polypeptide moiety it is possible to specifically adapt the polypeptide so as to make the molecule more susceptible to conjugation to the non-polypeptide moiety of choice, to optimize the conjugation pattern (e.g. to ensure an optimal distribution of non-polypeptide moieties on the surface of the adiponectin polypeptide and thereby, e.g., effectively shield epitopes and other surface parts of the polypeptide without significantly impairing the function thereof). For instance, by introduction of attachment groups, the adiponectin polypeptide is boosted or otherwise altered in the content of the specific amino acid residues to which the relevant non-polypeptide moiety binds, whereby a more efficient, specific and/or extensive conjugation is achieved. By removal of one or more attachment groups it is possible to avoid conjugation to the non-polypeptide moiety in parts of the polypeptide in which such conjugation is disadvantageous, e.g. to an amino acid residue located at or near a functional site of the polypeptide (since conjugation at such a site may result in inactivation or reduced activity of the resulting conjugate due to impaired receptor recognition). Further, it may be advantageous to remove an attachment group located closely to another attachment group in order to avoid heterogeneous conjugation to such groups.

It will be understood that the amino acid residue comprising an attachment group for a non-polypeptide moiety, either it be removed or introduced, is selected on the basis of the nature of the non-polypeptide moiety and, in most instances, on the basis of the conjugation method to be used. For instance, when the non-polypeptide moiety is a polymer molecule, such as a polyethylene glycol or polyalkylene oxide derived molecule, amino acid residues capable of functioning as an attachment group may be selected from the group consisting of lysine, cysteine, aspartic acid, glutamic acid and arginine. When the non-polypeptide moiety is a sugar moiety the attachment group is an in vivo glycosylation site, preferably an N-glycosylation site.

Alternatively or additionally, the position to be modified is identified on the basis of an analysis of an adiponectin protein sequence family. More specifically, the position to be modified can be one, which in one or more members of the family other than the parent adiponectin, is occupied by an amino acid residue comprising the relevant attachment group (when such amino acid residue is to be introduced) or which in the parent adiponectin, but not in one or more other members of the family, is occupied by an amino acid residue comprising the relevant attachment group (when such amino acid residue is to be removed).

In order to determine an optimal distribution of attachment groups, the distance between amino acid residues located at the surface of the adiponectin polypeptide is calculated on the basis of a 3D structure of the adiponectin polypeptide. More specifically, the distance between the CB's of the amino acid residues comprising such attachment groups, or the distance between the functional group (NZ for lysine, CG for aspartic acid, CD for glutamic acid, SG for cysteine) of one and the CB of another amino acid residue comprising an attachment group are determined. In case of glycine, CA is used instead of CB. In the adiponectin polypeptide part of a conjugate of the invention, any of said distances is preferably more than 8 A, in particular more than IOA in order to avoid or reduce heterogeneous conjugation.

Furthermore, in the adiponectin polypeptide,part of a conjugate of the invention attachment groups located at the receptor-binding site of adiponectin has preferably been removed, preferably by substitution of the amino acid residue comprising such group.

A still further generally applicable approach for modifying an adiponectin polypeptide is to shield, and thereby destroy or otherwise inactivate an epitope present in the parent adiponectin, by conjugation to a non-polypeptide moiety. Epitopes of human adiponectin may be identified by use of methods known in the art, also known as epitope mapping, see, e.g. Romagnoli et al., J. Biol Chem, 1999, 380(5):553-9, DeLisser HM, Methods Mol Biol, 1999, 96:11-20, Van de Water et al., Clin Immunol Immunopathol, 1997, 85(3):229-35, Saint-Remy JM, Toxicology, 1997, 119(1):77-81, and Lane DP and Stephen CW, Curr Opin Immunol, 1993, 5(2):268-71. One method is to establish a phage display library expressing random oligopeptides of e.g. 9 amino acid residues. IgG1 antibodies from specific antisera towards human adiponectin are purified by immunoprecipitation and the reactive phages are identified by immunoblotting. By sequencing the DNA of the purified reactive phages, the sequence of the oligopeptide can be determined followed by localization of the sequence on the 3D-structure of the adiponectin. Alternatively, epitopes can be identified according to the method described in U.S. Pat. No. 5,041,376. The thereby identified region on the structure constitutes an epitope that then can be selected as a target region for introduction of an attachment group for the non-polypeptide moiety. Preferably, at least one epitope, such as two, three or four epitopes of human recombinant adiponectin are shielded by a non-polypeptide moiety according to the present invention. Accordingly, in one embodiment, the conjugate of the invention has at least one shielded epitope as compared to wild type human adiponectin.

In case of removal of an attachment group, the relevant amino acid residue comprising such group and occupying a position as defined above is preferably substituted with a different amino acid residue that does not comprise an attachment group for the non-polypeptide moiety in question.

In case of introduction of an attachment group, an amino acid residue comprising such group is introduced into the position, preferably by substitution of the amino acid residue occupying such position. However, such introduction of an attachment group may also be through addition of an amino acid residue to the N- or C-terminal of the polypeptide, such as the globular, collagen, or non-homologuous domain of the polypeptide.

The exact number of attachment groups available for conjugation and present in the adiponectin polypeptide is dependent on the effect desired to be achieved by conjugation. The effect to be obtained is, e.g., dependent on the nature and degree of conjugation (e.g. the identity of the non-polypeptide moiety, the number of non-polypeptide moieties desirable or possible to conjugate to the polypeptide, where they should be conjugated or where conjugation should be avoided, etc.). For instance, if reduced immunogenicity is desired, the number (and location of) attachment groups should be sufficient to shield most or all epitopes. This is normally obtained when a greater proportion of the adiponectin polypeptide is shielded. Effective shielding of epitopes is normally achieved when the total number of attachment groups available for conjugation is in the range of 1-10 attachment groups. Functional in vivo half-life is i.a. dependent on the molecular weight of the conjugate and the number of attachment groups needed for providing increased half-life thus depends on the molecular weight of the non-polypeptide moiety in question.

In one embodiment, the conjugate of the invention has a molecular weight of at least 67 kDa, in particular at least 70 kDa as measured by SDS-PAGE according to Laenmli, U.K., Nature Vol 227 (1970), p680-85.

In order to avoid too much disruption of the structure and function of the parent human adiponectin the total number of amino acid residues to be altered in accordance with the present invention (as compared to the amino acid sequence shown in SEQ ID NO:6) typically does not exceed 15. Preferably, when an analog is desired, the adiponectin polypeptide comprises an amino acid sequence, which differs in 1-15 amino acid residues from the amino acid sequence shown in SEQ ID NO:6, such as in 1-11, 1-8 or in 2-8 amino acid residues, e.g. in 1-5 or in 2-5 amino acid residues from the amino acid sequence shown in SEQ ID NO:6. Thus, normally the adiponectin polypeptide comprises an amino acid sequence that differs from the amino acid sequence shown in SEQ ID NO:6 in 1, 2, 3, 4, 5, 6, 7, 8, 9, 10, 11, 12, 13, 14 or 15 amino acid residues. Typically, the above numbers represent either the total number of introduced or the total number of removed amino acid residues comprising an attachment group for the relevant non-polypeptide moiety, or the total number of introduced and removed amino acid residues comprising such group.

In the conjugate of the invention it is preferred that at least about 50% of all conjugatable attachment groups, such as at least about 80% and preferably all of such groups are occupied by the relevant non-polypeptide moiety. Accordingly, in a preferred embodiment the conjugate of the invention comprises, e.g., 1-10 non-polypeptide moieties.

Conjugate of the Invention, Wherein the Non-Polypeptide Moiety is a Molecule that has Lysine as an Attachment Group

In one embodiment the first non-polypeptide moiety has lysine as an attachment group, and thus the adiponectin polypeptide is one that comprises an amino acid sequence that differs from that of wildtype human adiponectin in at least one introduced and/or at least one removed lysine residue. While the non-polypeptide moiety may be any of those binding to a lysine residue, e.g. the ε-amino group thereof, such as a polymer molecule, a lipophilic group, an organic derivatizing agent or a carbohydrate moiety, it is preferably any of the polymer molecule mentioned in the section entitled “Conjugation to a polymer molecule”, in particular a branched or linear PEG or polyalkylene oxide. Most preferably, the polymer molecule is PEG and the activated molecule to be used for conjugation is SS-PEG, NPC-PEG, aldehyd-PEG, mPEG-SPA, mPEG-SCM, mPEG-BTC from Shearwater Polymers, Inc, SC-PEG from Enzon, Inc., tresylated mPEG as described in U.S. Pat. No. 5,880,255, or oxycarbonyl-oxy-N-dicarboxyimide-PEG (U.S. Pat. No. 5,122,614). Normally, for conjugation to a lysine residue the non-polypeptide moiety has a molecular weight of about 5, 10, 20, or 40 kDa.

The lysine residue(s) may be replaced with any other amino acid residue, but is preferably replaced by an arginine or a glutamine residue in order to give rise to the least structural difference.

Conjugate of the Invention Wherein the Non-Polypeptide Moiety Binds to a Cysteine Residue

While the first non-polypeptide moiety according to this aspect of the invention may be any molecule which, when using the given conjugation method has cysteine as an attachment group (such as a carbohydrate moiety, a lipophilic group or an organic derivatizing agent), it is preferred that the non-polypeptide moiety is a polymer molecule. The polymer molecule may be any of the molecules mentioned in the section entitled “Conjugation to a polymer molecule”, but is preferably selected from the group consisting of linear or branched polyethylene glycol or polyalkylene oxide. Typically, the polymer molecule is VS-PEG. The conjugation between the polypeptide and the polymer may be achieved in any suitable manner, e.g. as described in the section entitled “Conjugation to a polymer molecule”, e.g. in using a one step method or in the stepwise manner referred to in said section. When the adiponectin polypeptide comprises only one conjugatable cysteine residue, this is preferably conjugated to a first non-polypeptide moiety with a molecular weight of from 1 to 20 kDa or more, either directly conjugated or indirectly through a low molecular weight polymer (as disclosed in WO 99/55377). However, the conjugation of a cysteine to a first non-polypeptide moiety having a molecular weight of at least 5 kDa is also an embodiment of the invention. When the conjugate comprises two or more first non-polypeptide moieties, normally each of these has a molecular weight of 5, 10, or 20 kDa.

Conjugate of the Invention Wherein the Non-Polypeptide Moiety Binds to an Acid Group

In case of removal of an amino acid residue, the amino acid sequence of the adiponectin polypeptide differs from that of human wildtype adiponectin in at least one removed aspartic acid or glutamic acid residue, such as 1-5 removed residues, in particular 14 or 1-3 removed aspartic acid or glutamic acid residues. The aspartic acid or glutarnic acid residue(s) may be replaced with any other amino acid residue, but is preferably replaced by an arginine or a glutamine residue.first non-polypeptide moiety can be any non-polypeptide moiety with such property, it is presently preferred that the non-polypeptide moiety is a polymer molecule or an organic derivatizing agent having an acid group as an attachment group, in particular a polymer molecule such as PEG, and the conjugate is prepared, e.g., as described by Sakane and Pardridge, Pharmceutical Research, Vol. 14, No. 8, 1997, pp 1085-1091. Normally, for conjugation to an acid group the non-polypeptide moiety has a molecular weight of about 5, 10, or 20 kDa.

Conjugate of the Invention Comprising a Second Non-Polypeptide Moiety

In addition to a first non-polypeptide moiety (as described in the preceding sections), the conjugate of the invention may comprise a second non-polypeptide moiety of a different type as compared to the first non-polypeptide moiety. Preferably, in any of the above described conjugates wherein the first non-polypeptide moiety is, e.g., a polymer molecule such as PEG, a second non-polypeptide moiety is a sugar moiety, in particular an N-linked sugar moiety. Such site is e.g. any of those described in the immediately preceding section entitled “Conjugate of the invention wherein the non-polypeptide moiety is a sugar moiety”.

It will be understood that in order to obtain an optimal distribution of attached first and second non-polypeptide moieties, the adiponectin polypeptide may be modified in the number and distribution of attachment groups for the first as well as the second non-polypeptide moiety so as to have e.g. at least one removed attachment group for the first non-polypeptide moiety and at least one introduced attachment group for the second non-polypeptide moiety or vice versa.

Conjugate of the Invention Wherein the Non-Polypeptide Moiety is a Sugar Moiety

When the conjugate of the invention comprises at least one sugar moiety attached to an in vivo glycosylation site, in particular an N-glycosylation site, this is a new in vivo glycosylation site introduced into the adiponectin polypeptide. The in vivo glycosylation site may be an O-glycosylation site, but is preferably an N-glycosylation site.

For instance, an in vivo glycosylation site is introduced into a position of the parent adiponectin occupied by an amino acid residue exposed to the surface of the molecule, preferably with more than 25% of the side chain exposed to the solvent, in particular more than 50% exposed to the solvent (these positions are identified in the Experimentals/Methods section herein). The N-glycosylation site is introduced in such a way that the N-residue of said site is located in said position. Analogously, an O-glycosylation site is introduced so that the S or T residue making up such site is located in said position. Still more preferably, the in vivo glycosylation site is introduced into a position wherein only one mutation is required to create the site (i.e. where any other amino acid residues required for creating a functional glycosylation site is already present in the molecule). Non-polypeptide moiety of the conjugate of the invention As indicated further above the non-polypeptide moiety of the conjugate of the invention is preferably selected from the group consisting of a polymer molecule, a lipophilic compound, a sugar moiety (by way of in vivo glycosylation) and an organic derivatizing agent. All of these agents may confer desirable properties to the polypeptide part of the conjugate, in particular reduced immunogenicity and/or increased functional in vivo half-life and/or increased serum half-life. The polypeptide part of the conjugate may be conjugated to only one type of non-polypeptide moiety, but may also be conjugated to two or more different types of non-polypeptide moieties, e.g. to a polymer molecule and a sugar moiety, to a lipophilic group and a sugar moiety, to an organic derivating agent and a sugar moiety, to a lipophilic group and a polymer molecule, etc. The conjugation to two or more different non-polypeptide moieties may be done simultaneous or sequentially. The choice of non-polypeptide moiety/ies, e.g. depends on the effect desired to be achieved by the conjugation. For instance, sugar moieties have been found particularly useful for reducing immunogenicity, whereas polymer molecules such as PEG are of particular use for increasing functional in vivo half-life and/or serum half-life. Using a polymer molecule as a first non-polypeptide moiety and a sugar moiety as a second non-polypeptide moiey may result in reduced immunogenicity and increased functional in vivo or serum half-life.

Methods of Preparing a Conjugate of the Invention

In the following sections “Conjugation to a lipophilic compound”, “Conjugation to a polymer molecule”, “Conjugation to a sugar moiety” and “Conjugation to an organic derivatizing agent” conjugation to specific types of non-polypeptide moieties is described.

In a further aspect the invention relates to a method for preparing a conjugate comprising an adiponectin polypeptide, and a first non-polypeptide moiety covalently attached to the adiponectin polypeptide, wherein the adiponectin polypeptide is reacted with the first non-polypeptide moiety to which it is to be conjugated under conditions conducive for the conjugation to take place, and the conjugate is recovered.

In further aspects the invention relates to a method for preparing a conjugate comprising an adiponectin polypeptide, and a first non-polypeptide moiety covalently attached to the adiponectin polypeptide, as described above in connection with the first, second, third, and fourth group of conjugate(s) of the invention.

Conjugation to a Lipophilic Compound

For conjugation to a lipophilic compound the following polypeptide groups may function as attachment groups: the N-terminal or C-terminal of the polypeptide, the hydroxy groups of the amino acid residues Ser, Thr or Tyr, the ε-amino group of Lys, the SH group of Cys or the carboxyl group of Asp and Glu. The polypeptide and the lipophilic compound may be conjugated to each other, either directly or by use of a linker. The lipophilic compound may be a natural compound such as a saturated or unsaturated fatty acid, a fatty acid diketone, a terpene, a prostaglandin, a vitamine, a carotenoide or steroide, or a synthetic compound such as a carbon acid, an alcohol, an amine and sulphonic acid with one or more alkyl-, aryl-, alkenyl- or other multiple unsaturated compounds. The conjugation between the polypeptide and the lipophilic compound, optionally through a linker may be done according to methods known in the art, e.g. as described by Bodanszky in Peptide Synthesis, John Wiley, New York, 1976 and in WO 96/12505.

Conjugation to a Polymer Molecule

The polymer molecule to be coupled to the polypeptide may be any suitable polymer molecule, such as a natural or synthetic homo-polymer or heteropolymer, typically with a molecular weight in the range of 300-200,000 Da, such as lkDa to 20okDa.

Examples of homo-polymers include a polyol (i.e. poly-OH), a polyamine (i.e. poly-NH₂) and a polycarboxylic acid (i.e. poly-COOH). A hetero-polymer is a polymer, which comprises one or more different coupling groups, such as, e.g., a hydroxyl group and an amine group.

Examples of suitable polymer molecules include polymer molecules selected from the group consisting of polyalkylene oxide (PAO), including polyalkylene glycol (PAG), such as polyethylene glycol (PEG) and polypropylene glycol (PPG), branched PEGs, poly-vinyl alcohol (PVA), poly-carboxylate, poly-(vinylpyrolidone), polyethylene-o-maleic acid anhydride, polystyrene-co-malic acid anhydride, dextran including carboxymethyl-dextran, or any other biopolymer suitable for reducing immunogenicity and/or increasing functional in vivo half-life and/or serum half-life. Generally, polyalkylene glycol-derived polymers are biocompatible, non-toxic, non-antigenic, non-immunogenic, have various water solubility properties, and are easily excreted from living organisms.

PEG is the preferred polymer molecule to be used, since it has only few reactive groups capable of cross-linking compared, e.g., to polysaccharides such as dextran, and the like. In particular, monofunctional PEG, e.g monomethoxypolyethylene glycol (rnPEG), is of interest since its coupling chemistry is relatively simple (only one reactive group is available for conjugating with attachment groups on the polypeptide). Consequently, the risk of cross-linking is eliminated, the resulting polypeptide conjugates are more homogeneous and the reaction of the polymer molecules with the polypeptide is easier to control.

To effect covalent attachment of the polymer molecule(s) to the polypeptide, the hydroxyl end groups of the polymer molecule must be provided in activated form, i.e. with reactive functional groups (examples of which include primary amino groups, hydrazide (HZ), thiol, succinate (SUC), succinimidyl succinate (SS), succinimidyl succinamide (SSA), succinimidyl proprionate (SPA), succinimidy carboxymethylate (SCM), benzotriazole carbonate (BTC), N-hydroxysuccinimide (NHS), aldehyde, nitrophenylcarbonate (NPC), and tresylate (TRES)). Suitably activated polymer molecules are commercially available, e.g. from Shearwater Polymers, Inc., Huntsville, Ala., USA. Alternatively, the polymer molecules can be activated by conventional methods known in the art, e.g. as disclosed in WO 90/13540. Specific examples of activated linear or branched polymer molecules for use in the present invention are described in the Shearwater Polymers, Inc. 1997 and 2000 Catalogs (Functionalized Biocompatible Polymers for Research and pharmaceuticals, Polyethylene Glycol and Derivatives, incorporated herein by reference). Specific examples of activated PEG polymers include the following linear PEGs: NHS-PEG (e.g. SPA-PEG, SSPA-PEG, SBA-PEG, SS-PEG, SSA-PEG, SC-PEG, SG-PEG, and SCM-PEG), and NOR-PEG), BTC-PEG, EPOX-PEG, NCO-PEG, NPC-PEG, CDI-PEG, ALD-PEG, TRES-PEG, VS-PEG, IODO-PEG, and MAL-PEG, and branched PEGs such as PEG2-NHS and those disclosed in U.S. Pat. No. 5,932,462 and U.S. Pat. No. 5,643,575, both of which references are incorporated herein by reference. Furthermore, the following publications, incorporated herein by reference, disclose useful polymer molecules and/or PEGylation chemistries: U.S. Pat. No. 5,824,778, U.S. Pat. No. 5,476,653, WO 97/32607, EP 229,108, EP 402,378, U.S. Pat. No. 4,902,502, U.S. Pat. No. 5,281,698, U.S. Pat. No. 5,122,614, U.S. Pat. No. 5,219,564, WO 92/16555, WO 94/04193, WO 94/14758, WO 94/17039, WO 94/18247, WO 94/28024, WO 95/00162, WO 95/11924, WO95/13090, WO 95/33490, WO 96/00080, WO 97/18832, WO 98/41562, WO 98/48837, WO 99/32134, WO 99/32139, WO 99/32140, WO 96/40791, WO 98/32466, WO 95/06058, EP 439 508, WO 97/03106, WO 96/21469, WO 95/13312, EP 921 131, U.S. Pat. No. 5,736,625, WO 98/05363, EP 809 996, U.S. Pat. No. 5,629,384, WO 96/41813, WO 96/07670, U.S. Pat. No. 5,473,034, U.S. Pat. No. 5,516,673, EP 605 963, U.S. Pat. No. 5,382,657, EP 510 356, EP 400 472, EP 183 503 and EP 154 316.

The conjugation of the polypeptide and the activated polymer molecules is conducted by use of any conventional method, e.g. as described in the following references (which also describe suitable methods for activation of polymer molecules): Harris and Zalipsky, eds., Poly(ethylene glycol) Chemistry and Biological Applications, AZC, Washington; R.F. Taylor, (1991), “Protein immobilisation. Fundamental and applications”, Marcel Dekker, N.Y.; S.S. Wong, (1992), “Chemistry of Protein Conjugation and Crosslinking”, CRC Press, Boca Raton; G.T. Hermanson et al., (1993), “Immobilized Affinity Ligand Techniques”, Academic Press, N.Y.). The skilled person will be aware that the activation method and/or conjugation chemistry to be used depends on the attachment group(s) of the adiponectin polypeptide as well as the functional groups of the polymer (e.g. being amino, hydroxyl, carboxyl, aldehyde or sulfydryl). The PEGylation may be directed towards conjugation to all available attachment groups on the polypeptide (i.e. such attachment groups that are exposed at the surface of the polypeptide) or may be directed towards specific attachment groups, e.g. the N-terminal amino group (U.S. Pat. No. 5,985,265). Furthermore, the conjugation may be achieved in one step or in a stepwise manner (e.g. as described in WO 99/55377).

It will be understood that the PEGylation is designed so as to produce the optimal molecule with respect to the number of PEG molecules attached, the size and form (e.g. whether they are linear or branched) of such molecules, and where in the polypeptide such molecules are attached. For instance, the molecular weight of the polymer to be used may be chosen on the basis of the desired effect to be achieved. For instance, if the primary purpose of the conjugation is to achieve a conjugate having a high molecular weight (e.g. to reduce renal clearance) it is usually desirable to conjugate as few high Mw polymer molecules as possible to obtain the desired molecular weight. When a high degree of epitope shielding is desirable this may be obtained by use of a sufficiently high number of low molecular weight polymer (e.g. with a molecular weight of about 5,000 Da) to effectively shield all or most epitopes of the polypeptide. For instance, 2-8, such as 3-6 such polymers may be used. In connection with conjugation to only a single attachment group on the protein (as described in U.S. Pat. No. 5,985,265), it may be advantageous that the polymer molecule, which may be linear or branched, has a high molecular weight, e.g. about 20 kDa.

Normally, the polymer conjugation is performed under conditions aiming at reacting all available polymer attachment groups with polymer molecules. Typically, the molar ratio of activated polymer molecules to polypeptide is 1000-1, in particular 200-1, preferably 100-1, such as 10-1 or 5-1 in order to obtain optimal reaction. However, also equimolar ratios may be used. It is also contemplated according to the invention to couple the polymer molecules to the polypeptide through a linker. Suitable linkers are well known to the skilled person. A preferred example is cyanuric chloride (Abuchowski et al., (1977), J. Biol. Chem., 252, 3578-3581; U.S. Pat. No. 4,179,337; Shafer et al., (1986), J. Polym. Sci. Polym. Chem. Ed., 24, 375-378.

Subsequent to the conjugation residual activated polymer molecules are blocked according to methods known in the art, e.g. by addition of primary amine to the reaction mixture, and the resulting inactivated polymer molecules removed by a suitable method.

The general technology described in WO 99/55377 is also applicable in respect of producing the conjugates of the present invention. Accordingly, in a further aspect the invention relates to a method for stepwise attachment of polyethylene glycol (PEG) moieties in series to an adiponectin polypeptide, comprising the steps of:

reacting an adiponectin polypeptide with a low molecular weight heterobifunctional or homobifunctional PEG moiety having the following formula: W—CH₂CH₂O(CH₂CH₂O)ₙCH₂CH₂—X, where W and X are groups that independently react with an amine, sulfhydryl, carboxyl or hydroxyl functional group to attach the low molecular weight PEG moiety to the adiponectin polypeptide; and

reacting the low molecular weight PEG moiety attached to the adiponectin polypeptide with a monofunctional or bifunctional PEG moiety to attach the monofunctional or bifunctional PEG moiety to a free terminus of the low molecular weight PEG moiety and form a PEG-adiponectin polypeptide conjugate. The n is an integer, which will depend on the weight of the low molecular weight PEG moiety. In one embodiment the monofunctional or bifunctional PEG moiety has the following formula: Y—CH₂CH₂O(CH₂CH₂O)ₙCH₂CH₂-Z, wherein Y is reactive to a terminal group on the free terminus of the low molecular weight PEG moiety attached to the adiponectin polypeptide and Z is —OCH3 or a group reactive with X to form a bifunctional conjugate. In a further embodiment the monofunctional or bifunctional PEG moiety is methoxy PEG, branched PEG, hydrolytically or enzymatically degradable PEG, pendant PEG, or dendrimer PEG. In a further embodiment W and X are independently selected from the group consisting of orthopyridyl disulfide, maleimides, vinylsulfones, iodoacetamides, hydrazides, aldehydes, succinimidyl esters, epoxides, amines, thiols, carboxyls, active esters, benzotriazole carbonates, p-nitrophenol carbonates, isocyanates, and biotin. In a further embodiment the low molecular weight PEG moiety has a molecular weight in a range of about 100 to 5,000 daltons, one example being OPSS-PEG-hydrazide. In a further embodiment the monofunctional or bifunctional PEG moiety has a molecular weight in a range of about 100 daltons to 200 kilodaltons. In a further embodiment the low molecular weight PEG moiety and/or the monofunctional or bifunctional PEG moiety is a copolymer of polyethylene glycol, such copolymer of polyethylene glycol is typically, selected from the group consisting of polyethylene glycol/polypropylene glycol copolymers and polyethylene glycol/poly (lactic/glycolic acid) copolymers. In a further embodiment the method further comprises a step of purifying the PEG-adiponectin polypeptide conjugate following the stepwise attachment of two PEG moieties in series to an adiponectin polypeptide. The term “OPSS-PEG-hydrazide in combination with mPEG-ALD” as used above and throughout this description is intended to means that the stepwise technologi disclosed in WO 99/55377 may be used. The disclosure of WO 99/55377 is incorporated herein by reference.

Covalent in vitro coupling of a carbohydrate moiety to amino acid residues of adiponectin polypeptide may be used to modify or increase the number or profile of carbohydrate substituents. Depending on the coupling mode used, the carbohydrate(s) may be attached to a) arginine and histidine (Lundblad and Noyes, Chemical Reagents for Protein Modification, CRC Press Inc. Boca Raton, Fla.), b) free carboxyl groups (e.g. of the C-terminal amino acid residue, asparagine or glutamine), c) free sulfhydryl groups such as that of cysteine, d) free hydroxyl groups such as those of serine, threonine, tyrosine or hydroxyproline, e) aromatic residues such as those of phenylalanine or tryptophan or f) the amide group of glutamine. These amino acid residues constitute examples of attachment groups for a carbohydrate moiety, which may be introduced in the adiponectin polypeptide. Suitable methods of in vitro coupling are described in WO 87/05330 and in Aplin eti al., CRC Crit Rev. Biochem., pp. 259-306, 1981. The in vitro coupling of carbohydrate moieties or PEG to protein- and peptide-bound Gln-residues can also be carried out by transglutaminases (TGases), e.g. as described by Sato et al., 1996 Biochemistry 35, 13072-13080 or in EP 725145

Coupling to a Sugar Moiety

In order to achieve in vivo glycosylation of an adiponectin polypeptide that has been modified by introduction of one or more glycosylation sites (see the section “Conjugates of the invention wherein the non-polypeptide moiety is a sugar moiety”), the nucleotide sequence encoding the polypeptide part of the conjugate must be inserted in a glycosylating, eucaryotic expression host. The expression host cell may be selected from fungal (filamentous fungal or yeast), insect, mammalian animal cells, from transgenic plant cells or from transgenic animals. Furthermore, the glycosylation may be achieved in the human body when using a nucleotide sequence encoding the polypeptide part of a conjugate of the invention or a polypeptide of the invention in gene therapy. In one embodiment the host cell is a mammalian cell, such as an CHO cell, BHK or HEK cell, e.g. HEK293, or an insect cell, such as an SF9 cell, or a yeast cell, e.g. Saccharomyces cerevisiae, Pichia pastoris or any other suitable glycosylating host, e.g. as described further below. Optionally, sugar moieties attached to the adiponectin polypeptide by in vivo glycosylation are further modified by use of glycosyltransferases, e.g. using the glycoAdvance™ technology marketed by Neose, Horsham, PA, USA. Thereby, it is possible to, e.g., increase the sialyation of the glycosylated adiponectin polypeptide following expression and in vivo glycosylation by CHO cells.

Coupling to an Organic Derivatizing Agent

Covalent modification of the adiponectin polypeptide may be performed by reacting (an) attachment group(s) of the polypeptide with an organic derivatizing agent. Suitable derivatizing agents and methods are well known in the art. For example, cysteinyl residues most commonly are reacted with α-haloacetates (and corresponding amines), such as chloroacetic acid or chloroacetamide, to give carboxymethyl or carboxyamidomethyl derivatives. Cysteinyl residues also are derivatized by reaction with bromotrifluoroacetone, α-bromo-β-(4-imidozoyl)propionic acid, chloroacetyl phosphate, N-alkylmaleimides, 3-nitro-2-pyridyl disulfide, methyl 2-pyridyl disulfide, p-chloromercuribenzoate, 2-chloromercuri-4-nitrophenol, or chloro-7-nitrobenzo-2-oxa-1,3-diazole. Histidyl residues are derivatized by reaction with diethylpyrocarbonateat pH 5.5-7.0 because this agent is relatively specific for the histidyl side chain. Para-bromophenacyl bromide also is useful; the reaction is preferably performed in 0.1 M sodium cacodylate at pH 6.0.Lysinyl and amino terminal residues are reacted with succinic or other carboxylic acid anhydrides. Derivatization with these agents has the effect of reversing the charge of the lysinyl residues. Other suitable reagents for derivatizing α-amino-containing residues include imidoesters such as methyl picolinimidate; pyridoxal phosphate; pyridoxal; chloroborohydride; trinitrobenzenesulfonic acid; O-methylisourea; 2,4-pentanedione; and transaminase-catalyzed reaction with glyoxylate. Arginyl residues are modified by reaction with one or several conventional reagents, among them phenylglyoxal, 2,3-butanedione, 1,2-cyclohexanedione, and ninhydrin. Derivatization of arginine residues requires that the reaction be performed in alkaline conditions because of the high pKa of the guanidine functional group. Furthermore, these reagents may react with the groups of lysine as well as the arginine guanidino group. Carboxyl side groups (aspartyl or glutamyl or C-terminal amino acid residue) are selectively modified by reaction with carbodimides (R—N═C═N—R′), where R and R′ are different alkyl groups, such as 1-cyclohexyl-3-(2-morpholinyl4-ethyl) carbodiinide or 1-ethyl-3-(4-azonia-4,4-dimethylpentyl) carbodiimide. Furthermore, aspartyl and glutamyl residues are converted to asparaginyl and glutaminyl residues by reaction with ammonium ions.

Blocking Offunctional Site

Excessive polymer conjugation can lead to a loss of activity of the adiponectin polypeptide to which the polymer is conjugated. This problem can be eliminated, e.g., by removal of attachment groups located at the functional site or by blocking the functional site prior to conjugation. These latter strategies constitute further embodiments of the invention (the first strategy being exemplified further above, e.g. by removal of lysine residues which may be located close to a functional site). More specifically, according to the second strategy the conjugation between the adiponectin polypeptide and the non-polypeptide moiety is conducted under conditions where the functional site of the polypeptide is blocked by a helper molecule capable of binding to the functional site of the polypeptide. Typically, the helper molecule is one, which specifically recognizes a functional site of the polypeptide, such as a receptor. Alternatively, the helper molecule may be an antibody, in particular a monoclonal antibody recognizing the adiponectin polypeptide. In particular, the helper molecule may be a neutralizing monoclonal antibody.

The polypeptide is allowed to interact with the helper molecule before effecting conjugation. This ensures that the functional site of the polypeptide is shielded or protected and consequently unavailable for derivatization by the non-polypeptide moiety such, as a polymer. Following its elution from the helper molecule, the conjugate between the non-polypeptide moiety and the polypeptide can be recovered with at least a partially preserved functional site.

The subsequent conjugation of the polypeptide having a blocked functional site to a polymer, a lipophilic compound, an organic derivatizing agent or any other compound is conducted in the normal way, e.g. as described in the sections above entitled “Conjugation to . . . .”

Irrespectively of the nature of the helper molecule to be used to shield the functional site of the polypeptide from conjugation, it is desirable that the helper molecule is free from or comprises only a few attachment groups for the non-polypeptide moiety of choice in part(s) of the molecule, where the conjugation to such groups will hamper the desorption of the conjugated polypeptide from the helper molecule. Hereby, selective conjugation to attachment groups present in non-shielded parts of the polypeptide can be obtained and it is possible to reuse the helper molecule for repeated cycles of conjugation. For instance, if the non-polypeptide moiety is a polymer molecule such as PEG, which has the epsilon amino group of a lysine or N-terminal amino acid residue as an attachment group, it is desirable that the helper molecule is substantially free from conjugatable epsilon amino groups, preferably free from any epsilon amino groups. Accordingly, in a preferred embodiment the helper molecule is a protein or peptide capable of binding to the functional site of the polypeptide, which protein or peptide is free from any conjugatable attachment groups for the non-polypeptide moiety of choice.

In a further embodiment the helper molecule is first covalently linked to a solid phase such as column packing materials, for instance Sephadex or agarose beads, or a surface, e.g. reaction vessel. Subsequently, the polypeptide is loaded onto the column material carrying the helper molecule and conjugation carried out according to methods known in the art, e.g. as described in the sections above entitled “Conjugation to . . . . ”. This procedure allows the polypeptide conjugate to be separated from the helper molecule by elution. The polypeptide conjugate is eluated by conventional techniques under physico-chemical conditions that do not lead to a substantive degradation of the polypeptide conjugate. The fluid phase containing the polypeptide conjugate is separated from the solid phase to which the helper molecule remains covalently linked. The separation can be achieved in other ways: For instance, the helper molecule may be derivatised with a second molecule (e.g. biotin) that can be recognized by a specific binder (e.g. streptavidin). The specific binder may be linked to a solid phase thereby allowing the separation of the polypeptide conjugate from the helper molecule-second molecule complex through passage over a second helper-solid phase column which will retain, upon subsequent elution, the helper molecule-second molecule complex, but not the polypeptide conjugate. The polypeptide conjugate may be released from the helper molecule in any appropriate fashion. De-protection may be achieved by providing conditions in which the helper molecule dissociates from the functional site of the adiponectin to which it is bound. For instance, a complex between an antibody to which a polymer is conjugated and an anti-idiotypic antibody can be dissociated by adjusting the pH to an acid or alkaline pH.

Conjugation of a Tagged Adiponectin Polypeptide

In an alternative embodiment the adiponectin polypeptide is expressed, as a fusion protein, with a tag, i.e. an amino acid sequence or peptide stretch made up of typically 1-30, such as 1-20 or 1-15 or 1-10 amino acid residues. Besides allowing for fast and easy purification, the tag is a convenient tool for achieving conjugation between the tagged polypeptide and the non-polypeptide moiety. In particular, the tag may be used for achieving conjugation in microtiter plates or other carriers, such as paramagnetic beads, to which the tagged polypeptide can be immobilised via the tag. The conjugation to the tagged polypeptide in, e.g., microtiter plates has the advantage that the tagged polypeptide can be immobilised in the microtiter plates directly from the culture broth (in principle without any purification) and subjected to conjugation. Thereby, the total number of process steps (from expression to conjugation) can be reduced. Furthermore, the tag may function as a spacer molecule ensuring an improved accessibility to the immobilised polypeptide to be conjugated. The conjugation using a tagged polypeptide may be to any of the non-polypeptide moieties disclosed herein, e.g. to a polymer molecule such as PEG.

The identity of the specific tag to be used is not critical as long as the tag is capable of being expressed with the polypeptide and is capable of being immobilised on a suitable surface or carrier material. A number of suitable tags are commercially available, e.g. from Unizyme Laboratories, Denmark. For instance, the tag may be any of the following sequences:

His-His-His-His-His-His

Met-Lys-His-His-His-His-His-His

Met-Lys-His-His-Ala-His-His-Gln-His-His

Met-Lys-His-Gln-His-Gln-His-Gln-His-Gln-His-Gln-His-Gln

(vectors useful for providing such tags are available from Unizyme Laboratories, Denmark) or any of the following:

EQKLI SEEDL (a C-terinal tag described in Mol. Cell. Biol. 5:3610-16, 1985)

DYKDDDDK (a C- or N-terminal tag)

YPYDVPDYA

Antibodies against the above tags are commercially available, e.g. from ADI, Aves Lab and Research Diagnostics.

The subsequent cleavage of the tag from the polypeptide may be achieved by use of commercially available enzymes.

Also, the polypeptide may be expressed with a tag, e.g. as described in the section further above entitled “Conjugation of a tagged adiponectin polypeptide”.

It will be understood that any of the polypeptides of the invention disclosed herein may be used to prepare a conjugate of the invention, i.e. be covalently coupled to any of the non-polypeptide moieties disclosed herein. In particular, when a polypeptide of the invention is expressed in a glycosylating microorganism the polypeptide may be provided in glycosylated form.

Methods of Preparing an Adiponectin Polypeptide for Use in the Invention

The polypeptide of the present invention or the polypeptide part of a conjugate of the invention, optionally in glycosylated form, may be produced by any suitable method known in the art. Such methods include constructing a nucleotide sequence encoding the polypeptide and expressing the sequence in a suitable transformed or transfected host. However, polypeptides of the invention may be produced, albeit less efficiently, by chemical synthesis or a combination of chemical synthesis or a combination of chemical synthesis and recombinant DNA technology.

In a further aspect the invention relates to a nucleotide sequence encoding the adiponectin polypeptide part of a conjugate of the invention.

In a further aspect the invention relates to a nucleotide sequence encoding the adiponectin polypeptide fragment of the invention.

In a further aspect the invention relates to an expression vector comprising a nucleotide sequence encoding the adiponectin polypeptide part of a conjugate of the invention.

In a further aspect the invention relates to an expression vector comprising a nucleotide sequence encoding the adiponectin polypeptide fragment of the invention.

In a further aspect the invention relates to a host cell comprising a nucleotide sequence encoding the adiponectin polypeptide part of a conjugate of the invention or an expression vector comprising a nucleotide sequence encoding the adiponectin polypeptide part of a conjugate of the invention.

In a further aspect the invention relates to a host cell comprising a nucleotide sequence encoding the adiponectin polypeptide fragment of the invention or an expression vector comprising a nucleotide sequence encoding the adiponectin polypeptide fragment of the invention.

In one embodiment the nucleotide sequence comprises a sequence selected from any one of SEQ ID NO:14, 15 or 16, as well as sequences having at least 70% homology with any one of SEQ ID NO:14, 15 or 16. More preferred are sequences having at least 80% homology, such as 90%, 92%, 95% or 98% homology with any one of SEQ ID NO:14, 15 or 16.

In another embodiment the nucleotide sequence comprises a sequence selected from any one of SEQ ID NO:62, 63, 64, 65, 66, 67, 68, 69, 70, or 71, as well as sequences having at least 70% homology with any one of SEQ ID NO:62, 63, 64, 65, 66, 67, 68, 69, 70, or 71. More preferred are sequences having at least 80% homology, such as 90%, 92%, 95% or 98% homology with any one of SEQ ID NO:62, 63, 64, 65, 66, 67, 68, 69, 70, or 71.

In a further embodiment the host cell is selected from a yeast cell, a bacterial cell, eg E.coli, a mammalian cell, eg a CHO, BHK, HEK293 cell or an SF9 cell. In a further embodiment the host cell is selected from a bacterial cell, such as E.coli. In a further embodiment the host cell is selected from a mammalian cell, such as CHO K1. Further embodiments of a suitable host cell of the invention is disclosed below.

The nucleotide sequence of the invention encoding an adiponectin polypeptide may be constructed by isolating or synthesizing a nucleotide sequence encoding the parent adiponectin, e.g. with the amino acid sequence shown in SEQ ID NO:6, and then changing the nucleotide sequence so as to effect introduction (i.e. insertion or substitution) or deletion (i.e. removal or substitution) of the relevant amino acid residue(s).

The nucleotide sequence may conveniently be modified by site-directed mutagenesis in accordance with well-known methods.

Alternatively, the nucleotide sequence is prepared by chemical synthesis, e.g. by using an oligonucleotide synthesizer, wherein oligonucleotides are designed based on the amino acid sequence of the desired polypeptide, and preferably selecting those codons that are favored in the host cell in which the recombinant polypeptide will be produced. For example, several small oligonucleotides coding for portions of the desired polypeptide may be synthesized and assembled by PCR, ligation or ligation chain reaction (LCR). The individual oligonucleotides typically contain 5′ or 3′ overhangs for complementary assembly.

Once assembled (by synthesis, site-directed mutagenesis or another method), the nucleotide sequence encoding the adiponectin polypeptide is inserted into a recombinant vector and operably linked to control sequences necessary for expression of the adiponectin in the desired transformed host cell.

It should of course be understood that not all vectors and expression control sequences function equally well to express the nucleotide sequence encoding a polypeptide variant described herein. Neither will all hosts function equally well with the same expression system. However, one of skill in the art may make a selection among these vectors, expression control sequences and hosts without undue experimentation. For example, in selecting a vector, the host must be considered because the vector must replicate in it or be able to integrate into the chromosome. The vector's copy number, the ability to control that copy number, and the expression of any other proteins encoded by the vector, such as antibiotic markers, should also be considered. In selecting an expression control sequence, a variety of factors should also be considered. These include, for example, the relative strength of the sequence, its controllability, and its compatibility with the nucleotide sequence encoding the polypeptide, particularly as regards potential secondary structures. Hosts should be selected by consideration of their compatibility with the chosen vector, the toxicity of the product coded for by the nucleotide sequence, their secretion characteristics, their ability to fold the polypeptide correctly, their fermentation or culture requirements, and the ease of purification of the products coded for by the nucleotide sequence.

The recombinant vector may be an autonomously replicating vector, i.e. a vector which exists as an extrachromosomal entity, the replication of which is independent of chromosomal replication, e.g. a plasmid. Alternatively, the vector is one which, when introduced into a host cell, is integrated into the host cell genome and replicated together with the chromosome(s) into which it has been integrated.

The vector is preferably an expression vector, in which the nucleotide sequence encoding the polypeptide of the invention is operably linked to additional segments required for transcription of the nucleotide sequence. The vector is typically derived from plasmid or viral DNA. A number of suitable expression vectors for expression in the host cells mentioned herein are commercially available or described in the literature. Useful expression vectors for eukaryotic hosts, include, for example, vectors comprising expression control sequences from SV40, bovine papilloma virus, adenovirus and cytomegalovirus. Specific vectors are, e.g., pcDNA3.1(+)\Hyg (Invitrogen, Carlsbad, Calif., USA) and pCI-neo (Promega, La Jola, Calif., USA). Useful expression vectors for bacterial hosts include known bacterial plasmids, such as plasmids from E. coli, including pBR322, pET3a and pET12a (both from Novagen Inc., Wis., USA), wider host range plasmids, such as RP4, phage DNAs, e.g., the numerous derivatives of phage lambda, e.g., NM989, and other DNA phages, such as M13 and filamentous single stranded DNA phages. Useful expression vectors for yeast cells include the 2μ plasmid and derivatives thereof, the POT1 vector (U.S. Pat. No. 4,931,373), the pJSO37 vector described in (Okkels, Ann. New York Acad. Sci. 782, 202-207, 1996) and pPICZ A, B or C (Invitrogen). Useful vectors for insect cells include pVL941, pBG311 (Cate et al., “Isolation of the Bovine and Human Genes for Mullerian Inhibiting Substance And Expression of the Human Gene In Animal Cells”, Cell, 45, pp. 685-98 (1986), pBluebac 4.5 and pMelbac (both available from Invitrogen).

Other vectors for use in this invention include those that allow the nucleotide sequence encoding the polypeptide to be amplified in copy number. Such amplifiable vectors are well known in the art. They include, for example, vectors able to be amplified by DHFR amplification (see, e.g., Kaufman, U.S. Pat. No. 4,470,461, Kaufman and Sharp, “Construction Of A Modular Dihydrofolate Reductase cDNA Gene: Analysis Of Signals Utilized For Efficient Expression”, Mol. Cell. Biol., 2, pp. 1304-19 (1982)) and glutamine synthetase (“GS”) amplification (see, e.g., U.S. Pat. No. 5,122,464 and EP 338,841).

The recombinant vector may further comprise a DNA sequence enabling the vector to replicate in the host cell in question. An example of such a sequence (when the host cell is a mammalian cell) is the SV40 origin of replication. When the host cell is a yeast cell, suitable sequences enabling the vector to replicate are the yeast plasmid 2μ replication genes REP 1-3 and origin of replication.

The vector may also comprise a selectable marker, e.g. a gene the product of which complements a defect in the host cell, such as the gene coding for dihydrofolate reductase (DHFR) or the Schizosaccharomyces pombe TPI gene (described by P. R. Russell, Gene 40, 1985, pp. 125-130), or one which confers resistance to a drug, e.g. ampicillin, kanamycin, tetracyclin, chloramphenicol, neomycin, hygromycin or methotrexate. For filamentous fungi, selectable markers include amdS, pyrG, arcB, niaD, sC.

The term “control sequences” is defined herein to include all components, which are necessary or advantageous for the expression of the polypeptide of the invention. Each control sequence may be native or foreign to the nucleic acid sequence encoding the polypeptide. Such control sequences include, but are not limited to, a leader, polyadenylation sequence, propeptide sequence, promoter, enhancer or upstream activating sequence, signal peptide sequence, and transcription terminator. At a minimum, the control sequences include a promoter.

A wide variety of expression control sequences may be used in the present invention. Such useful expression control sequences include the expression control sequences associated with structural genes of the foregoing expression vectors as well as any sequence known to control the expression of genes of prokaryotic or eukaryotic cells or their viruses, and various combinations thereof.

Examples of suitable control sequences for directing transcription in mammalian cells include the early and late promoters of SV40 and adenovirus, e.g. the adenovirus 2 major late promoter, the MT-1 (metallothionein gene) promoter, the human cytomegalovirus immediate-early gene promoter (CMV), the human elongation factor 1α (EF-1α) promoter, the Drosophila minimal heat shock protein 70 promoter, the Rous Sarcoma Virus (RSV) promoter, the human ubiquitin C (UbC) promoter, the human growth hormone terminator, SV40 or adenovirus E1b region polyadenylation signals and the Kozak consensus sequence (Kozak, M. J Mol Biol Aug. 20, 1987; 196(4):947-50).

In order to improve expression in mammalian cells a synthetic intron may be inserted in the 5′ untranslated region of the nucleotide sequence encoding the polypeptide of interest. An example of a synthetic intron is the synthetic intron from the plasmid pCI-Neo (available from Promega Corporation, Wis., USA).

Examples of suitable control sequences for directing transcription in insect cells include the polyhedrin promoter, the P10 promoter, the Autographa californica polyhedrosis virus basic protein promoter, the baculovirus immediate early gene 1 promoter and the baculovirus 39K delayed-early gene promoter, and the SV40 polyadenylation sequence.

Examples of suitable control sequences for use in yeast host cells include the promoters of the yeast α-mating system, the yeast triose phosphate isomerase (TPI) promoter, promoters from yeast glycolytic genes or alcohol dehydogenase genes, the ADH2-4c promoter and the inducible GAL promoter.

Examples of suitable control sequences for use in filamentous fungal host cells include the ADH3 promoter and terminator, a promoter derived from the genes encoding Aspergillus oryzae TAKA amylase triose phosphate isomerase or alkaline protease, an A. niger α-amylase, A. niger or A. nidulans glucoamylase, A. nidulans acetamidase, Rhizomucor miehei aspartic proteinase or lipase, the TPI1 terminator and the ADH3 terminator. Examples of suitable control sequences for use in bacterial host cells include promoters of the lac system, the trp system, the TAC or TRC system and the major promoter regions of phage lambda.

The nucleotide sequence of the invention encoding an adiponectin polypeptide, whether prepared by site-directed mutagenesis, synthesis or other methods, may or may not also include a nucleotide sequence that encode a signal peptide. The signal peptide is present when the polypeptide is to be secreted from the cells in which it is expressed. Such signal peptide, if present, should be one recognized by the cell chosen for expression of the polypeptide. The signal peptide may be homologous (e.g. be that normally associated with human adiponectin) or heterologous (i.e. originating from another source than human adiponectin) to the polypeptide or may be homologous or heterologous to the host cell, i.e. be a signal peptide normally expressed from the host cell or one which is not normally expressed from the host cell. Accordingly, the signal peptide may be prokaryotic, e.g. derived from a bacterium such as E. coli, or eukaryotic, e.g. derived from a mammalian, or insect or yeast cell.

The presence or absence of a signal peptide will, e.g., depend on the expression host cell used for the production of the polypeptide, the protein to be expressed (whether it is an intracellular or extracellular protein) and whether it is desirable to obtain secretion. For use in filamentous fungi, the signal peptide may conveniently be derived from a gene encoding an Aspergillus sp. amylase or glucoamylase, a gene encoding a Rhizomucor miehei lipase or protease or a Humicola lanuginosa lipase. The signal peptide is preferably derived from a gene encoding A. oryzae TAKA amylase, A. niger neutral α-amylase, A. niger acid-stable amylase, or A. niger glucoamylase. For use in insect cells, the signal peptide may conveniently be derived from an insect gene (cf. WO 90/05783), such as the lepidopteran Manduca sexta adipokinetic hormone precursor, (cf. U.S. Pat. No. 5,023,328), the honeybee melittin (Invitrogen), ecdysteroid UDPglucosyltransferase (egt) (Murphy et al., Protein Expression and Purification 4, 349-357 (1993) or human pancreatic lipase (hpl) (Methods in Enzymology 284, pp. 2⁶²-272, 1997).

A preferred signal peptide for use in mammalian cells is that of human adiponectin apparent from the examples hereinafter or the murine Ig kappa light chain signal peptide (Coloma, M (1992) J. Imun. Methods 152:89-104). For use in yeast cells suitable signal peptides have been found to be the α-factor signal peptide from S. cereviciae. (cf. U.S. Pat. No. 4,870,008), the signal peptide of mouse salivary amylase (cf. 0. Hagenbuchle et al., Nature 289, 1981, pp. 643-646), a modified carboxypeptidase signal peptide (cf. L. A. Valls et al., Cell 48, 1987, pp. 887-897), the yeast BAR1 signal peptide (cf. WO 87/02670), and the yeast aspartic protease 3 (YAP3) signal peptide (cf. M. Egel-Mitani et al., Yeast 6, 1990, pp. 127-137).

Any suitable host may be used to produce the adiponectin polypeptide, including bacteria, fungi (including yeasts), plant, insect, mammal, or other appropriate animal cells or cell lines, as well as transgenic animals or plants. Examples of bacterial host cells include grampositive bacteria such as strains of Bacillus, e.g. B. brevis or B. subtilis, Pseudomonas or Streptomyces, or gramnegative bacteria, such as strains of E. coli. The introduction of a vector into a bacterial host cell may, for instance, be effected by protoplast transformation (see, e.g., Chang and Cohen, 1979, Molecular General Genetics 168: 111-115), using competent cells (see, e.g., Young and Spizizin, 1961, Journal of Bacteriology 81: 823-829, or Dubnau and Davidoff-Abelson, 1971, Journal of Molecular Biology 56: 209-221), electroporation (see, e.g., Shigekawa and Dower, 1988, Biotechniques 6: 742-751), or conjugation (see, e.g., Koehler and Thorne, 1987, Journal of Bacteriology 169: 5771-5278).

Examples of suitable filamentous fungal host cells include strains of Aspergillus, e.g. A. oryzae, A. niger, or A. nidulans, Fusarium or Trichoderna. Fungal cells may be transformed by a process involving protoplast formation, transformation of the protoplasts, and regeneration of the cell wall in a manner known per se. Suitable procedures for transformation of Aspergillus host cells are described in EP 238 023 and U.S. Pat. No. 5,679,543. Suitable methods for transforming Fusarium species are described by Malardier et al., 1989, Gene 78: 147-156 and WO 96/00787. Yeast may be transformed using the procedures described by Becker and Guarente, In Abelson, J. N. and Simon, M. I., editors, Guide to Yeast Genetics and Molecular Biology, Methods in Enzymology, Volume 194, pp 182-187, Academic Press, Inc., New York; Ito et al., 1983, Journal of Bacteriology 153: 163; and Hinnen et al., 1978, Proceedings of the National Academy of Sciences USA 75: 1920.

Examples of suitable yeast host cells include strains of Saccharomyces, e.g. S. cerevisiae, Schizosaccharomyces, Klyveromyces, Pichia, such as P. pastoris or P. methanolica, Hansenula, such as H. Polymorpha or Yarrowia. Methods for transforming yeast cells with heterologous DNA and producing heterologous polypeptides therefrom are disclosed by Clontech Laboratories, Inc, Palo Alto, Calif., USA (in the product protocol for the Yeastmaker™ Yeast Tranformation System Kit), and by Reeves et al., FEMS Microbiology Letters 99 (1992) 193-198, Manivasakam and Schiestl, Nucleic Acids Research, 1993, Vol. 21, No. 18, pp. 44144415 and Ganeva et al., FEMS Microbiology Letters 121 (1994) 159-164.

Examples of suitable insect host cells include a Lepidoptora cell line, such as Spodoptera frugiperda (Sf9 or Sf21) or Trichoplusioa ni cells (High Five) (U.S. Pat. No. 5,077,214). Transformation of insect cells and production of heterologous polypeptides therein may be performed as described by Invitrogen.

Examples of suitable mammalian host cells include Chinese hamster ovary (CHO) cell lines, (e.g. CHO-K1; ATCC CCL-61), Green Monkey cell lines (COS) (e.g. COS 1 (ATCC CRL-1650), COS 7 (ATCC CRL-1651)); mouse cells (e.g. NS/O), Baby Hamster Kidney (BHK) cell lines (e.g. ATCC CRL-1632 or ATCC CCL-10), and human cells (e.g. HEK 293 (ATCC CRL-1573)), as well as plant cells in tissue culture. Additional suitable cell lines are known in the art and available from public depositories such as the American Type Culture Collection, Rockville, Md. Also, the mammalian cell, such as a CHO cell, may be modified to express sialyltransferase, e.g. 2,3-sialyltransferase or 2,6-sialyltransferase, e.g. as described in U.S. Pat. No. 5,047,335, in order to provide improved glycosylation of the adiponectin polypeptide.

Methods for introducing exogeneous DNA into mammalian host cells include calcium phosphate-mediated transfection, electroporation, DEAE-dextran mediated transfection, liposome-mediated transfection, viral vectors and the transfection methods described by Life Technologies Ltd, Paisley, UK using Lipofectamin 2000 and Roche Diagnostics Corporation, Indianapolis, USA using FuGENE 6. These methods are well known in the art and e.g. described by Ausbel et al. (eds.), 1996, Current Protocols in Molecular Biology, John Wiley & Sons, New York, USA. The cultivation of mammalian cells are conducted according to established methods, e.g. as disclosed in (Animal Cell Biotechnology, Methods and Protocols, Edited by Nigel Jenkins, 1999, Human Press Inc, Totowa, N.J. , USA and Harrison M A and Rae I F, General Techniques of Cell Culture, Cambridge University Press 1997).

In the production methods of the present invention, the cells are cultivated in a nutrient medium suitable for production of the polypeptide using methods known in the art. For example, the cell may be cultivated by shake flask cultivation, small-scale or large-scale fermentation (including continuous, batch, fed-batch, or solid state fermentations) in laboratory or industrial fermenters performed in a suitable medium and under conditions allowing the polypeptide to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). If the polypeptide is secreted into the nutrient medium, the polypeptide can be recovered directly from the medium. If the polypeptide is not secreted, it can be recovered from cell lysates. Preferably a medium containing calcium is used, such as DMEM/F-12(1:1) medium Cat no 21041(Invitrogen). However, media without calcium may also be used, such as DMEM Cat no 21068 (Invitrogen).

The resulting polypeptide may be recovered by methods known in the art. For example, the polypeptide may be recovered from the nutrient medium by conventional procedures including, but not limited to, centrifugation, filtration, extraction, spray drying, evaporation, or precipitation.

The polypeptides may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see, e.g., Protein Purification, J. -C. Janson and Lars Ryden, editors, VCH Publishers, New York, 1989).

In connection with the preparation of the conjugate(s), or adiponectin polypeptide fragment of the invention a novel method of preparing a desired adiponectin polypeptide (such as any one of SEQ ID NO:2-8, 10-13, and 17-61), in a mammalian cell has been established. Basically, a cDNA encoding the signal peptide apM1(1-17) wherein the last two C-terminal amino acids are HD and an adiponectin polypeptide having a Gly residue as the N-terminal amino acid was prepared as described in the examples. Using the SignalP V1.1 World Wide Web Server to predict a suitable signal peptide, we discovered that the creation of an HDG amino acid sequence at the C-terminal part of the signal peptide and the desired adiponectin polypeptide resulted in a cleavage site after the G of HDG. By constructing a cDNA as outlined in the examples using the naturally occurring Glycin residues in the collagenous domain of apM1, cleavage sites can be established after e.g. G42, G45, G48, G51, G54, G57, G60, or G63 of the collagenous domain of apM1 (thus leaving the N-terminal amino acid residues: 143, H46, H49, A52, R55, R58, T61, or E64, respectively), in this respect the signal peptide should include His and Asp as the last two C-terminal amino acids, or alternatively, if a cleavage site should be established after G57, or G60, the signal peptide may include His as the last C-terminal amino acid, and make use of the Asp56, or Asp 59, respectively. However, any desired fragment of the adiponectin polypeptide may be prepared, in which case the last three C-terminal amino acids of the signal peptide should be HDG; a non-limiting example is preparation of apM1(101-244) by preparing a cDNA sequence encoding a signal peptide wherein the last three C-terminal amino acids are HDG, and apM1(101-244) thus making it possible for the mammalian cells, such as any one of those mentioned above, preferably CHO cells, to cleave between the C-terminal G of the signal peptide and KIO of apM1(101-244). Thus, a signal peptide wherein the last three C-terminal amino acids of the signal peptide are HDG, also covers the above mentioned situations where a G or DG is the N-terminal amino acid(s) of the adiponectin polypeptide.

Accordingly, in a further aspect the present invention relates to a method of preparing an adiponectin polypeptide, comprising

a) preparing a nucleotide sequence encoding: a signal peptide and the adiponectin polypeptide, wherein the last three C-terminal amino acids of the signal peptide are HDG,

b) inserting the nucleotide sequence into a vector,

c) transfecting the vector into a mammalian cell, and

d) expressing and optionally secreting the adiponectin polypeptide.

In a further aspect the present invention relates to a method of preparing an adiponectin polypeptide, comprising

a) preparing a nucleotide sequence encoding: a signal peptide and the adiponectin polypeptide, wherein the last three C-terminal amino acids of the signal peptide are HDG,

b) inserting the nucleotide sequence into a vector,

c) transfecting the vector into a mammalian cell,

d) expressing and optionally secreting the adiponectin polypeptide, and

e) obtaining the adiponectin polypeptide.

In one embodiment step d involves expressing and secreting the adiponectin polypeptide.

Obtaining the adiponectin polypeptide in step e, typically comprises recovering and purifying the expressed and optionally secreted adiponectin polypeptide. Such methods of recovering or purifying are available to the skilled person, and suitable examples are outlined above.

In a further embodiment the nucleotide sequence is selected from RNA, DNA, or cDNA, preferably cDNA. In a further embodiment the RNA, DNA, or cDNA comprises a sequence selected from SEQ ID NO:9, 14, 15, or 16, as well as sequences having at least 70% homology with any one of SEQ ID NO:9, 14, 15 or 16. More preferred are sequences having at least 80% homology, such as 90%, 92%, 95% or 98% homology with any one of SEQ ID NO:9, 14, 15 or 16.

In a further embodiment the RNA, DNA, or cDNA comprises a sequence selected from SEQ ID NO:62, 63, 64, 65, 66, 67, 68, 69, 70, or 71, as well as sequences having at least 70% homology with any one of SEQ ID NO:62, 63, 64, 65, 66, 67, 68, 69, 70, or 71. More preferred are sequences having at least 80% homology, such as 90%, 92%, 95% or 98% homology with any one of SEQ ID NO:62, 63, 64, 65, 66, 67, 68, 69, 70, or 71.

In a further embodiment the signal peptide is selected from the sequence MLLLGAVLLLLALPGHDG, or MLLLQALLFLLILPSHDG, preferably MLLLGAVLLLLALPGHDG.

In a further embodiment the vector is an expressions vector, such as a plasmid or viral DNA.

Any of the above mentioned mammalian cells are suitable as the host cell expressing the adiponectin polypeptide. In a further embodiment the mammalian cell is selected from a CHO cell.

Other Methods of the Invention

In a still further aspect the invention relates to a method of reducing immunogenicity and/or of increasing functional in vivo half-life and/or serum half-life of an adiponectin polypeptide, which method comprises introducing an amino acid residue constituting an attachment group for a non-polypeptide moiety into a position exposed at the surface of the protein that does not contain such group and removing an amino acid residue constituting an attachment group for a non-polypeptide moiety and subjecting the resulting modified polypeptide to conjugation with the non-polypeptide moiety.

In one embodiment the non-polypeptide moiety is selected from the group consisting of a polymer molecule, a sugar moiety, a lipophilic group and an organic derivatizing agent.

Preferably, the amino acid residue to be introduced and/or removed is as defined in the present application.

In a further aspect the invention relates to a composition comprising any one of the above conjugates, or adiponectin polypeptide fragments. Such composition is typically, selected from a pharmaceutical composition as described below, but may be a bulk composition, such as a freeze dried bulk composition, or liquid composition.

Pharmaceutical Composition and Uses of a Conjugate or Adiponectin Polypeptide Fragment of the Invention

In the following sections reference is only made to a conjugate of the invention, however, in connection with the description of a pharmaceutical composition the phrase, conjugate, also includes adiponectin polypeptides as well as fragments. The conjugate of the invention is administered at a dose typically in the range of 0.001 mg/kg to 0.5 mg/kg body weight. The exact dose to be administered depends on the circumstances. Normally, the dose should be capable of preventing or lessening the severity or spread of the condition or indication being treated. It will be apparent to those of skill in the art that an effective amount of a conjugate or composition of the invention depends, inter alia, upon the disease, the dose, the administration schedule, whether the conjugate or composition is administered alone or in conjunction with other therapeutic agents, the serum half-life of the compositions, and the general health of the patient.

The conjugate of the invention can be used “as is” and/or in a salt form thereof. Suitable salts include, but are not limited to, salts with alkali metals or alkaline earth metals, such as sodium, potassium, lithium, calcium and magnesium, as well as e.g. zinc salts. These salts or complexes may by present as a crystalline and/or amorphous structure.

The conjugate of the invention is preferably administered in a composition including a pharmaceutically acceptable carrier or excipient. “Pharmaceutically acceptable” means a carrier or excipient that does not cause any untoward effects in patients to whom it is administered. Such pharmaceutically acceptable carriers and excipients are well known in the art.

The conjugate of the invention can be formulated into pharmaceutical compositions by well-known methods. Suitable formulations are described in U.S. Pat. No. 5,183,746, Remington's Pharmaceutical Sciences by E. W. Martin, 18^{th} edition, A. R. Gennaro, Ed., Mack Publishing Company [1990]; Pharmaceutical Formulation Development of Peptides and Proteins, S. Frokjaer and L. Hovgaard, Eds., Taylor & Francis [2000]; and Handbook of Pharmaceutical Excipients, 3rd edition, A. Kibbe, Ed., Pharmaceutical Press [2000]).

The pharmaceutical composition of the conjugate of the invention may be formulated in a variety of forms, including liquid, gel, lyophilized, pulmonary dispersion, or any other suitable form, e.g. as a compressed solid. The preferred form will depend upon the particular indication being treated and will be apparent to one of skill in the art.

The pharmaceutical composition containing the conjugate of the invention may be administered orally, intravenously, intracerebrally, intramuscularly, intraperitoneally, intradermally, subcutaneously, intranasally, intrapulmonary, by inhalation, or in any other acceptable manner, e.g. using PowderJect or ProLease technology. The preferred mode of administration will depend upon the particular indication being treated and will be apparent to one of skill in the art.

Parenterals

An example of a pharmaceutical composition is a solution designed for parenteral administration. Although in many cases pharmaceutical solution formulations are provided in liquid form, appropriate for immediate use, such parenteral formulations may also be provided in frozen or in lyophilized form. In the former case, the composition must be thawed prior to use. The latter form is often used to enhance the stability of the active compound contained in the composition under a wider variety of storage conditions, as it is recognized by those skilled in the art that lyophilized preparations are generally more stable than their liquid counterparts. Such lyophilized preparations are reconstituted prior to use by the addition of one or more suitable pharmaceutically acceptable diluents such as sterile water for injection or sterile physiological saline solution.

In case of parenterals, they are prepared for storage as lyophilized formulations or aqueous solutions by mixing, as appropriate, the conjugate having the desired degree of purity with one or more pharmaceutically acceptable carriers, excipients or stabilizers typically employed in the art (all of which are termed “excipients”), for example buffering agents, stabilizing agents, preservatives, isotonifiers, non-ionic detergents, antioxidants and/or other miscellaneous additives.

Buffering agents help to maintain the pH in the range which approximates physiological conditions. They are typically present at a concentration ranging from about 2 mM to about 50 mM. Suitable buffering agents for use with the present invention include both organic and inorganic acids and salts thereof such as citrate buffers (e.g., monosodium citrate-disodium citrate mixture, citric acid-trisodium citrate mixture, citric acid-monosodium citrate mixture, etc.), succinate buffers (e.g., succinic acid-monosodium succinate mixture, succinic acid-sodium hydroxide mixture, succinic acid-disodium succinate mixture, etc.), tartrate buffers (e.g., tartaric acid-sodium tartrate mixture, tartaric acid-potassium tartrate mixture, tartaric acid-sodium hydroxide mixture, etc.), fumarate buffers (e.g., fumaric acid-monosodium fumarate mixture, fumaric acid-disodium fumarate mixture, monosodium fumarate-disodium fumarate mixture, etc.), gluconate buffers (e.g., gluconic acid-sodium glyconate mixture, gluconic acid-sodium hydroxide mixture, gluconic acid-potassium glyuconate mixture, etc.), oxalate buffer (e.g., oxalic acid-sodium oxalate mixture, oxalic acid-sodium hydroxide mixture, oxalic acid-potassium oxalate mixture, etc.), lactate buffers (e.g., lactic acid-sodium lactate mixture, lactic acid-sodium hydroxide mixture, lactic acid-potassium lactate mixture, etc.) and acetate buffers (e.g., acetic acid-sodium acetate mixture, acetic acid-sodium hydroxide mixture, etc.). Additional possibilities are phosphate buffers, histidine buffers and trimethylamine salts such as Tris. Preservatives are added to retard microbial growth, and are typically added in amounts of about 0.2%-1% (w/v). Suitable preservatives for use with the present invention include phenol, benzyl alcohol, meta-cresol, methyl paraben, propyl paraben, octadecyldimethylbenzyl ammonium chloride, benzalkonium halides (e.g. benzalkonium chloride, bromide or iodide), hexamethonium chloride, alkyl parabens such as methyl or propyl paraben, catechol, resorcinol, cyclohexanol and 3-pentanol.

Isotonicifiers are added to ensure isotonicity of liquid compositions and include polyhydric sugar alcohols, preferably trihydric or higher sugar alcohols, such as glycerin, erythritol, arabitol, xylitol, sorbitol and mannitol. Polyhydric alcohols can be present in an amount between 0.1% and 25% by weight, typically 1% to 5%, taking into account the relative amounts of the other ingredients.

Stabilizers refer to a broad category of excipients which can range in function from a bulking agent to an additive which solubilizes the therapeutic agent or helps to prevent denaturation or adherence to the container wall. Typical stabilizers can be polyhydric sugar alcohols (enumerated above); amino acids such as arginine, lysine, glycine, glutamine, asparagine, histidine, alanine, omithine, L-leucine, 2-phenylalanine, glutamic acid, threonine, etc., organic sugars or sugar alcohols, such as lactose, trehalose, stachyose, mannitol, sorbitol, xylitol, ribitol, myoinisitol, galactitol, glycerol and the like, including cyclitols such as inositol; polyethylene glycol; amino acid polymers; sulfur-containing reducing agents, such as urea, glutathione, thioctic acid, sodium thioglycolate, thioglycerol, α-monothioglycerol and sodium thiosulfate; low molecular weight polypeptides (i.e. <10 residues); proteins such as human serum albumin, bovine serum albumin, gelatin or immunoglobulins; hydrophilic polymers such as polyvinylpyrrolidone; monosaccharides such as xylose, mannose, fructose and glucose; disaccharides such as lactose, maltose and sucrose; trisaccharides such as raffinose, and polysaccharides such as dextran. Stabilizers are typically present in the range of from 0.1 to 10,000parts by weight based on the active protein weight.

Non-ionic surfactants or detergents (also known as “wetting agents”) may be present to help solubilize the therapeutic agent as well as to protect the therapeutic polypeptide against agitation-induced aggregation, which also permits the formulation to be exposed to shear surface stress without causing denaturation of the polypeptide. Suitable non-ionic surfactants include polysorbates (20, 80, etc.), polyoxamers (184, 188 etc.), Pluronic® polyols, polyoxyethylene sorbitan monoethers (Tween®-20, Tween®-80, etc.).

Additional miscellaneous excipients include bulking agents or fillers (e.g. starch), chelating agents (e.g. EDTA), antioxidants (e.g., ascorbic acid, methionine, vitamin E) and cosolvents. The active ingredient may also be entrapped in microcapsules prepared, for example, by coascervation techniques or by interfacial polymerization, for example hydroxymethylcellulose, gelatin or poly-(methylmethacylate) microcapsules, in colloidal drug delivery systems (for example liposomes, albumin microspheres, microemulsions, nano-particles and nanocapsules) or in macroemulsions. Such techniques are disclosed in Remington's Pharmaceutical Sciences, supra.

Parenteral formulations to be used for in vivo administration must be sterile. This is readily accomplished, for example, by filtration through sterile filtration membranes.

Sustained Release Preparations

Suitable examples of sustained-release preparations include semi-permeable matrices of solid hydrophobic polymers containing the conjugate, the matrices having a suitable form such as a film or microcapsules. Examples of sustained-release matrices include polyesters, hydrogels (for example, poly(2-hydroxyethyl-methacrylate) or poly(vinylalcohol)), polylactides, copolymers of L-glutamic acid and ethyl-L-glutamate, non-degradable ethylene-vinyl acetate, degradable lactic acid-glycolic acid copolymers such as the ProLease® technology or Lupron Depot® (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for long periods such as up to or over 100 days, certain hydrogels release proteins for shorter time periods. When encapsulated polypeptides remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

Pulmonary Delivery

Conjugate formulations suitable for use with a nebulizer, either jet or ultrasonic, will typically comprise the conjugate dissolved in water at a concentration of, e.g., about 0.01 to 25 mg of conjugate per mL of solution, preferably about 0.1 to 10 mg/mL. The formulation may also include a buffer and a simple sugar (e.g., for protein stabilization and regulation of osmotic pressure), and/or human serum albumin ranging in concentration from 0.1 to 10 mg/ml. Examples of buffers that may be used are sodium acetate, citrate and glycine. Preferably, the buffer will have a composition and molarity suitable to adjust the solution to a pH in the range of 3 to 9. Generally, buffer molarities of from 1 mM to 50 mM are suitable for this purpose. Examples of sugars which can be utilized are lactose, maltose, mannitol, sorbitol, trehalose, and xylose, usually in amounts ranging from 1% to 10% by weight of the formulation.

The nebulizer formulation may also contain a surfactant to reduce or prevent surface induced aggregation of the protein caused by atomization of the solution in forming the aerosol. Various conventional surfactants can be employed, such as polyoxyethylene fatty acid esters and alcohols, and polyoxyethylene sorbitan fatty acid esters. Amounts will generally range between 0.001% and 4% by weight of the.formulation. An especially preferred surfactant for purposes of this invention is polyoxyethylene sorbitan monooleate.

Specific formulations and methods of generating suitable dispersions of liquid particles of the invention are described in WO 9420069, U.S. Pat. No. 5,915,378, U.S. Pat. No. 5,960,792, U.S. Pat. No. 5,957,124, U.S. Pat. No. 5,934,272, U.S. Pat. No. 5,915,378, U.S. Pat. No. 5,855,564, U.S. Pat. No. 5,826,570 and U.S. Pat. No. 5,522,385 which are hereby incorporated by reference.

Three specific examples of commercially available nebulizers suitable for the practice of this invention are the Ultravent nebulizer, manufactured by Mallinckrodt, Inc., St. Louis, Mo., the Acorn II nebulizer, manufactured by Marquest Medical Products, Englewood, Colorado, and the AERx pulmonary drug delivery system manufactured by Aradigm Corporation, Hayward, California.

Conjugate formulations for use with a metered dose inhaler device will generally comprise a finely divided powder. This powder may be produced by lyophilizing and then milling a liquid conjugate formulation and may also contain a stabilizer such as human serum albumin (HSA). Typically, more than 0.5% (w/w) HSA is added. Additionally, one or more sugars or sugar alcohols may be added to the preparation if necessary. Examples include lactose maltose, mannitol, sorbitol, sorbitose, trehalose, xylitol, and xylose. The amount added to the formulation can range from about 0.01 to 200% (w/w), preferably from approximately 1 to 50%, of the conjugate present. Such formulations are then lyophilized and milled to the desired particle size.

The properly sized particles are then suspended in a propellant with the aid of a surfactant. The propellant may be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2-tetrafluoroethane, or combinations thereof. Suitable surfactants include sorbitan trioleate and soya lecithin. Oleic acid may also be useful as a surfactant. This mixture is then loaded into the delivery device. An example of a commercially available metered dose inhaler suitable for use in the present invention is the Ventolin metered dose inhaler, manufactured by Glaxo Inc., Research Triangle Park, N.C.

Such conjugate formulations for powder inhalers will comprise a finely divided dry powder containing conjugate and may also include a bulking agent, such as lactose, sorbitol, sucrose, or mannitol in amounts which facilitate dispersal of the powder from the device, e.g., 50% to 90% by weight of the formulation. The particles of the powder shall have aerodynamic properties in the lung corresponding to particles with a density of about 1 g/cm² having a median diameter less than 10 micrometers, preferably between 0.5 and 5 micrometers, most preferably of between 1.5 and 3.5 micrometers.

An example of a powder inhaler suitable for use in accordance with the teachings herein is the Spinhaler powder inhaler, manufactured by Fisons Corp., Bedford, Mass. The powders for these devices may be generated and/or delivered by methods disclosed in U.S. Pat. Nos. 5,997,848, US 5,993,783, US 5,985,248, US 5,976,574, US 5,922,354, US 5785049 and US 55654007.

The pharmaceutical composition containing the conjugate of the invention may be administered by a wide range of mechanical devices designed for pulmonary delivery of therapeutic products, including but limited to nebulizers, metered dose inhalers, and powder inhalers, all of which are familiar to those of skill in the art.

Some specific examples of commercially available devices suitable for the practice of this invention are the Ultravent nebulizer, manufactured by Mallinckrodt, Inc., St. Louis, Mo.; the Acorn II nebulizer, manufactured by Marquest Medical Products, Englewood, Colorado; the Ventolin metered dose inhaler, manufactured by Glaxo Inc., Research Triangle Park, North Carolina; the Spinhaler powder inhaler, manufactured by Fisons Corp., Bedford, Mass.; the “standing cloud” device of Inhale Therapeutic Systems, Inc., San Carlos, California; the AIR inhaler manufactured by Alkermes, Cambridge, Mass.; and the AERx pulmonary drug delivery system manufactured by Aradigm Corporation, Hayward, California.

The pharmaceutical composition of the invention may be administered in conjunction with other therapeutic agents. These agents may be incorporated as part of the same pharmaceutical composition or may be administered separately from the conjugate of the invention, either concurrently or in accordance with any other acceptable treatment schedule.

In a further aspect the conjugate or adiponectin polypeptide fragment of the invention is administered together with insulin, eg human recombinant insulin. In addition, the conjugate, or adiponectin polypeptide fragment, or pharmaceutical composition of the invention may be used as an adjunct to other therapies.

In a further aspect the invention relates to a pharmaceutical composition comprising a conjugate of the invention and a pharmaceutically acceptable diluent, carrier or adjuvant.

In a further aspect the invention relates to a pharmaceutical composition comprising an adiponectin polypeptide fragment of the invention and a pharmaceutically acceptable diluent, carrier or adjuvant.

The adiponectin polypeptide fragment as part of the pharmaceutical composition may be selected from any one of the aspects or embodiments disclosed in the above sections “Adiponectin polypeptide fragment(s) of the invention” and “Calcium composition aspects”. Moreover, the conjugate as part of the pharmaceutical composition may be selected from any one of the aspects or embodiments disclosed in the above sections “First group of conjugate(s) of the invention”, “Second group of conjugate(s) of the invention”, “Third group of conjugate(s) of the invention”, and “Fourth group of conjugate(s) of the invention”, and “Calcium composition aspects”.

Accordingly, this invention provides compositions and methods for treating type 1 diabetes; impaired glucose tolerance; type 2 diabetes; syndrome X; obesity; cardiovascular disease, such as atherosclerosis; dyslipidemia; or for lowering body weight without reducing food intake; rheumatoid arthritis; Crohn's disease; systemic lupus erythematosus; Sjögren's disease; cachexia; septic shock; myasthenia gravis; post-traumatic brain damage; myocardial infarction; post-surgical brain-damage; and other destructive processes related to stress or activation of the inflammatory system; in particular IGT, type 2 diabetes, syndrome X, dyslipidemia, septic shock, or cardiovascular disease, such as atherosclerosis.

In a further aspect the invention relates to a method of treating a mammal with type 1 diabetes, IGT, type 2 diabetes, syndrome X, obesity, or dyslipidemia, or for lowering body weight of a mammal without reducing food intake, which method comprises administering an effective amount of a conjugate or adiponectin polypeptide fragment of the invention.

In a further aspect the invention relates to a method of treating a mammal with rheumatoid arthritis, Crohn's disease, systemic lupus erythematosus, Sjögren's disease, cachexia, septic shock, diabetes, myasthenia gravis, post-traumatic brain damage, myocardial infarction, post-surgical brain-damage, and other destructive processes related to stress or activation of the inflammatory system, which method comprises administering an effective amount of a conjugate or adiponectin polypeptide fragment of the invention.

In a further aspect the invention relates to use of a conjugate or adiponectin polypeptide fragment of the invention for the manufacture of a medicament for treatment of type 1 diabetes.

In a further aspect the invention relates to use of a conjugate or adiponectin polypeptide fragment of the invention for the manufacture of a medicament for treatment of IGT.

In a further aspect the invention relates to use of a conjugate or adiponectin polypeptide fragment of the invention for the manufacture of a medicament for treatment of type 2 diabetes.

In a further aspect the invention relates to use of a conjugate or adiponectin polypeptide fragment of the invention for the manufacture of a medicament for treatment of syndrome X.

In a further aspect the invention relates to use of a conjugate or adiponectin polypeptide fragment of the invention for the manufacture of a medicament for treatment of obesity.

In a further aspect the invention relates to use of a conjugate or adiponectin polypeptide fragment of the invention for the manufacture of a medicament for treatment of dyslipidemia.

In a further aspect the invention relates to use of a conjugate or adiponectin polypeptide fragment of the invention for the manufacture of a medicament for treatment of septic shock.

In a further aspect the invention relates to use of a conjugate or adiponectin polypeptide fragment of the invention for the manufacture of a medicament for lowering body weight of a mammal without reducing food intake.

In a further aspect the invention relates to use of a conjugate or adiponectin polypeptide fragment of the invention for the manufacture of a medicament for treament of rheumatoid arthritis, Crohn's disease, systemic lupus erythematosus, Sjögren's disease, cachexia, myasthenia gravis, post-traumatic brain damage, myocardial infarction, post-surgical brain-damage, and other destructive processes related to stress or activation of the inflammatory system. Any one of the specified conditions, diseases or disorders is considered separate embodiments of the invention, and as such can be the subject of individual claims.

In fact, we have discovered that the adiponectin polypeptide of the invention, including the composition comprising the adiponectin polypeptide trimer stabilized with calcium ions, and the adiponectin polypeptide fragments of the invention have excellent effect as a TNF-alpha inhibitor and were able to inhibit LPS-induced TNF-alpha production in a monocytic cell line. It is quite unexpected that the present adiponectin polypeptides have this effect, and it means that the present adiponectin polypeptides will be effective as medicaments in the treatment of diseases, disorders, or conditions caused by expression or release of TNF-alpha in a human cell, such as septic shock.

Accordingly, in a further aspect the invention relates to use of an adiponectin polypeptide or conjugate such as any one of those mentioned in the above sections “Adiponectin polypeptide fragment(s) of the invention”, “First group of conjugate(s) of the invention”, “Second group of conjugate(s) of the invention”, “Third group of conjugate(s) of the invention”, and “Fourth group of conjugate(s) of the invention”, and “Calcium composition aspects” for preparing a medicament for treatment of a disease, disorder, or condition caused by expression or release of TNF-alpha in a human cell, wherein said medicament inhibits expression or release of TNF-alpha.

In a further aspect the invention relates to use of an isolated complex comprising

a) a conjugate comprising an adiponectin polypeptide trimer wherein the adiponectin polypeptide trimer contains three adiponectin polypeptide monomers, and one first polymer covalently attached to any one of the three monomers of the adiponectin polypeptide trimer in such a way that the resulting trimer only contains one polymer, and

b) calcium ions,

for preparing a medicament for treatment of a disease, disorder, or condition caused by expression or release of TNF-alpha in a human cell, wherein said medicament inhibits expression or release of TNF-alpha.

Diseases, disorders, or conditions which are caused by expression or release of TNF-alpha in a human cell are for instance, type 1 diabetes, IGT, type 2 diabetes, syndrome X, obesity, or dyslipidemia in any suitable animal, preferably mammal, and in particular human, or for lowering body weight of a mammal, in particular a human, without reducing food intake, rheumatoid arthritis, Crohn's disease, systemic lupus erythematosus, Sjögren's disease, cachexia, septic shock, myasthenia gravis, post-traumatic brain damage, myocardial infarction, post-surgical brain-damage, and other destructive processes related to stress or activation of the inflammatory system. As a test assay for measuring inhibitory effect of a composition or a conjugate comprising an adiponectin polypeptide, the assay described in example 23 may be used.

The invention is further described in the following examples. The examples should not, in any manner, be understood as limiting the generality of the present specification and claims.

Experimental

Structures:

There exist no experimental structures of any part of human adiponectin. An experimental structure determined by X-ray crystallography of the globular part of the homologous protein from mouse have been reported by Shapiro & Scherer (1998) Current Biology, 8, 335-338. They report the structure of an asymmetrical homotrimer of the region equivalent to V11 0 to N244 in sequence id no 1 determined to a resolution of 2.1 Å. In this region the mouse sequence has 12 differences as compared to the human sequence, i.e. 91% sequence identity (see also scheme 1). The structure showed an assymmetrical trimer of beta-sandwich structured monomers, each monomer having a ten-strand jelly-roll folding topology identical to the known TNF (tumor necrosis factor) structures (e.g. Banner et.al. (1993) Cell, 73, 431445). In the structure several regions were not detected, most probably due to dynamical behaviour of these regions that are located in loop regions and at the C-terminal. These regions were (using the residue numbering of the homologous human protein as shown in sequence id no I and in brackets are shown the original numbering from the structure file deposited in PDB (Berman et al. (2000) Nucleic Acids Research, 28, 235-242) having accession code 1C28): In molecule A: K192{A195}, G217{A220}-L224{A227}, N244{A247}. In molecule B: E120{B123}-V125{B128}, A181{B184}, N193{B196}, G217{B220}-N230{B233}, T243{B246}-N244{B247}. In molecule C: V125{C128}-N127{C130}, Y167{C170}-K169{C172}, Y186{C189}-D195{C198}, V215{C218}-V229{C232}, T243{C246}-N244{C247} (see also scheme 1).

Besides the known structure of mouse adiponectin there have been reported a structure of another molecule homologous to human adiponectin. Bogin et.al. (2002) Structure, 10, 165-173, reports the structure of the globular part of the homologous Collagen X. In this region this molecule has 59 residues identical to human adiponectin i.e. 44% sequence identity (see scheme 3). They report a well resolved symmetrical trimer molecule where the most noticeable differences to the reported murine adiponectin are the presence of four calcium ions and one sodium ion. These ions are located in the region where the murine adiponectin are disordered in the structure. Three of the calcium ions are related by symmetry and are coordinated by the sidechains of two aspartic acid residues (D626 and D634), by the backbone carbonyl oxygen of E627 and by one water molecule. The fourth calcium ion is positioned on the three fold symmetry axis and is also coordinated by the side chain of the three copies of D634 as well as the same three water molecules which are also coordinated to the other three calcium ions. The sodium ion is also placed on the three fold symmetry axis 5.98 Å from the central calcium ion, coordinated by the backbone oxygen atom of the three copies of Q635 and to four water molecules.

From our experiments (cf. examples 24 and 25) we have concluded that human adiponectin, such as fragment apM1(82-244) is stabilized on a trimeric form in the presence of calcium ions, and that destabilizing, such as by lowering the pH in the presence of phosphate ions, results in a destabilized trimer. As the residues coordinating the calcium ions are conserved between collagen X and human adiponectin, we conclude that D187 and D195 in the globular domain of human adiponectin are involved in the binding of calcium ions, and that mutation in one or both of these positions results in reduced affinity to calcium ions. Without being bound by theory, we believe that adiponectin in additon to binding calcium ions, probably also binds a sodium ion in the same manner as Collagen X. A buried conserved histidine residue (H163) located close to the symmetry axis at a distance of app. 10 Å from the metal ions can be an important player in the metal ion binding and general stability of the trimer. Under normal conditions this residue is neutral, but at low pH and low calcium ion concentrations this residue could be protonated and thereby destabilize the core of the trimer resulting in a structure similar to the experimentally determined murine adiponectin structure where the structural parts surrounding the metal ion binding sites becomes flexible and unstructured. On the other hand the binding of the metal ions (i.e. high metal ion concentrations) can lower the pKa of the histidine to a level where it will not get protonated even at low pH.

There exist a few experimental structures of collagen like molecules, all of which are based on synthetically produced collagen like fragments. A summary of the known structures can be found in the SCOP data base Murzin et.al. (1995). “SCOP: a structural classification of proteins database for the investigation of sequences and structures”. J. Mol. Biol. 247, 536-540. In this work the structure reported by Berisio et.al. (2002) Protein Sci. 11, 262-270 are used for modelling of the collagen part of human adiponectin.

Methods:

Accessible Surface Area (ASA)

The computer program Access (B. Lee and F. M. Richards, J. Mol.Biol. 55: 379400 (1971)) version 2 (Copyright (c) 1983 Yale University) are used to compute the accessible surface area (ASA) of the individual atoms in the structure. This method typically uses a probe-size of 1.4A and defines the Accessible Surface Area (ASA) as the area formed by the centre of the probe. Prior to this calculation all water molecules and all hydrogen atoms should be removed from the coordinate set, as should other atoms not directly related to the protein.

Fractional ASA of Side Chain

The fractional ASA of the side chain atoms is computed by division of the sum of the ASA of the atoms in the side chain with a value representing the ASA of the side chain atoms of that residue type in an extended ALA-x-ALA tripeptide. See Hubbard, Campbell & Thornton (1991) J. Mol. Biol. 220, 507-530. For this example the CA atom is regarded as a part of the side chain of Glycine residues but not for the remaining residues. The following table are used as standard 100% ASA for the side chain:

| | | |
|---|---|---|
| Ala | 69.23 | Å² |
| Arg | 200.35 | Å² |
| Asn | 106.25 | Å² |
| Asp | 102.06 | Å² |
| Cys | 96.69 | Å² |
| Gln | 140.58 | Å² |
| Glu | 134.61 | Å² |
| Gly | 32.28 | Å² |
| His | 147.00 | Å² |
| Ile | 137.91 | Å² |
| Leu | 140.76 | Å² |
| Lys | 162.50 | Å² |
| Met | 156.08 | Å² |
| Phe | 163.90 | Å² |
| Pro | 119.65 | Å² |
| Ser | 78.16 | Å² |
| Thr | 101.67 | Å² |
| Trp | 210.89 | Å² |
| Tyr | 176.61 | Å² |
| Val | 114.14 | Å² |

Residues not detected in the structure are typically defined as having 100% exposure as they are thought to reside in flexible regions.

Determining Distances Between Atoms:

The distance between atoms is most easily determined using molecular graphics software e.g.

InsightII v. 98.0, MSI INC, or InsightII v 2000.1, Accelrys INC.

Homology Modelling

Homology modelling based on sequence alignment to the sequence or sequences of one or more known structures are performed using the software Modeller 98, MSI INC, or Modeler 2000.1, Accelrys INC.

Modelling of the Globular Part of Human Adiponectin and Determination of Surface Accessibility:

Based on the known structure of the globular part of the murine adiponectin molecule a structure alignment to the human sequence was constructed as shown in Scheme 1. From this alignment a series of 20 model structures was build using Modeller 98 using the input files shown in Scheme 2. Only the part from V110 to N244 was modelled. To simplify further analysis the individual monomers was restrained to be identical using the subroutine “defsym” thereby resulting in a symmetrical trimer as is also seen for most of the homologous TNF like structures. The structure with the lowest “MODELLER OBJECTIVE FUNCTION” having a value of 9004 was structure number 05. This structure was selected as the best representative after an analysis with PROCHECK ver. 3.4 (Laskowski et.al. (1993). J. Appl. Cryst., 26, 283-291) showed an acceptable geometry for all residues, with no residues in the disallowed region of the Ramachandran plot.

In this example, we determined the relative surface accessibility of the side chains in the first monomer molecule of the model number 05 in the context of the intact trimer. The surface accessibility for the other monomers was also calculated and the values correlated generally well with the values for the first molecule except in some loop regions, typically those where the residue position had no structure determined in at least one of the monomers in the template. As the quality of the modelling in these regions are expected to be of a lower quality, and as these residues in the template structure at least in one monomer have showed extreme flexibility, any position which is undetermined in at least one of the template monomers are defined as having 100% surface accessibility. These residues are E120, T121, Y122, V123, T124, I125, P126, N127, Y167, M168, K169, A181, Y186, D187, Q188, Y189, Q190, E191, N192, N193, V194, D195, V215, Y216, G217, E218, G219, E220, R221, N222, G223, L224, Y225, A226, D227, N228, D229, N230, T243, N244 (here as in the rest of the example the residue numbering of sequence id no 1 is used). The residues A108 and Y109 were not included in the model, and are also defined as having 100% surface accessibility.

Surface Exposure:

The following list describe the result of the surface area calculation, revised with respect to the above residues defined as having 100% surface accessibility.

Performing fractional ASA calculations on the first monomer of the 05 model resulted in the following residues having 0% of their side chain exposed to the surface: S113, A114, F115, S116, V117, G118, F132, Y143, G148, F150, C152, G156, L157, Y158, F160, Y162, V166, V173, S174, L175, L183, A197, V201, L202, L203, L205, V211, L213, S232, T235, G236.

The following residues had more than 25% of their side chain exposed to the surface: A108, Y109, V110, Y111, R112, E120, T121, Y122, V123, T124, I125, P126, N127, M128, R131, T133, K134, 1135, Q139, N141, D144, G145, S146, T147, K149, H151, N153, P155, Y167, M168, K169, D170, K178, D179, K180, A181, F184, Y186, D187, Q188, Y189, Q190, E191, N192, N193, V194, D195, H204, E206, V207, G208, Q210, V215, Y216, G217, E218, G219, E220, R221, N222, G223, L224, Y225, A226, D227, N228, D229, N230, H241, D242, T243, N244.

The following residues had more than 50% of their side chain exposed to the surface: A108, Y109, V110, Y111, E120, T121, Y122, V123, T124, I125, P126, N127, M128, R131, Q139, N141, D144, G145, S146, N153, Y167, M168, K169, K178, D179, K180, A181, Y186, D187, Q188, Y189, Q190, E191, N192, N193, V194, D195, E206, V207, G208, V215, Y216, G217, E218, G219, E220, R221, N222, G223, L224, Y225, A226, D227, N228, D229, N230, H241, T243, N244.

The following residues had 100% of their side chain exposed to the surface: A108, Y109, E120, T121, Y122, V123, T124, 1125, P126, N127, Y167, M168, K169, A181, Y186, D187, Q188, Y189, Q190, E191, N192, N193, V194, D195, V215, Y216, G217, E218, G219, E220, R221, N222, G223, L224, Y225, A226, D227, N228, D229, N230, T243, N244.

Modelling of a Calcium Bound Truncated Form of Human Adiponectin (E82-N244) and Determination of Surface Accessibility:

In order to model the globular domain of human adiponectin in the context of the collagen part and in the context of bound metal ions as in the known structure of collagen X, a modelling of the fragment E82-N244 has been performed. The modelling was based on the known crystal structure of the globular part of the murine adiponectin molecule Shapiro & Scherer (1998) Current Biology, 8, 335-338, the crystal structure of the globular part of collagen X (Bogin et.al.,2002, Structure, 10, 165-173), and on the crystal structure of the collagen triple helix [(Pro-Pro-Gly)₁₀]₃ as reported by Berisio et.al. (2002) Protein Sci. 11, 262-270.

The structure of the globular part of collagen X reports only one of the monomers in the symmetrical trimer. The other two can be constructed by application of the appropriate symmetry operations using e.g. the software Swiss-PdbViewer v.3.7 (Guex et al., 1997, Electrophoresis 18,2714-2723).

From the collagen structure, the monomers labelled A, B and C was used in the modelling.

An alignment of the amino acid sequences of the above structures to the human adiponectin sequence was constructed as shown in Scheme 3 and was the basis of the modelling. Prior to the modelling the murine adiponectin structure and the collagen X structure was structurally aligned using Modeler 2000.1 and the collagen structure was placed in an orientation relative to the two other molecules that was app. 100 Å away to minimize any bias on the resulting structures from the original configuration of the template structures.

A modelling strategy where extra constraints was added to keep the globular part in a symmetrical trimer structure was enforced by constraining residues V29-N244 in each of the trimers to be in the same conformation by use of the DEFINE_SYMMETRY command in the Modeler software. The three individual calcium ions was also constrained to each other.

From this alignment a series of 20 model structures was build using Modeller 2000.1 with the input files shown in Scheme 4. The structure with the lowest “MODELLER OBJECTIVE FUNCTION” having a value of 17751 was structure number 03. This structure was selected as the best representative after an analysis with PROCHECK ver. 3.4 (Laskowski et.al. (1993). J. Appl. Cryst., 26, 283-291) showed an acceptable geometry for all residues, with only few residues in the disallowed region of the Ramachandran plot, and most of these belonging to the collagen part.

In this example, we determined the absolute and relative surface accessibility of the side chains in all three monomer molecules of the model number 03 in the context of the intact trimer and including the five metal ions. Besides the few residues having contact to the collagen stalk the surface accessibility for the three monomers was generally quite identical in the globular region (V110-N244) having an average difference in side chain accessible surface area of 1.0 Å2.

Surface Exposure:

Performing fractional ASA calculations on the three monomers of the model 03 resulted in the following residues having 0% of their side chain exposed to the surface in all three monomers: G87, G90, G93, S113, A114, F115, S116, V117, G118, F132, Y143, G148, C152, G156, L157, Y158, F160, V166, S174, L175, D187, D195, S198, V201, L202, L205, V211, L213, G223, A226, S232, F234, G236, F237.

The following residues had more than 25% of their side chain exposed to the surface in at least one of the monomers: E82, T83, G84, V85, P86, A88, E89, P91, R92, F94, P95, I97, Q98, R100, K101, E103, P104, G105, E106, G107, A108, Y109, Y111, E120, T121, Y122, V123, I125, N127, M128, R131, T133, K134, 1135, Q139, N141, D144, G145, S146, T147, K149, H151, N153, P155, Y167, K169, K178, D179, K180, M182, Q188, E191, N192, H204, E206, V207, G208, G217, E218, G219, E220, R221, Y225, D227, N228, D229, H241, D242, T243, N244.

The following residues had more than 50% of their side chain exposed to the surface in at least one of the monomers: E82, T83, G84, V85, P86, A88, E89, P91, R92, F94, P95, 197, Q98, R100, K101, E103, P104, G105, E106, G107, A108, Y109, Y111, E120, T121, Y122, I125, N127, M128, R131, N141, G145, S146, N153, K169, K178, D179, K180, E191, N192, E206, V207, G208, G217, E218, G219, R221, N228, D229, H241, T243, N244.

| Scheme 1 | |
|---|---|
| Sequence alignment the globular domain of human adiponectin (residues V110-N244) to murine | |
| adiponectin. The residues present in each of the three independent monomers in the structure | |
| of the murine protein are shown on the lines labelled 1C28_A, 1C28_B and 1C28_C. | |
| Human: | VYRSAFSVGL ETYVTIPNMP IRFTKIFYNQ QNHYDGSTGK |
| Mouse: | MYRSAFSVGL ETRVTVPNVP IRFTKIFYNQ QNHYDGSTGK |
| 1C28_A: | MYRSAFSVGL ETRVTVPNVP IRFTKTFYNQ QNHYDGSTGK |
| 1C28_B: | MYRSAFSVGL ------PNVP IRFTKIFYNQ QNHYDGSTGK |
| 1C28_C: | MYRSAFSVGL ETRVT---VP IRFTKIFYNQ QNHYDGSTGK |
| Human: | FHCNIPGLYY FAYHITVYMK DVKVSLFKKD KAMLFTYDQY |
| Mouse: | FYCNIPGLYY FSYHITVYMK DVKVSLFKKD KAVLFTYDQY |
| 1C28_A: | FYCNIPGLYY FSYHITVYMK DVKVSLFKKD KAVLFTYDQY |
| 1C28_B: | FYCNIPGLYY FSYHITVYMK DVKVSLFKKD K-VLFTYDQY |
| 1C28_C: | FYCNIPGLYY FSYHITV--- DVKVSLFKKD KAVLFT---- |
| Human: | QENNVDQASG SVLLHLEVGD QVWLQVYGEG ERNGLYADND |
| Mouse: | QEKNVDQASG SVLLHLEVGD QVWLQVYGDG DHNGLYADNV |
| 1C28_A: | QE-NVDQASG SVLLHLEVGD QVWLQVY--- -----YADNV |
| 1C28_B: | QEK-VDQASG SVLLHLEVGD QVWLQVY--- ---------- |
| 1C28_C: | ------QASG SVLLHLEVGD QVWLQ----- ---------- |
| Human: | NDSTFTGFLL YHDTN |
| Mouse: | NDSTFTGFLL YHDTN |
| 1C28_A: | NDSTFTGFLL YHDT- |
| 1C28_B: | -DSTFTGFLL YHD-- |
| 1C28_C: | NDSTFTGFLL YHD-- |

```
Scheme 2
Input files for Modeler 98
# model.top
#
# Modelling of Human ACRP based on X-ray structure of Mouce ACRP:
# 1C28.PDB Mouse trimer enforcing trifold symmetry.
#
INCLUDE
SET OUTPUT_CONTROL = 1 1 1 1
SET ALNFILE = ‘align.pir’
SET PDB_EXT = ‘.pdb’
SET KNOWNS = ‘1C28’
SET SEQUENCE = ‘ACRP_HUMAN’
SET MODEL = ‘ACRP_HUMAN.pdb’
SET ATOM_FILES_DIRECTORY = ‘./’
SET CSRFILE = ‘model.rsr’
SET STARTING_MODEL = 1
SET ENDING_MODEL = 20
SET FINAL_MALIGN3D = 1
#
# Don't use spline functions to convert restraints
#
  SET SPLINE_ON_SITE = off
#
# Set through variable target function
#
  SET_LIBRARY_SCHEDULE = 1
#
# use all restraints to define Hot-spots
#
  SET RSTRS_REFINED = 2
#
# Write report on individual optimizations
#
SET OUTPUT = ‘NO_REPORT SHORT’
SET MD_LEVEL ‘refine1’
CALL ROUTINE = ‘model’
STOP
SUBROUTINE ROUTINE = ‘special_restraints’
    CALL ROUTINE = ‘defsym’
    RETURN
END_SUBROUTINE
SUBROUTINE ROUTINE = ‘defsym’
    SET RES_TYPES = ‘ALL’
    SET ATOM_TYPES = ‘ALL’
    SET SELECTION_STATUS = ‘INITIALIZE’
    SET SELECTION_SEARCH = ‘SEGMENT’
    SET SYMMETRY_WEIGHT = 0.5
    PICK_ATOMS PICK_ATOMS_SET = 2, SELECTION_SEGMENT = ‘1:’ ‘135:’
    PICK_ATOMS PICK_ATOMS_SET = 3, SELECTION_SEGMENT = ‘136:’ ‘270:’
    DEFINE_SYMMETRY ADD_SYMMETRY = on off
    PICK_ATOMS PICK_ATOMS_SET = 2, SELECTION_SEGMENT = ‘136:’ ‘270:’
    PICK_ATOMS PICK_ATOMS_SET = 3, SELECTION_SEGMENT ‘271:’ ‘405:’
    DEFINE_SYMMETRY ADD_SYMMETRY = on off
    PICK_ATOMS PICK_ATOMS SET = 2, SELECTION_SEGMENT = ‘271:’ ‘405:’
    PICK_ATOMS PICK_ATOMS_SET = 3, SELECTIONSEGMENT = ‘1:’ ‘135:’
    DEFINE_SYMMETRY ADD_SYMMETRY = on off
    RETURN
END_SUBROUTINE
Alignment file ‘align.pir’:
>P1;1C28
structureX:1C28_ABC.pdb:113 :A:245 :C:undefined:undefined:−1.00:−1.00
MYRSAFSVGLETRVTVPNVPIRFTKIFYNQQNHYDGSTGKFYCNIPGLYYFSYHIT
VYMKDVKVSLFKKDKAVLFTYDQYQE-NVDQASGSVLLHLEVGDQVWLQVY----Y
ADNVNDSTFTGFLLYHDT-/
MYRSAFSVGL------PNVPIRFTKIFYNQQNHYDGSTGKFYCNIPGLYYFSYHIT
VYMKDVKVSLFKKDK-VLFTYDQYQEK--VDQASGSVLLHLEVGDQVWLQVY---------
-----DSTFTGFLLYHD--/
MYRSAFSVGLETRVT---VPIRFTKIFYNQQNHYDGSTGKFYCNIPGLYYFSYHIT
V---DVKVSLFKKDKAVLFT----------QASGSVLLHLEVGDQVWLQ------------
----NDSTFTGFLLYHD--*
>P1;ACRP_HUMAN
sequence:ACRP_HUMAN: : : : : : :−1.00:−1.00
VYRSAFSVGLETYVTIPNMPIRFTKIFYNQQNHYDGSTGKFHCNIPGLYYFAYHIT
VYMKDVKVSLFKKDKAMLFTYDQYQENNVDQASGSVLLHLEVGDQVWLQVYGEGERNGLY
ADNDNDSTFTGFLLYHDTN/
VYRSAFSVGLETYVTIPNMPIRFTKIFYNQQNHYDGSTGKFHCNIPGLYYFAYHIT
VYMKDVKVSLFKKDKAMLFTYDQYQENNVDQASGSVLLHLEVGDQVWLQVYGEGERNGLY
ADNDNDSTFTGFLLYHDTN/
VYRSAFSVGLETYVTIPNMPIRFTKIFYNQQNHYDGSTGKFHCNIPGLYYFAYHIT
VYMKDVKVSLPKKDKAMLFTYDQYQENNVDQASGSVLLHLEVGDQVWLQVYGEGERNGLY
ADNDNDSTFTGFLLYHDTN*
```

| Scheme 3 | |
|---|---|
| Sequence alignment of residues E82-N244 of human adiponectin to the globular | |
| parts of murine adiponectin and collagen X. The residues present in each of | |
| the three independent monomers in the structure of the murine protein are | |
| shown on the lines labelled 1C28_A, 1C28_B and 1C28_C. The sequence from the | |
| collagen X structure is labelled 1GR3. The sequence of the collagen template | |
| structure is labelled 1K6F. | |
| Human: | ---ETGVPGA EGPRGFPGIQ GRKGEPGEGA Y |
| Mouse: | ---------- ---------- ---------- - |
| 1C28_A: | ---------- ---------- ---------- - |
| 1C28_B: | ---------- ---------- ---------- - |
| 1C28_C: | ---------- ---------- ---------- - |
| 1GR3: | ---------- ---------- ---------- - |
| 1K6F: | PPGPPGPPGP PGPPGPPGPP GPPGPPGPP- - |
| Human: | VYRSAFSVGL ETYVTIPNMP IRFTKIFYNQ QNHYDGSTGK |
| Mouse: | MYRSAFSVGL ETRVTVPNVP IRFTKIFYNQ QNHYDGSTGK |
| 1C28_A: | MYRSAFSVGL ETRVTVPNVP IRFTKIFYNQ QNHYDGSTGK |
| 1C28_B: | MYRSAFSVGL ------PNVP IRFTKIFYNQ QNHYDGSTGK |
| 1C28_C: | MYRSAFSVGL ETRVT---VP IRFTKIFYNQ QNHYDGSTGK |
| 1GR3: | MPVSAFTVIL SKAYPAIGTP IPFDKILYNR QQHYDPRTGI |
| Human: | FHCNIPGLYY FAYHITVYNK DVKVSLFKKD KAMLFTYDQY |
| Mouse: | FYCNIPGLYY FSYHITVYMK DVKVSLFKKD KAVLFTYDQY |
| 1C28_A: | FYCNIPGLYY FSYHITVYMK DVKVSLFKKD KAVLFTYDQY |
| 1C28_B: | FYCNIPGLYY FSYHITVYMK DVKVSLFKKD K-VLFTYDQY |
| 1C28_C: | FYCNIPGLYY FSYHITV--- DVKVSLFKKD KAVLFT---- |
| 1GR3: | FTCQIPGIYY FSYHVHVKGT HVWVGLYKNG TPVMYTYDEY |
| Human: | QENNVDQASG SVLLHLEVGD QVWLQVYGEG ERNGLYADND |
| Mouse: | QEKNVDQASG SVLLHLEVGD QVWLQVYGDG DHNGLYADNV |
| 1C28_A: | QE-NVDQASG SVLLHLEVGD QVWLQVY--- -----YADNV |
| 1C28_B: | QEK-VDQASG SVLLHLEVGD QVWLQVY--- ---------- |
| 1C28_C: | ------QASG SVLLHLEVGD QVWLQ----- ---------- |
| 1GR3: | TKGYLDQASG SAIIDLTEND QVWLQLPNAE S-NGLYSSEY |
| Human: | NDSTFTGFLL YHDTN |
| Mouse: | NDSTFTGFLL YHDTN |
| 1C28_A: | NDSTFTGFLL YHDT- |
| 1C28_B: | -DSTFTGFLL YHD-- |
| 1C28_C: | NDSTFTGFLL YHD-- |
| 1GR3: | VHSSFSGFLV APM- |

```
Scheme 4
Input files for Modeler 2000.1
#
# Modelling of Human ACRP
#
INCLUDE
SET OUTPUT_CONTROL = 1 1 1 1
SET ALNFILE = ‘align.pir’
SET PDB_EXT = ‘.pdb’
SET KNOWNS = ‘1C28’ ‘1GR3_ABC_ions’ ‘1K6F_ABC’
SET SEQUENCE = ‘ACRP_HUMAN’
SET MODEL = ‘ACRP_HUMAN.pdb’
SET ATOM_FILES_DIRECTORY = ‘./’
SET CSRFILE = ‘model.rsr’
SET HETATM_IO = ON
READ_ALIGNMENT FILE = ALNFILE, ALIGN_CODES = KNOWNS SEQUENCE
CHECK_ALIGNMENT
WRITE_ALIGNMENT FILE = ‘Malign.ali’
SET STARTING_MODEL = 1
SET ENDING_MODEL = 20
SET INITIAL_MALIGN3D = 0
SET FINAL_MALIGN3D = 0
SET GENERATE_METHOD = ‘transfer_xyz’
SET OUTPUT_CONTROL = 1 1 1 1 1
SET LIBRARY_SCHEDULE = 1
SET MAX_VAR_ITERATIONS = 1000
SET MD_LEVEL = ‘refine_3’
CALL ROUTINE = ‘model’
STOP
SUBROUTINE ROUTINE = ‘special_restraints’
    CALL ROUTINE = ‘defsym’
    RETURN
END_SUBROUTINE
SUBROUTINE ROUTINE = ‘defsym’
    SET RES_TYPES = ‘ALL’
    SET ATOM_TYPES = ‘ALL’
    SET SELECTION_STATUS = ‘INITIALIZE’
    SET SELECTION_SEARCH = ‘SEGMENT’
    SET SYMMETRY_WEIGHT = 0.5
    PICK_ATOMS PICK_ATOMS_SET = 2, SELECTION_SEGMENT = ‘29:’ ‘163:’
    PICK_ATOMS PICK_ATOMS_SET = 3, SELECTION_SEGMENT = ‘193:’ ‘327:’
    DEFINE_SYMMETRY ADD_SYMMETRY = on off
    PICK_ATOMS PICK_ATOMS_SET = 2, SELECTION_SEGMENT = ‘193:’ ‘327:’
    PICK_ATOMS PICK_ATOMS_SET = 3, SELECTION_SEGMENT = ‘357:’ ‘491:’
    DEFINE_SYMMETRY ADD_SYMMETRY = on off
    PICK_ATOMS PICK ATOMS_SET = 2, SELECTION SEGMENT = ‘357:’ ‘491:’
    PICK_ATOMS PICK_ATOMS_SET = 3, SELECTION_SEGMENT = ‘29:’ ‘163:’
    DEFINE_SYMMETRY ADD_SYMMETRY = on off
    PICK_ATOMS PICK ATOMS_SET = 2, SELECTION_SEGMENT = ‘164:’ ‘164:’
    PICK ATOMS PICK_ATOMS_SET = 3, SELECTION_SEGMENT = ‘328:’ ‘328:’
    DEFINE_SYMMETRY ADD_SYMMETRY = on off
    PICK_ATOMS PICK_ATOMS_SET = 2, SELECTION_SEGMENT = ‘328:’ ‘328:’
    PICK_ATOMS PICK_ATOMS_SET = 3, SELECTION_SEGMENT = ‘492:’ ‘492:’
    DEFINE_SYMMETRY ADD_SYMMETRY = on off
    PICK_ATOMS PICK ATOMS_SET = 2, SELECTION SEGMENT = ‘492:’ ‘492:’
    PICK_ATOMS PICK_ATOMS_SET = 3, SELECTION_SEGMENT = ‘164:’ ‘164:’
    DEFINE_SYMMETRY ADD_SYMMETRY = on off
    RETURN
END_SUBROUTINE
Alignment file ‘align.pir’:
>P1;1C28
structureX:1C28_ABC_edited2.pdb:113 :A:245 :C: : : :
-------------------------------
MYRSAFSVGLETRVTVPNVPIRFTKIFYNQQNHYDGSTGKFYCNIPGLYYFSYHITVYMK
DVKVSLFKKDKAVLFTYDQYQE-NVDQASGSVLLHLEVGDQVWLQVY--------YADNV
NDSTFTGFLLYHDT-/-/
-------------------------------
MYRSAFSVGL------PNVPIRFTKIFYNQQNHYDGSTGKFYCNIPGLYYFSYHITVYMK
DVKVSLFKKDK-VLFTYDQYQEK-VDQASGSVLLHLEVGDQVWLQVY-------------
-DSTFTGFLLYHD--/-/
-------------------------------
MYRSAFSVGLETRVT---VPIRFTKIFYNQQNHYDGSTGKFYCNIPGLYYFSYHITV---
DVKVSLFKKDKAVLFT----------QASGSVLLHLEVGDQVWLQ---------------
NDSTFTGFLLYHD--/-/
-/-*
>P1;1GR3_ABC_ions
structureX:1GR3_ABC_ions_edited.pdb:549 :A:902 :D: : : :
-------------------------------
MPVSAFTVILSKAYPAIGTPIPFDKILYNRQQHYDPRTGIFTCQIPGIYYFSYHVHVKGT
HVWVGLYKNGTPVMYTYDEYTKCYLDQASGSAIIDLTENDQVWLQLPNAES-NGLYSSEY
VHSSFSGFLVAPM--/3/
-------------------------------
MPVSAFTVILSKAYPAIGTPIPFDKILYNRQQHYDPRTGIFTCQIPGIYYFSYHVHVKGT
HVWVGLYKNGTPVMYTYDEYTKGYLDQASGSAIIDLTENDQVWLQLPNAES-NGLYSSEY
VHSSFSGFLVAPM--/3 /
-------------------------------
MPVSAFTVILSKAYPAIGTPIPFDKILYNRQQHYDPRTGIFTCQIPGIYYFSYHVHVKGT
HVWVGLYKNGTPVMYTYDEYTKGYLDQASGSAIIDLTENDQVWLQLPNAES-NGLYSSEY
VHSSFSGFLVAPM--/3/
3/9*
>P1;1K6F_ABC
structureX:1KGF_ABC_edited2.pdb:1 :A:29 :C: : : :
PPGPPGPPGPPGPPGPPGPPGPPCPPGPP--
------------------------------------------------------------
------------------------------------------------------------
---------------/-/
PPGPPGPPGPPGPPGPPGPPGPPGPPGPP--
------------------------------------------------------------
------------------------------------------------------------
---------------/-/
PPCPPCPPGPPGPPCPPGPPGPPCPPCPP--
------------------------------------------------------------
------------------------------------------------------------
/-/*
>P1;ACRP_HUMAN
sequence:ACRP_HUMAN: : : : : : :−1.00:−1.00
---ETGVPGAEGPRGFPGIQGRKGEPGEGAY
VYRSAFSVGLETYVTIPNMPIRFTKIFYNQQNHYDGSTGKFHCNIPGLYYFAYHITVYMK
DVKVSLFKKDKAMLFTYDQYQENNVDQASGSVLLHLEVGDQVWLQVYGEGERNGLYADND
NDSTFTGFLLYHDTN/3/
---ETGVPGAEGPRGFPGIQGRKGEPCEGAY
VYRSAFSVGLETYVTIPNMPIRFTKIFYNQQNHYDGSTGKFHCNIPGLYYFAYHITVYMK
DVKVSLFKKDKAMLFTYDQYQENNVDQASGSVLLHLEVGDQVWLQVYGEGERNGLYADND
NDSTFTGFLLYHDTN/3/
---ETGVPGAEGPRGFPGIQGRKCEPGEGAY
VYRSAFSVCLETYVTIPNMPIRFTKIFYNQQNHYDGSTGKFHCNIPGLYYFAYHITVYMK
DVKVSLFKKDKAMLFTYDQYQENNVDQASGSVLLHLEVGDQVWLQVYGEGERNGLYADND
NDSTFTGFLLYHDTN/3/
3/9*
```

Test Assay A: Determination of Adiponectin's Effect on Glucose Uptake in C2C12 Cells.

In order to investigate if an adiponectin polypeptide or a conjugate is able to enhance the glucose uptake in muscle cells, both the basal level and the insulin stimulated level, we used the C2C12 cell line (ATCC, Rockville, Md.). Briefly, quadruple samples of C2C12 cells (10⁵/well) were differentiated in 12-well plates in 1 ml DMEM medium, supplemented with 5% horse serum, at 37° C. for 4 days. Differentiated C2C12 cells were then incubated in different concentrations of adiponectin polypeptide or conjugate for 24 hours, preferably for 4 hours. After washing, the wells were stimulated in the presence/absence of 100 nM insulin for 30 minutes. The wells were washed and incubated for 15 minutes in the presence of 0.5 μCi/Ml ³H-D-Glucose. Glucose uptake was terminated by aspiration of the solution. Cells were then washed three times, and radioactivity associated with the cells was determined by cell lysis in 0.1 M NaOH, followed by scintillation counting. Aliquots of cell lysates were used for protein determination.

Test Assay B: Measurement of inhibition of LPS-induced TNF-alpha production.

In order to investigate if an adiponectin polypeptide or a conjugate is able to inhibit LPS-induced TNF-alpha production we used the monocytic cell line THP-1 (ATCC, Rockville, Md.). Briefly, triplicate samples of THP-1 cells (10⁵/well) were incubated in 96 well-plates at 37° C. with titrated amounts of adiponectin (highest concentration 500 nM (25.5 μg/ml) in serum free cell culture medium (RPMI-1640, containing 10 mM HEPES).

Following 18 h pre-incubation with adiponectin the cultures were incubated for additional 4 h with a final concentration of 0.5 μg/ml lipopolysaccharide (LPS) (List Biologicals) and then 50 μl supernatant where withdrawn and frozen at −20° C. for subsequent analysis of TNF-alpha.

The diluted cell culture supernatants where analyzed for TNF-alpha content using a standard ELISA (R&D), and the IC50 of adiponectin where calculated using a 4-parameter non linear regression data analysis.

Test Assay C: Measurement of Glucose Production in Primary Hepatocytes.

Single-cell suspensions of hepatocytes are obtained from perfusions of Sprague-Dawley rats using the procedure of Berry and Friend (J. Cell. Biol. 43, 506-520, 1969) and the perfusion mixture of Leffert et al. (Methods Enzymol. 58, 536-544, 1979), alternatively pig hepatocytes may also be used. The cells are plated on tissue culture plastic for 6 h at a density of 2×10⁵ cells per well in a 24-well culture plate that is pre-coated with rat-tail collagen I. During plating cells are cultured in RPMI 1640 medium supplemented with 10% FBS, penicillin/streptomycin, 10 microg/ml insulin and 10 microM dexamethasone. After allowing for adherence, the media is changed to RPMI with 5 mM glucose, 0.4% FCS, and no insulin or dexamethasone. The cells are allowed to equilibrate overnight in this low-glucose media. The following morning this media is refreshed, insulin and/or a conjugate of the invention is added and treatment lasted another 24 h. After stimulation, glucose production is measured by incubating the cells for 6 h in glucose-free RPMI containing 5 mM each of alanine, valine, glycine, pyruvate and lactate. Glucose is subsequently measured with a Trinder assay (Sigma). Reduction of glucose production is a clear indication that the tested conjugates increases insulin sensitivity.

### EXAMPLE 1

Expression/Secretion of apM1(100-244) in CHOK1 Cells

In order to get the globular domain of human adiponectin (apMl), preceded by the last 8 amino acids of the collagenous region, secreted from CHOK1 cells the following cDNA is constructed: In brief, by using a 5′ primer (PBR 196; 5′-CGCGGATCCACCATGCTGTTGCTGGGAGCTGTTCTAC TGCTATTAGCTCTGCCCGGTCATGACGGCAGGAAAGGAGAACCTGGAGAA-3′), encoding the signal peptide of apM1 (M1-D17) and 8 amino acids of the collagenous region (G99-E106), together with a 3′ primer (PBR 189; 5′-ATATATCCCAAGCTTTCAGTTGGTGTCATGGTAGA-3′) in a PCR reaction containing QUICK-Clone cDNA (Human fat cell derived; #7128-1, Clontech, USA) as template, a cDNA fragment encoding the signal peptide of apMl (M1-D17), the nine last amino acids of the collagenous region (G99-G107) followed by the entire globular domain (A108-N244) is isolated. The SignalP World Wide Web server (http://www.cbs.dtu.dk/services/SignalP/) predicts the presence and location of signal peptide cleavage between G99 and R100. After treatment of the PCR fragment with BamHI and HindIII the fragment is inserted into a vector designated pcDNA3.1(−)Hygro/Intron (a derivative of pcDNA3.1(−)Hygro (Invitrogen, USA) in which a chimeric intron obtained from pCI-neo (Promega, USA) has been inserted between the. BamHI and NheI sites in the MCS of the vector). The correct DNA sequence of the inserted PCR fragment is confirmed by usage of an ABI PRISM 3100 Genetic Analyzer.

This plasmid is then transfected into CHO K1 cells by usage of Fugene 6 (Roche, USA) as transfection agent. In order to select for stable CHO K1 producers the medium (from now on containing 360 μg/ml Hygromycin (Gibco, USA)) is exchanged every day until a confluent monolayer of primary stable transfectants is obtained. 24 hours later the culture medium is harvested and assayed by Western blotting for the presence of the apM1(100-244) protein. As detecting antibody can be used a polyclonal (rabbit) anti-Acrp30 antibody (Affinity BioReagents, USA; # PA1-054). PA1-054 immunizing peptides correspond to amino acid residues 18-32 and 187-200 from mouse Acrp30 protein. E(18) D D V T T T E E L A P A L V(32) and F(187) T Y D Q Y Q E K N V D Q A(200). The immunizing peptide located in the globular domain of Acrp30 only differs in one position from the corresponding sequence in the apM1 protein (K195N). Preparation of anti-apM1 rabbit antiserum can also easily be done by immunizing rabbits with a synthetic peptide having the sequence: CY(225)ADNDNDSTFTGFLLYHDTN(244). Hereafter the stable pool is cloned by the limited dilution method in order to isolate the highest producing CHO K1 clones.

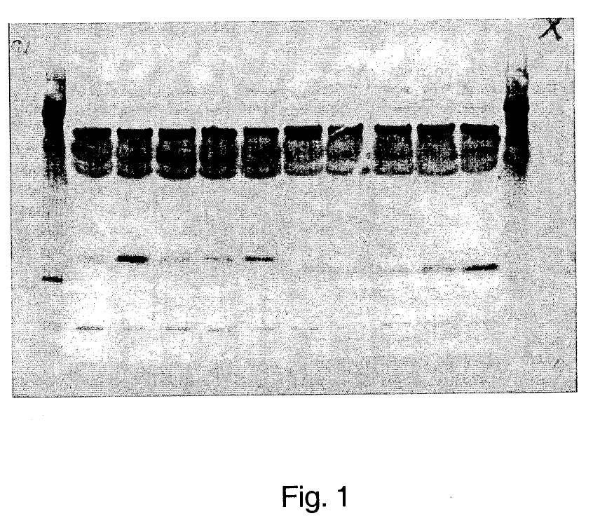

Using the commercial polyclonal (rabbit) anti-Acrp30 antibody (Affinity BioReagents, USA) as detecting antibody in a Western blot (FIG. 1) it was possible to show the expression of apM1(100-244) from the stable pool (lane 7) and from 4 selected stable clones (lanes 8-11). Clone B49 (lane 11) was used for serum-free production of apM1(100-244) in Roller Bottles (see Example 5).

### EXAMPLE 2

Expression/Secretion of apM1(82-244) in CHOK1 Cells

In order to get the globular domain of human adiponectin (apM1), preceded by the last 26 amino acids of the collagenous region, secreted from CHOKI cells the following cDNA is constructed: In brief, by using a 5′ primer (PBR 195; 5′-CGCGGATCCACCATGCTGTTGCTGGGAGCTGTTCTAC TGCTATTAGCTCTGCCCGGTCATGACGGTGAAACCGGAGTACCCGGGGCT-3′), encoding the signal peptide of apMl (M1-D17) and 8 amino acids of the collagenous region (G81-A88), together with a 3′ primer (PBR 189; 5′-ATATATCCCAAGCTTTCAGTTGGTGTCATGGTAGA-3′) in a PCR reaction containing QUICK-Clone cDNA (Human fat cell derived; # 7128-1, Clontech, USA) as template, a cDNA fragment encoding the signal peptide of apM1 (M1-D17), the 27 last amino acids of the collagenous region (G81-G107) followed by the entire globular domain (A108-N244) is isolated. The SignalP World Wide Web server (http://www.cbs.dtu.dklservices/SignalP/) predicts the presence and location of signal peptide cleavage between G81 and E82. After treatment of the PCR fragment with BamHI and HindIII the fragment is inserted into a vector designated pcDNA3. 1(−)Hygro/Intron (a derivative of pcDNA3.1(−)Hygro (Invitrogen, USA) in which a chimeric intron obtained from pCI-neo (Promega, USA) has been inserted between the BamHI and NheI sites in the MCS of the vector. The correct DNA sequence of the inserted PCR fragment is confirmed by usage of an ABI PRISM 3100 Genetic Analyzer.

This plasmid is then transfected into CHO K1 cells by usage of Fugene 6 (Roche, USA) as transfection agent. In order to select for stable CHO K1 producers the medium (from now on containing 360 μg/ml Hygromycin (Gibco, USA)) is exchanged every day until a confluent monolayer of primary stable transfectants is obtained. 24 hours later the culture medium is harvested and assayed by Western blotting for the presence of the apM 1(82-244) protein. As detecting antibody can be used a polyclonal (rabbit) anti-Acrp30 antibody (Affinity BioReagents, USA; #PA1-054). PA1-054 immunizing peptides correspond to amino acid residues 18-32 and 187-200 from mouse Acrp30 protein. E(18) D D V T T T E E L A P A L V(32) and F(187) T Y D Q Y Q E K N V D Q A(200). The immunizing peptide located in the globular domain of Acrp30 only differs in one position from the corresponding sequence in the apMl protein (K195N). Preparation of anti-apMl rabbit antiserum can also easily be done by immunizing rabbits with a synthetic peptide having the sequence: CY(225)ADNDNDSTFTGFLLYHDTN(244). Hereafter the stable pool is cloned by the limited dilution method in order to isolate the highest producing CHO K1 clones.

Using the commercial polyclonal (rabbit) anti-Acrp30 antibody (Affinity BioReagents, USA) as detecting antibody in a Western blot (FIG. 1) it was possible to show the expression of apM1(82-244) from the stable pool (lane 2) and from 4 selected stable clones (lanes 3-6). Clone A6 (lane 3) was used for serum-free production of apM1(82-244) in Roller Bottles (see Example 5)

### EXAMPLE 3

Expression/Secretion of apM1(58-244) in CHOK1 Cells

In order to get the globular domain of human adiponectin (apM1), preceded by the last 50 amino acids of the collagenous region, secreted from CHOKI cells the following cDNA is constructed: In brief, by using a 5′ primer (PBR 203; 5′-CGCGGATCCACCATGCTGTTGCTGGGAGCTGTTCTACTGCTATTAGCTCTGC CCGGTCATGACGGCAGAGATGGCACCCCTGGTGAG-3′), encoding the signal peptide of apM1 (M1-D17) and 8 amino acids of the collagenous region (G57-E64), together with a 3′ primer (PBR 189; 5′-ATATATCCCAAGCTTTCAGTTGGTGTCATGGTAGA-3′) in a PCR reaction containing QUICK-Clone cDNA (Human fat cell derived; # 7128-1, Clontech, USA) as template, a cDNA fragment encoding the signal peptide of apM1(M1-D17), the 51 last amino acids of the collagenous region (G57-G107) followed by the entire globular domain (A108-N244) is isolated. The SignalP World Wide Web server (http://www.cbs.dtu.dk/services/SignalP/) predicts the presence and location of signal peptide cleavage between G57 and R58. After treatment of the PCR fragment with BamHI and HindIII the fragment is inserted into a vector designated pcDNA3.1(−)Hygro/Intron (a derivative of pcDNA3.1 (−) Hygro (Invitrogen, USA) in which a chimeric intron obtained from pCI-neo (Promega, USA) has been inserted between the BamHI and NheI sites in the MCS of the vector). The correct DNA sequence of the inserted PCR fragment is confirmed by usage of an ABI PRISM 3100 Genetic Analyzer.

This plasmid is then transfected into CHO Ki cells by usage of Fugene 6 (Roche, USA) as transfection agent. In order to select for stable CHO KI producers the medium (from now on containing 360 μg/ml Hygromycin (Gibco, USA)) is exchanged every day until a confluent monolayer of primary stable transfectants is obtained. 24 hours later the culture medium is harvested and assayed by Western blotting for the presence of the apMi(58-244) protein. As detecting antibody can be used a polyclonal (rabbit) anti-Acrp30 antibody (Affinity BioReagents, USA; # PA1-054). PA1-054 immunizing peptides correspond to amino acid residues 18-32 and 187-200 from mouse Acrp3O protein. E(18) D D V T T T E E L A P A L V(32) and F(187) T Y D Q Y Q E K N V D Q A(200). The immunizing peptide located in the globular domain of Acrp30 only differs in one position from the corresponding sequence in the apM1 protein (K195N). Preparation of anti-apM1 rabbit antiserum can also easily be done by immunizing rabbits with a synthetic peptide having the sequence: CY(225)ADNDNDSTFTGFLLYHDTN(244). Hereafter the stable pool is cloned by the limited dilution method in order to isolate the highest producing CHO K1 clones.

Expression/Secretion of apM1(58-244) in CHOK1 Cells by Usage of an UCOE Expression Vector

In order to increase the expression level of apM 1(58-244) the construct generated above is digested with NheI and PmeI in order to excise a fragment containing the chimeric intron and the cDNA encoding apM1(58-244). This fragment is then inserted between the NheI and PmeI sites of the expression vector CET 720 (obtained from Cobra Therapeutics Limited, UK), which contains a ubiquitous chromatin opening element (UCOE, cf also WO 00/05393) in front of the CMV promoter.

This plasmid is then transfected into CHO K1 cells by usage of Fugene 6 (Roche, USA) as transfection agent. The following day the medium is exchanged to medium containing 12.5 μg/ml Puromycin (Sigma) in order to select for stable clones. In the next period the selection medium is exchanged every day until a confluent primary selection pool is obtained. At this time a 24-hours medium sample is taken out and assayed by Western blotting for the presence of the apM1(58-244) protein. As detecting antibody is used the polyclonal (rabbit) anti-Acrp3O antibody (Affinity BioReagents, USA; #PA1-054). A relatively strong band, representing the apM1(58-244) protein, is now seen on the Western blot.

### EXAMPLE 4

Expression/Secretion of apM1(52-244) in CHOK1 Cells

In order to get the globular domain of human adiponectin (apM1), preceded by the last 56 amino acids of the collagenous region, secreted from CHOKI cells the following cDNA is constructed: In brief, by using a 5′ primer (PBR 202; 5′-CGCGG ATCCA CCATG CTGTT GCTGG GAGCT GTTCT ACTGC TATTA GCTCT GCCCG GTCAT GACGG GGCCC CAGGC CGTGA TGGCAGA-3′), encoding the signal peptide of apMl (M1-D17) and 8 amino acids of the collagenous region (G51-R58), together with a 3′ primer (PBR 189; 5′-ATATATCCCAAGCTTTCAGTTGGTGTCATGGTAGA-3′) in a PCR reaction containing QUICK-Clone cDNA (Human fat cell derived; # 7128-1, Clontech, USA) as template, a cDNA fragment encoding the signal peptide of apM1 (M1-D17), the 57 last amino acids of the collagenous region (G51-G107) followed by the entire globular domain (A108-N244) is isolated. The SignalP World Wide Web server (http://www.cbs.dtu.dk/services/SignalP/) predicts the presence and location of signal peptide cleavage between G51 and A52. After treatment of the PCR fragment with BamHI and HindIII the fragment is inserted into a vector designated pcDNA3. 1(−)Hygro/Intron (a derivative of pcDNA3.1 (−)Hygro (Invitrogen, USA) in which a chimeric intron obtained from pCI-neo (Promega, USA) has been inserted between the BamHI and NheI sites in the MCS of the vector). The correct DNA sequence of the inserted PCR fragment is confirmed by usage of an ABI PRISM 3100 Genetic Analyzer.

This plasmid is then transfected into CHO K1 cells by usage of Fugene 6 (Roche, USA) as transfection agent. In order to select for stable CHO K1 producers the medium (from now on containing 360 μg/ml Hygromycin (Gibco, USA)) is exchanged every day until a confluent monolayer of primary stable transfectants is obtained. 24 hours later the culture medium is harvested and assayed by Western blotting for the presence of the apM1(52-244) protein. As detecting antibody can be used a polyclonal (rabbit) anti-Acrp30 antibody (Affinity BioReagents, USA; # PA 1-054). PA 1-054 immunizing peptides correspond to amino acid residues 18-32 and 187-200 from mouse Acrp30 protein. E(18) D D V T T T E E L A P A L V(32) and F(187) T Y D Q Y Q E K N V D Q A(200). The immunizing peptide located in the globular domain of Acrp30 only differs in one position from the corresponding sequence in the apM1 protein (K195N). Preparation of anti-apMl rabbit antiserum can also easily be done by immunizing rabbits with a synthetic peptide having the sequence: CY(225)ADNDNDSTFFGFLLYHDTN(244). Hereafter the stable pool is cloned by the limited dilution method in order to isolate the highest producing CHO K1 clones.

Expression/Secretion of apM1(52-244) in CHOK1 Cells by Usage of an UCOE Expression Vector

In order to increase the expression level of apM1(52-244) the construct generated above is digested with NheI and PmeI in order to excise a fragment containing the chimeric intron and the cDNA encoding apM1(52-244). This fragment is then inserted between the NheI and PmeI sites of the expression vector CET 720 (obtained from Cobra Therapeutics Limited, UK), which contains a ubiquitous chromatin opening element (UCOE, cf also WO 00/05393) in front of the CMV promoter.

This plasmid is then transfected into CHO KI cells by usage of Fugene 6 (Roche, USA) as transfection agent. The following day the medium is exchanged to medium containing 12.5 μg/ml Puromycin (Sigma) in order to select for stable clones. In the next period the selection medium is exchanged every day until a confluent primary selection pool is obtained. At this time a 24-hours medium sample is taken out and assayed by Western blotting for the presence of the apM1(52-244) protein. As detecting antibody is used the polyclonal (rabbit) anti-Acrp3O antibody (Affinity BioReagents, USA; # PA1-054). A relatively strong band, representing the apM 1(52-244) protein, is now seen on the Western blot.

### EXAMPLE 5

Seeding of Roller Bottles and Serum-Free Production of apM1 Polypeptide Fragments

At confluency in a T-175 flask, apMl polypeptide fragment producing CHO KI cells are transferred to a roller bottle (1700 cm²) in 300 ml DMEM/F-12 medium (Life Tecnologies # 31330) supplemented with 10% FBS and penicillin/streptomycin (P/S). Medium is exchanged every second day until the bottle is nearly confluent (typically after 4 days). The medium is then changed to 300 ml serum-free UltraCHO medium (BioWhittaker # 12-724) supplemented with 1/1000 EX-CYTE (Serologicals Proteins # 81129N) and P/S. Due to the relatively high protein content in the UltraCHO medium (300 μg/ml) this medium can not be used as a production medium. However, it has appeared that the usage of this medium leads to a very thick cell-layer giving a higher yield in the final production medium. After 4 days (where the medium is exchanged every second day) the roller bottle is ready for production and the medium is shifted to the production medium: DMEM/F-12 medium without phenol red (Life Technologies # 21041; contains 116 mg/l CaCl₂) supplemented with 1/100 ITSA (Life Technologies # 51300-044) [ITSA: Insulin (1.0 g/L)−Transferrin (0.55 g/L)—Selenium (0.67 mg/L) supplement for Adherent cultures], 1/1000 EC-CYTE and P/S. During the production period the medium is exchanged every day.

### EXAMPLE 6

Expression/Secretion of apM1(101-244) in CHOK1 Cells

In order to get the globular domain of human adiponectin (apM1), preceded by the last 7 amino acids of the collagenous region, secreted from CHOK1 cells the following cDNA is constructed: In brief, by using a 5′ primer (PBR 206; 5′-CGCGGATCCACCATGCTG TTGCTGGGAGCTGTTCTACTGCTATTAGCTCTGCCCGGTCATGACGGCAAAGGAGAACCTG GAGAA-3′) encoding the signal peptide of apM1 (M1-D17) followed by a glycine and 6 amino acids of the collagenous region (K101-E106), together with a 3′ primer (PBR 189; 5′-ATATATCCCAAGCTTTCAGTTGGTGTCATGGTAGA-3′) in a PCR reaction containing a plasmid designated PF446, harbouring the full-length apM1 cDNA, as template, a cDNA fragment encoding the signal peptide of apM1 (M1-D17), a glycine and the seven last amino acids of the collagenous region (K101-G107) followed by the entire globular domain (A108-N244) is isolated. The SignalP World Wide Web server (http://www.cbs.dtu.dk/services/SignalP/) predicts the presence and location of signal peptide cleavage between the glycine and K101. After treatment of the PCR fragment with BamHI and HindIII the fragment is inserted into a vector designated pcDNA3.1 (−)Hygro/Intron (a derivative of pcDNA3.1(−)Hygro (Invitrogen, USA) in which a chimeric intron obtained from pCI-neo (Promega, USA) has been inserted between the BamHI and NheI sites in the MCS of the vector. The correct DNA sequence of the inserted PCR fragment is confirmed by usage of an ABI PRISM 3100 Genetic Analyzer.

In order to increase the expression level of apM1(101-244) the construct generated above is digested with NheI and PmeI in order to excise a fragment containing the chimeric intron and the apM1(101-244) cDNA. This fragment is then inserted between the NheI and PmeI sites of the expression vector CET 720 (obtained from Cobra Therapeutics Limited, UK), which contains a ubiquitous chromatin opening element (UCOE) in front of the CMV promoter.

This plasmid is then transfected into CHO K1 cells by usage of Fugene 6 (Roche, USA) as transfection agent. The following day the medium is exchanged to medium containing 12.5 μg/ml Puromycin (Sigma) in order to select for stable clones. In the next period the selection medium is exchanged every day until a confluent primary selection pool is obtained. At this time a 24-hours medium sample is taken out and by Western blotting the presence of the apM1(101-244) protein is verified. As detecting antibody is used the polyclonal (rabbit) anti-Acrp3O antibody (Affinity BioReagents, USA; #PAI-054).

The stable pool is directly expanded into three Roller Bottles for serum free production. The protein is then purified for charaterization.

### EXAMPLE 7

Purification of CHO-Expressed apM1(100-244)

1 L of serum free produced CHO cell culture medium is ultrafiltrated on a Millipore Labscale system using a Biomax 5 membrane. Buffer shift to 20 mM Tris, 50 mM NaCl, pH 8.0 (Buffer A). Final volume 100 ml. This solution is applied to a 20 ml Q Sepharose FF (Pharmacia) column previously equilibrated with 5 column volumes buffer A. Following application the column is washed with 3 column volumes Buffer A and eluted with a linear gradient over 20 column volumes from Buffer A to Buffer A including containing 500 mM NaCl. 2 ml fractions are collected and pooled from A280 and SDS-PAGE analysis. The pool containing apM1(100-244) are concentrated to 2 ml and buffer changed to 50 mM Tris, 100 mM NaCl pH 7.5 using a Viva spin column (5 kDa cut off). Typical yields range from 0.5-2 mg apM1(100-244) from 1 l culture medium. Further purification is obtained by gel permeation chromatography applying the 2 ml concentrated eluate from the anion exchange column to a Sephacryl S-200 HR (16/60 Hi prep material, Pharmacia) previously equilibrated in 20 mM Tris, 100 mM NaCl. Fractions are analyzed by SDS-PAGE and pooled according to purity. The material is >90% pure as judged by SDS-PAGE. The pooled fractions are concentrated on a Viva spin column (5 kDa cut off) and frozen at −80° C.

### EXAMPLE 8

Purification of CHO-Expressed apM1(82-244)

Serum free culture medium is clarified on a 0.22 μm filter. The medium is thereafter concentrated to 10 times by ultrafiltration on a Millipore Labscale system using a Biomax 10 membrane, and diafiltered against 3 volumes of 20 mM Tris pH 7.4 (buffer A). Initial purification is performed by anion exchange chromatography. 200 mL of the resulting solution is applied to a 25 mL Q Sepharose FF (Amersham Biosciences) column previously equilibrated with 4 column volumes of buffer A. Following application, the column is eluted in a linear gradient over 20 column volumes from buffer A to 20% buffer A containing 1 M NaCl. Fractions of 10 mL are collected. The chromatographic system is a Vision BioCAD from PerSeptive Biosystems detecting at 280 nm. A chromatographic peak eluting at ca. 11 mS is identified by SDS-PAGE analysis (non-reducing, treated 5 minutes at 95° C. in SDS sample buffer) to contain a protein at molecular weight slightly less than 20 kDa. Fractions containing this protein are pooled. Further purification is obtained by hydrophobic interaction chromatography. The pool after anion exchange is added (NH₄)₂SO₄ from a 3.5 M stock solution, to a concentration of 0.9 M, and applied to a 8 mL Butyl 650S (TosoHaas) column previously equilibrated with 5 column volumes of 0.9 M (NH₄)₂SO₄ and 20 mM NaH₂PO₄ adjusted to pH 7.2 with NaOH (buffer A). The column is eluted in a linear gradient over 15 column volumes from buffer A to buffer B (20 mM NaH₂PO₄ adjusted to pH 7.2 with NaOH). Fractions of 8 mL column volume are collected. A chromatographic peak eluting at ca. 120 mM (NH₄)₂SO₄ is identified by SDS-PAGE analysis (non-reducing, treated 5 minutes at 95° C. in SDS sample buffer) to contain an almost pure protein at slightly less than 20 kDa. Relevant fractions are pooled, and OD₂₈₀ measured to 0.33, corresponding to a concentration of 0.26 mg/mL using a theoretical molar absorbance of 1.28. The purified protein is frozen at −80° C.

### EXAMPLE 9

Alternative Generic Method for Purification of CHO-Expressed apM1 Fragments and Analogs, such as apM 1(82-244), apM 1(100-244), apM 1(101-244) or S146C-apM 1(82-244)

Serum free culture medium is clarified on a 0.22 μm filter. The medium is thereafter concentrated 10 times by ultrafiltration, and diafiltered against 3 volumes of 20 mM Tris adjusted to pH 7.4 with HCl, using for example a Millipore Labscale system with a Biomax 10 membrane. Initial purification is performed by anion exchange chromatography. Up to 10 column volumes of the diafiltrate is applied to a Q Sepharose FF (Amersham Biosciences) column previously equilibrated with 5 column volumes of 1 mM CaCl₂, 20 mM Tris adjusted to pH 7.4 with HCl (buffer A₁). Following application, the column is eluted in a linear gradient over 20 column volumes from buffer A₁ to buffer B₁ (buffer A₁ containing 0.2 M NaCl). Fractions of about 0.5 column volumes are collected. The chromatographic system may be an Akta Purifier (Amersham Biosciences) detecting at 280 nm. SDS-PAGE analysis (non-reducing, treated 5 minutes at 95° C. in SDS sample buffer) is used to select fractions containing the desired compound. Such fractions contain a protein band corresponding to the molecular weight of the apM1 fragment or analog monomer. The selected fractions are pooled. Further purification is obtained by hydrophobic interaction chromatography. The pool after anion exchange is added 1 volume 5 M NaCl, to a final concentration of 2.5 M NaCl, and applied to a similarly sized Butyl 650S (TosoHaas) column previously equilibrated with 5 column volumes of 2.5 M NaCl, 1 mM CaCl₂, 20 mM Tris adjusted to pH 7.6 with HCl (buffer A₂). The column is eluted in a linear gradient over 15 column volumes from buffer A₂ to buffer B₂ (1 mM CaCl₂, 20 mM Tris adjusted to pH 7.4 with HCl). Fractions of about 0.5 column volume are collected. SDS-PAGE analysis as described above is used for the selection of fractions containing the target compound. The selected fractions are pooled. The pool after hydrophobic interaction chromatography is then concentrated to the desired concentration (e.g. 0.5-1 mg/mL) and diafiltered against 4 volumes of 2 mM CaCl₂, 10 mM sodium citrate, 150 mM sodium chloride adjusted to pH 6.8 with hydrochloric acid. If not used immediately, the purified protein is frozen at −80° C.

Characterization of apM1(82-244)

The purified apM1(82-244) was subjected to automated N-terminal amino acid sequence determination following immobilisation onto a PVDF membrane in a ProSorb device.

The following N-terminal amino acid sequence was found.

Glu-Thr-Gly-Val-(hydroxy-Pro/Pro)-Gly-Ala-Glu-Gly-Pro-Arg-Gly-Phe-(hydroxy-Pro/Pro)-Gly-Ile-Gln-Gly-Arg-(glyco-hydroxy-Lys?/Lys)-Gly-Glu-(hydroxy-Pro/Pro)-

Initially, it should be noted that hydroxy-Pro is positively identified during amino acid sequencing which is not the case for glycosylated hydroxy-Lys (glyco-hydroxy-Lys).

This means that at the positions where (hydroxy-Pro/Pro) are indicated both hydroxy-Pro and Pro are found and positively identified. It also means that at the position where (glyco-hydroxy-Lys?/Lys) is indicated Lys is found and positively identified while the glyco-hydroxy-Lys is suggested based on the presence of additional specific but unidentifiable signals (see below).

The amino acid sequence above is identical to the N-termiinal amino acid sequence of apM1(82-244) but the following comments are necessary.

The Pro-residue in position 86 is to a large extent found in the hydroxylated form as a hydroxy-Pro-residue. However, the hydroxylation is partial as Pro is also easily detected although in lesser amount than hydroxy-Pro.

The Pro-residue in position 91 is found not to be hydroxylated as hydroxy-Pro is not detected.

The Pro-residue in position 95 is almost exclusively found in the hydroxylated form as a hydroxy-Pro-residue. However, Pro is also detected although in very small amounts. The Pro-residues in position 104 is almost exclusively found in the non-hydroxylated form as a Pro-residue. However, hydroxylation is present as hydroxy-Pro is also detected although in very small amounts.

The status of the Lys-residue in position 101 is difficult to assess but the amount of Lys is less than expected. In addition to the lower signal for Lys, several un-identifiable signals are found which potentially represents glyco-hydroxy-Lys. Our interpretation of the data is that LyslOl is partially hydroxylated and the hydroxy-Lys subsequently glycosylated. Hydroxy-Lys is normally only encountered in the glycosylated form as glyco-hydroxy-Lys. Purified apM 1(82-244) was also subjected to MALDI-TOF mass spectrometry and found to contain two components with the masses 18457 Da and 18800 Da, respectively. Reduction of the sample prior to analysis did not alter this.

The theoretical mass of apM1(82-244) is 18424 Da and the mass difference of 33 Da to the form with mass 18457 Da could be explained by hydroxylation of Pro-residues while the additional mass difference of 343 Da to the form with mass 18800 Da could be explained by hydroxylation and subsequent glycosylation of a Lys-residue. Hydroxy-Lys residues are normally only found in the glycosylated form with a glucose-galactose disaccharide attached.

The data obtained by MALDI-TOF mass spectrometry is supported by the result of the N-terminal amino acid sequence determination.

Two other pieces of information can be deducted from the MALDI-TOF mass spectrometry. The first observation is that the potential N-glycosylation site at amino acid residue Asn230 in the globular domain of apM1(82-244) is not utilised as that would have been detected as a significant increase of mass compared to the theoretical mass.

The second information is that the single Cys-residue at position 152 in apM1(82-244) is not modified by attachment of thiol-reactive compounds as the mass of apM1(82-244) does not change upon reduction.

In summary, apM1(82-244) is partially hydroxylated on the three Pro-residues and partially glyco-hydroxylated on the Lys101-residue in the collagen-like part of the molecule.

Interestingly, the hydroxylated and glycosylated component, seen in a spectrum of the apM1(82-244) fragment, is estimated to constitute about 60% relative to the non-hydroxy-glycosylated component.

Characterization of apM1(100-244)

Purified apM1(100-244) was subjected to MALDI-TOF mass spectrometry and found to contain one major component with a mass of 16718 Da. The apM1(100-244) has a theoretical mass of 16715 Da. A small component of 17067 Da is also seen and this component originates from hydroxylated and glycosylated K101. Relative to the major component (16718 Da) this component is estimated to constitute below 5%.

Characterization of apM1(101-244)

By MALDI-TOF mass spectrometry a component with mass 16558.7 Da can be identified as apM(101-244) which has a therotical mass of 16558.4 Da. No component is seen that could represent hydroxylated and glycosylated lysine in position 101 proving that having K101 as the N-terminal amino acid leads to a fragment without any hydroxy-glycosylation at K101.

### EXAMPLE 10

N-terminal PEGylation of apM1(100-244) with 20 kDa PEG

apM1(100-244) used in this example is at a protein concentration of 1.5 mg/ml in 100 mM phosphate buffer at pH 5.0. PEG-aldehyde mW 20 kDa obtained from Shearwater Polymers, Inc. is added as solid. A 1 M NaCNBH₃ stock solution in 100 mM phosphate buffer at pH 5.0 is used. The experiment is carried out as follows: 20 μl 1 M NaCNBH₃ stock solution is added to an Eppendorf tube containing 1 ml of apM1(100-244) solution at 4° C. After mixing, 8 mg of PEG-aldehyde mW 20 kDa is added and the solution mixed. The reaction is allowed to continue for 10 h at 4° C. At this time the reaction is stopped by addition of 200 Ul 100 mM HCL. As judged by SDS-PAGE approximately 90% of the apM1(100-244) is mono PEGylated. Further purification is carried out using a Superose 6 column (Pharmacia) equilibrated in 100 mM phosphate buffer at pH 5.0. The fractions containing mono PEGylated material are pooled based on A280 and SDS-PAGE.

### EXAMPLE 11

N-Terminal PEGylation of apM1(82-244) with 5 and 12 kDa PEG

apM1(82-244) used in this example is at a protein concentration of 1.5 mg/ml in a 10 mM sodium acetate buffer, 1 mM CaCl₂, 200 mM NaCl at pH 5.0. Two different mPEG-aldehyde reagents (5 kDa or 12 kDa) from Shearwater Corporation have been employed. The activated PEG is added as a solid to the protein solution to obtain a 5 molar surplus (5 mol PEG per mol protein). The NaCNBH₃ reagent is added to the protein solution from a 1 M stock solution in 10 mM sodium acetate, 1 mM CaCl₂, 200 mM NaCl at pH 5.0.

The experiments is carried out as follows: 10 μl 1 M NaCNBH₃ stock solution is added to the Eppendorf tube containing 0.5 ml (0.75 mg) of apM1(82-244) at 4° C. and the resulting solution is mixed. Then either 1.1 mg of 5 kDa mPEG-aldehyde or 2.6 mg of 12 kDa mPEG-aldehyde at 4° C. is added and the solution is mixed. The reaction mixture is placed on a rocking platform at 4° C. and allowed to continue for 6-7 hours using the 5 kDa PEG or 4-5 hours using the 12 kDa PEG.

The degree of PEGylation of the apM1(82-244) trimer can be evaluated using an analytical Superdex 200 (pre-packed 1.0 cm ID×30 cm column from Amersham Biosciences) SEC method run under native conditions using the following buffer as mobile phase: 10 mM sodium acetate, 1 mM CaCl₂, 200 mM NaCl at pH 5.0. A flow rate of 1 ml/min and UV-detection at 214 nm is employed.

Using the above PEGylation conditions the protein mixture consists mainly of un-PEGylated apM1(82-244) trimer (approx. 40-50%), apM1(82-244) trimer having one PEG (approx. 40-50%) and apM1(82-244) trimer having two PEGs (approx. 10-20%).

The degree of PEGylation is highly dependent of the reaction time at 4° C. for both 5 kDa and 12 kDa PEG. The yield of apM1(82-244) trimer having three PEGs increases significantly with time. For both PEG sizes the yield of apM1(82-244) trimer having three PEGs is >50% after 20 hours reaction time at 4° C.

The apM1(82-244) trimer having one, two, or three PEGs can be further purified on a semi-preparative SEC column (2.6 cm ID×60 cm) using the resin Superdex 200 prep grade (Amersham Biosciences). A sample volume ≦2 ml is loaded on a pre-equilibrated SEC column and a flow rate of 4 ml/min is used. The mobile phase consists of 10 mM sodium acetate, 1 mM CaCl₂, 200 mM NaCl at pH 5.0. The fractions containing the apM1(82-244) trimer having one, two, and three PEGs are each individually pooled based on the results from the analytical SEC method.

Residual free PEG this can be removed on an anion exchanger. First, the sample is ultrafiltrated and then diafiltrated using for example Vivaspin 20 ml modules (from Vivascience), with a 10 kDa cut-off membrane, against a 1 mM CaCl₂, 20 mM Tris buffer adjusted to pH 7.4 with HCl prior to the anion exchange chromatography step. The diafiltrate is applied to a Q Sepharose FF (Amersham Biosciences) column previously equilibrated with 5 column volumes of 1 mM CaCl2, 20 mM Tris adjusted to pH 7.4 with HCl (buffer A). Following application, the column is eluted in a linear gradient over 20 column volumes from buffer A to buffer B (buffer A containing 0.2 M NaCl). Fractions containing the PEGylated apM1(82-244) are pooled based on results from the analytical SEC method and/or SDS-PAGE analysis. Since the SDS-PAGE analysis is runned under denatured conditions (non-reducing, treated 10 minutes at 70° C. in SDS sample buffer) this method is only used for PEGylated apM1(82-244) samples without remaining free PEG.

### EXAMPLE 12

Construction and Expression of T121C-apM1(82-244).

Using apM 1(82-244)/pcDNA3.1(−)Hygro/Intron as template, two PCR reactions are performed with two overlapping primer-sets [PBR195 (5′-CGCGGATCCACCATGCTGTTGCTGGGAGCTGTT CTACTGCTATTAGCTCTGCCCGGTCATGACGGTGAAACCGGAGTACCCGGGGCT-3′)/PBR211 (5′-GATAGTAACGTAGCACTCCAATCCCACACT-3′) and PBR210 (5′-AGTGTG GGATTGGAGTGCTACGTTACTATC-3′)/PBR193 (5′-ATATATCCCAAGCTTTCAGTTGGTGTC ATGGTAGAG-3′) resulting in two fragments of 375 and 390 base pairs, respectively. These two fragments are assembled in a third PCR reaction with the flanking primers PBR195 and PBR193. The resulting gene is inserted into the mammalian expression vector pcDNA3.1(−)Hygro/Intron and confirmed by DNA sequencing to have the correct base changes leading to T121C-apM1(82-244). This construct is transfected into CHOK1 cells and stable pool is selected with Hygromycin. The title analog, T121C-apM1(82-244), is detected on western blot by usage of the polyclonal (rabbit) anti-Acrp30 antibody (Affinity BioReagents, USA; PA1-054).

### EXAMPLE 13

Construction and Expression of S146C-apM1(82-244).

Using apM1(82-244)/pcDNA3.1(−)Hygro/Intron) as template, two PCR reactions are performed with two overlapping primer-sets [PBR195 (5′-CGCGGATCCACCATGCTGTTGCTGGGAGCT GTTCTACTGCTATTAGCTCTGCCCGGTCATGACGGTGAAACCGGAGTACCCGGGGCT-3′)/PBR213 (5′-GTGGAATTTACCAGTGCAGCCATCATAGTG-3′)and PBR212 (5′-CACT ATGATGGCTGCACTGGTAAATTCCAC-3′)/PBR193 (5′-ATATATCCCAAG CTTTCAGTTGGTGTCATGGTAGAG-3′) resulting in two fragments of 453 and 312 base pairs, respectively. These two fragments are assembled in a third PCR reaction with the flanking primers PBR195 and PBR193. The resulting gene is inserted into the mammalian expression vector pcDNA3.1(−)Hygro/Intron and confirmed by DNA sequencing to have the correct base changes leading to S146C-apM1(82-244). This construct is transfected into CHOKI cells and stable pool is selected with Hygromycin. The title analog, S146C-apM1(82-244), is detected on western blot by usage of the polyclonal (rabbit) anti-Acrp30 antibody (Affinity BioReagents, USA; PA1-054).

### EXAMPLE 14

Construction and Expression of T243C-apMi(82-244).

Using apM1(82-244)/pcDNA3.1(−)Hygro/Intron as template, a PCR reaction is performed with two primers PBR195 (5′-CGCGGATCCACCATGCT GTTGCTGGGAGCTGTTCTACTGCTATTAGCTCTGCCCGGTCATGACGGT GAAACCGGAGTACCCGGGGCT-3′ and PBR214 (5′-ATATATCCCAAGCT TTCAGTTGCAGTCATGGTAGA-3′) resulting in a fragment of 735 bp. This fragment is inserted into the mamnmalian expression vector pcDNA3.1(−)Hygro/Intron and confirmed by DNA sequencing to have the correct base changes leading to T243C-apM 1(82-244). This cDNA, together with the upstream intron, is moved to the UCOE vector CET720. This construct is transfected into CHOK1 cells and stable pool is selected with Puromycin. The title analog, T243C-apM1(82-244), is detected on western blot by usage of the polyclonal (rabbit) anti-Acrp30 antibody (Affinity BioReagents, USA; PA1-054).

### EXAMPLE 15

Construction and Expression of N127C-apM1(82-244).

Using apM1(82-244)/pcDNA3. 1(−)Hygro/Intron as template, two PCR reactions are performed with two overlapping primer-sets [PBR195 (5′-CGCG GATCCACCATGCTGTTGCTGGGAGCTGTTCTACTGCTATTAGCTCTG CCCGGTCATGACG GTGAAACCGGAGTACCCGGGGCT-3′)/PBR225 (5-GCG AATGGGCATGCAGGGGATAGTAACGTA-3′) and PBR224 (5-TACGTTACT ATCCCCTGCATGCCCATTCGC-3′)/PBR 193 (5′-ATATATCCCAAGCTTTCA GTTGGTGTCATGGTAGAG-3′) resulting in two fragments of 393 and 372 base pairs, respectively. These two fragments are assembled in a third PCR reaction with the flanking primers PBR195 and PBR193. The resulting gene is inserted into the mammalian expression vector pcDNA3.1(−)Hygro/Intron and confirmed by DNA sequencing to have the correct base changes leading to N127C-apM1(82-244). This cDNA, together with the upstream intron, is moved to the UCOE vector CET720. This construct is transfected into CHOKI cells and stable pool is selected with Puromycin. The title analog, N127C-apM1(82-244), is being evaluated on western blot by usage of the polyclonal (rabbit) anti-Acrp3O antibody (Affinity BioReagents, USA; PA1-054).

### EXAMPLE 16

Construction and Expression of N141C-apM1(82-244).

Using apM 1(82-244)/pcDNA3.1(−)Hygro/Intron as template, two PCR reactions are performed with two overlapping primer-sets [PBR195 (5′-CGCG GATCCACCATGCTGTTGCTGGGAGCTGTTCTACTGCTATTAGCTCTG CCCGGTCATGACG GTGAAACCGGAGTACCCGGGGCT-3′)/PBR227 (5-GCC ATCATAGTGGCATTGCTGATTGTAGAA-3′) and PBR226 (5-TTCTACAAT CAGCAATGCCACTATGATGGC-3′)/PBR 193 (5′-ATATATCCCAAGCTTTCA GTTGGTGTCATGGTAGAG-3′) resulting in two fragments of 435 and 330 base pairs, respectively. These two fragments are assembled in a third PCR reaction with the flanking primers PBR195 and PBR193. The resulting gene is inserted into the mammalian expression vector pcDNA3.1(−)Hygro/Intron and confirmed by DNA sequencing to have the correct base changes leading to N141C-apM1(82-244). This cDNA, together with the upstream intron, is moved to the UCOE vector CET720. This construct is transfected into CHOKI cells and stable pool is selected with Puromycin. The title analog, N141C-apM1(82-244), is being evaluated on western blot by usage of the polyclonal (rabbit) anti-Acrp3O antibody (Affinity BioReagents, USA; PA1-054).

### EXAMPLE 17

Construction and Expression of N228C-apM1(82-244).

Using apM1(82-244)/pcDNA3.1(−)Hygro/Intron as template, a PCR reaction is performed with two primers PBR195 (5′-CGCGGATCCACCATGCT GTTGCTGGGAGCTGTTCTACTGCTATTAGCTCTGCCCGGTCATGACGGT GAAACCGGAGTACCCGGGGCT-3′ and PBR231 (5′-ATATATCCCAAGCTT TCAGTTGGTGTCATGGTAGAGAAGAAAGCCTGTGAAGGTGGAGTCATT GTCGCAATCAGCATAGAG-3′) resulting in a fragment of 735 bp. This fragment is inserted into the mammalian expression vector pcDNA3.1 (−)Hygro/Intron and confirmed by DNA sequencing to have the correct base changes leading to N228C-apM1(82-244). This cDNA, together with the upstream intron, is moved to the UCOE vector CET720. This construct is transfected into CHOKI cells and stable pool is selected with Puromycin. The title analog, N228C-apM1(82-244), is being evaluated on western blot by usage of the polyclonal (rabbit) anti-Acrp30 antibody (Affinity BioReagents, USA; PA1-054).

### EXAMPLE 18

Construction and Expression of Y111N-apM1(82-244).

Using apM 1(82-244)/pcDNA3.1 (−)Hygro/Intron as template, two PCR reactions are performed with two overlapping primer-sets [PBR195 (5′-CGCG GATCCACCATGCTGTTGCTGGGAGCTGTTCTACTGCTATTAGCTCTG CCCGGTCATGACG GTGAAACCGGAGTACCCGGGGCT-3′)/PBR217 (5-GAA TGCTGAGCGGTTTACATAGGCACCTTC-3′) and PBR216 (5-GAAGGTGCC TATGTAAACCGCTCAGCATTC-3′)/PBR 193 (5′-ATATATCCCAAGCTTTCA GTTGGTGTCATGGTAGAG-3′) resulting in two fragments of 345 and 420 base pairs, respectively. These two fragments are assembled in a third PCR reaction with the flanking primers PBR195 and PBR193. The resulting gene is inserted into the mammalian expression vector pcDNA3.1(−)Hygro/Intron and confirmed by DNA sequencing to have the correct base changes leading to Y111N-apM1(82-244). This cDNA, together with the upstream intron, is moved to the UCOE vector CET720. This construct is transfected into CHOK1 cells and stable pool is selected with Puromycin. The N-linked glycosylated title analog, Y111N-apM1(82-244), is detected on western blot by usage of the polyclonal (rabbit) anti-Acrp30 antibody (Affinity BioReagents, USA; PAI-054). A 100% glycosylation is seen.

### EXAMPLE 19

Construction and Expression of Y122N-apM1(82-244).

Using apM 1(82-244)/pcDNA3.1 (−)Hygro/Intron as template, two PCR reactions are performed with two overlapping primer-sets [PBR195 (5′-CGCG GATCCACCATGCTGTTGCTGGGAGCTGTTCTACTGCTATTAGCTCTGCCCGGTCATGACG GTGAAACCGGAGTACCCGGGGCT-3′)/PBR219 (5-GTT GGGGATAGTAACGTTAGTCTCCAATCC-3′) and PBR218 (5-GGATTGGAG ACTAACGTTACTATCCCCAAC-3′)/PBR 193 (5′-ATATATCCCAAGCTTTCA GTTGGTGTCATGGTAGAG-3′) resulting in two fragments of 381 and 384 base pairs, respectively. These two fragments are assembled in a third PCR reaction with the flanking primers PBR195 and PBR193. The resulting gene is inserted into the mammalian expression vector pcDNA3.1(−)Hygro/Intron and confirmed by DNA sequencing to have the correct base changes leading to Y122N-apM1(82-244). This cDNA, together with the upstream intron, is moved to the UCOE vector CET720. This construct is transfected into CHOKI cells and stable pool is selected with Puromycin. The title analog, Y122N-apM1(82-244), is being evaluated on western blot by usage of the polyclonal (rabbit) anti-Acrp30 antibody (Affinity BioReagents, USA; PA1-054).

### EXAMPLE 20

Construction and Expression of D144N+S146T-apM1(82-244).

Using apM1(82-244)/pcDNA3.1(−)Hygro/Intron as template, two PCR reactions are performed with two overlapping primer-sets [PBR195 (5′-CGCG GATCCACCATGCTGTTGCTGGGAGCTGTTCTACTGCTATTAGCTCTG CCCGGTCATGACG GTGAAACCGGAGTACCCGGGGCT-3′)/PBR221 (5-GAA TTTACCAGTAGTGCCGTTATAGTGGTT-3′) and PBR220 (5-AACCACTAT AACGGCACTACTGGTAAATTC-3′)/PBR 193 (5′-ATATATCCCAAGCTTTCA GTTGGTGTCATGGTAGAG-3′) resulting in two fragments of 450 and 315 base pairs, respectively. These two fragments are assembled in a third PCR reaction with the flanking primers PBR195 and PBR193. The resulting gene is inserted into the mammalian expression vector pcDNA3.1(−)Hygro/Intron and confirmed by DNA sequencing to have the correct base changes leading to D144N+S146T-apM1(82-244). This cDNA, together with the upstream intron, is moved to the UCOE vector CET720. This construct is transfected into CHOK1 cells and stable pool is selected with Puromycin. The title analog, D144N+S146T-apM1(82-244), is being evaluated on western blot by usage of the polyclonal (rabbit) anti-Acrp30 antibody (Affinity BioReagents, USA; PA1-054).

### EXAMPLE 21

Construction and Expression of R13 1N-apM1(82-244).

Using apM1(82-244)/pcDNA3.1(−)Hygro/Intron as template, two PCR reactions are performed with two overlapping primer-sets [PBR195 (5′-CGCG GATCCACCATGCTGTTGCTGGGAGCTGTTCTACTGCTATTAGCTCTG CCCGGTCATGACG GTGAAACCGGAGTACCCGGGGCT-3′)/PBR223 (5-GAA GATCTTGGTAAAGTTAATGGGCATGTT-3′) and PBR222 (5-AACATGCCC ATTAACTTTACCAAGATCTTC-3′)/PBR193 (5′-ATATATCCCAAGCTTTCA GTTGGTGTCATGGTAGAG-3′) resulting in two fragments of 408 and 357 base pairs, respectively. These two fragments are assembled in a third PCR reaction with the flanking primers PBR195 and PBR193. The resulting gene is inserted into the mammalian expression vector pcDNA3.1(−)Hygro/Intron and confirmed by DNA sequencing to have the correct base changes leading to R13 1N-apM1(82-244). This cDNA, together with the upstream intron, is moved to the UCOE vector CET720. This construct is transfected into CHOK1 cells and stable pool is selected with Puromycin. The N-linked glycosylated title analog, R131N-apM1(82-244), is detected on western blot by usage of the polyclonal (rabbit) anti-Acrp30 antibody (Affinity BioReagents, USA; PA1-054). A 100% glycosylation is seen.

### EXAMPLE 22

Generic Method for Cys-Pegylation of Analogs of apM1 Fragments with an Introduced Cys (Cys-apM1), such as T121 C-apM 1(82-244), S146C-apM 1(82-244), or T243C-apM 1(82-244)

The Cys-apM 1 (as a trimer) used in this example is at a protein concentration of 0.5 mg/ml in a 20 mM Tris buffer, 1 mM CaCl₂, 100 mM NaCl, pH 7.4. Prior to PEGylation the Cys-apMl sample is reduced with DTT to ensure that the introduced cysteine residues can react and then the DTT is subsequent removed on a desalting column as described below. Two different Cys-specific PEG reagents from Shearwater Corporation have been employed. There are mPEG-OPSS and mPEG-vinylsulfone in different sizes (5, 10 or 20 kDa activated PEG). The activated PEG is added as a solid to the protein solution to obtain a 25 molar surplus (25 mol PEG per mol protein).

The experiments is carried out at room temperature (20-25° C.) as follows: 20 μl 0.5 M DTT stock solution is added to the Eppendorf tube containing 0.5 ml (0.25 mg) of Cys-apM1 and the resulting solution is mixed. After 30 min reaction time at room temperature the DTT is removed on an equilibrated NAP-5 (Amersham Biosciences) desalting column using 20 mM Tris, 1 mM CaCl₂, 100 mM NaCl, pH 7.4 as buffer. The sample is diluted 2 times over the desalting column giving a total eluate volume of 1.0 ml. Before PEGylation, the sample is concentrated using a Vivaspin 2 ml module (from Vivascience), with a 10 kDa cut-off membrane, to obtain a protein concentration of 0.5 mg/ml. To the Cys-apM1 solution is then added either 1.8 mg of 5 kDa, 3.6 mg of 10 kDa or 7.2 mg of 20 kDa Cys-specific PEG reagent and the solution is mixed. The reaction mixture is placed on a rocking platform and allowed to continue for 1 hour at room temperature.

The degree of PEGylation of the Cys-apM 1 can be evaluated using an analytical Superdex 200 (pre-packed 1.0 cm ID×30 cm column from Amersham Biosciences) SEC method runned under native conditions using the following buffer as mobile phase: 10 mM sodium acetate, 1 mM CaCl₂, 200 mM NaCl at pH 5.0. A flow rate of 1 ml/min and UV-detection at 214 nm is employed.

The Cys-apM1 having one, two, or three PEGs can be further purified on a semi-preparative SEC column (2.6 cm ID×60 cm) using the resin Superdex 200 prep grade (Amersham Biosciences). A sample volume <2 ml is loaded on a pre-equilibrated SEC column and a flow rate of 4 ml/min is used. The mobile phase consists of 10 mM sodium acetate, 1 mM CaCl₂, 200 mM NaCl at pH 5.0. The fractions containing the Cys-apMl material having one, two, or three PEGs are pooled based on the results from the analytical SEC method.

When the PEGylated sample after the SEC column contains trace amount of residual free PEG this can be removed on an anion exchanger. First, the sample is ultrafiltrated and then diafiltrated using for example Vivaspin 20 ml modules (from Vivascience), with a 10 kDa cut-off membrane, against a 1 mM CaCl₂, 20 mM Tris buffer adjusted to pH 7.4 with HCl prior to the anion exchange chromatography step. The diafiltrate is applied to a Q Sepharose FF (Amersham Biosciences) column previously equilibrated with 5 column volumes of 1 mM CaCl₂, 20 mM Tris adjusted to pH 7.4 with HCl (buffer A). Following application, the column is eluted in a linear gradient over 20 column volumes from buffer A to buffer B (buffer A containing 0.2 M NaCl). Fractions containing the PEGylated Cys-apMl are pooled based on results from the analytical SEC method and/or SDS-PAGE analysis. Since the SDS-PAGE analysis is runned under denatured conditions (non-reducing, treated 10 minutes at 70° C. in SDS sample buffer) this method is only used for PEGylated Cys-apM1 samples without remaining free PEG.

### EXAMPLE 23

apM1(82-244) inhibits TNF-Alpha Release from LPS-Stimulated Monocytic Cells

In order to investigate if adiponectin(82-244) also is able to inhibit LPS-induced TNF-alpha production we used the monocytic cell line THP-1 (ATCC, Rockville, Md.). Briefly, triplicate samples of THP-1 cells (10⁵/well) were incubated in 96 well-plates at 37° C. with titrated amounts of adiponectin (highest concentration 500 nM (25,5 μg/ml) in serum free cell culture medium (RPMI-1640, containing 10 mM HEPES)

Following 18 h pre-incubation with adiponectin the cultures were incubated for additional 4 h with a final concentration of 0.5 μg/ml lipopolysaccharide (LPS) (List Biologicals) and then 50 μl supernatant where withdrawn and frozen at −20° C. for subsequent analysis of TNF-alpha.

The diluted cell culture supernatants where analyzed for TNF-alpha content using a standard ELISA (R&D), and the IC50 of adiponectin where calculated using a 4-parameter non linear regression data analysis.

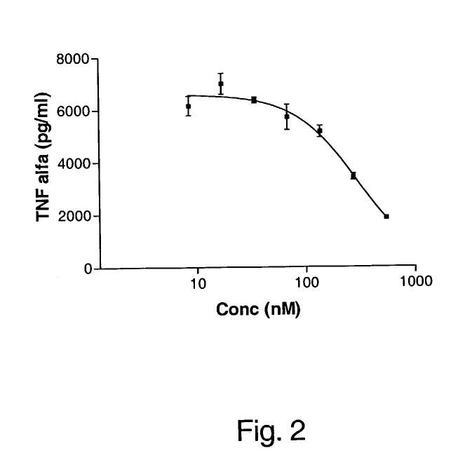

The results are shown in FIG. 2.

### EXAMPLE 24

Adiponectin Trimer Complex is De-Stabilized by Lowering the pH, but not in the Presence of Ca²⁺ Ions.

Purified apM1(82-244), produced in CHO-KI (see example 2 and 8), was present in a buffer containing 120 mM (NH₄)₂SO₄+20 mM NaH₂PO₄ (pH 6.8). In order to examine the effect of lowering the pH without Ca²⁺ ions present or in the presence of Ca²⁺ ions the sample was diluted in six different buffers, in the ratio 1 volume sample to 4 volumes buffer. The buffers were prepared by mixing from stock solutions of acetic acid, sodium hydroxide and calcium chloride to the following proportions (pH was measured before addition of sample):

| Buffer no.: | 1 | 2 | 3 | 4 | 5 | 6 |
|---|---|---|---|---|---|---|
| Acetic acid (mM) | 50 | 50 | 50 | 50 | 50 | 50 |
| Sodium hydroxide (mM) | 5 | 25 | 45 | 5 | 25 | 45 |
| Calcium chloride (mM) | 0 | 0 | 0 | 10 | 10 | 10 |
| Resulting pH | 3.6 | 4.8 | 5.6 | 3.6 | 4.6 | 5.7 |

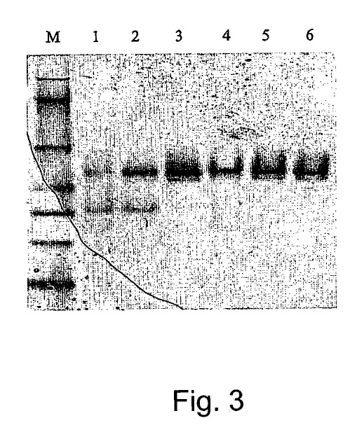

The resulting six different samples were examined on a coomassie stained Novex 8-16% Tris-Glycine gel (Invitrogen; Cat. No. EC60452) run under native conditions (FIG. 3, lane 1-6, respectively).

As seen in FIG. 3 the adiponectin trimer complex is de-stabilized at pH 3.6 and pH 4.8 in the absence of Ca²⁺ ions, whereas in the presence of 10 mM CaCl₂ no severe effect on the stability of the adiponectin trimer complex is observed at these low pH values. (In FIG. 3, M is SeeBlue Plus2).

### EXAMPLE 25

De-Stabilized Adiponectin Trimer Complex can be Recovered by Addition of Ca²⁺ Ions.

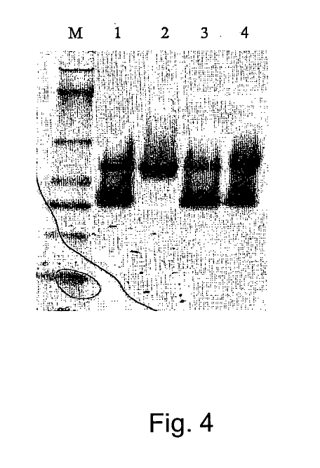

Purified apM 1(82-244), produced in CHO-K1 (see example 2 and 8), had been gel filtrated on a Superdex 75 column and buffer-shifted to near isotonic buffer: 100 mM NaCl, 20 mM NaH₂PO₄, 10 mM NaOH (pH 6.8). After storage at −20° C. for several weeks the material was verified to have a heterogeneous appearance on a coomassie stained Novex 8-16% Tris-Glycine gel (Invitrogen; Cat. No. EC60452) run under native conditions (FIG. 4, lane 1). Supplying the material (with heterogeneous appearance) with 20 mM CaCl₂, 20 mM MgCl₂ and 20 mM ZnCl₂, respectively, showed that only CaCl2 was able to re-stabilize the adiponectin trimer (FIG. 4, lane 2, 3 & 4, respectively). (In FIG. 4, M is SeeBlue Plus2).

These results indicate that the phosphate present in the isotonic buffer has withdrawn the Ca²⁺ ions, present in the adiponectin trimer, during storage at −20° C. During storage at freezing temperature, the pH of the solution apparently also could have decreased a bit, leading to an easier withdrawal of Ca²⁺ ions. Then by supplying the solution with Ca²⁺ ions, in the form of CaCl₂, the adiponectin trimer complex is recovered again. Addition of Mg²⁺ or Zn²⁺ ions showed no effect.

### EXAMPLE 26

Acute Treatment of db/db Mice with apM1(82-244) Fragment Transiently Normalizes Blood Glucose Level

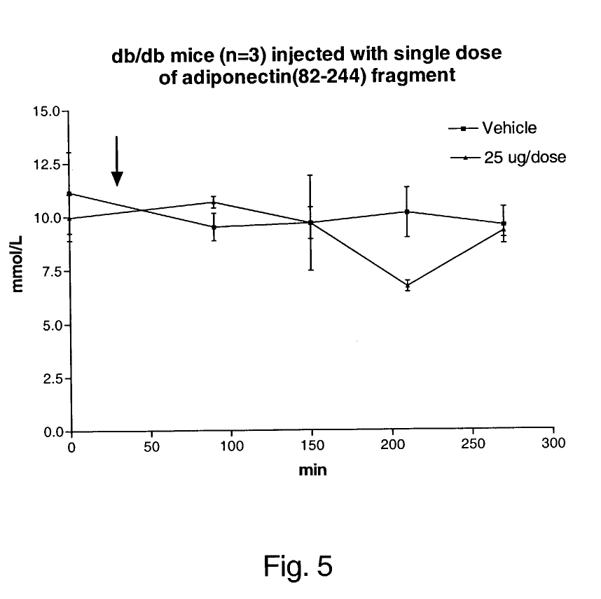

db/db mice were from 56 to 66 days old at the initiation of the experiment (approx. 36 g). All mice were maintained on a 12:12 light:dark cycle, fed standard rodent diet ad libitum, and had unlimited access to water. At t=0 the blood glucose levels were measured from tail nick samples in two groups, Group #1 & Group #2 (n=3 for each group), using a Glucometer Elite Monitor (Bayer Corporation). At t=30 min, intraperitoneal (IP) injections were as follows: Group #1: Vehicle (200 ILl buffer: 2 mM CaCl₂, 10 mM NaCltrate, 150 mM NaCl, pH 7,4) and Group #2: 25 μg apM1(82-244) (adiponectin(82-244)) fragment in 200 μl buffer. At 90, 150, 210, 270 minutes the blood glucose levels were determined. The resulting blood glucose levels are shown in FIG. 5. As seen in the graph, a single dose of 25 μg adiponectin(82-244) fragment transiently normalizes the blood glucose level (6.7 mmol/L) at t=210 minutes.

While the foregoing invention has been described in some detail for purposes of clarity and understanding, it will be clear to one skilled in the art from a reading of this disclosure that various changes in form and detail can be made without departing from the true scope of the invention. It is understood that the examples and embodiments described herein are for illustrative purposes only and that various modifications or changes in light thereof will be suggested to persons skilled in the art and are to be included within the spirit and purview of this applica-tion and scope of the appended claims. For example, all the techniques and apparatus described above may be used in various combinations. All publications, patents, patent applications, and/or other documents cited in this application are incorporated herein by reference in their entirety for all purposes to the same extent as if each individual publication, patent, patent application, and/or other document were individually indicated to be incorporated herein by reference in its entirety for all purposes.

We claim:

1. A conjugate comprising an adiponectin polypeptide, and a first non-polypeptide moiety covalently attached to the adiponectin polypeptide, wherein the adiponectin polypeptide comprises an amino acid residue having an attachment group for said first non-polypeptide moiety, wherein said amino acid residue has been introduced in a position that in the parent adiponectin is occupied by a surface exposed amino acid residue.

2. The conjugate of claim 1, wherein the adiponectin polypeptide comprises a globular domain.

3. The conjugate of claim 2, wherein the attachment group is introduced in the globular domain.

4. The conjugate of any one of the claims 2-3 wherein the adiponectin polypeptide comprises a collagen domain.

5. The conjugate of any one of the claims 14 wherein the amino acid residue having the attachment group for said first non-polypeptide moiety is selected from a lysine, aspartic acid, glutamic acid or cysteine residue.

6. The conjugate of claim 5 wherein the amino acid residue having the attachment group is a cysteine residue.

7. The conjugate of any one of the claims 1-6, wherein the first non-polypeptide moiety is selected from a polymer molecule, a lipophilic compound, and an organic derivatizing agent.

8. The conjugate of any one of the claims 1-7, wherein the first non-polypeptide moiety is a polymer, preferably linear or branched polyethylene glycol.

9. The conjugate of claim 8 wherein the polymer has a molecular weight of from lkDa to 200 kDa, such as 5 kDa to 40 kDa.

10. The conjugate of claim 6 or 9, wherein the polymer molecule is selected from the group consisting of mPEG(MAL), mPEG2(MAL), PEG-vinylsulphone, OPSS-PEG, or OPSS-PEG-hydrazide in combination with mPEG-ALD.

11. The conjugate of any one of the claims 14, wherein the attachment group is selected from an N- or O-glycosylation site, such as an N-glycosylation site.

12. The conjugate of claim 11, wherein a sugar moiety is attached to the N- or O-glycosylation site, such as the N-glycosylation site.

13. The conjugate of any one of the claims 1-12 wherein only one first non-polypeptide moiety is attached to the adiponectin polypeptide.

14. The conjugate of any one of the preceding claims, wherein the parent adiponectin polypeptide is selected from any one of the SEQ ID NO:1-8, 10-12, or 13, such as SEQ ID NO:3, 4, 5, 6, 10, 11, 12, or 13.

15. The conjugate of any one of the claims 1-10, wherein the adiponectin polypeptide is selected from an adiponectin polypeptide fragment comprising a globular domain and a collagen domain, wherein the globular domain comprises an amino acid sequence as indicated in SEQ ID NO:1 from position A108 to N244, and wherein the collagen domain comprises from 7 amino acids corresponding to position K101 as indicated in SEQ ID NO:1 to 66 amino acids corresponding to position G42 as indicated in SEQ ID NO:1, and wherein the collagen domain comprises a lysine which is hydroxylated and glycosylated, wherein the adiponectin polypeptide fragment comprises a mutation selected from any one of A108C, Y109C, V110C, Y111C, R112C, E120C, T121C, Y122C, V123C, T124C, I125C, P126C, N127C, M128C, R131C, T133C, K134C, I135C, Q139C, N141C, D144C, G145C, S146C, T147C, K149C, H151C, N153C, P155C, Y167C, M168C, K169C, D170C, K178C, D179C, K180C, A181C, F184C, Y186C, Q188C, Y189C, Q190C, E191C, N192C, N193C, V194C, H204C, E206C, V207C, G208C, Q210C, V215C, Y216C, G217C, E218C, G219C, E220C, R221C, N222C, L224C, Y225C, D227C, N228C, D229C, N230C, H241C, D242C, T243C, or N244C.

16. The conjugate of claim 15, wherein the adiponectin polypeptide fragment comprises a mutation selected from any one of T121C, N127C, N141C, S146C, N228C, or T243C.

17. The conjugate of claim 15 or 16, wherein the globular domain comprises an amino acid sequence from position A108 to N244 as indicated in SEQ ID NO:1, and wherein the collagen domain comprises from 8 amino acids corresponding to position R100 as indicated in SEQ ID NO:1 to 50 amino acids corresponding to position R58 as indicated in SEQ ID NO:1.

18. The conjugate of claim 16, wherein the adiponectin polypeptide fragment is selected from any one of the sequences SEQ ID NO:17, 18, 19, 20, 21, 22, 23, 24, 25, 26, 27, 28, 29, 30, 31, 32, 33, 34, 35, 36, 37, 38, 39,40, 41, 42, 43,44, 45, 46,47, 48,49, 50, 51, or 52.

19. The conjugate of any one of the claims 14, or 12, wherein the adiponectin polypeptide is selected from an adiponectin polypeptide fragment comprising a globular domain and a collagen domain, wherein the globular domain comprises an amino acid sequence as indicated in SEQ ID NO:1 from position A108 to N244, and wherein the collagen domain comprises from 7 amino acids corresponding to position K101 as indicated in SEQ ID NO:1 to 66 amino acids corresponding to position G42 as indicated in SEQ ID NO:1, and wherein the collagen domain comprises a lysine which is hydroxylated and glycosylated, wherein the adiponectin polypeptide fragment comprises a mutation selected from any one of A108N+V110T, Y109N+Y111T, V110N+R112T, Y111N, Y 111 N+S113T, R112N+A114T, L119N+T121S, L119N, E120N+Y122T, T121N+V123T, Y122N, Y122N+T124S, T124N+P126T, P126N+M128T, P129T, M128N+I130T, I130N+F132T, R131N, R131N+T133S, T133N+I135T, K134N+F136T, I135N+Y137T, F136N+N138T, Y137N+Q139T, Q140T, Q139N+N141T, Q140N+H142T, Y143T, H142N+D144T, D144N, D144N+S146T, G145N, G145N+T147S, S146N+G148T, T147N+K149T, K149N+H151T, H151N+N153T, P155T, P155N+L157T, Y159N+A161T, I164N+V166T, T165N+Y167T, Y167N+K169T, M168N+D170T, K169N+V171T, D170N+K172T, V171N+V173T, K172N, K172N+S174T, F176N+K178T, K177N+D179T, K178N+K180T, D179N+A181T, K180N+M182T, A181N+L183T, M182N+F184T, F184N+Y186T, Y186N+Q188T, Q188N+Q190T, Y189N+E191T, Q190N+N192T, E191N+N193T, V194T, or V194N+Q196T.

20. The conjugate of claim 19, wherein the globular domain comprises an amino acid sequence from position A108 to N244 as indicated in SEQ ID NO:1, and wherein the collagen domain comprises from 8 amino acids corresponding to position R100 as indicated in SEQ ID NO:1 to 50 amino acids corresponding to position R58 as indicated in SEQ ID NO:1.

21. The conjugate of claim 19 or 20, wherein the adiponectin polypeptide fragment comprises a mutation selected from any one of Y111N, Y122N, P129T, R131N, D144N+S146T, G145N, H111N+N153T, P155T, K178N+K180T, such as Y111N, Y122N, R131N, D144N+S146T, H151N+N153T, K178N+K180T, preferably Y111N, Y122N, R131N, D144N+S146T.

22. The conjugate of claim 21, wherein the adiponectin polypeptide fragment is selected from any one of the sequences SEQ ID NO:53, 54, 55, 56, 57, 58, 59, 60, or 61.

23. The conjugate of any one of the claims 1-22, further comprising a second non-polypeptide moiety selected from the group consisting of a polymer molecule, a lipophilic compound, a sugar moiety and an organic derivatizing agent, provided that it is different from the first non-polypeptide moiety.

24. An adiponectin polypeptide comprising an amino acid residue having an attachment group for a first non-polypeptide moiety, wherein said amino acid residue has been introduced in a position that in the parent adiponectin is occupied by a surface exposed amino acid residue.

25. The adiponectin polypeptide of claim 24, wherein the adiponectin polypeptide comprises a globular domain.

26. The adiponectin polypeptide of claim 25, wherein the attachment group is introduced in the globular domain.

27. The adiponectin polypeptide of any one of the claims 25 or 26 wherein the adiponectin polypeptide comprises a collagen domain.

28. The adiponectin polypeptide of any one of the claims 24-27 wherein the amino acid residue having the attachment group for said first non-polypeptide moiety is selected from a lysine, aspartic acid, glutamic acid or cysteine residue.

29. The adiponectin polypeptide of claim 28 wherein the amino acid residue having the attachment group is a cysteine residue.

30. The adiponectin polypeptide of claim 29, wherein the adiponectin polypeptide is selected from an adiponectin polypeptide fragment comprising a globular domain and a collagen domain, wherein the globular domain comprises an amino acid sequence as indicated in SEQ ID NO:1 from position A108 to N244, and wherein the collagen domain comprises from 7 amino acids corresponding to position K101 as indicated in SEQ ID NO:1 to 66 amino acids corresponding to position G42 as indicated in SEQ ID NO:1, and wherein the collagen domain comprises a lysine which is hydroxylated and glycosylated, wherein the adiponectin polypeptide fragment comprises a mutation selected from any one of A108C, Y109C, V110C, Y111C, R112C, E120C, T121C, Y122C, V123C, T124C, I125C, P126C, N127C, M128C, R131C, T133C, K134C, I135C, Q139C, N141C, D144C, G145C, S146C, T147C, K149C, H151C, N153C, P155C, Y167C, M168C, K169C, D170C, K178C, D179C, K180C, A181C, F184C, Y186C, Q188C, Y189C, Q190C, E191C, N192C, N193C, V194C, H204C, E206C, V207C, G208C, Q210C, V215C, Y216C, G217C, E218C, G219C, E220C, R221C, N222C, L224C, Y225C, D227C, N228C, D229C, N230C, H241C, D242C, T243C, or N244C.

31. The adiponectin polypeptide of claim 30, wherein the adiponectin polypeptide fragment comprises a mutation selected from any one of T121C, N127C, N141C, S146C, N228C, or T243C.

32. The adiponectin polypeptide of claim 30 or 31, wherein the globular domain comprises an amino acid sequence from position A108 to N244 as indicated in SEQ ID NO:1, and wherein the collagen domain comprises from 8 amino acids corresponding to position R100 as indicated in SEQ ID NO:1 to 50 amino acids corresponding to position R58 as indicated in SEQ ID NO:1.

33. The adiponectin polypeptide of claim 31, wherein the adiponectin polypeptide fragment is selected from any one of the sequences SEQ ID NO:17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, or 52.

34. The adiponectin polypeptide of any one of the claims 24-27, wherein the attachment group is selected from an N- or O-glycosylation site, such as an N-glycosylation site.

35. The adiponectin polypeptide of claim 34 wherein the adiponectin polypeptide is selected from an adiponectin polypeptide fragment comprising a globular domain and a collagen domain, wherein the globular domain comprises an amino acid sequence as indicated in SEQ ID NO:1 from position A108 to N244, and wherein the collagen domain comprises from 7 amino acids corresponding to position K101 as indicated in SEQ ID NO:1 to 66 amino acids corresponding to position G42 as indicated in SEQ ID NO:1, wherein the adiponectin polypeptide fragment comprises a mutation selected from any one of A108N+V110T, Y109N+Y111T, V110N+R112T, Y111N, Y111N+S113T, R112N+A114T, L119N+T121S, L119N, E120N+Y122T, T121N+V123T, Y122N, Y122N+T124S, T124N+P126T, P126N+M128T, P129T, M128N+I130T, I130N+F132T, R131N, R131N+T133S, T133N+I135T, K134N+F136T, I135N+Y137T, F136N+N138T, Y137N+Q139T, Q140T, Q139N+N141T, Q140N+H142T, Y143T, H142N+D144T, D144N, D144N+S146T, G145N, G145N+T147S, S146N+G148T, T147N+K149T, K149N+H151T, H151N+N153T, P155T, P155N+L157T, Y159N+A161T, I164N+V166T, T165N+Y167T, Y167N+K169T, M168N+D170T, K169N+V171T, D170N+K172T, V171N+V173T, K172N, K172N+S174T, F176N+K178T, K177N+D179T, K178N+K180T, D179N+A181T, K180N+M182T, A181N+L183T, M182N+F184T, F184N+Y186T, Y186N+Q188T, Q188N+Q190T, Y189N+E191T, Q190N+N192T, E191N+N193T, V194T, or V194N+Q196T.

36. The adiponectin polypeptide of claim 35, wherein the globular domain comprises an amino acid sequence from position A108 to N244 as indicated in SEQ ID NO:1, and wherein the collagen domain comprises from 8 amino acids corresponding to position R100 as indicated in SEQ ID NO:1 to 50 amino acids corresponding to position R58 as indicated in SEQ ID NO:1.

37. The adiponectin polypeptide of claim 35 or 36, wherein the adiponectin polypeptide fragment comprises a mutation selected from any one of Y111N, Y122N, P129T, R131N, D144N+S146T, G145N, H151N+N153T, P155T, K178N+K180T, such as Y111N, Y122N, R131N, D144N+S146T, H151N+N153T, K178N+K180T, preferably Y111N, Y122N, R131N, D144N+S146T.

38. The adiponectin polypeptide of claim 37, wherein the adiponectin polypeptide fragment is selected from any one of the sequences SEQ ID NO:53, 54, 55, 56, 57, 58, 59, 60, or 61.

39. The conjugate of any one of claims 1-23, or the adiponectin polypeptide of any one of claims 24-38 wherein the adiponectin polypeptide is produced in a mammalian cell, eg a CHO, BHK, HEK293 cell or an SF9 cell.

40. An adiponectin polypeptide fragment comprising any one of SEQ ID NO:2, 3, 4, 5, 10, 11, 12, or 13, as well as sequences that differs from any one of the specified sequences, in one or more substitution(s), preferably from one to eleven, wherein the adiponectin polypeptide is produced in a mammalian cell, eg a CHO, BHK, HEK293 cell or an SF9 cell.

41. The adiponectin polypeptide fragment of claim 40 comprising one to four lysine residue(s) selected from any one of the positions K65, K68, K77, or K101 relative to SEQ ID NO:1, wherein the lysine residue(s) is hydroxylated and glycosylated, such as one lysine residue selected from K101 relative to SEQ ID NO:1 that is hydroxylated and glycosylated.

42. An adiponectin polypeptide fragment comprising a globular domain and a collagen domain,
wherein the globular domain comprises an amino acid sequence as indicated in SEQ ID NO:1 from position A108 to N244 as well as sequences that differs from the amino acid sequence in one or more substitution(s), and
wherein the collagen domain comprises from 7 amino acids corresponding to position K101 as indicated in SEQ ID NO:1 to 66 amino acids corresponding to position G42 as indicated in SEQ ID NO:1, and wherein the collagen domain comprises a lysine which is hydroxylated and glycosylated.

43. The adiponectin polypeptide fragment of claim 42 wherein the collagen domain comprises from 7 amino acids corresponding to position K101 as indicated in SEQ ID NO:1 to 50 amino acids corresponding to position R58 as indicated in SEQ ID NO:1, such as from 8 amino acids corresponding to position R100 as indicated in SEQ ID NO:1 to 50 amino acids corresponding to position R58 as indicated in SEQ ID NO:1.

44. The adiponectin polypeptide fragment of claim 42 or 43 selected from any one of SEQ ID NO:3, 4, 5, 10, 11, 12, or 13.

45. An isolated complex comprising a) an adiponectin polypeptide trimer wherein the adiponectin polypeptide trimer contains three adiponectin polypeptide monomers, and b) calcium ions.

46. An isolated complex comprising a) a conjugate comprising an adiponectin polypeptide trimer wherein the adiponectin polypeptide trimer contains three adiponectin polypeptide monomers, and a first non-polypeptide moiety covalently attached to any one of the three adiponectin polypeptide monomers, and b) calcium ions.

47. The isolated complex of claim 45 or 46 wherein the adiponectin polypeptide trimer is expressed and recovered from mammalian host cells, or yeast host cells.

48. The isolated complex of claim 45 or 46 wherein the adiponectin polypeptide trimer is expressed and recovered from bacterial host cells.

49. The isolated complex of any one of claims 45-48 wherein the adiponectin polypeptide comprises an amino acid sequence having a globular domain having at least 80% identity to the globular domain of apMl (shown in sequence id no 6) and optionally comprising a collagen domain, such as having at least 90% identity to the globular domain of apMl, typically having at least 92% identity to the globular domain of apM 1.

50. The isolated complex of claim 49 wherein the adiponectin polypeptide is selected from any one of the SEQ ID NO:1-6, 10-12, or 13, and sequences having at least 80% identity to any one of the SEQ ID NO:1-6, 10-12, or 13, respectively.

51. The isolated complex of any one of claims 46-50 wherein the first non-polypeptide moiety is selected from a polymer, or a sugar moiety.

52. An isolated complex comprising a) a conjugate consisting of an adiponectin polypeptide trimer wherein the adiponectin polypeptide trimer contains three adiponectin polypeptide monomers, and one polymer covalently attached to any one of the three adiponectin polypeptide monomers, in such a way that the resulting adiponectin polypeptide trimer only contains one polymer and b) calcium ions.

53. The isolated complex of claim 52 wherein the polymer is covalently attached to an amino acid residue selected from a lysine, a cysteine, or an N-terminal amino acid residue.

54. The isolated complex of claim 52 or 53 wherein the polymer comprises a linear or branched polyethylene glycol.

55. A liquid composition comprising an isolated complex of any one of claims 45-54.

56. A method of preparing an isolated complex of any one of claims 45-55, the method comprising bringing calcium ions in contact with the adiponectin polypeptide and optionally reacting the adiponectin polypeptide with the first non-polypeptide moiety.

57. The isolated complex of any one of claims 45-54 wherein the adiponectin polypeptide monomer is selected from the adiponectin polypeptide or adiponectin polypeptide fragment of any one of claims 24-44.

58. A pharmaceutical composition comprising an isolated complex of any one of claims 45-55, and a pharmaceutically acceptable carrier.

59. A pharmaceutical composition comprising a conjugate of any one of claims 1-23, and a pharmaceutically acceptable carrier.

60. A pharmaceutical composition comprising an adiponectin polypeptide or adiponectin polypeptide fragment of any one of claims 24-44, and a pharmaceutically acceptable carrier.

61. The composition of any one of claims 58-60 wherein the composition is selected from a liquid composition, such as a liquid solution, e.g. an aqueous solution.

62. A nucleotide sequence encoding the adiponectin polypeptide part of a conjugate of any one of claims 1-23, or the adiponectin polypeptide or adiponectin polypeptide fragment of any one of claims 24-44, such as a RNA, DNA, or cDNA.

63. The nucleotide sequence of claim 62 wherein the sequence is selected from any one of SEQ ID NO:14, 15, 16, 62, 63, 64, 65, 66, 67, 68, 69, 70, or 71 as well as sequences having at least 70% homology with any one of SEQ ID NO:14, 15, 16, 62, 63, 64, 65, 66, 67, 68, 69, 70, or 71, respectively.

64. An expression vector comprising a nucleotide sequence of claim 62 or 63.

65. A host cell comprising a nucleotide sequence of claim 62 or 63, or an expression vector of claim 64.

66. The host cell of claim 65, which is selected from a yeast cell, a bacterial cell, eg E. Coli, a mammalian cell, eg a CHO, BHK, HEK293 cell, or an SF9 cell, preferably a mammalian cell.

67. A method for preparing a conjugate of any one of claims 1-23, wherein the adiponectin polypeptide is reacted with the molecule to which it is to be conjugated under conditions conducive for the conjugation to take place, and the conjugate is recovered.

68. A method of preparing an isolated complex comprising 1) an adiponectin polypeptide or a conjugate comprising an adiponectin polypeptide, and a first non-polypeptide moiety covalently attached to the adiponectin polypeptide, and 2) calcium ions, the method comprising
a) preparing a nucleotide sequence encoding: a signal peptide and the adiponectin polypeptide,
b) inserting the nucleotide sequence into a vector,
c) transfecting the vector into a mammalian cell,
d) expressing and optionally secreting the adiponectin polypeptide,
e) recovering the complex, and optionally
f) reacting the adiponectin polypeptide with the molecule to which it is to be conjugated under conditions conducive for the conjugation to take place, and recovering the conjugate;
provided that any one of steps d), e), or f) is carried out in a calcium ion rich environment.

69. The method of claim 68 wherein any one of steps d), e), or f) is carried out in a molar surplus of calcium ions relative to the adiponectin polypeptide.

70. A method of preparing an adiponectin polypeptide, comprising
a) preparing a nucleotide sequence encoding: a signal peptide and the adiponectin polypeptide, wherein the last three C-terminal amino acids of the signal peptide are HDG,
b) inserting the nucleotide sequence into a vector,
c) transfecting the vector into a mammalian cell, and
d) expressing and optionally secreting the adiponectin polypeptide.

71. The method of claim 70 further comprising the step
e) obtaining the adiponectin polypeptide.

72. A method for preparing a conjugate the method of claim 71 further comprising
f) reacting the adiponectin polypeptide with the molecule to which it is to be conjugated under conditions conducive for the conjugation to take place, and recovering the conjugate.

73. Use of the conjugate of any one of claims 1-23, or the adiponectin polypeptide or adiponectin polypeptide fragment of any one of claims 24-44, or the complex of any one of claims 45-54, or 57, or the composition of claim 55 for the manufacture of a medicament for treatment of IGT, type 2 diabetes, syndrome X, dyslipidemia, septic shock, or cardiovascular disease, such as atherosclerosis.

74. A method of treating a mammal with type 1 diabetes; impaired glucose tolerance; type 2 diabetes; syndrome X; obesity; cardiovascular disease, such as atherosclerosis; dyslipidemia; or for lowering body weight without reducing food intake; rheumatoid arthritis; Crohn's disease; systemic lupus erythematosus; Sjögren's disease; cachexia; septic shock; myasthenia gravis; post-traumatic brain damage; myocardial infarction; post-surgical brain-damage; and other destructive processes related,to stress or activation of the inflammatory system; in particular IGT, type 2 diabetes, syndrome X, dyslipidemia, septic shock, or cardiovascular disease, such as atherosclerosis, which method comprises administering to a mammal an effective amount of the conjugate of any one of claims 1-23, or the adiponectin polypeptide or adiponectin polypeptide fragment of any one of claims 24-44, or the complex of any one of claims 45-54, or 57, or the composition of claim 55.

75. Use of the conjugate of any one of claims 1-23, or the adiponectin polypeptide or adiponectin polypeptide fragment of any one of claims 24-44, or the complex of any one of claims 45-54, or 57, or the composition of claim 55, for preparing a medicament for treatment of a disease, disorder, or condition caused by expression or release of TNF-alpha in a human cell, wherein said medicament inhibits expression or release of TNF-alpha.

76. The use of claim 75 wherein said disease, disorder, or condition is selected from septic shock, and other destructive processes related to stress or activation of the inflammatory system.
